(19) United States
(12) Patent Application Publication
(10) Pub. No.: US 2026/0232228 A1
(43) Pub. Date: Aug. 13, 2026

(54) SYSTEMS AND METHODS FOR ANALYZING SPECTRAL DATA TO DETERMINE ANALYTE CONCENTRATIONS IN A BODY

(71) Applicant: MedWatch Technologies, Inc., Las Vegas, NV (US)

(72) Inventors: Bruce Matichuk, Edmonton (CA); Mike E. Moore, Las Vegas, NV (US)

(21) Appl. No.: 19/412,733

(22) Filed: Dec. 8, 2025

Related U.S. Application Data
(60) Provisional application No. 63/729,107, filed on Dec. 6, 2024.
(60) Provisional application No. 63/781,326, filed on Mar. 31, 2025.
(60) Provisional application No. 63/836,246, filed on Jun. 30, 2025.

(51) Int. Cl. A61B 5/145; A61B 5/00; A61B 5/1455; G16H 50/30

(52) U.S. Cl. CPC A61B 5/14532; A61B 5/1455; A61B 5/7264; G16H 50/30

(57) ABSTRACT

The methods can include receiving calibrating spectral data from a non-invasive measurement device, known measurement data that is indicative of a concentration of a particular biomolecule in the body of the wearer and was obtained contemporaneously with the calibrating spectral data; generating a signature for the wearer by at least determining, via a neural network, one or more correspondences between or among the calibrating spectral data and the known measurement data; and storing the signature. The methods can include receiving present spectral data from the non-invasive measurement device and processing the present spectral data using the signature to estimate a present concentration of the particular biomolecule in the body.

### CROSS REFERENCE TO RELATED APPLICATIONS

The present claims priority from U.S. Provisional Patent Application No. 63/729,107, filed Dec. 6, 2024, entitled “Systems and Methods for Analyzing Spectral Data to Determine Analyte Concentrations in a Body,” and from U.S. Provisional Patent Application No. 63/781,326, filed Mar. 31, 2025, entitled “Devices, Systems, and Methods for Analyzing Combined Spectral And Microwave Data to Determine Analyte Concentrations in a Body,” and from U.S. Provisional Patent Application No. 63/836,246, filed Jun. 30, 2025, entitled “Devices, Systems, and Methods for Analyzing Combined Spectral And Microwave Data to Determine Analyte Concentrations in a Body,” the disclosures of which are incorporated herein by reference in their entirety.

### BACKGROUND

Current estimates are that close to 9% of the world population is affected by diabetes, which is expected to rise to 10% by 2045. In addition to diagnosed diabetes cases, an estimated 352.1 million people worldwide are pre-diabetic, a figure that is expected to rise significantly in the coming years. In the case of diabetes, high Blood Glucose (BG) levels (e.g., hyperglycemia) are toxic and cause serious health complications due to damage to the vessels that supply blood to vital organs. Hyperglycemia increases the risk of heart disease and stroke, kidney disease, vision problems, and nerve problems. Further, conditions like diabetes can be a contributor to many other diseases and/or conditions such as cardiovascular disease, nerve damage (neuropathy), problems with nausea, vomiting, diarrhea, constipation, erectile dysfunction, kidney damage (nephropathy), eye damage (retinopathy) potentially leading to blindness, cataracts, glaucoma, foot damage leading to toe, foot or leg amputation, skin conditions including bacterial and fungal infections, hearing impairment, Alzheimer's disease, or depression.

Diabetes is a chronic metabolic disease characterized by elevated blood glucose levels. Chronic diabetes is classified as either Type 1 (T1D), which is characterized by insufficient production of insulin and constitutes about 10% of diabetes cases, or Type 2 (T2D), which is characterized by the body's ineffectual use of insulin and constitutes about 90% of diabetes cases. According to the CDC's 2020 National Diabetes Statistics Report, approximately 34.2 million individuals in the US, or 10.5% of the US population, have been diagnosed with diabetes. Typically, diabetes management is a balancing act of: routine blood glucose testing, strict monitoring of diet and physical activity, and, for most T2D patients, oral and injected non-insulin treatments. As indicated above, if blood glucose levels are too high, repeat episodes of hyperglycemia may lead to heart disease, stroke, kidney disease, blindness, or nerve damage.

A key contributor to the remarkably high risks of morbidity and mortality associated with diabetes is poor glycemic control. Glycemic control is assessed by measuring a patient's glycated hemoglobin, or HbA1C, as an indication of their average blood glucose levels over the preceding 2 to 3 months. Unfortunately, more than 50% of T2D patients fail to achieve target blood glucose levels, which is defined as an HbA1C less than 7.0%. In recent years, the management of diabetes, and more specifically, glycemic control, has been aided with the advent of new technologies for assessing glycemia, such as continuous glucose monitoring (CGMs). In this regard, it has been determined that a patient's time in range (TIR), which can be presented as “% of glucose readings” or “hours per day” that the patient's glucose is within the ideal range (e.g., 70-180 mg/dL) has emerged as an important metric of glycemic control, and if a user can increase their TIR, he or she can increase the longevity of their wellbeing. However, glycemia and/or diabetes monitoring is an important facet of health management, but blood glucose monitoring is only effective if it is done regularly and accurately.

Particularly, regular blood glucose monitoring is an essential task in managing diabetes, and those suffering from diabetes are more likely to manage their condition if their blood glucose measurements are shared with health professionals. Therefore, the shared disclosure of monitoring data can be a key consideration, particularly with respect to T2D. For example, T2D can be treated with diet, exercise, rest, and healthy eating (e.g., avoiding high glycemic foods), but to date, there has been limited success for systems that allow for self-monitoring, and/or provide a platform by which health care professionals can participate in this management.

The most common and widespread method of glucose monitoring is self-monitoring of blood glucose (SMBG), and existing SMBG methods (or at least SMBG methods that are sufficiently accurate) require regular, invasive testing glucose levels. That is to say, SMBG typically involves intermittently obtaining a capillary blood sample from a fingertip puncture (or a puncture to another body part) and electrochemically analyzing the sample with a glucometer. Frequent self-monitoring of blood glucose levels is an important activity in treating diabetes allowing a person to modify their diet and exercise regimen to ensure that normal blood glucose levels are maintained. SMBG has been shown to improve glycemic control and empowerment of people with diabetes. However, performing SMBG is a burdensome, cumbersome, and painful task.

Two conventional methods for monitoring blood glucose levels include lancing the skin to obtain blood, or employing subcutaneous needle device that can semi-continuously read glucose levels. The lancet method is burdensome and expensive requiring the painful piercing of the skin in order to obtain a blood sample, which sample may then be contacted to an electrochemical test strip by which a blood glucose measurement may be taken. Unfortunately, monitoring with conventional electrochemical-based test strips is expensive, today typically costing $1.00 for each test and requiring the user to lance their finger to obtain a drop of blood for the test.

Accordingly, despite the many benefits of regular glucose monitoring, SMBG has several limitations. The finger pricking required to obtain SMBG samples is associated with pain and discomfort, which negatively impacts patient compliance. Additionally, SMBG is non-continuous. Particularly, people with diabetes typically perform SMBG at 3-5 set time points during the day (e.g., fasting, pre-prandial, postprandial) or when they experience symptoms of dysglycemia. Nevertheless, even if SMBG is performed frequently, it is recognized that clinically significant fluctuations in blood glucose may be missed due to the periodic sampling, and because of this an accurate TIR is often difficult to determine.

As, indicated, an alternative to SMBG for glucose monitoring is the use of a Continuous Glucose Monitoring (CGM) system. For instance, in alternative instances, instead of using a lancing methodology, a subcutaneous monitoring device can be used to determine and/or monitor glucose methods. Newly emerging continuous glucose monitors employ a device containing electronics and having a small-short, analyte-containing needle that penetrates the surface of the skin. These devices are not securely attachable to the body, are uncomfortable because of the sub-cutaneous, needle-like structure, and can frequently come off and/or can be easily separated from the user. Further, such devices typically need to be removed prior to bathing or swimming.

Further still, the subcutaneous method is expensive (typically costing $10/day or more) and inconvenient. Specifically, CGM generally utilizes a circular substrate to which an adhesive is added so as to attach the device to the skin of the body. It often requires a subcutaneous needle or other type of analyte containing component that needs to be inserted into the skin. To date, most CGM systems detect glucose levels in dermal interstitial fluid through a glucose oxidase-impregnated electrochemical needle/sensor that is subcutaneously placed by the user. In contrast to the static measurement provided by SMBG, CGMs provide patients and healthcare providers with both nearly real-time glucose level snapshots and glycemic trends by the measuring interstitial fluid glucose concentration every 1-15 minutes, depending on the system. As a result, CGM shows improved glycemic control and increased patient satisfaction with use in people with diabetes, but it also has several drawbacks.

Particularly, even though lancing the skin is not required when using a CGM device, this newly emerging system still employs a small-short needle that needs to penetrate the surface of the skin. Hence, such devices for the subcutaneous electrochemical monitoring of interstitial fluid can also be invasive and create discomfort both of which have hindered the adoption and continued use of CGMs in people with diabetes. Further, these devices can frequently come off or separate from the user and cannot be used bathing or swimming. Additionally, they are even more expensive than test-strip monitoring, typically costing $10/day or more. Although non-invasive devices and methods for analyzing various physical signals representative of user health, like blood glucose signal levels, have been extensively studied, there have been few breakthroughs made and brought to commercial viability.

### SUMMARY

In view of the forgoing, what is needed, therefore, is a wearable sensing and/or monitoring device, and its associated systems and sub-systems, which are configured for non-invasively detecting, measuring, and/or analyzing the health of a wearer of the device. To that end, the disclosed technology can detect a change in the body, such a change in the presence of one or more biological markers and/or physical variables within one or more body tissues. The changes to such biological markers and/or physical variables can be analyzed (e.g., according to the methods disclosed herein) to determine a corresponding health status of the wearer. Particularly, the disclosed devices, systems, and methods can test one or more body tissues of a wearer to measure and/or monitor biomolecule (e.g., glucose) levels using electromagnetic radiation. Alternatively, or in addition, the disclosed technology can analyze test data (e.g., spectral or electromagnetic data) to determine the presence and/or concentration of a particular analyte (e.g., glucose) within the body of the wearer. Accordingly, the disclosed technology can overcome some or all of the aforementioned problems and shortcomings in existing attempts to monitor and manage glucose, e.g., blood glucose, or other analytes.

Further, the disclosed technology includes devices, systems, and/or methods for testing and determining an effect that various different molecules, such as metabolites, have on one or more tissues of the body. Specifically, the disclosed technology can determine the effects of biomolecules with regard to provoking or influencing a diseased condition within the body. For example, in the case of the biomolecule of interest being glucose, the disclosed technology can determine and/or estimate one or more diseased conditions, such as the potential for hyperglycemia, diabetes, and/or pre-diabetes.

More particularly, the devices and systems disclosed herein are particularly important because of the high expense to health and living caused by diseases such as diabetes. For instance, estimates indicate that about 37 million people in the U.S. suffer from T1D and T2d and an additional 96 million people suffer from pre-diabetes. Estimates further indicates that third-party payors pay approximately $1,800-$3,000 for glucose monitoring devices, such as invasive finger-prick monitoring devices, which many users end up not using because of the invasive nature of such devices and systems. When monitoring devices are not used, the user (or patient) may ultimately require one or more hospitalizations, which could dramatical increase severity and cost as diabetes progresses.

Accordingly, the disclosed technology relates to Non-Invasive, Continuous, Glucose Monitoring System (NICGMS) to detect glucose levels in a non-invasive manner, such as without any needles, lancets, or other invasive technologies. Instead, the disclosed technology measures analyte presence and/or concentrations (e.g., glucose levels) using electromagnetic radiation and optics. Such devices, systems, and methods can be performed in a manner that is highly accurate, painless, safe, and non-invasive, whereby biological elements, such as analytes, metabolites, or biological agents (e.g., glucose) can be detected, measured, and monitored. The analytes, metabolites, or biological agents can be detected, measured, and monitored in situ (e.g., within a wearer's blood, skin, interstitial tissue, or other body tissue). Throughout this disclosure, such detection and measurement techniques and methods are described with respect to spectral or optical data, but the disclosed technology is not so limited and can include, as non-limiting examples, radio and/or microwave frequency emission. Regardless, the disclosed technology can be configured to detect and/or measure the biomolecule itself. Alternatively or in addition, the disclosed technology can be configured to detect and/or measure effects on, in, or around one or more structures, tissues, and/or fluids of the wearer's body (e.g., surrounding that element or biomolecule), such as by detecting and determining waveforms reflected from such structure(s), tissue(s), and/or fluid(s) in response to an outputted waveform being emitted and directed toward the corresponding structure(s), tissue(s), and/or fluid(s). For example, the disclosed technology can include performing a diffuse reflectance spectrum analysis to determine the presence and/or level or concentration of a biomolecule (e.g., a glucose) in, on, or around a subject's blood, tissue(s), body structure(s), and/or spaces therebetween. Detection of the molecule and its effects can be based at least in part on optical and/or long waveform data (e.g., based at least in part on any changes to wavelength and frequency of the return energy, as compared to the outputted energy) and/or based at least in part on any changes in the blood, tissue(s), body structure(s), and/or spaces therebetween due to the presence of the biomolecule, as observed.

Accordingly, a non-invasive, continuous biomolecule detecting and measuring device is disclosed. For example, the sensing and/or monitoring devices set forth herein are configured to determine a health condition based on the detection of various biological agents, such as within the blood, skin, organs, and/or the spaces therebetween (e.g., within the interstitial fluid). The disclosed technology can track various biometrics of the user with respect to one or more determined conditions, such as heart rate, heart rate variability, blood pressure, oxygen saturation, respiration rate, sleep levels, and/or activity levels, as non-limiting examples. Various configurations are contemplated. For example, the wearable system can include a device and/or apparatus that can be worn on the wearer's wrist, arm, finger, back, abdomen, ankle, or leg, as non-limiting examples, and/or can be carried on a wearer's person, worn on a chain or strap, or attached to some other part of the wearer's body, such as via an associated patch or band-like apparatus (e.g., bracelet, watch, ring).

Data indicative of the reflected and/or refracted energy data (e.g., light energy reflected by the wearer's body structure(s), tissue(s), and/or fluid(s) in response to light being emitted thereon at one or more predetermined wavelengths and/or for a predetermined duration corresponding to each predetermined wavelength) can be transmitted to a computer system (e.g., a remote computing system). The disclosed technology can include performing an analysis on the reflected and/or refracted energy data (e.g., an energy diffuse reflectance spectrum analysis) to thereby detect and/or otherwise determine the level (e.g., amount, concentration) of the biomolecule of interest and/or one or more effects of the biomolecule on the health or condition of the wearer. The disclosed technology can include a machine learning (ML) system (e.g., an artificial intelligence (AI) system) configured to perform one or more analysis steps. As a non-limiting example, the ML system can comprise a multi-layer artificial neural network (ANN), and can read, determine, and/or predict one or more bioagent (e.g., glucose) levels from data indicative of one or more measurements made on the structure(s), skin, blood, interstitial fluid(s), and/or other biological tissues. Alternatively or in addition, the ML system can be configured to determine effects of such bioagent levels on the wearer's body. Although certain aspects of the disclosed technology may be expressly disclosed herein with respect to an ANN, the disclosed technology is not so limited and can include, implement, use, and/or apply any type of ML technology and/or system, which can be or include an ANN, any other type of ML technology and/or system, or any combination thereof.

As described more fully herein, the data indicative of one or more measurements made on the structure(s), skin, blood, interstitial fluid(s), and/or other biological tissues can reference detection of the biological agents themselves (e.g., presence of the biological agents, a change in concentration of the biological agents), which can demarcate a change in a determinable biological condition. Alternatively or in addition, the data indicative of one or more measurements made on the structure(s), skin, blood, interstitial fluid(s), and/or other biological tissues can reference detection of a change in the structure(s), skin, blood, interstitial fluid(s), and/or other biological tissues including, surrounding, or near the biological markers (e.g., due to the presence of the biological markers).

The disclosed technology includes a system comprising one or more processors and memory having instructions stored thereon that, when executed by the one or more processors, cause the system to perform one or more steps or actions. The instructions can cause the system to receive, from a non-invasive measurement device, calibrating spectral data associated with a wearer of the non-invasive measurement device. The instructions can cause the system to receive, from an invasive measurement device, known measurement data associated with the wearer of the non-invasive measurement device, the known measurement data (i) being indicative of a concentration of a particular biomolecule in a body of the wearer and (ii) having been obtained contemporaneously with the calibrating spectral data. The instructions can cause the system to generate a signature for the wearer by at least determining, via a neural network, one or more correspondences between or among the calibrating spectral data and the known measurement data, and the instructions can cause the system to store the signature.

The spectral data can be indicative of one or more responses by skin, tissue, and/or bodily fluids of the wearer to a plurality of light emissions emitted according to a particular pattern of frequencies and durations.

The signature can comprise a mapping of parameters, wherein at least some of the parameters correspond to the one or more responses by the skin, the tissue, and/or the bodily fluids of the wearer to the plurality of light emissions.

The instructions, when executed by the one or more processors, can further cause the system to receive, from the non-invasive measurement device, present spectral data associated with the wearer of the non-invasive measurement device and process the present spectral data using the signature to thereby estimate a present concentration of the particular biomolecule in the body of the wearer.

Collected, received, determined, or any other information can be sent to, provided to, or otherwise be made available to the wearer, stored on a remote server, and/or be sent to, or otherwise shared with, health professionals, healthcare computing systems, or any other third party or third-party computing system. As discussed, a wearer is typically more likely to regularly monitor their biomolecule (e.g., glucose) levels when he or she knows that others will be viewing, monitoring, and/or tracking his or her monitoring compliance. Accordingly, the disclosed technology can transmit information (e.g., real-time) to one or more users of the system (or one or more third parties). The disclosed technology can also include providing a user interface and/or application for tracking other health information, such as information relating to food consumption, activities, or other observances relating to a person's health. Such information can provide a more complete understanding of the wearer's health state and can be analyzed (e.g., by the wearer, by health professional, by a computing system such as a computing system disclosed herein) to make health condition assessments, predictions, recommendations, and/or prophylactic or treatment plans.

Various details of the disclosed technology are set forth in the following description and the accompanying drawings. Other features and advantages will be apparent from the description and drawings, and from the claims.

### BRIEF DESCRIPTION OF THE DRAWINGS

These and other aspects are described in detail herein with reference to the following drawings.

FIG. 1 illustrates an example biometric sensing and monitoring system, in accordance with the disclosed technology.

FIG. 2A illustrates an exploded view of an example biometric sensing and monitoring device, in accordance with the disclosed technology.

FIG. 2B illustrates a bottom perspective view an example biometric sensing and monitoring device, in accordance with the disclosed technology.

FIG. 2C illustrates a partial bottom view of the example biometric sensing and monitoring device displayed in FIG. 2B, in accordance with the disclosed technology.

FIG. 2D illustrates a partial bottom view of the example biometric sensing and monitoring device displayed in FIG. 2B, in accordance with the disclosed technology.

FIG. 2E illustrates an example printed circuit board, in accordance with the disclosed technology.

FIG. 2F illustrates an example printed circuit board, in accordance with the disclosed technology.

FIG. 3A illustrates a perspective view of an example patch attachment structure and an example biometric sensing and monitoring device, in accordance with the disclosed technology.

FIG. 3B illustrates a perspective view of an example biometric sensing and monitoring device positioned within an example patch attachment structure, in accordance with the disclosed technology.

FIG. 3C illustrates a perspective view of an example open patch attachment structure and an example biometric sensing and monitoring device, in accordance with the disclosed technology.

FIG. 3D illustrates a perspective view of an example biometric sensing and monitoring device positioned within an example open patch attachment structure, in accordance with the disclosed technology.

FIG. 4A schematically illustrates an example biometric sensing and monitoring device attached to the body of a wearer, in accordance with the disclosed technology.

FIG. 4B schematically illustrates an example biometric sensing and monitoring device performing a biomolecule sensing operation, in accordance with the disclosed technology.

FIG. 5A presents a bottom perspective view of an example biometric sensing and monitoring device for non-invasively detecting the presence and concentration of a biomolecule of interest, in accordance with the disclosed technology.

FIG. 5B illustrates exploded views of an example sensing and monitoring device, in accordance with the disclosed technology.

FIG. 5C illustrates an example photoplethysmography (PPG) sensor array, in accordance with the disclosed technology.

FIG. 5D schematically illustrates an example biometric sensing and monitoring device including a PPG sensor array that is performing a PPG sensing operation, in accordance with the disclosed technology.

FIG. 5E illustrates example biometric sensing and monitoring devices non-invasively detecting the presence and concentration of a biomolecule of interest, in accordance with the disclosed technology.

FIG. 6A illustrates an example biometric sensing and monitoring device being worn by a wearer, in accordance with the disclosed technology.

FIG. 6B illustrates an example biometric sensing and monitoring device configured as a watch-like device for being worn around the wrist of the wearer, in accordance with the disclosed technology.

FIG. 7 illustrates an example process for detected and communicating detected information, in accordance with the disclosed technology.

FIG. 8 illustrates an example an example graphical user interface for presentation on a display screen of an example computing device, in accordance with the disclosed technology.

FIG. 9A illustrates an overview of examples methods and processes for estimating or otherwise determining the presence/non-presence of a biomolecule of interest in a particular person's body based at least in part on spectral data obtained from the particular person's body, in accordance with the disclosed technology.

FIG. 9B illustrates a flow diagram of an example process for generating a signature, in accordance with the disclosed technology.

FIG. 10 illustrates a flow diagram of an example process for triggering a new signature generation process in response to detecting lack of skin contact between a device and a wearer of the device, in accordance with the disclosed technology.

FIG. 11 illustrates a flow diagram of an example process for determining the presence and/or concentration of one or more biomolecules within a wearer's body, in accordance with the disclosed technology.

FIG. 12 illustrates a flow diagram of an example process for answering a question based on information and/or correspondences stored in one or more data structure(s), in accordance with the disclosed technology.

FIG. 13A illustrates an example LSTM Composite Autoencoder, in accordance with the disclosed technology.

FIG. 13B illustrates an example model with corresponding input and output shapes for various layers of the model, in accordance with the disclosed technology.

FIG. 14 illustrates a flow diagram of an example process for determining the presence and/or concentration of one or more biomolecules within a wearer's body, in accordance with the disclosed technology.

FIG. 15 illustrates a flow diagram of an example process for testing a generated model, in accordance with the disclosed technology.

FIG. 16 illustrates a flow diagram of an example process for testing and updating a generated model over time, in accordance with the disclosed technology.

FIG. 17 illustrates a flow diagram of an example process for analyzing and/or monitoring spectral data for and/or across a population of individuals, in accordance with the disclosed technology.

FIG. 18 illustrates a flow diagram of an example process for determining one or more actions, in accordance with the disclosed technology.

FIG. 19A illustrates a chart illustrating the Error Surveillance Grid associated with pairings of spectral data and known blood glucose data as determined by an early prototype neural network, in accordance with the disclosed technology.

FIG. 19B illustrates a chart illustrating the Error Surveillance Grid associated with pairings of spectral data and known blood glucose data as determined by the early prototype neural network and subsequently refined via example error detection and correction steps, in accordance with the disclosed technology.

Like reference symbols in the various drawings indicate like elements.

### DETAILED DESCRIPTION

This document describes devices, systems, and their methods of use for employing a novel wearable device to non-invasively test and monitor one or more physical attributes and/or health metrics of a wearer of the device and/or associated apparatuses. Particularly, a system and method employing a novel wearable device to non-invasively sense, test, and monitor one or more physical attributes of a wearer in relation to one or more sensed biomolecules is presented. In some implementations, the biomolecule being sensed and tested may be a metabolite, such as glucose, and the wearable device may be configured for testing and monitoring values, such as levels and/or concentrations, of the metabolite, in the tissues, vessels, and surrounding fluids of the body, for instance, using electromagnetic radiation, e.g., light, RF, and/or microwaves. These devices, systems, and their methods of use, such as employing light, RF, or microwave energy to measure the presence and effects of glucose on the body tissues is painless, safe, and inexpensive, and thereby overcome the deficiencies in the aforementioned solutions that are more invasive and/or non-continuous in their monitoring and management of glucose levels. Moreover, the disclosed technology relates in particular to methods and processes for determining relationships, correlations, correspondences, and the like between and/or among various data points and/or data types to determine or estimate a concentration of a biomolecule of interest (e.g., blood glucose) based on non-invasively collected spectral data.

The disclosed technology will be described more fully hereinafter with reference to the accompanying drawings. This disclosed technology can, however, be embodied in many different forms and should not be construed as limited to the examples set forth herein. The components described hereinafter as making up various elements of the disclosed technology are intended to be illustrative and not restrictive. Many suitable components that would perform the same or similar functions as components described herein are intended to be embraced within the scope of the disclosed electronic devices and methods. Such other components not described herein may include, but are not limited to, for example, components developed after development of the disclosed technology.

In the following description, numerous specific details are set forth. But it is to be understood that examples of the disclosed technology can be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Throughout the specification and the claims, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

Unless otherwise specified, the use of the ordinal adjectives “first,” “second,” “third,” etc., to describe a common object, merely indicate that different instances of like objects are being referred to and are not intended to imply that the objects so described should be in a given sequence, either temporally, spatially, in ranking, or in any other manner.

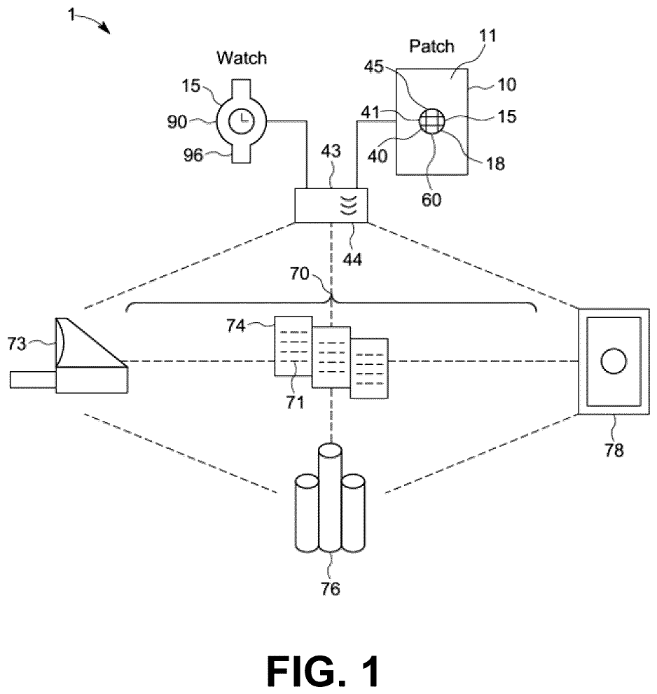

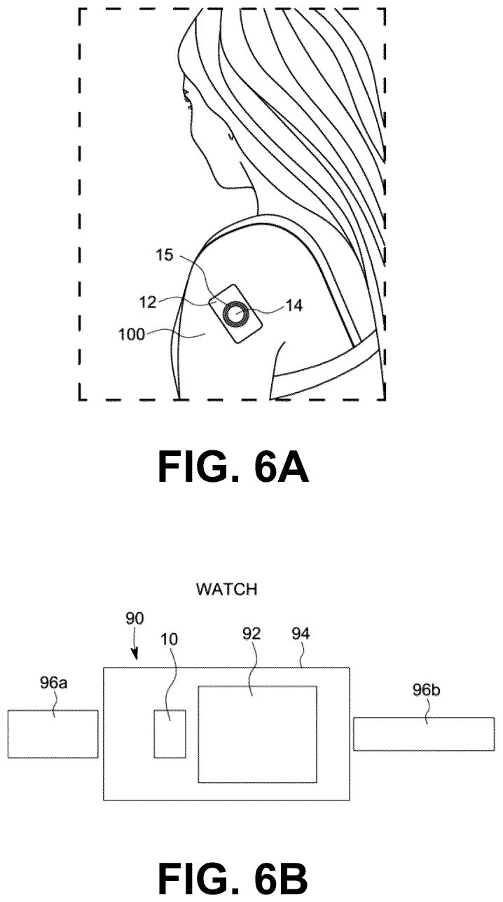

As can be seen with respect to FIG. 1, a system 1 is herein provided for detecting and determining the presence of a biomolecule, such as glucose, in one or more biological tissues of the body and/or in the interstitial spaces therebetween. Generally, the system 1 can include one or more biometric sensing and/or monitoring devices 15 that can be associated with an attachment structure 12 to form a sensing and monitoring apparatus 10. The attachment structure can be configured as a patch 11, and the patch 11 may include an attachment element 13, such as an adhesive, by which the patch 11 can be adhered to the body of the user 100 (e.g., as depicted in FIG. 6A). Alternatively or in addition, the biometric sensing and/or monitoring device 15 can be in the form of a watch-like device 90, and the attachment structure 12 can be configured as a band 96 to which the watch-like device 90 can be coupled so as to be worn around the wrist of a user (e.g., as depicted in FIG. 6B).

In either instance, the wearable biomolecule monitoring and tracking apparatus 10 can include a sensor device 15 having one or more sensor units 18. Each sensor unit 18 can include a number of sensor sub-systems, such one or more energy emitter and/or receiver arrays 20 (e.g., for sensing and generating biomolecule and/or biometric data), a communications module 43 (e.g., including a transmitter 44a and/or receiver 44b) for transmitting the collected biomolecule and/or biometric data, and one or more of an on-board computing system 45 and/or an off-board computing system 70 (e.g., including an analytics system 71). The on-board computing system 45 can be configured to process data, such as pre-processing, or collected sensed energy data (e.g., reflected spectral radiation data). For instance, the onboard computing system 45 can include an analog to digital converter 41 for converting raw sensed energy data, e.g., reflected electromagnetic radiation data, into digital signal read data (e.g., which can then be transmitted to an offboard computing system 70 such as for display and/or further processing thereby).

For instance, as described in greater detail herein below, the sensor unit 18 may include a sensor array 20 having a number of energy emitters 22 and energy receivers 23. The energy emitter(s) 22 may be configured for directing energy, such as electromagnetic radiation, e.g., light, into the tissue of a wearer of the apparatus 10. As such, some of the emitted electromagnetic radiation of various wavelengths may be absorbed by the skin tissues, and various components thereof, some will be refracted, and some will be reflected back. The energy receiver(s) 23, therefore, may be configured for collecting the raw, non-absorbed electromagnetic radiation, e.g., reflected radiation, which can then be converted, e.g., by an analog to digital converter 41, into digital signal read data. Consequently, the onboard computing system 45 may be configured for processing the electromagnetic and/or read data, converting it to processed read data, and then may further process the read data so as to determine one or more values of the biomolecule being assessed.

Once processed, the biometric sensing and monitoring device 15 may transmit the determined values, raw electromagnetic radiation, and/or digital signal read data, e.g., wirelessly, to an associated computing system 70 and/or to a mobile computing device 78 for subsequent processing and/or display thereby. In other instances, such as where more complex computational processing is desirable to be implemented, the sensing and monitoring device 15 may transmit the raw data, the digital signal read data, and/or processed value data to an external computing system 70, such as to a server system 74 and/or an associated client computing device 73, for additional processing of the data. Such transmission may be through a wired or wireless network connection. In particular implementations, such transmission may be performed via a suitably configured wireless communications protocol, such as Wi-Fi, Bluetooth®, Bluetooth Low Energy® (BLE), RFID, Zigbee®, and the like.

Accordingly, in various implementations, a computing system 70 of the system 1, whether it be implemented as an on-board processing unit 45 of the sensor device, a remote, e.g., cloud-based, server 74, a client computing device 73, e.g., desktop computer, and/or mobile smart phone 78, may include or otherwise be associated with an analytics system 71. The analytics system 71 can be configured to detect and/or quantify one or more biomolecules within the tissues of the body, and with respect thereto, can be configured to determine one or more characteristics of the biomolecule of interest, or characteristics of one or more states of the wearer of the sensing and monitoring apparatus 10, such as in response to the determined presence of the biomolecule. In such instances, the analytics system 71, can include one or more processing modules, which can include one or more of processors.

The system 1 can include a processing module for receiving or otherwise accessing the raw or preprocessed sensed data, e.g., digital signal raw data, and processing that data to thereby determine a characteristic of one or more sensed biomolecules in the body of a user (or wearer) and/or a characteristic of one or more states of that user, such as one or more state characteristics caused by, or experienced in conjunction with, the presence of that biomolecule. For example, the analytics module 71 can include one or more processing engines configured to access or receive raw and/or digital signal read data and convert the digital signal data into spectral data. The processing engine(s) can analyze the spectral data to determine a presence and/or a value (e.g., amount, concentration) of one or more biomolecules within the tissue of the user, such as based on a spectral analysis of that data.

Further, sets of processing engines may then further analyze the resultant data so as to determine an effect of the body tissue, which may be based in part on the spectral data, as well as in part on other sensed and/or observed data, such as trend data, so as to produce suspected biomolecule effect result data. Additional sets of processing engines may then use one or more of the determined spectral data and the suspected biomolecule effect result data to build a data structure, such as a data structure including a number of different raw and/or read data obtained from a number of different reads derived from the user, or a plurality of users, over time so as to generate a model of past and/or predicted effect data based on the value of the one or more biomolecules, raw and read data, and spectral analyses related thereto. From these data, a further set of processing engines may then determine, or otherwise predict, a characteristic state of the user, such as based on the one or more trends or models, for instance, where the determined characteristic state data represents a state of the user based on the value of the one or more biomolecules.

In particular embodiments, the data to be collected by the sensing and/or monitoring device 15 may be collected in conjunction with one or more other biomolecule sensing devices such as for more directly measuring and/or determining the values, e.g., levels and concentrations of the biomolecule of interest. For instance, a finger prick may be employed to lance the skin and draw blood, a blood sample can be associated with an analyte stick, and then the analyte and blood may be inserted into an analyte, e.g., glucose monitor, where by an actual reading of the level and concentration of blood glucose may be determined. This biomolecule level and concentration data may then be transmitted into the computing system 70 or 78, such as wirelessly, or it may be entered manually by the user, such as by entering the information via the mobile application running on their associated smart phone. Other forms of biomolecule monitoring devices, such as the analyte-needle patch like devices described herein below, may also be used to test and input biomolecule levels into the system, such as in conjunction with the sensor devices and methods disclosed herein.

Hence, in various implementations, the wearable monitoring and/or tracking device 15 can test for the presence, concentration, level, and/or reactivity of a biomarker, such as glucose or other metabolite, in a tissue of the body, in a non-invasive manner, such as using electromagnetic radiation detection and/or spectral analysis. As described herein, the electromagnetic radiation often refers to light waves, but may also refer to Radio Frequency (RF) or microwave waveforms as well. In the case of RF and/or microwaves for use in detecting and/or characterizing the presence of a biomolecule within a tissue, the emitter and/or receiver of the sensor unit 18 may be implemented in connection with a suitably configured antenna array, and the rate of emittance, e.g., bursts, may be short with low amplitude, so as to prevent damage to the underlying tissues.

In any of these instances, the wearable sensing and/or monitoring device 15 can be configured to emit energy into a living tissue of a body, such as at a low amplitude so as to not over energize the tissues, for the purpose of testing for biomarkers or metabolites in the wearer's blood, skin, and/or interstitial spaces therebetween. In doing so, a number of techniques can be employed so as to detect one or more of the presence, concentration, and/or effects of a biomolecule within the tissues of the body of a user 100, and the response to of those tissues to the presence of that biomolecule can also be determined. The analytical techniques that can be employed in a manner consistent with the present disclosure include, but are not limited to: polarimetry, photoacoustic spectroscopy, bio-electrical impedance spectroscopy, thermal emission, optics, acoustics, and other technologies. Such optical and acoustic techniques can be employed herein in a highly accurate, painless, and safe noninvasive detection and measurement process. Relevant determination methods that may be employed by the analytics system 71 may include optical coherence tomography, microwave spectroscopy, near-infrared spectroscopy (NIRS), midinfrared spectroscopy (MIRS), Raman spectroscopy, and/or visible laser light spectroscopy. In various embodiments, Green, Red, Infrared, and/or Near Infrared spectroscopy may be advantageously employed for their simplicity and effectiveness relative to other techniques. Another advantage is that Visible, IR, and NIR components are reasonably priced and can penetrate the skin to adequate depths to enable accurate analysis.

Accordingly, in view of the above, the devices, systems, and their methods of using electromagnetic radiation, as described herein, for detecting and testing for biomolecules within the skin, are both painless and effective for continuous use over a prolonged period of time, thereby overcoming the limitations of other potential invasive and non-invasive solutions. Specifically, electromagnetic waves, e.g., light, RF, microwave etc., have characteristic levels of absorption and/or reflectance within the skin that may be dependent on the length and frequency of the underlying waveform, whether it be visible or non-visible light or a radio frequency. This phenomenon may, in part, be a basis for performing spectral analyses, as described herein. For example, exposing a tissue to specific wavelengths and detecting the absorption and/or reflection levels allows the analytics system to determine the presence of specific elements, and further analysis allows for the detection and/or prediction of the results, on the body, due to the presence of those specific elements, e.g., biomolecules, within the tissue.

Particularly, combinations of elements produce a combination of absorption and/or reflectance patterns that can be used to determine the presence of molecules based on their elemental composition and the various spectral patterns they produce. By comparing the characteristics of these patterns, and/or their light intensities, with the absorption response in the body tissues, it is possible to determine the presence and values of certain biomolecules within body tissues along with their levels and concentration. This computation, however, may include the simultaneous analysis of a number of different factors, from the same or a number of different individuals or groups, using a plurality of detection devices and methodologies, which may all need to be evaluated when performing an accurate determination and/or measurement. In various embodiments, a data structure by which all of these various datapoints may be compared and weighted may be built, such as in the performance of the referenced evaluations. Consequently, the analytics system may embody, or otherwise employ, an artificial intelligence module, by which to perform the various evaluations herein disclosed.

For example, in some implementations, a wearable sensing and/or monitoring device of the system may employ electromagnetic radiation emittance and sensing along with diffuse reflectance spectrum analysis together with an artificial intelligence system that can analyze a plethora of data of the individual, over time, as compared to a number of other such individuals, so as to develop and/or use one or more trends and models by which a given particular set of data may be evaluated. More specifically, raw waveform, read, spectral, and other data of the individual can be combined within a data structure, such as a multi-layer artificial neural network, to directly read and/or determine biomolecule, e.g., glucose, levels from, or within, the skin of an individual wearing the biometric apparatus. These techniques are advantageous over previously implemented devices and techniques because the apparatuses employed herein are non-invasive and relatively comfortable to wear, such that continuous readings or measurements may be made over time, and during exercise and showering, which has heretofore not been possible, making these calculations impossible for other such non-continuous sensing devices.

For instance, as indicated above, the present devices are advantageous over other solutions previously sought, because the measurements obtained by the present devices are performed in a non-invasive manner, such as without requiring needle pricks or finger sticks, which are typically used for detecting and/or taking blood glucose levels. Thus, the present devices and systems increase compliance and useability, while avoiding the drawbacks of previous sensing device implementations. Particularly, the manner by which biomarkers are presently detected by devices currently on the market suffer from major drawbacks in that they are painful, expensive, and inconvenient. Such finger-prick devices, by their nature, do not allow for continuous sensing and monitoring, and because of the pain involved, they evidence poor compliance. Accordingly, the analytics techniques disclosed herein could not be performed with such devices.

More particularly, presently available devices are painful to use in that they require an invasive pricking of the skin, so as to intermittently test blood retrieved thereby. Other iterations involve the intrusion of a needle-like appendage that must be inserted and remain within the skin. These devices are uncomfortable to wear, should be removed before showering, and, therefore, also evidence sub-optimal compliance. Another factor negatively effecting compliance is due to the fact that the use and maintenance of such devices are expensive, such as averaging about $120/mo. Hence, previous devices are expensive, inconvenient to use, last only one or two weeks, and cannot readily be removed and/or adjusted. Such devices often further suffer from largely being “dumb” devices with limited data collection and have virtually no on-board analysis. Additionally, because these devices typically have sensors that need to be replaced every 7-14 days, some with a transmitter that also needs to be replaced every 3-4 months, their use creates a problem of increased waste of medical materials.

Specifically, there are two main types of invasive semi-continuous glucose sensing devices that are currently being promoted. One such type of device is the aforementioned glucose monitoring device that requires the invasive finger prick in order to test the blood for glucose. The other type of device includes a sensor unit that needs to be inserted within the skin of the user. In this regard, this device does not measure glucose levels directly like the finger-prick blood glucose monitoring device does. Rather, it utilizes an amperometric electrochemical signal that is generated when glucose, present in the interstitial fluid, reacts with an inserted chemical coated sensor, which reaction thereby converts the interstitial glucose to glucose oxidase that it then detects. In this regard, such amperometric sensor devices measure glucose in the interstitial fluid through an electrochemical reaction facilitated by a glucose-specific enzyme, i.e., glucose oxidase, which is coated onto the inserted glucose sensor, and reacts with glucose present in the space so as to convert it to glucose oxidase.

The herein disclosed non-invasive, continuous sensing devices operate on a completely different technological basis than the aforementioned amperometric sensor devices. With respect to the devices, systems, and their methods of use disclosed herein, in certain iterations, the present optical and/or acoustic based sensor arrays use electromagnetic, e.g., light or RF or microwave, absorbance and spectral analysis to determine bio-marker values, such as where the biomarkers are present and observable in the interstitial fluid and/or blood. As indicated, in various embodiments, the presence of the biomolecule may be detected and/or predicted directly via on-board analysis, e.g., of a spectral array, but in other embodiments, the presence of the biomolecules and their effects may be determined by a model generated by a suitably trained Artificial Intelligence (AI) module, such as by building and employing an Artificial Neural Network (ANN), as explained herein below.

More specifically, it has been determined herein that the presence of molecules, such as glucose, within the blood and interstitial fluids, may affect the absorbance and/or reflection of electromagnetic radiation, e.g., light, being directed at it, such as through the skin and tissues of the body. For instance, molecules, e.g., glucose, affect light absorbance and reflection in a uniform, and characteristic manner, such as by reflecting emitted light back at a characteristic wavelength and frequency. When glucose is in the blood and/or interstitial fluids, the skin color and/or composition changes, such as on an infrared wavelength scale, which can then be observable by the detecting and sensing devices of the disclosure.

For example, when lights of particular wavelengths are directed into the skin both in and not in the presence of a biomolecule of interest, the light reflected back changes in its characteristics, e.g., characteristic frequencies, based on whether the biomolecule is present or not, and further based on the reactance of the skin thereto. These characteristic frequencies can allow a sensing device of the disclosure to detect the presence of these molecules, as well as their quantity and/or concentration in the interstitial fluid and/or blood. Consequently, in various embodiments, if a light (or a laser or an RF or microwave emission, etc.) is directed into the skin, at a particular frequency and wavelength, a characteristic pattern of energy absorbance and/or energy reflectance may be sensed and obtained, such as by a specifically attuned electromagnetic radiation sensing device, e.g., energy receiver.

This pattern can then be used to determine the presence and quantity of given biomolecules, e.g., glucose, in the blood and/or interstitial fluid, as well as to predict the effect such a biomolecule will have on the body. For instance, the presence and quantity of glucose, or other biomolecules, may be determined by comparing a test light spectrum of a sample with a known light spectrum where the presence and quantity of biomolecules, e.g., glucose, is known, e.g., based on how well the test sample light absorbance and reflection pattern approximates the known light absorbance and reflection pattern where glucose is present. This process can be performed using a laser; however, such an implementation is difficult because currently available lasers are large, bulky, high energy absorbing equipment that is not readily transportable or mobile.

Consequently, miniaturized light sources, e.g., light emitting diodes, have been developed and it has been determined that, like lasers, these light sources, when directing light into the skin also produce such characteristic light absorbance and reflection wave forms. This was unexpected in that such light sources are not as intense, nor accurate, as using a laser, but are accurate enough to give relative readings that can be analyzed in accordance with the methods disclosed herein. By performing the processes herein described, using low intensity light sources, such as generated from light emitting diodes, it has been found that accurate blood and interstitial biomolecule values and levels can be determined in a manner that will allow a subject to monitor and control the body's response to those biomolecules.

Pursuant to these findings, these miniaturized light sources have been formed herein into arrays, where each light sources may be configured to emit light of one or more particularized wavelengths, which light when directed into the skin can excite a reaction thereby. This reaction in turn creates a characteristic absorbance and reflection pattern of the light, which differs in the presence of various biomolecules, and thus, can be sensed by the accompanied light detection mechanisms, e.g., photodiodes, and used to determine one or more conditions or states of the body. This reflection pattern may be due in part to the interaction of light with the biomolecule and/or the reaction to the skin composition in response thereto. Collectively, as described herein, the light emitters and light sensors can be miniaturized and fit into a portable sensor unit that can be positioned on the skin so as to continuously, and non-invasively monitor biomolecule, e.g., glucose, levels, such as based on light absorbance and/or reflection patterns, regardless of how active the wearer is and/or what activities in which they engage, be it walking, running, or even swimming.

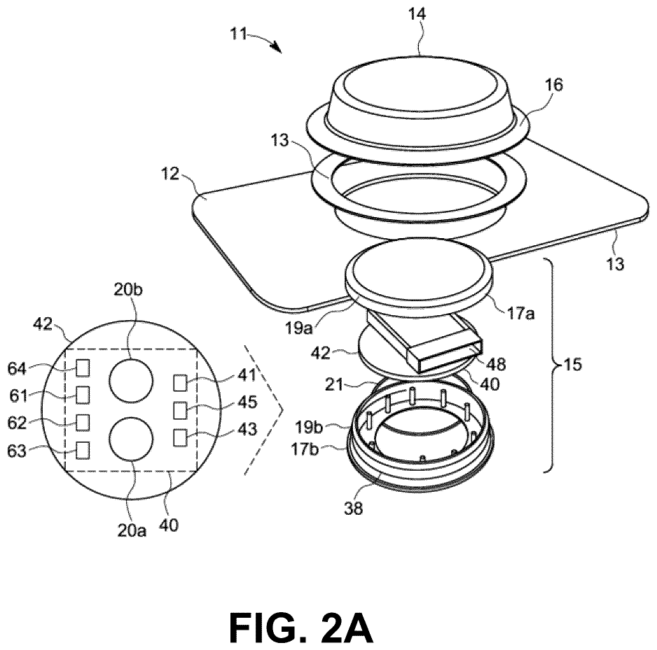

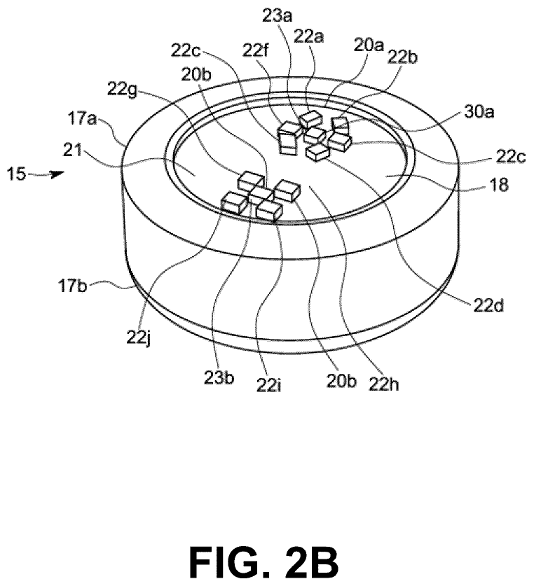

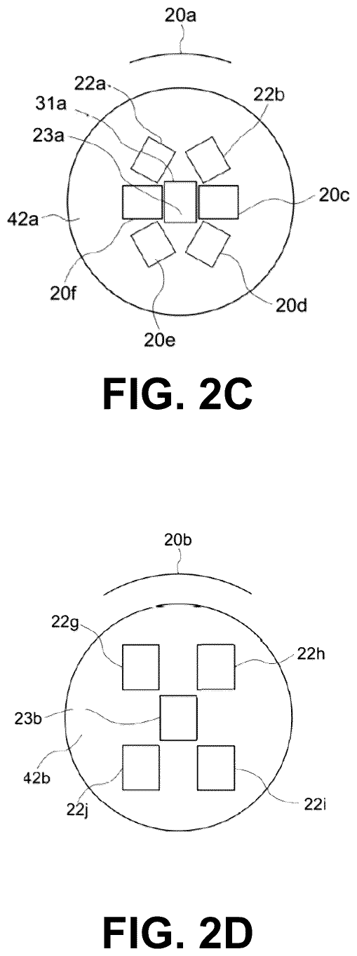

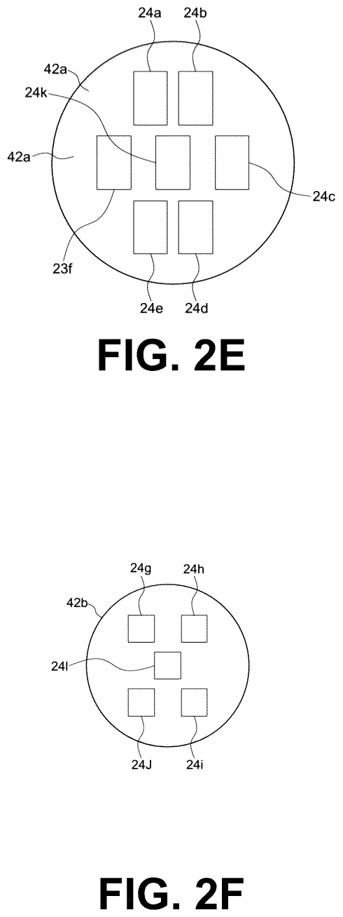

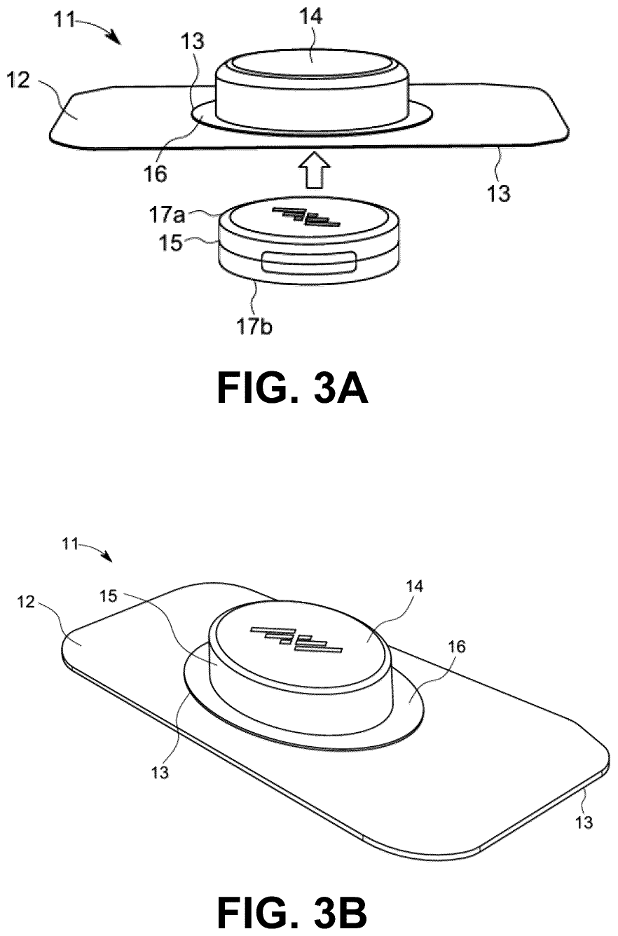

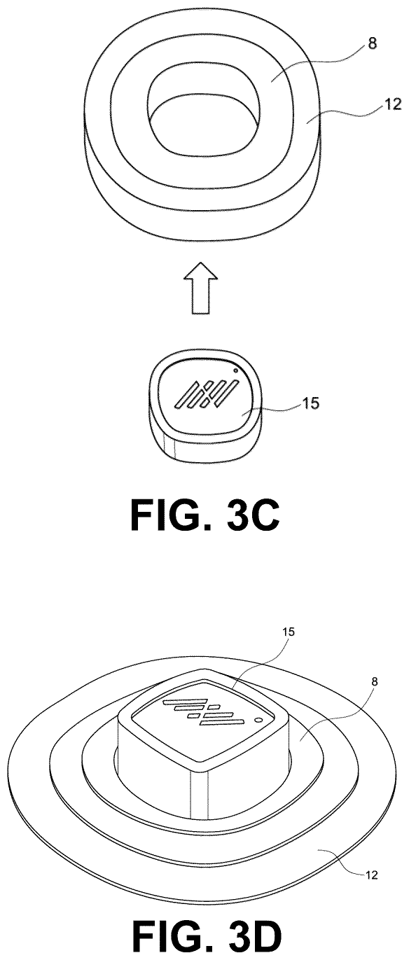

Accordingly, in one aspect, the disclosure is directed to a biometric sensing and/or monitoring apparatus 10, as illustrated in FIG. 2A. In this instance, the apparatus 10 is shown in an exploded view, and is configured as a patch 11. The patch 11 may be configured to include a sensing and monitoring device 15, as shown in FIG. 2B, an encasement member 14, for encasing the sensing and monitoring device 15 and holding it in place, and an attachment structure 12 for attaching the patch 11 to the body of an individual, as depicted in FIG. 6A. As can be seen with reference to FIGS. 2A and 3A, the encasement member 14 is configured for being coupled to the attachment structure 12 so as to form the patch 11. This coupling can be effectuated by a variety of different mechanism, but as depicted the coupling can be effectuated with an adhesive sealing element 13, which can function to both couple the encasement member 14 to the attachment structure 12, as well as to form a waterproof sealing therebetween.

As shown in FIG. 3A, once the encasement member 14 is coupled to the attachment structure 12 the patch 11 is formed, then the sensing and monitoring device 15 may be inserted into the patch 11, such as by being inserted into encasement member 14, as shown in FIG. 3B. Likewise, once the sensing and monitoring device 15 is inserted into the encasement member 14, so as to be encased thereby, the biometric apparatus 10 is formed, as seen in FIG. 3B. The attachment structure 12 can then be securely attached to the body 100 of the individual, such as through the addition of a further attachment element 13, such as a bio-acceptable adhesive, to a skin facing surface of the attachment structure 12. The adhesive in this instance may be such that it securely effectuates the attachment of the patch 11 to the body 100, as shown in FIG. 6A, but is capable of allowing the apparatus 10 to be removed with the application of a removing force.

As indicated, the encasement member 14 is configured for receiving the sensing and monitoring device 10 therein. So being, the encasement member may be configured as a dome or disc 14. Particularly the encasement dome or disc 14 may be composed of a plurality of horizontally extended flat surfaces that are offset from one another by a bounding surface or wall. Essentially, a first flat surface may form the top of the dome, and a second flat surface can form the bottom or base of the dome, such as where the bottom base surface further acts as an interface 16 by which the dome 14 may be coupled with the attachment structure 12, as shown in FIG. 2A.

As depicted the first and second flat surfaces of the dome are parallel but offset from one another by the bounding surface, which bounding surface is positioned substantially normal to the first and second flat surfaces. In this configuration, the first, e.g., top, surface and the second, e.g., bottom, surface do not overlap in their extension. Together the top flat surface and the substantially perpendicular bounding surface form the dome 14 and further define an opening of a cavity of the encasement member, into which cavity the sensing and monitoring device 15 may be fitted, as shown in FIG. 3A. In this regard, the height of the bounding surface, as well as the distance across one bounding surface to an opposed bounding surface, e.g., the diameter, should be such that the sensing and monitoring device 15 may be securely and snuggly fitted within the dome 14, hence, the dome 14 may be slightly larger than the sensing device 15, such as by 1 mm or less. Further, as indicated, the base surface 16 of the dome 14 may form an attachment interface, extending laterally outwards from the bounding surface, by which the encasement member 14 may be coupled to the attachment structure 12 in a manner that the opening of the cavity is coincident with a corresponding opening of the attachment structure 11, as shown in FIG. 3A.

Likewise, the attachment structure 12 may have any suitable shape and/or configuration so long as it is capable of attaching the sensing and monitoring device 15 in close proximity to the skin of the wearer, such as via a suitably configured coupling with the dome member 14, as shown in FIG. 3B. For such purposes, the attachment structure 12 may have an elongated surface member that includes a top surface, such as for interfacing with the dome member 14, as well as a bottom surface for interfacing with the body of an individual who is to wear the apparatus 1. This elongated surface member may have an interior portion and an exterior portion, such as where the exterior portion is defined by a perimeter bounding member.

As such, the exterior perimeter bounding member of the attachment structure 12 may form a single circumferential, outer perimeter, such as in the shape of a circle, or may be composed off opposite sides, so as to form a triangle, square, rectangle, and the like, e.g., dependent on the number of sides. Further, the elongated surface member 12 may include an inner perimeter portion defining an opening, e.g., sized so as to be coincident with the opening of the dome 14, such that the sensing and monitoring device 15 may be fitted therethrough so as to both be inserted into the dome 14 and abut the skin of the wearer, when the apparatus 10 is placed on the body. In some embodiments, this opening in the interior portion of the attachment structure 12 may be covered with a transmissive covering so that the sensing device 15 does not actually touch the skin. As indicated, the bottom portion of the elongated surface 12 may be coupled with an attachment element, such as an adhesive 13, for coupling the overall apparatus 10 to the body of the wearer.

Accordingly, in view of the above, in another aspect, the disclosure is directed to a sensing and monitoring device 15 as set forth in FIG. 2B, such as for determining a characteristic of one or more biomolecules present within a living tissue of an individual. In particular implementations, the device 15 may further be configured for determining, or otherwise predicting, a state, or a change in a state, of the individual, such as based on a value of the one or more biomolecules being present within the tissue. As depicted in FIG. 2B, the sensor and/or monitoring device 15 is shown sensor-side up, so as to show the details of the device, but in use, the sensor-side would be face down so that the sensor elements would be facing the skin of the body tissue, such as when the sensor device 15 is inserted within an encasement member 14 of a patch 11, and the patch is applied to the skin, as shown in FIG. 6A.

As illustrated, the hardware of the monitoring and sensing device 15 may include a housing 17 that retains a sensing and/or monitoring unit 18 therewithin. The housing 17 may be composed of an upper 17a and a lower 17b housing or cover member, and as illustrated, may include a protective glass 21, such as at a skin interface of the bottom cover member 17b. This transmissive portion, e.g., window, is useful because it allows for, or otherwise facilitates, passage of electromagnetic radiation, e.g., light and/or sound, of determined wavelengths, being emitted from the sensor unit 18, to pass therethrough. However, although the bottom cover 17b may include one or more windows 21, in other instances, the bottom cover 17b may be formed of a transmissive material, such as where the entire bottom portion is composed of the transmissive material, such as protective glass.

Since the sensing and/or monitoring device 15 is configured to be worn for a prolonged period of time contacting the skin, the cover members 17a, 17b may be formed of a material that is non-toxic, non-irritative, bio-compatible, and/or may otherwise be capable of being pressed against the skin of a user without adverse effects. In certain instances, the housing may be 3D printed with acrylic resin that becomes rigid when hardened. The housing may have any suitable shape and configuration. However, for ease of use, and to promote a thin profile, the housing 17 may have a circular, disk-like shape. Nevertheless, in various instances, the housing 17 may be in the shape of a triangle, square, rectangle, and the like, so long as the form factor is capable of containing the electronics of the device, and yet maintaining a stream-lined, low profile.

As indicated, the housing 17 is configured for retaining a sensor and/or monitoring unit 18 therewithin. So being, the housing may have a top housing or cover member 17a and a bottom housing or cover member 17b that are configured for being coupled together so as to encase the sensor unit 18 as well as the other electronic components 60 of the sensing and monitoring device 15. Particularly, each top 17a and bottom 17b member of the sensor device housing 17 may have both an extended planar surface as well as a perimeter surface 19, such as where the perimeter surface may include one or more bounding walls 19a, 19b that extend substantially normal to an outer edge of the extended planar top and bottom surface members 17a and 17b. In such instances, as can be seen with respect to FIG. 2A, when the corresponding bounding walls of the top and bottom cover members are coupled together a cavity is formed therebetween.

Accordingly, as can be seen with respect to FIG. 2B, the sensing and/or monitoring device 15 may include a housing 17 for containing the sensor unit 18 and the associated electronics 60 within the device. As depicted the housing 17 includes a top member 17a and a bottom member 17b having extended top and bottom surfaces, which are further defined by respective perimeter portions 19a, 19b. As can be seen with respect to FIG. 2A the top and bottom members 17a, 17b along with the perimeter portions 19a, 19b have interior surfaces, e.g., defining the inside of the cavity, and also have outward surfaces. Consequently, in this regard, the perimeter portions 19a and 19b may be configured as bounding members.

As depicted, the perimeter portions 19a, 19b of the top and bottom members are configured to extended normal to the relatively flat surfaces of the top and bottom members 17a, 17b. In this manner, when the top member 17a and the bottom member 17b are coupled together, via the coupling of the perimeter portion bounding members 19a and 19b, a cavity is formed between the top 17a, bottom 17b, and bounding 19a, 19b members, in which cavity the sensor unit 18, and other electronic components 60 of the sensing and/or monitoring device 15, may be retained. As depicted, the top 17a and bottom 17b surfaces are substantially flat, and the cavity is formed by the height of one or more of the perimeter bounding portions 19a, 19b. However, in various embodiments, the surface of the top and bottom members 17a, 17b may be curved, e.g., radially, such that the perimeter portions 19a, 19b, are minimally extended, and thus, do not really serve a bounding function. Rather, as the top 17a and bottom 17b members are coupled together, the sensor unit 18, and associated electronic components 60, can be retained within a natural cavity that is formed by the opposed, corresponding curvatures of the top and bottom surface members 17a, 17b. In such an instance, the perimeter portions 19a, 19b may be minimally extended, if at all.

Further, as depicted in FIG. 2B, the sensing and monitoring device 17 is configured as a circular disc-like member. However, in various embodiments, the sensing and monitoring device 15 may also have a square or rectangular-like configuration, in which case each of the bounding members 19a and 19b may be composed of a plurality of opposed perimeter walls, such as to form a square or rectangle or other edged shape. For instance, other shapes may also be formed, such as by increasing the number, shape, and orientation of the bounding walls. In any of these configurations, the top 17a and bottom 17b members may include a coupling mechanism, such as for latching the top 17a and bottom 17b members together to form the housing 17. In such instances, the coupling mechanism 39 may be configured as a tongue and groove, as opposed teeth, such as in a configuration approximating opposed “L” shapes, as corresponding latches, clips, snaps, hook and loop, opposed posts of differing diameters, such that one post fits within the other, and the like, such that the two members of the housing 17 may be snaped together. In particular embodiments, the two members of the housing 17 may be coupled together by a fastener, such as a rivet, screw, bolt, an adhesive, such as glue, and the like. In various embodiments, a compressible member 38, such as an O-ring, may be included between the corresponding latches so as to be compressed by the latching and thereby form a waterproof sealing between the two members.

As further can be seen with respect to FIG. 2A, the biomolecule sensing and/or monitoring device 15 can include a sensing unit 18, a power unit 48, a printed circuit board arrangement (PCBA) 42, along with one or more electronics modules 60, all of which may be fitted within the housing 17. As shown in FIG. 2B, an essential feature of the sensing device 15 is the sensor unit 18 having one or more sensor arrays 20, such as where each sensor array 20 may include one or more energy emitters 22 and receivers 23, all of which can be coupled to the PCB 42 and be powered by the power unit 48. Because the device is configured to be worn on the body 100 of a wearer for a prolonged period of time, without being removed for showering, swimming, and the like, so as to ensure the continuous monitoring of the body tissues, it is useful for the device to have a thin, circular profile, such as for ease and longevity of wearing.

Consequently, the dimensions of the housing 17, sensor unit 18, power source 48, and on-board electronics 60 herein disclosed have all been miniaturized to be both small, but also, in some embodiments, circular; although the configuration may also be square or rectangular. Hence, the sensor unit 18, including the emitters 22 and receivers 23, in conjunction with the PCBA 42, have been adapted so as to have a circular form factor, but the shape will be dependent on the shape of the overall housing of the device. However, with respect to the size of the housing 17, the size may be dependent on the collective size of the included sensor and electronic components necessary for performing the disclosed activities. In various instances, the sensor device may be less than 10 mm, such as from about 1 or 2 mm to about 8 or 10 mm, such as about 3 or 4 mm to about 6 or 7 mm, including about 5 mm in height. Likewise, the sensor device may have a cross-wise length from about 2 mm to 5 mm or 10 mm or 12 mm to about 40 mm or 50 mm, such as from about 15 mm or 20 mm to about 25 mm or 30 mm, such as dependent if the form factor is circular or square. In particular iterations, such as where the sensing and/or monitoring device 10 has a circular form factor, the diameter of the overall device may be less than about 5 cm, such as less than about 4 cm, or less than about 3 cm, such as less than about 2 cm or less. Likewise, the height may be less than 5 cm, such as less than 4 cm or 3 cm, for instance, less than 2 cm, less than 1 cm, such as about 5 mm.

As can be seen with respect to FIG. 2B, a key component of the biometric sensor and/or monitoring device 15 is the sensor unit 18 that is configured to include a plurality of miniaturized sensor arrays 20. For instance, the biometric sensor unit 18 may include a plurality, e.g., two, sensor arrays 20a and 20b. Each sensor array 20a, 20b, may include one or more electromagnetic radiation emitters 22 and one or more electromagnetic radiation receivers 23, each being arranged on a printed circuit board 42 within in the housing 17. As indicated, the electromagnetic radiation emitter may be configured as a photoemitter 22, which may be adapted for directing light into the body of the wearer of the device 15. Likewise, the electromagnetic radiation receiver may be configured as a photodiode 23, which may be adapted for receiving residual light waves reflected back from the skin.

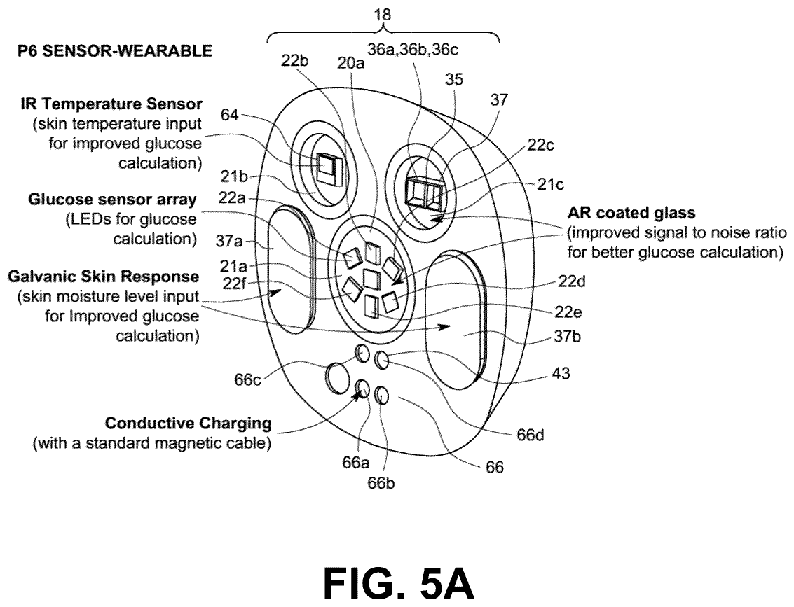

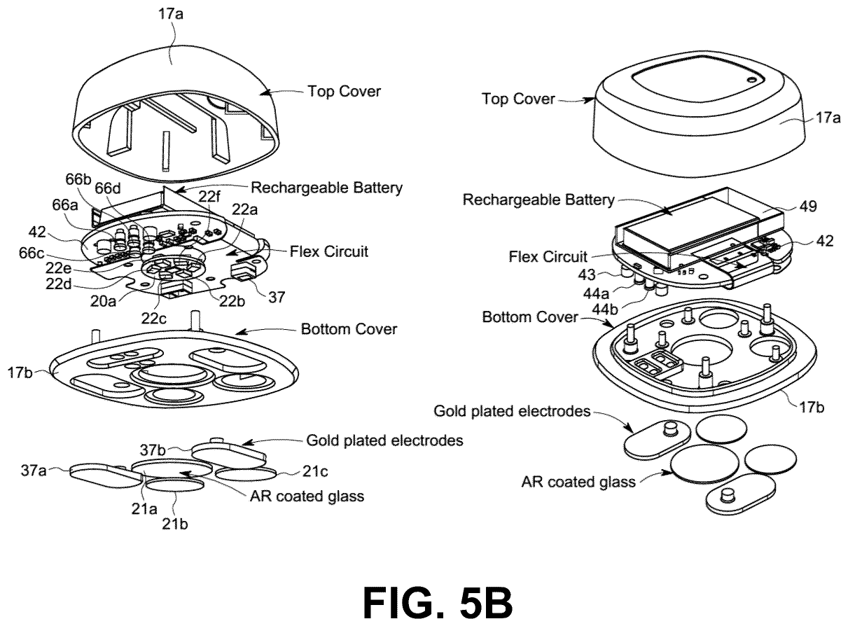

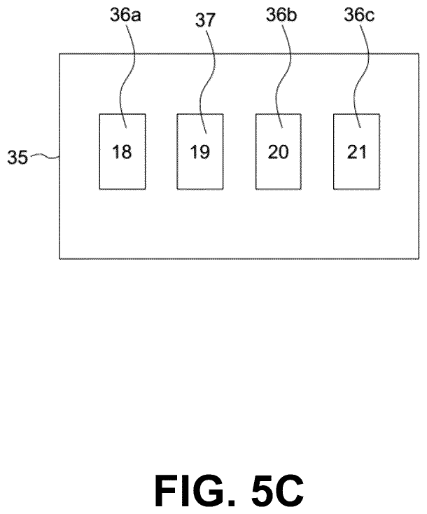

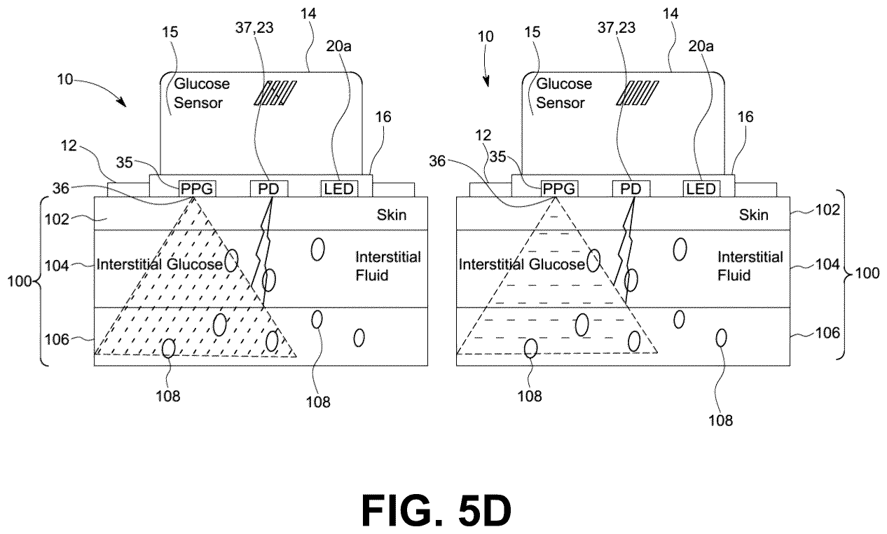

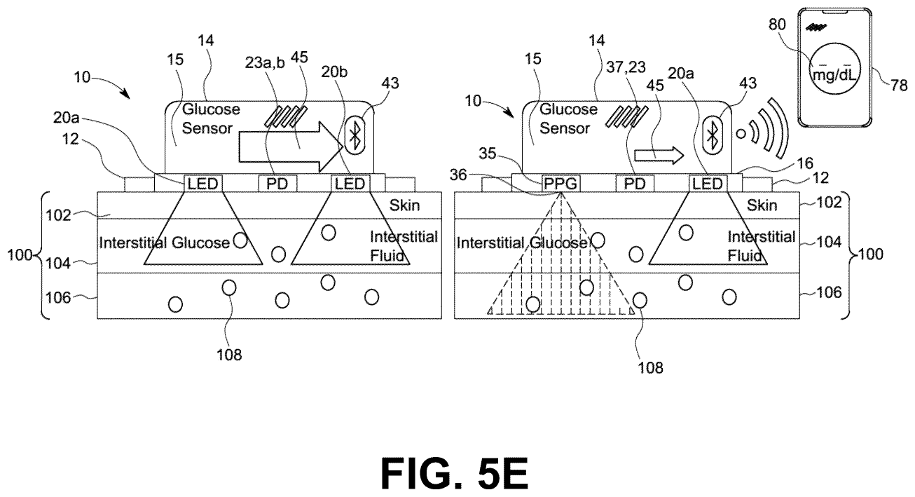

Further, as shown in FIG. 2B, an electronics module 60 of the sensor and monitoring device 15 may include a sensor unit 18 that may be positioned on the PCB 42. The sensor unit 18 may be composed of a sensor array 20, which sensor array 20 may include an energy, e.g., electromagnetic radiation, emitter 22 as well as an energy, e.g., electromagnetic radiation detector 23. As depicted in FIG. 2B, the displayed sensor unit 18 includes a first array 20a of six energy emitters 22a, 22b, 22c, 22d, 22e, and 22f surrounding a first energy receiver 23a, as depicted in FIG. 2C, and further includes a second array 20b of four energy emitters 22g, 22h, 22i, and 22j surrounding a second energy receiver 23b, as depicted in FIG. 2D. Although depicted with two sensor arrays 20a and 20b, one more of these arrays may be substituted out for other sensor arrays, having different configurations, as illustrated in FIG. 5A, or may be substituted out for different electronic components all together. In particular embodiments, a sensor unit 18 may have more than two arrays 20, such as three, or four, or five, etc., where each array may have 1, 2, 3 to 5 to 10, or more energy emitters, and/or 1, 2, 3-10, or more energy receivers.

Hence, in particular embodiments, the sensor unit 18 may include one or more, e.g., a plurality, of arrays having one or more emitters. The emitters may be configured as photoemitters, or they may be adapted for emitting other types of radiation, such as RF or microwaves. For instance, an emitter array may include collection of one or more light emitting diodes, LEDs, for the emitting of light waves may be included. However, in other embodiments, an emitter array may include collection of one or more antennas, such as for the emission of radio or microwaves. Consequently, in certain instances, in one or more of the emitters of one or more of the array may be a sound or microwave generating device, in which instance, the receiving unit may be configured for receiving, sensing, and/or determining reflected and/or refracted sound or microwaves.

Accordingly, the sensor unit 18 may further include one or more arrays 20 of energy receiving elements 23, such as including one or more receivers for receiving and sensing radiation, e.g., electromagnetic radiation, that is reflected and/or refracted back from tissues that have been irradiated with light waves, sound waves, and the like. In particular embodiments, as can be seen with respect to FIG. 2B, the energy receiver 23 can be positioned in close proximity of the energy emitters 22, such as where the energy emitters 22 virtually surround the energy receiver 23, for example, in a circular-like configuration, such as depicted by the array 20a, or in a square-like configuration, such as depicted by the array 20b. Other configurations are also possible, depending on the number of emitters and receivers as well as their ratios. In particular implementations, the sensor unit 18 may be configured as a light sensor such that the one or more emitters 22 are configured as photoemitters, in which case, the sensor unit 18 will also include one or more, e.g., a plurality of, diodes, e.g., photodiodes. Together an emitter, e.g., light emitting diode, in combination with a receiver, e.g., photodiode, may form a sensor or sensor unit herein dependent on the context in which they are recited.

As can be seen with respect to FIGS. 2C and 2D, the sensor arrays 20a and 20b of FIG. 2B are set forth. Specifically, FIG. 2C depicts the six-emitter photo-array 20a of FIG. 2B, while FIG. 2D depicts the four-emitter photo-array 20b. As can be seen with regard to the photosensor array 20a of the sensor unit 18, as set forth in FIG. 2C, the displayed array 20a includes six photoemitters 22a, 22b, 22c, 22d, 22e, and 22f. In this instance, the six photoemitters are configured in a roughly circular pattern, with a single photoreceiver, e.g., photodiode 23a roughly centered in the middle of the ring of photoemitters. This configuration is useful because it allows light from different emitters 20a-f to emit light of different wavelengths, while at the same time as ensuring that their reflected light waves will all impinge upon the centralized photoreceiver 23a. However, in alternative embodiments, more photoreceivers 23 per array may be included so that the emitter to receiver ratio is 6:5, 5:2, 5:3, 4:1, 3:2, 2:1, 1:1, and the like. Further, the configuration of one or more of the photoemitters 22 to one or more of the photoreceivers 23 may be such that one or more dyads are perpendicular to one another.

In various embodiments, the sensor array 20a of FIG. 2C may be composed of photoemitters 22 that are configured to emit light waves. In particular embodiments, the photoemitters 22a-f may be composed of light emitting diodes (LEDs), such as where the LEDs 22a-f surround one or more light receivers, e.g., photodiodes 23a. One or more, e.g., half or all, of the light emitting diodes 22a-f may be configured for emitting light of the same wavelength, or they may be configured for emitting light of different wavelengths. For instance, in one particular embodiment, the six photoemitters of FIG. 2C may be configured for emitting light of different wavelengths, such as, in one exemplary iteration where a first photoemitter, e.g., 22a, is configured for emitting light of a first wavelength, such as within the range from about 1000 nm and about 1100 nm, the second photoemitter, 22b, is configured for emitting light of a second wavelength, such as within the range from about 1100 nm and about 1200 nm, the third photoemitter, e.g., 22c, is configured for emitting light of a third wavelength, such as within the range from about 1200 nm and about 1300 nm, the fourth photoemitter, e.g., 22d, is configured for emitting light of a fourth wavelength, such as within the range from about 1300 nm to about 1450 nm, the fifth photoemitter, e.g., 22e, is configured for emitting light of a fifth wavelength, such as within the range from about 1450 nm to about 1550 nm, and the sixth photoemitter, e.g., 22f, is configured for emitting light of a sixth wavelength, such as within the range from about 1550 nm to about 1650 nm, or more. In this configuration, the one or more photoreceivers may be configured for receiving and detecting light waves in the range from about 1000 nm to about 1700 nm. It is noted that any of the wavelengths of the emitted light may vary by about ±10 nm to about ±25 nm, about ±50 nm, about ±100 nm, and the like. Further, the order and combination of the emitters, along with the range of wavelengths they emit, can be arranged in any logical order, although, in various instances, sequential emission has advantageous of priming the tissue being observed, and in increasing the speed of computational processing. However, in other instances, a random illumination sequence of disparate wavelengths also has advantages.

Further, as depicted in FIG. 2D, the photoemitters 22 are aligned in a canted orientation with respect to the central photoreceiver 23. Specifically, the photoemitters 22a and 22b are canted in a positive direction so as to from the shape of an arrowhead with respect to the photoreceiver 23a. Likewise, the photoemitters 22d and 22e are canted in a negative direction so as to from the shape of a “V” with respect to the photoreceiver 23a. Likewise, the photoemitters 22c and 22f are rotated so as to be 90 degrees from a centerline running through the middle of the photoemitters 22c and 22f and photoreceiver 23, such as to form an “L” or a “T” shape with respect thereto. Other arrangements, e.g., degree of angle running through the centerlines of a respective photoemitter 22/photoreceiver 23 dyad can also be used, such as from a 1-180 degree rotation one with respect to the other, such as determined to be beneficial for receiving a maximal reflectance of emitted energy waves back from the skin and/or so as to reduce the signal to noise ratio. In various instances, the photoemitter 22/photoreceiver can be orientated to be aligned vertically with one another, e.g., 90 degrees, or 45 degrees, e.g., canted, with respect to one another, parallel to one another, or any angle there in between, as determined to be beneficial with regard to increasing signal reception and reducing noise.

Further, as can further be seen with regard to the photosensor array 20b of the sensor unit 18, as set forth in FIG. 2D, the displayed array 20b includes four photoemitters 22g, 22h, 22i, and 22j. In this instance, the four photoemitters are configured in a roughly square pattern, with a single photoreceiver, e.g., photodiode 23b roughly centered in the middle of the box of photoemitters. This configuration is also useful because it illustrates another configuration that allows light from different emitters 20g-j to emit light of different wavelengths, while at the same time ensuring that their reflected light waves will all impinge upon the centralized photoreceiver 23b. However, hereto, in alternative embodiments, more photoreceivers 23 per array may be included so that the emitter to receiver ratio is 4:1, 4:3, 3:2, 2:1, 1:1, and the like. Further, the configuration of one or more of the photoemitters 22 to one or more of the photoreceivers 23 may be such that one or more dyads are perpendicular to one another.

Like above, in various embodiments, the sensor array 20b of FIG. 2D may also be composed of photoemitters 22g-j that are configured to emit light waves. In particular embodiments, the photoemitters 22g-j may be composed of light emitting diodes (LEDs), such as where the LEDs 22g-j surround one or more light receivers, e.g., photodiodes 23b. One or more, e.g., half or all, of the light emitting diodes 22g-j may be configured for emitting light of the same wavelength, or they may be configured for emitting light of different wavelengths. For instance, in one particular embodiment, the four photoemitters of FIG. 2D may be configured for emitting light of different wavelengths, such as, in one exemplary iteration, where the seventh photoemitter, e.g., 22g, is configured for emitting light of a seventh wavelength, such as within the range from about 550 nm to about 650 nm, the eighth photoemitter, e.g., 22h, is configured for emitting light of an eighth wavelength, such as within the range from about 650 nm to about 850 nm, the ninth photoemitter, 22i, is configured for emitting light of a ninth wavelength, such as within the range from about 850 to about 940 nm, and the tenth photoemitter, e.g., 22j, is configured for emitting light of a tenth wavelength, such as within the range from about 940 nm to about 1000 nm. In this configuration, the one or more photoreceivers may be configured for receiving and detecting light waves in the range from about 500 nm to about 1100 nm. It is noted here as well that any of the wavelengths of the emitted light may vary by about ±10 nm to about ±25 nm, about ±50 nm, about ±100 nm, and the like. Further, the order and combination of the emitters, along with the range of wavelengths they emit, can be arranged in any logical order, with the same advantages set forth above.

As depicted in FIG. 2D, the photoemitters 22 are aligned in parallel with the central photoreceiver 23. However, in various embodiments, any one of the photoemitters 22g, 22h, 22i, and 22j may be canted toward one another so as to form a “V” shape, or its reverse, or they may be rotated 90 degrees with respect to the photodiode, so as to form an “L” shape, as depicted in the array 20a of FIGS. 2B and 2C. Other arrangements, e.g., degree of angle running through the centerlines of a respective photoemitter 22/photoreceiver 23 dyad can also be used, such as from a 1-180 degree rotation one with respect to the other, such as determined to be beneficial for receiving a maximal reflectance of emitted energy waves back from the skin and/or so as to reduce the signal to noise ratio. As set forth above, in various instances, the photoemitter 22/photoreceiver 23 can be orientated to be aligned vertically with one another, e.g., 90 degrees, or 45 degrees, e.g., canted, with respect to one another, parallel to one another, or any angle there in between, as determined to be beneficial with regard to increasing signal reception and reducing noise.

As referenced above, each cover member of the housing 17 can be formed of a solid surface, where one or more of the surfaces is transmissive to electromagnetic radiation, such as where one of the cover members, e.g., a bottom cover member 17b, includes a window 21. For instance, in certain embodiments, a bottom surface of the cover member 17b, may include a window or other opening therein that is transmissive, e.g., transparent, to light from the emitters as well as that being reflected back from the skin tissues, and where the emitter is a sound generator, the window may be transmissive to sound waves and/or microwaves. In particular iterations, the housing 17, or a window thereof 21, may be made of reinforced glass in a manner that will allow the emitters 22, e.g., LEDs of the sensor pad of the PCBA 42, to direct light from the light-emitting diodes (LEDs) through the window 21 or the bottom of the cover member 17b to the skin.

Consequently, it is useful for the interior of the housing to be configured to position the PCBA 42, specifically, the sensor arrays 20a and 20b associated therewith, in a manner so that the photoemitter(s) 22 and photoreceiver(s) 23 are proximate the transparent window 21 opposite the user's body tissue. Specifically, since the measurements to be taken employ emitted electromagnetic radiation, which may be in the form of light waves, the arrangement of the energy emitters 22 and energy receivers 23 on the circuit board 42 in relation to the transmissive portion of the bottom cover should be such that light, and/or other electromagnetic radiation, is allowed to easily pass from within to outside of the housing 17 and back again. In this regard, the one or more photoemitters 22 are configured to illuminate, the user's tissue below the transparent window 21, and likewise, each of the one more photoreceivers are configured to receive a return of the light reflected back from the user's tissue below the transparent window. In such instances, the energization and/or control of the photoemitters may be such that the electromagnetic radiation emitted thereby is at a predetermined frequency, amplitude and/or intensity, as well as duration and/or interval of light emission.

Hence, the sensor unit 18 may include or otherwise be coupled with, a control unit, such as including a microcontroller, for controlling and/or modulating the characteristics of the electromagnetic radiation waveforms being emitted. This modulation may be performed intermittently, such as in response to a feedback loop becoming out of line, or in accordance with a determined pattern of modulation. In any of these instances, the one or more emitters and/or receivers will be activated by the controller so as to produce a determined pattern of emittance, such as where the pattern is determined to provoke a necessary response from the tissues and their constituents so as to better determine the values and characteristics of one or more biomolecules of interest and the body's response thereto.

In various instances, the light of different wavelengths can be emitted in a sequential manner and at a time periodicity so that the photoreceivers are capable of receiving, detecting, and distinguishing between the emitted lights of different wavelengths. Likewise, it is useful that the amplitude and intensity of the light being emitted is not too great so as to overwhelm, and thus drown or wash out the photoreceiver and/or the body tissue being observed. For these purposes, it is useful to calibrate the sensing device with the body, and then to modulate the level of intensity of the light so that although the body responds to the light of emittance, the response is not so great as to overwhelm the ability of the body to respond to individual light waves, such as in a characteristic manner. Therefore, the light and/or orientation of emitter/receiver dyad should be attuned to the degree of the body's response, such as through modulation of the angle and/or amplitude of the wave, such as through regulating the angle of transmission and/or reception, voltage, capacitance, and/or charge of each respective photoemitter and/or photoreceiver. In particular instances, the modulation of discharge of the emitters should be in accordance with a system generated pattern of emittance, which pattern sets forth all the parameters of emittance, of which emitters will be activated, when, for how long, for what duration, at what intensity, and/or in what sequence. This may be optimized body to body and molecule to molecule.

Consequently, in various embodiments, the pattern of light is generated by an analytics system, whereby a series of energy waves may be generated and directed into the skin, a response thereto is perceived, e.g., by one or more photoreceivers, the results are analyzed, and one or more of the characteristics being modulated is notated and then changed. For instance, a first pattern can be implemented, such as for calibration, and after the results thereof have been analyzed, then a new pattern may be formed, and a new series of emittance may be initiated. In this manner, the emitters, the wavelengths emitted thereby, their order and sequence of emittance, as well as their amplitude and/or intensity can all be finetuned to the particular biomolecule being observed as well as to the particular body that is responding thereto. All of this data may be observed, classified, and tagged, and can then be used to build a data structure, as disclosed herein, whereby each variable forms a node in the data structure, one or more correlations may be made between the various variables related to the system configurations and the results obtained, and the identified correlations can then be weighted. From this data structure a predictive model can be generated and implemented, and one or more determinations can be made, such as for generating a new pattern for configuring the system for the next round of emittance and monitoring.

The referenced modulation may be with respect to the number of photoemitters being employed, the wavelengths of energy being emitted from each emitter, their amplitude and intensity, the sequences of emitters being activated, such as with regard to their wavelengths, amplitudes, durations, angle of emittance, and the like. The modulation may be configured so that all variables are equal across emitters, such that their emissions are uniform, or in other instances, they may be non-uniform. For instance, the modulation may be variable with regard to a multiplicity of waveform characteristics being generated and directed into the skin. In various embodiments, the modulation of the emitters includes the controlling of the charging, capacitance, and/or voltage of the respective control and activation circuits of each of the photoemitters and/or receivers.

As different photoemitters 22 may emit light of different wavelengths, frequencies, amplitudes, and the like, it may be useful to distinguish light being emitted by an emitter 22, and light being received by a receiver 23. Specifically, it is useful to distinguish between the different wavelengths of light being emitted and received by different photoemitters 22 and different photoreceivers 23. For these purposes, in order to prevent light from one emitter 22 directly impinging on to a receiver 23, thereby flooding the receiver with ambient light, a light sink may be employed so as to form a barrier around one or more of the emitters 22 and/or receivers 23, such as each emitter and each receiver independently, or as one or more groups. Employment of such a light barrier is useful because it helps to prevent the photodiodes from being washed out, whereby the photoreceivers are overloaded with energy and cannot distinguish light of specific wavelengths, such as reflected light.

Therefore, in particular instances, a light sink is provided whereby the light sink is configured to surround an emitter and prevent undesired light penetration. The light sink or barrier may be formed of a non-transmissive material that circumscribes one or more emitters 22 or receivers 23, such as a metal, plastic, rubber, or other like, foam material. In various instances, the light barrier or sink may be a compressible material, such as a rubber or foam material that both surrounds the emitter and/or receiver, and is non-transmissive to various frequencies of light, such as to prevent non-reflected, infrared light impinging on the photoreceiver. For instance, in a particular embodiment, each photoreceiver 23 may be partially or completely surrounded, e.g., circumscribed, by a light barrier, such as made of rubber, which is configured to isolate the receiver from light being emitted from one or more, e.g., all, of the emitters.

Likewise, as can be seen with respect to FIGS. 2E and 2F, in various embodiments, it is useful to also modulate the transmissive qualities of the emitters 22 and or receivers 23. For instance, one or more of the emitters 22 and/or receivers 23 may include a filter such that only certain wavelengths of light are allowed to be emitted and/or received thereby. Hence, FIGS. 2E and 2F present iterations of one or more filter assemblies that can be employed in conjunction with the device set forth in FIG. 2B. Specifically, as can be seen with reference to FIG. 2B, a sensor unit 18 of the disclosure may include an arrangement of photoemitters 22a-f around a central photodiode 23a. In this embodiment, although collectively, the emitters can generate and emit light streams in a broad range of wavelengths e.g., from 200 nm to 500 nm to 1000 nm to 1700 nm, or more, each emitter may be coupled with a filter so that it only emits a small sub-range as compared to what the range would be if not filtered and/or in regard to the collective range of all emitters together. Therefore, to modulate this emission, a series of one or more filters 24 may be employed, such that only light of a narrow band will be allowed to be emitted through the filters 24 overlaying each discrete emitter 22a-f Therefore, in such instances, due to one or more of the bandpass filters 24a-f, the photoreceiver 23 will only receive light of discrete wavelengths, e.g., for which it may be programed or otherwise specifically attuned. The photoreceiver 23 may also be coupled with a filter 24 so as to ensure only light of desired wavelengths are capable of being detected.

Consequently, in an alternative embodiment, the reverse configuration may also be employed, such as where a broadband emitter replaces the receiver 23a. For instance, a single, broadband emitter 24 may be employed, such as where the emitter 24 is capable of emitting light in a broad range of wavelengths, such as from 200 nm to 500 nm to 1000 nm to 1700 nm or more. In such an instance, as depicted in FIGS. 2E and 2F, emitters 22a-22f may be replaced with narrow band photoreceivers 22a-22f, where each photoreceiver is configured for detecting discrete light waves. Consequently, in such an instance, a series of photodiodes 22a-22f are positioned around a central photoemitter 23a. A filter 24a-22f may then be placed over each photodiode 22a-f such that each photodiode can receive and detect a very narrow range of wavelengths. For example, in one embodiment, the filters 24a-24f may be configured for filtering light at the following frequencies: 1050 nm, 1200 nm, 1300 nm, 1450 nm, 1550 nm and 1650k, such as where each filter is configured to filter light at one or more of those ranges. Other arrangements are possible. For example, in one particular alternative embodiment, where the six photoreceivers 22a-f have been replaced with photodiodes 22a-22f, the first photodiode filter, 24a may filter light in the range from about 1000 nm and about 1100 nm, the second photodiode filter 24b may filter within the range from about 1100 nm and about 1200 nm, the third photodiode filter 24c may filter light in the range from about 1200 nm and about 1300 nm, the fourth photodiode filter 24d may filter light in the range from about 1300 nm to about 1450 nm, the fifth photodiode filter 24e may filter light in the range from about 1450 nm to about 1550 nm, and the sixth photodiode 24f may filter within the range from about 1550 nm to about 1650 nm.

The same is true with respect to FIG. 2E, where an alternate sensor arrangement is set forth whereby the photoemitters 22g-j have been replaced with photodiodes and are configured around a central broadband photoemitter 23b, which is capable of emitting light that spans the range from about 200 nm to about 1000 nm. In this embodiment, the central photoemitter is configured to emit a broad range of wavelengths from about 500 nm to about 1000 nm. Likewise, to modulate this emission, a series of bandpass filters 24g-j have been positioned over photodiodes 22g-j, such that only light of a narrow band will be allowed to be received through the filters 24 overlaying each discrete photodiode 22g-j. Therefore, each photoreceiver will only receive light of discrete wavelengths, e.g., for which it may be programed. For instance, in one alternative configuration, the seventh photodiode, e.g., 22g, may include a filter 24g that is configured for filtering light of a seventh wavelength, such as within the range from about 550 nm to about 650 nm, the eighth filter, e.g., 24h, is configured for filtering light of an eighth wavelength, such as within the range from about 650 nm to about 850 nm, the ninth filter, 24i, is configured for filtering light of a ninth wavelength, such as within the range from about 850 to about 940 nm, and the tenth filter, e.g., 24j, is configured for emitting light of a tenth wavelength, such as within the range from about 940 nm to about 1000 nm. In this configuration, the photoemitter 23b may be configured for emitting light waves in the range from about 500 nm to about 1100 nm.

In any of these embodiments and/or alternative configurations, once the reflected and/or refracted light is received, or otherwise collected by a corresponding photoreceiver, which in some instances may be filtered, a return signal may be generated, e.g., in response to collecting the reflected light. An on-board processing module 45, positioned on the printed circuit board 42, may then access and process the return signal to generate one or more digital read data. From this digital read data one or more characteristics of a biomolecule of interest may be determined, and/or one or more characteristics of a state of the wearer of the device, e.g., user, may also be determined, such as based on the body's observable response to the presence of that biomolecule within the tissues. In particularly instances, the body's response may be inferred from the spectral array produced by illuminating a portion of the body with one or more, e.g., a pattern, of waves of electromagnetic radiation.

Particularly, in various embodiments, the light sensors 23 may be configured as one or more miniaturized photodiodes that can receive reflected light and, in conjunction with the processing unit 45, can compare the emitted light to the returned light, and then use this information to detect, or otherwise determine, changes in color of the skin and surrounding tissues, such as caused by the presence of various molecules, e.g., glucose molecules, within the skin. For example, as indicated above, the presence of various molecules, such as glucose, within the blood, skin, and/or interstitial fluids may change the absorption and/or reflectance pattern of these structures in ascertainable, uniform ways. This uniformity may be within a single individual over time, or across multiple individuals. Consequently, continuous biomolecule measurements allow for patterns to emerge, which patterns can then be correlated with various conditions being experienced by the wearer of the device. These patterns can be correlated to such experienced conditions, and together, they may be correlated to the presence of one or more biomolecules being present within the body, which can be distinguished by the different spectral arrays produced thereby and observed by the wearing of the continuous biomolecule sensing and monitoring apparatus disclosed herein.

Specifically, in various instances, spectral patterns of light, sound, and/or microwaves, can be observed while both in and not in a particular state, such as while an individual is experiencing conditions pursuant to a condition like hyperglycemia, and when not experiencing such a condition. These patterns will change based on the state of the individual and the characteristics of the biomolecule(s) being present. For instance, it has been observed that these patterns change in the presence and non-presence of certain biomolecules, such as glucose. More particularly, these light absorption and reflectance patterns differ based on one or more of the conditions of the wearer, as well as the various different biomolecules being present within their tissues. This change in spectral pattern is observable and quantifiable, such as by bombarding a skin tissue with a number of different light waves and intensities in a number of different patterns so as to derive a host of different body responses to the different waveforms and intensities being emitted. Thus, in some embodiments, a number of light emitters may be employed, e.g., sequentially, so as to produce various different patterns of observable reflectance and/or absorbance, e.g., from a number of different photoreceivers, and in view of these different patterns the quantity and level of various molecules, e.g., of glucose, within the skin, and their effect on the body, can be calculated and determined.

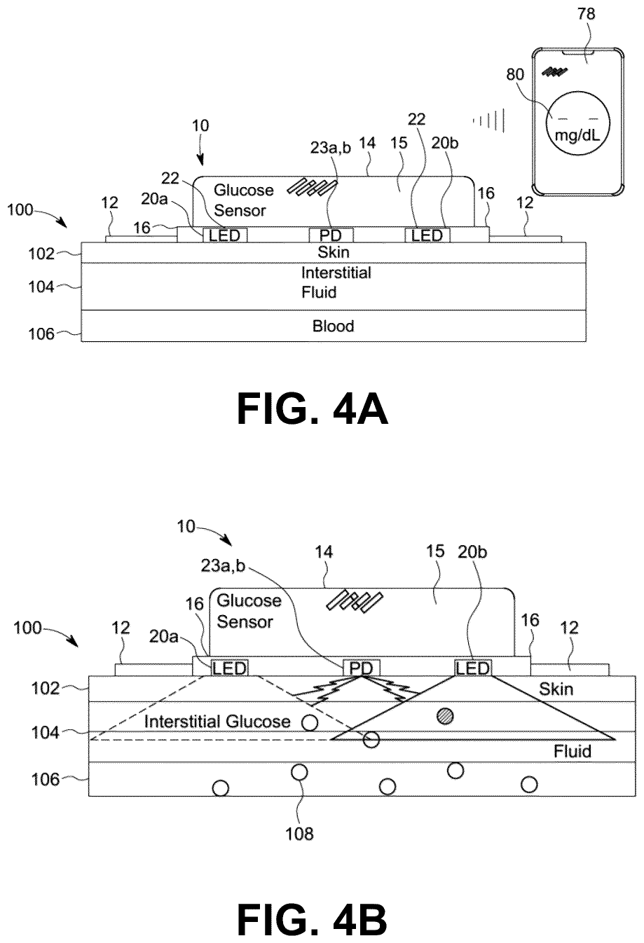

In provoking various different responses from the body, various different patterns of light emittance and reception, as well as light intensities and amplitudes from the electromagnetic light sources, can be employed. Therefore, according to another aspect of this disclosure, as can be seen with respect to FIGS. 4A and 4B, there is provided a method for sensing and monitoring a biomolecule present within the fluids surrounding the tissues of a body of an individual. Specifically, in various embodiments, the apparatuses, devices, and systems set forth herein may be employed to detect biomolecules, such as metabolites, e.g., glucose, within the interstitial fluids surrounding the skin cells and, in some instances, present in the blood flow.

In one example embodiment, a metabolite of interest to be sensed and observed is glucose, and through such continuous observation one or more states, e.g., glycemia, hyperglycemia, pre-diabetes, diabetes, and the like, can be detected and monitored. Likewise, through such continuous monitoring, the condition may more effectively be managed. It is to be noted that although herein below, and throughout this disclosure, glucose is often referenced as the molecule of interest to be detected and monitored, other metabolites, such as other sugars, e.g., fructose, alcohol, aldehydes, alkaloids, ketones, and the like, as well as their effects on one or more states of a body, can also be detected and monitored, and the concomitant effects on the body can likewise be managed, as herein described.

Accordingly, in view of the above, provided herein is a sensing and/or monitoring device 15, which can be coupled with one or more of an encasement member 14 and/or attachment member 12 that is configured for effectuating the attachment of the sensing and monitoring device 12 in a position on the skin whereby one or more electromagnetic waves may be emitted through the device housing 17 and into the skin. In certain instances, the attachment structure 12 may function to attach the sensor device 15 directly to the body itself, but in other instances, the interaction of the attachment structure 12 may be mediated through its coupling with an attachment encasement or framework member 14, which is configured for making attachment of the device 15 to the body more comfortable. In any of these instances, the attachment of the sensing device 15 to the body should be such that once placed thereon the sensing device 15 does not move with respect to the body, unless the entire apparatus 10 is being removed.

For instance, in certain instances, as can be seen with respect to FIGS. 3A-3B, the attachment structure 12 may be a planar member with an opening to which an encasement framework, such as in the shape of a dome 14, may be coupled so as to enclose the opening on one side. In such a configuration, as shown in FIG. 3A, the sensing and monitoring device 15 may be passed through the opening in the attachment structure 12 so as to be fitted snuggly into a cavity of the encasement member 14. Once fitted therein, the entire apparatus 10 may then be coupled to a user's body, such as through a suitably configured attachment element 13. Particularly, in one embodiment, an adhesive 13 may be applied between the skin facing surface of the attachment structure 12 and the body portion 100 to which it is to be attached. In certain other embodiments, the attachment structure 12 may be configured as patch, a bandage, a sleeve, or other substrate-like device.

More particularly, as described above with reference to FIGS. 3A and 3B, in one embodiment, the sensor and/or monitoring apparatus 10 may be composed of 3 main components: a framework or dome member 14, an attachment structure 12, and the sensing and/or monitoring device 15. In particular instances, the framework member, e.g., dome 14, is configured so as to immovably encase the sensing device 15, and to additionally be coupled to the attachment structure 12, such as via a suitably configured attachment element 13. The attachment element may be any suitable element for coupling the dome 14 to the attachment structure, for example, an adhesive, a fastener, e.g., a hook and loop fastener, a clip, button, a zipper, a band, a stretchable sleeve, and the like. In one particular embodiment, as can be seen with respect to FIG. 2A, the dome or disc member 14 may be coupled to the attachment structure 12 through the addition of an adhesive element or ring 13, such as a ring having adhesive on both sides. In such an embodiment, the encasement member 14 may have an attachment interface 16 that is an extended element at the base of the encasement dome 14, such as where the extended element is less than about 10 mm, less than about 8 mm, less than about 5 mm, less than about 3, or 2, or 1 mm, and the like, in length, and to which the attachment structure 12 may be coupled, such as through an intermediating adhesive element 13.

Together, the framework dome 14 and attachment structures 12 are configured for associating the sensing and monitoring device 15 on to a base, which base in most instances may be a living body part of a wearer. However, in actuality, the base can be any surface that is permissive for penetration by light and/or sound and within which resides volatile elements the presence of which can be measured, such as by reflectance and/or a change in spectral reflectance due to a volatile element being present within the base. Specifically, the attachment structure 12 may be configured for attaching the sensing and/or monitoring device 15 to a portion of a wearer's body, such as through an appropriately applied adhesive, tape, e.g., double sided tape, or by tying, clipping, latching, wrapping, and the like.

As discussed above, the attachment structure 12 may be configured for effectuating the coupling of the sensing device 15 to a base member, e.g., a body, part. In many instances, this coupling is facilitated through encasing the sensing device 15 within a domelike framework member 14, as shown in FIG. 3B. However, in other embodiments, the attachment structure 12 may perform the coupling without recourse to an encasement member 14, as set forth in FIGS. 3C and 3D. In such instances, the biometric sensing and/or monitoring apparatus 10 need only include the attachment structure 12 along with the sensing and monitoring device. For instance, as can be seen with respect to FIGS. 3C and 3D, in certain embodiments, a biometric sensing and/or monitoring apparatus 10 is provided, wherein the apparatus includes an attachment structure 12 that is donut shaped, such that the sensing and monitoring device 15 can be snuggly fitted in through a central opening in the surface of the attachment structure, as shown in FIG. 3C, and then the apparatus may be applied to the body 11, so as to securely hold the sensing device into substantial immovable proximity to the skin of the body. In certain instances, the securing of the sensing device 15 to the body in a manner that it is substantially inhibited from moving is by the inclusion of a stiff ring-like component 8, which gives structural firmness and support to the otherwise flexible attachment structure 12.

In one particular embodiment, as shown in FIGS. 3A and 3B, the attachment structure 12 may be configured as an elongated surface member containing, or otherwise being associated with, an adhesive layer 13. Specifically, in various embodiments, the attachment structure 12 may be an elongated, flexible member, such as configured as a patch. In particular embodiments, the elongated surface of the attachment structure 12 may include one or more structural elements 8, that are configured for giving the surface firmness and integrity. In particular instances, the structural support member 8 may extend laterally along an X and/or Y plane of the attachment structure 12. However, in other instances, the structural support member 8 may be a circular element that circumscribes the opening through which the sensing device 15 is inserted, as shown in FIGS. 3C and 3D. In any of the aforementioned embodiments, the attachment structure 12, with or without the framework member 14, may be adapted for keeping the sensor and monitoring device 15 in place on the body, e.g., on the back of the arm, in a substantially unmoving coupling during a prolonged period of time, e.g., a 7-, 14-, 21-28-day period, during which time period, the sensor and/or monitor may be actively or passively taking periodic or continuous measurements.

For instance, FIG. 4A generally illustrates the placement of a sensor and monitoring apparatus 10 on the skin of a subject. Particularly, as depicted, the sensing and/or monitoring device 15 of FIG. 2B may be encased within an encasement framework 14 that is configured as a dome or a disc. The dome 14 in turn is associated with an attachment structure, such as a patch member 12, which patch member 12 is used to securely position the apparatus 15 on the surface of a body part 100, such as the back of the arm, as shown in FIG. 6A. In such a position, the sensing and/or monitoring apparatus may be configured to detect, measure, and/or otherwise determine activity of a biomolecule, such as glucose, within the tissues, vessels, and/or interstitial spaces of the body of a subject.

For these purposes, as can be seen with respect to FIG. 4A, the sensing and monitoring apparatus 10 is placed such that a transmissive portion of the bottom 17b of the housing 17 of the sensing device 15 abuts the skin's 100 surface. In particular instances, the bottom member 17b of the housing 17 includes one or more windows 21 therein, which window(s) contacts the skin in a manner so that the encased sensing and monitoring device 15 has visual access to the body tissues. Specifically, as can be seen with respect to FIG. 4B, once firmly applied to the body portion, the sensor unit 18 within the housing 17 is engaged such that one or more light sources thereof, such as a plurality of arrays 20a, 20b having a plurality of electromagnetic radiation emitters 22, e.g., light emitting diodes, are energized and emit light waves that pass from the inside of the housing 17, out through the transmissive portion, e.g., window, 21 of the bottom surface 17b and into the layers of the skin 102 and 104. Likewise, the sensor array 20 includes a number of electromagnetic radiation receivers, such as a plurality of photodiodes 23 a, b, for detecting and receiving the electromagnetic radiation being reflected back from the skin 102 and interstitial fluids 104 therein.

As can be seen with respect to FIG. 4B, the sensor unit 18 is configured as a glucose monitor, and because glucose is a visible (e.g., green and/or red) and/or infrared, light-active component, it has been found, herein, that spectroscopy can be used to detect glucose in dermal tissue 102 and interstitial fluids 104. Consequently, in use, the sensor unit 18 of the biometric sensing and monitoring apparatus 10, initiates a first array of LED emitters 20a to direct a first series of lights (such as on or more of visible, green, red, near infrared, and/or infrared light) into the tissue 102 and interstitial fluid 104, as shown. This first emittance by array of LEDs 20a is demarcated by the broken lines. Then, the second array of LED emitters 20b is energized and emits a second series of lights into the tissue 102 and interstitial fluid 104. This second emittance is demarcated by the non-broken lines. Likewise, as shown, the sensor unit 15 includes one or more light based receivers, e.g., photodiodes 23 a, b, that are configured for receiving the various different wavelengths and intensities of the light reflected back from the tissues 102 and interstitial fluids 104. In this embodiment, the different arrays 20a and 20b emit light sequentially, and at a depth whereby the emitted light does not penetrate into the vessels and blood. However, in various other embodiments, one or more of the arrays may be configured for emitting light that impinges all the way down into the vessels and/or tissues, such as into the blood vessels, and where a multiplicity of light sources are activated at the same time, such as set forth in FIG. 5E. This configuration is useful, such as for determining blood glucose levels, such as depicted in FIG. 5D.

In any of these instances, the photodiodes 23, are configured to collect raw reflected electromagnetic radiation, as shown, from the different layers of the interstitial fluids throughout the skin layers, and then an associated analog to digital converter converts the raw reflected electromagnetic radiation data into digital signal read data. An on-board processor 45 may then analyze the raw light data that is reflected back from the various different layers of the skin, tissues, and vessels, and a communications module 43 may then transmit the data to an associated computing system 70 for analysis, e.g., spectral analysis, thereby, as depicted in FIG. 4B. Specifically, these spectral signals detected by the sensor's photodiodes are affected by a complex relationship that exists between various different biomolecules, such as glucose, being present in the interstitial fluid and/or the blood of the body's tissue, and the frequency, intensity, and exposure time of the light being directed into that tissue.

As can be seen with respect to FIG. 4B, this complex relationship represents a “glucose-mediated skin response,” from which a person's present glucose characteristics, e.g., levels and/or concentration, may be determined. In such implementations, the one or more photodiodes may be configured to receive at least a portion of a first light emitted and directed into the skin by one or more of the LEDs and reflected back therefrom into the sensor. In like manner, the photodiode, on-board, and/or other associated computing system, may further be configured to receive and/or analyze at least a portion of the second, third, fourth, fifth, sixth, tenth, etc. light being reflected and/or refracted back from the skin of the user.

Particularly, in various embodiments, the plurality of photo-arrays 20a and 20b, may include a number of LED light emitters, such as where the first LED array 20a includes six light emitters, and the second LED array 20b includes four light emitters, as embodied by the sensing device depicted in FIG. 2B. Likewise, each sensor array 20a, 20b may include one or a series of light sensors, e.g., photodiodes, wherein each light sensor is configured to receive light of the various wavelengths emitted from the ten (or more) LED emitters and reflected back from the body. Consequently, where a sensor array 20 includes a series of light sources for emitting a series of light of the same or different wavelengths, then the sensor array may further include one or more light receivers, where each light receiver may be configured for detecting and receiving reflected or refracted light in the same or similar wavelengths.

Accordingly, with respect to FIG. 4B, as set forth above, light from the various different, e.g., 10, LED emitters may be emitted and/or received at different predetermined or random frequencies, such as where the light emittance is sequential and in accordance with a pre-defined pattern. For example, each light emittance and/or reflectance/refraction may be pulsed, the durations of emittance, or pulsing, between the emitters, may be the same or variable, and/or the intensities of the light emitted may also be the same or variable. These different variables may be determined and formed into an emittance protocol as determined by the analytics system herein, such as based on the emittance patterns that are predicted to best determine the presence, concentration, and/or bodily effect of a biomolecule within the skin tissues. Consequently, the light to be emitted from the various light emitters may all be of the same or of different wavelengths, the duration of emittance may be the same between all emitters or different, e.g., the lights may be pulsed at different frequencies, and the sequence of light emittance may be sequential, e.g., based on wavelengths or durations, and likewise, the intensities may be the same or different, all of which may be determined by the analytics system and/or controlled by an on-board computing system and/or microcontrollers.

For instance, as indicated, the sensor unit(s) 18 and/or arrays 20 thereof may be coupled together with, and/or otherwise include one or more printed circuit boards 42, which in turn may be associated with a processing module 45 that may include one or more processing units 47 and/or microcontrollers. Particularly, the processing module 45 may include one or more semiconductor chips that may be configured as a system on a chip, and thus, may integrate or otherwise be coupled with the various light emitters, e.g., LEDs, light receivers, e.g., photodiodes, converters, and other electronic components herein disclosed.

Regardless of the specific configuration, the relative positioning of the emitters 22 (e.g., relative to adjacent or nearby emitters 22, relative to other components such as a photodiode or other energy receiver 23). For example, one, some, or all of a given group of emitters 22 can be positioned at a single, common distance away from a common (and/or nearest) energy receiver 23. Alternatively or in addition, one, some, or all of a given group of emitters 22 can be positioned at distances away from a common (and/or nearest) energy receiver 23. For example, a first emitter 22 can be positioned at a first distance from the energy receiver 23, a second emitter 22 can be positioned at a second distance from the energy receiver 23 that is greater than the first distance, a third emitter 22 can be positioned at a third stance from the energy receiver 23 that is greater than the second distance, and so forth.

Alternatively or in addition, one, some, or all of a given group of emitters 22 can be configured to focus its light output in different orientations and/or configurations. For example, one or more of the emitters 22 can include a specialized lens configured to orient and/or focus outputted light to one or more corresponding regions of skin (e.g., within an array area, which can be a particular area of skin defined by an outline of the emitters 22 of a given array 20 and/or a predefined area including the emitters 22 of the given array 20).

Alternatively or in addition, one, some, or all of a given group of emitters 22 can be positioned at a constant height (e.g., relative to an outermost surface of the monitoring apparatus 10 that is configured to contact the user's skin). Alternatively or in addition, one, some, or all of a given group of emitters 22 can be positioned at different heights. For example, a first emitter 22 can be offset a distance from the skin of the user when the monitoring apparatus 10 is being worn by the user, whereas a second emitter 22 can be positioned such that it is flush with, or approximately flush with, the outermost surface of the monitoring apparatus 10 that is configured to contact the user's skin and/or flush with, or approximately flush with, the user's skin (e.g., contacting the user's skin). Alternatively or in addition, one, some, or all of a given group of emitters 22 can protrude from or past the (otherwise) outermost surface of the monitoring apparatus 10 that is configured to contact the user's skin. Stated differently, one or more of the emitters 22 can protrude outwardly from the monitoring apparatus 10 such that the emitters 22 push slightly into the skin of the user when the monitoring apparatus 10 is being worn.

Further, in various embodiments, the processing module 45 may include, or otherwise be associated with, an AI module 72, such as incorporating a machine learning engine from which one or more models may be generated. For example, a first model may be generated and used to determine an emittance pattern and schedule, e.g., characterizing the conditions and variables of light emittance, and a second model may be generated to collect and analyze the returned spectral data. This data may then be employed to generate a third and/or fourth model by which an inference engine may then predictably determine a new pattern of emittance as well as determine a level, e.g., a concentration, of a biomolecule within the tissues of a wearer of the device and/or to predict a state of their being, such as based in part of the various different patterns of light emittance.

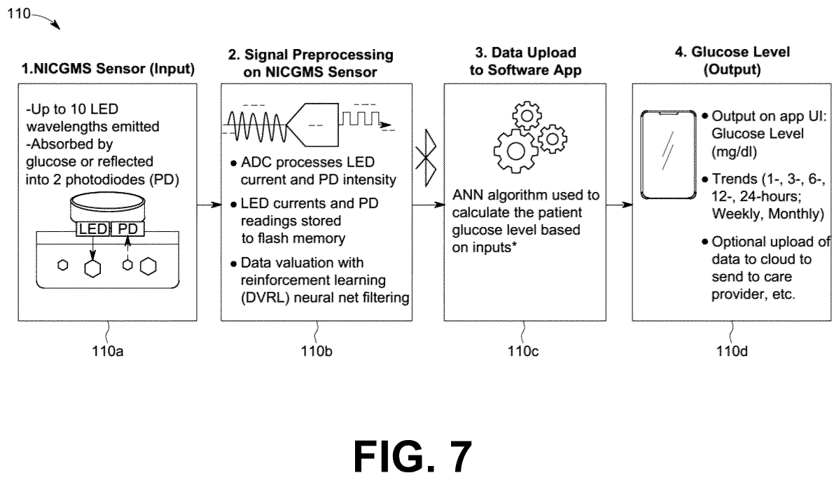

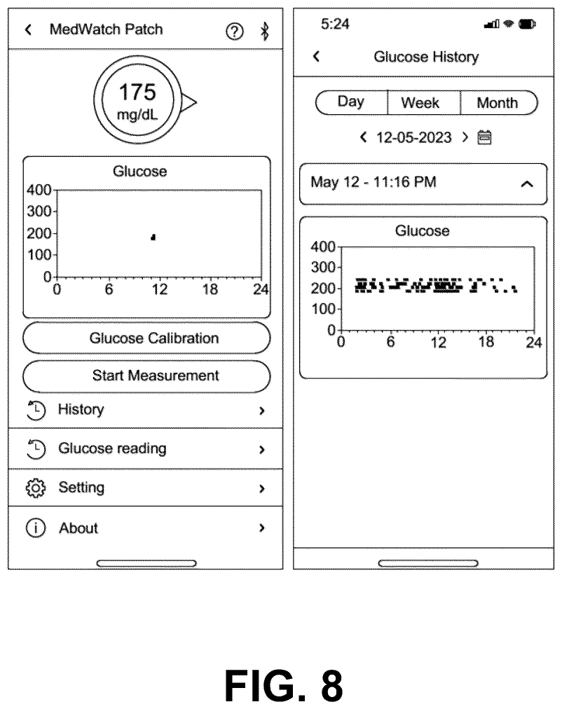

Hence, in one exemplary embodiment, as can be seen with respect to FIGS. 4A and 4B and also FIGS. 5D and 5E, a non-invasive glucose sensing and monitoring apparatus 10 of the disclosure may be attached to the skin 100 of a user and may be used to sense and monitor glucose levels and their effects on the body. Likewise, along with the systems herein disclosed, the effects of glucose on the body may not only be observed but can be managed to prevent and/or ameliorate the onset of adverse conditions such as hyperglycemia, pre-diabetes, diabetes, and the like. For instance, the raw collected and/or preprocessed (or processed) read data may be transmitted to a computing system 70 of the system 1, whereby the data may be further processed and/or displayed so that a user may view and track the data over time, such as through a biomolecule monitoring client application 80 running on their mobile device 78, as shown. See also FIGS. 7 and 8. Likewise, in view of the analyzed data, the system may give practical guidance to the user so as to better manage the one or more states, e.g., diabetes, identified.

Consequently, in view of the above, the methods herein disclosed generally include placing the biometric sensing and monitoring apparatus, such as set forth in FIG. 2B or 5A onto a body part, such as an arm, as set forth in FIG. 6A, and then employing a sensor 15 of the device to collect data on the user. In this case, as can be seen with respect to FIGS. 4B and 5D, the data may include light measurements of interstitial 104 and/or blood 106 glucose values. These measurements may then be analyzed by a computing system 70 and one or more characteristics of glucose are determined and calculated, such as an interstitial and/or blood glucose level, e.g., concentration, of the user. As described in greater detail herein below, these analyses and calculations may be performed by analytics system 71 such as including an AI module 72, for instance, implementing a machine learning and inference generating method based on the collected spectral data. The results of the analysis may then be outputted, such as via a mobile computing device 78 of the user, as shown in FIGS. 7 and 8. In various embodiments, the output may also be to a healthcare professional, whereby together with the user, compliance and health may be maintained and tracked over time.

Accordingly, in some implementations, the method may include detecting and measuring the values and levels of biomolecules, such as glucose, within the interstitial fluids 104 and/or blood 106, whereby the steps may include: emitting a first light at a first wavelength, a second light at a second wavelength, a third light at a third wavelength, a fourth light at a fourth wavelength, a fifth light at a fifth wavelength, a sixth light at a sixth wavelength all the way up to ten or more lights of ten or more wavelengths being emitted, such as from one or more arrays of one or more photoemitters. In particular embodiments, the emitter of the light may be from one or more light emitting diodes. Likewise, the method may further include receiving at least a portion of the first light being reflected back from the skin of the user, at least a portion of the second light being reflected back from the skin of the user, at least a portion of the third light being reflected back from the skin of the user, at least a portion of the fourth light being reflected back from the skin of the user, at least a portion of the fifth light being reflected back from the skin of the user, and at least a portion of the sixth light being reflected from the skin of the user, all the way up to receiving ten or more lights of ten or more wavelengths being reflected back and collected, such as from one or more arrays of one or more photoreceivers. In particular embodiments, the receiver of the light may include one or more photodiodes.

Further, once the light data has been collected the method may include determining a first reading corresponding to the amount of the first light being absorbed and/or reflected back by various components within the interstitial fluid, blood and/or within the skin, and a second reading corresponding to the amount of the second light being absorbed and/or reflected back by various components within the interstitial fluid, blood and/or within the skin, and a third reading corresponding to the amount of the third light being absorbed and/or reflected back by various components within the interstitial fluid, blood and/or within the skin, and a fourth reading corresponding to the amount of the fourth light by various components within the interstitial fluid, blood and/or within the skin, and a fifth reading corresponding to the amount of the fifth light being reflected back by various components within the interstitial fluid, blood and/or within the skin, and a sixth reading corresponding to the amount of the sixth light reflected back by various components within the interstitial fluid, blood and/or within the skin. These steps may be repeated for all light of all wavelengths being emitted into the skin, reflected back from the interstitial fluid, blood and/or within the skin, and received by one or more light receivers. Once the light data has been received, it may be analyzed by a computing system of the system so as to calculate one or more levels of one or more biomolecules within the tissues and fluids thereof, from which one or more states of the user of the apparatus may be determined, and/or one or more remedial actions may be suggested.

In particular embodiments, the biomolecule sensor and/or monitor 15 may include a processing module 45 including one or more processors, such as a plurality of processing elements 47, e.g., forming one or more processing engines 46, which may be coupled to or otherwise be associated with the plurality of energy emitters and energy sensors for collecting data therefrom. In such an instance, the on-board processing module 45 may be configured for one or more of pre-processing and/or processing the raw light and/or read data, and in some instances may calculate a first iteration of a biomolecule value calculation, such as including one or more of the presence, concentration, and/or activity of the biomolecule within the tissue. However, in other instances, these calculations may be performed, or may be continued to be performed, such as by an off-board computing system 70. Hence, in certain instances, the sensor unit 18 itself, or an associated computing system 70 associated therewith, may be configured to determine the presence and value of biomolecules, such as glucose, and the processing unit 45 and/or associated computing system 70 may be configured for determining tissue (or interstitial space) and/or blood glucose levels, e.g., based on the collected data. In certain instances, the processor module may be adapted to calculate or otherwise determine glucose (or other biomolecule) values, e.g., levels, concentrations, and the like, over time, such as using machine learning, e.g., based on collected and the user's historical health data.

Further, in various instances, to better determine biomolecule levels and/or states of the individual, e.g., with respect thereto, the continuous biomolecule monitoring apparatus 10 and/or device 15 may be associated with an external computing system 70, such as where the computer system 70 implements an artificial intelligence module 72 that is configured for receiving the raw sensed data, e.g., reflected light data, raw read and/or digital read data, pre- and processed data, and/or other associated data, which, once received by the computing system 70 may be analyzed, and once analyzed, a biomolecule value, level, concentration, and/or one or more other characteristics, may be calculated and determined. From the results of the analysis of this data, one or more states of the individual may be assessed. For instance, the Artificial Intelligence (AI) module may include a machine learning engine and/or an inference engine, such as where the machine learning engine is configured for training the system, and the inference engine is configured for making a prediction based on such training.

In such an instance, one or both of the machine learning and inference engines can be embodied by a data structure, such as including an Artificial Neural Network (ANN). Once the continuous biomolecule monitoring device is positioned and secured next to the skin, such as illustrated in FIGS. 4A-4B and/or 5D-5E, an internal mapping, called a signature, can be performed so as to identify the active region wherein measurements from the device will be taken. Specifically, in specific embodiments, performing the initial signature analysis may result in one or more neural net assessments of the topology of the skin, other body tissues, interstitial fluid(s), blood, and/or biomolecules therein.

Once the mapping has been performed, readings can be taken in a uniform manner. When secure, the topology continues to be relevant, and the measurements are appropriately accurate based on that topology. If the device is subsequently moved, then a new topology mapping can be performed, or the effects of the movement may be corrected mathematically by the AI module. The patch configuration, as represented in FIGS. 3B and 5B are configured to overcome this problem by localizing the device to a specific location, allowing the signature to be performed and the topographical map to be generated. Once the topology has been mapped, then a plurality of measurements may be taken, as described herein.

As presented in the embodiments of FIGS. 2B and 4A-B, the biometric sensing and/or monitoring device 15 may include a sensor unit 18 that includes a plurality of sensor arrays 20a and 20b. As depicted, the first array 20a includes six energy emitters 22a-f, and the second array 20b includes four energy emitters 22g-j. In particular implementations, the energy emitters 22 are composed of light emitting diodes. Likewise, each sensor array 20a, 20b includes one or more energy receivers 23a and 23b, such as at least one photo receiver. This dual array configuration is useful because it allows for a wide range of light waves to be emitted in sequential or other determined order, and thus, allows for a large number of spectral data to be generated and analyzed. However, in other instances, as depicted with respect to FIGS. 5A-5E, a photoplethysmography (PPG) sensor array may be included or may be substituted for one or more of the arrays 20a and/or 2b.

For instance, as can be seen with respect to FIG. 5A, in one embodiment, the sensing and monitoring device 18 may include the six-emitter, 1 photodiode sensor array 20a as depicted in FIG. 2B. However, in this embodiment, the four-emitter, 1 photodiode sensor array 20b has been replaced with a PPG sensor 35, as explained in greater detail herein below. It is noted that in the embodiment of FIG. 5A the sensor unit 18 further includes a number of other electronic components 60. In that regard, although such electronic components 60, as described below are with reference to the embodiment of FIG. 5A, any of these components may be included in addition to, or substitution for, any of the components set forth with regard to FIG. 2B.

Consequently, the substitution of a PPG array 35 for the four-emitter array of 20b, will allow for a variety of different data to be considered when taking the measurements, analyzing the results thereof, and when making the determinations recited herein. Specifically, as discussed above, use of one or more arrays of photoemitters, such as 3, 4, 6, 8, 10, 20, 30, 50, or more, is useful for generating a broad spectrum of visual, e.g., reflectance, data that can be produced by directing light of a plurality, e.g., 5, 10, 20, 30, 50, of different wavelengths into the skin. However, light of such wavelengths can be generated and emitted from a plurality of different emitters, which can all be miniaturized and positioned on a single array, e.g., 20a (or 20b). In addition to the photoplethysmography (PPG) array 35, as can be seen with respect to FIGS. 5A-5B, a second array, e.g., 20b (or 20a), may also be included, such as where the second array may be included in addition to a photoplethysmography (PPG) array 35.

Having these two arrays, e.g., a photo-array and a PPG array, together, is useful because they produce different data at different depths of the skin. Generally speaking, the photo-array produces skin reflectance data, which may be indicative of the presence of one or more biomolecules of interest, whereas the PPG array produces additional data pertaining to the heart and cardiac activity as well as the response of the skin thereto, which again may be affected by the presence of the biomolecules of interest under observation. These two different sensor configurations, therefore, collectively produce a more holistic view of what is going on within the tissues and how the body they are responding to the presence of various different biomolecules. Specifically, the resistivity of the skin and/or vessels, in the presence of biomolecules within the interstitial fluids and blood, during the cardiac cycle, produces a wealth of spectral data that can be analyzed and used to determine the characteristics of a number of biomolecules. This data can be used to generate a pulse-wave-velocity analysis by which cardiac function and/or vessel health may be assessed. Other cardiac relevant measurements and data can also be generated.

However, as can be seen with respect to FIGS. 2A and 5B, an important feature of the devices disclosed herein is the miniaturization of the various components of the arrays 20 and electronics 60 of the sensor device 15, as well as their arrangement on a single or double-sided printed circuit board 42. This arrangement is unique and useful because it allows for the form factor of the sensing and monitoring device 15 to be as small as possible, while at the same time as encasing the electronics 40 and componentry 60 necessary for taking a broad range of measurements so as to more accurately determine the presence and effects of biomolecules in the body. Hence, the various different components of the device have been designed to occupy a very compact internal space within the housing.

That being the case, the various electronics 40 and components 60 may be positioned on both sides of a double-sided printed circuit board 42 and may be run from a single or double power supply 48, which may be positioned on one side of the one or double-sided printed circuit board 42, while the other side of the double-sided printed circuit board 42 may include sensor arrays 20a and 20b and the other electronic components of the device. For instance, in various instances, the one or more electronic components 60 may include one or more sensor arrays 20, PPG sensors 35, galvanic skin response sensors 69, analog to digital converters 41, one or more processing modules 45, a communications module 43, temperature sensor 64, as well as one or more auxiliary electronic devices, including: an accelerometer 61, gyroscope 62, SPO₂ assembly 63, EKG electronics module 66, and the like. As shown, all of these components can be optimized for size and concisely arranged to be snuggly fitted within the housing.

For instance, similar to the embodiment set forth in FIG. 4B, the sensor unit 18 of FIG. 5A includes an electromagnetic energy emitting array 20a that includes a number of energy emitters 22 and a number of electromagnetic energy receivers 23, such as including one or more photodiodes. So being, like the sensor unit described with reference to FIG. 4B, the non-invasive, continuous biomolecule monitoring and sensing unit 18 of FIG. 5A may include a series of photo arrays 20a, 35 that direct visible, near infrared, and/or infrared light at the skin in a pattern of different light frequencies, intensities, and/or durations using the photo-light/PPG arrays. For example, one or more of the light array(s) may include one or more light emitting diodes (LEDs) and/or light receiving diodes, photodiodes. In various embodiments, the one or more photo arrays, e.g., 20A, may include 1, 2, 3, 4, 5, 6, 7, 8, 9, 10, 11, 12, 15, or 20, or even 30 or more (including numbers therebetween) photo emitters 22 and/or photodiodes 23, where each photo emitter 22 emits light and each photodiode 23 receives light, of a same or different wave length, sequentially or in a determined or random pattern, such as in a pulsing manner, where the durations may be the same or different, as described above with reference to FIG. 2B.

Particularly, in the iteration of FIGS. 5A and 5B, a first photo-array 20a of photo emitters may be included, such as where the photo-array includes a number of LEDs from 3, 4, to 5 to 6 up to 10 or more LEDs, such as where the photodiodes of the photo-array may include from 1 to 3, 4, up to 10 or more photodiodes. As depicted, the photo-array 20a includes 6 LEDs, but it could easily include up to 10 or more LEDs, which are positioned on a single array 20a. In other instances, however, the 6 to 10 or more LEDs may be distributed throughout two or more arrays, as shown in FIG. 2B. In this regard, as depicted the photo-array 20a includes 6 photoemitters, such as where the series of emitters includes a first, second, third, fourth, fifth, and sixth light emitting source, e.g., LEDs, such as where each light source emits light of a different wavelength. For instance, the 6 LED light array may be configured for emitting light of: a first wavelength, which may be about 1000 to about 1200 nm, or less, light of a second wavelength, which may be about 1300 nm more or less, light of a third wavelength, which may be about 1450 nm or more, light of an fourth wavelength, which may be about 1500 nm more or less, light of a fifth wavelength, which may be about 1550 nm or more, and/or light of a sixth wavelength, which may be about 1650 nm or more. In some embodiments, any one of the light sources may be an LED, and the wavelength of the emitted light may vary by about ±10 nm to about ±25 nm, about ±50 nm, about ±100 nm, and the like.

In various instances, along with the 6 LED light array, an additional 4 emitters may be included, such as on the same 6 LED array, making it a 10-emitter array, or the additional 4 emitters may be included, such as on an additional array, e.g., 20b, such as shown in FIG. 2B. In either instance, the additional 4 photoemitters may include a first, second, third, and fourth light emitting source, where each light source emits light of a different wavelength. For instance, the 4 emitter-array may be configured for emitting light of a first wavelength, which may be about 550-60 nm or more, light of a second wavelength, which may be about 650 nm more or less, light of a third wavelength, which may be about 850 nm or more, and light of a fourth wavelength, which may be about 940-50 nm more or less. It is to be noted, that any mixing and matching of emitters in differing combinations is permissible, and certain emitters may be omitted. In various instances, the one or more photo-arrays may include 10 emitters or more, 9 emitters, 8 emitters, 7 emitters, or less, and in any of these instances, the arrays may be separated into 2, 3, or 4 or more groupings of emitters emitting the same or different wavelengths. In various embodiments, a pulse photoemitter may be included, such as where the pulse photoemitter is configured to emit a first light at a first wavelength and a second light at a second wavelength, a third light at a third wavelength, a fourth light at a fourth wavelength, a fifth light at a fifth wavelength, a sixth light at a sixth wavelength, and so on, which lights may be emitted from the same or different photoemitters, from the same or different arrays.

Likewise, as described above, along with a series of one or more light emitter arrays 20a and/or 20b, each sensor unit 18 may further include one or more light sensors 23, such as one or more photodiodes. For example, each sensor array 20 may include one or more, e.g., two, photodiodes, such as a first photodiode that is configured for receiving and detecting light waves in the range of about 1000 nm-1700 nm, and/or a second photodiode that is configured for receiving and detecting light waves in the range of about 500 nm-to about 1100 nm. However, although two photodiodes have been described, only one or more than two may be included, such as three or four photodiodes, where each photodiode is attuned to sensing wavelengths equally split between 500 nm-1700 nm. In other embodiments, five or six or seven up to ten or more photodiodes may be included, such as where each photodiode is attuned to its own dedicated photoemitter wavelength.

In addition to the above, as set forth with respect to FIG. 5C, an additional array 35 may also be included, such as where the additional array is configured as PPG array 35. In various embodiments, one or more of the above-mentioned arrays may be omitted. The PPG sensor 35 may include an electromagnetic radiation emitter array 36 and an electromagnetic radiation receiver 37, which in this instance may be arranged linearly, e.g., in a lateral line, in relation with one another. In this regard the photoemitter array 36 may be composed of three emitters, such as LED emitters, which are configured for directing light into the tissues of the body. In a particular iteration, the three-emitter array 37 may be configured for emitting light of one or more of a green, red, near-infrared, and/or infrared wavelength, such as where at any given time the three LEDs 36a-b emit light of different wavelengths. However, the emitter 36/37/receiver array may include any reasonable number of emitters and receivers, deploying any suitable wavelengths for emission and collection.

One or more converters may also be included, such as an analog to digital converter 41, for instance, where the converter is configured to pre-process photoemitter currents and/or photodiode intensity analog data, e.g., raw reflected electromagnetic radiation data, and convert them into digital data. In various embodiments, the converter may be a two-way analog to digital, and digital to analog converter. Further, in various embodiments, a storage-devices 76, such as a flash storage, or other memory device, may be included, such as for the on-board storage of data, such as photodiode currents, photodiode readings, and the like. In certain instances, a PCBA 42 may be included, and all of the components may be powered by a power source 48, such as a rechargeable lithium-ion battery, which may be adapted to allow for “quick charging.” In such instances, the battery 49 may be recharged quickly, e.g., within 20-30 minutes, so as to fully recharge the battery, which battery may be of a capacity to last up to 7, such as 14, such as 21 days, up to about 30 or more days, e.g., per wear period. In certain embodiments, the power source 48 may be configured for wireless charging, e.g., conductive or inductive charging, and thus, the power source 48 (as well as one or more of the emitters) may include or otherwise be associated with an antenna array including one or more antennas.

Particularly, as can be seen with respect to FIG. 5A, in various embodiments, the sensor unit 18, specifically, the electronics module 40, may further include a number of other components, such as a temperature sensor 64, and/or thermistor, an accelerometer 61, and/or gyroscope 62, SPO₂ assembly 63, such as for collecting temperature, oxygenation, and/or movement data of the wearer. The inclusion of a temperature sensor 64, is useful for allowing the sensor to monitor the temperature of the skin under, e.g., at the surface of, the sensor. For example, skin temperature data is useful for determining a reaction of the skin, e.g., with respect to its resistivity, in view of both the biomolecule of interest as well as the experienced temperature. In various embodiments, an electronic or digital shutoff may be included, so as to ensure the skin temperature does not exceed 42° C., such as for 8 hours or more, so as to warn the user of any excessive thermal exposure.

Accordingly, as can be seen with respect to FIG. 5A, the non-invasive, biometric sensing and measuring device 15 may be configured as an all-around sensing and monitoring apparatus 10. For instance, in particular embodiments, the sensing and/or monitoring device 18 may include a plurality of sensing devices, in any combination as described above, for generating a number of sensed data that may be considered when determining characteristics of one or more biomolecules as well as the conditions of the body. Particularly, as can be seen, the sensing device 18, may include one or more sensor arrays, such as including a first sensor array 20a including a plurality of photoemitters and photodiodes, as described above, and a second sensor array 35, which is composed of a photoplethysmography (PPG) sensor.

For example, as set forth above, one or more of the four or six emitter arrays, as depicted in FIG. 2B, may be removed and, may be replaced with a photoplethysmography (PPG) sensor 35, as depicted in FIG. 5A. Particularly, as depicted, a PPG array 35 has been substituted for the four-emitter sensor array 20b of FIG. 2A. Including a PPG sensor 35 in addition to a photo-array, such as the six-emitter photo-array 20a of FIG. 2B, is useful because it allows additional sensed data to be obtained and considered when determining a value of a biomolecule under consideration. In this embodiment, the six-emitter photo-array 20a is useful for detecting the presence of and a value of a biomolecule of interest, whereas an addition of a PPG sensor array 35 may be useful along with the six-emitter array 20a for determining changes in skin resistivity caused by the presence of glucose in the interstitial fluid. Further, the PPG sensor 35 may further be useful for detecting and/or determining a change in fluids, such as the change in flow and/or blood volume, e.g., caused by the pressure of circulating blood, for detecting element of the cardiac cycle, and for determining pulse-wave value, and the like. Additionally, in particular embodiments, the PPG sensor may be included as an addition to the above referenced four 20b and six 20a emitter arrays of FIG. 2B. In any of these instances, the PPG sensor 35 may be configured as a miniaturized, solid state electronic sensing device that reads reflected light data and in response thereto generates a PPG readout that can be used for performing improved biomolecule, e.g., glucose, calculations. In this regard, the PPG generated data is useful for determining physiological health and/or pathological conditions of the cardiovascular system, both of which are further useful in determining an accurate value of biomolecules, such as glucose, within the blood and/or interstitial spaces.

Hence, in many instances, it is useful to include a PPG sensor array 35 in addition to one or more of the 4 or 6 or other photoemitter/one or more photoreceiver arrays, as described herein. For instance, as can be seen with respect to FIGS. 5A, 5B, a sensor unit 15 may include a plurality of sensing devices 20, 35 for collecting a plethora of bodily information, all of which may be considered when determining the presence and effect of various biomolecules within the body. These sensing devices, as set forth with respect to FIG. 5A, are illustrated in an exploded configuration in FIG. 5B, so as to better see how the configuration of the layout may be assembled together. This layout is important because it has been optimized so as to be in a very small form factor, such as for ease and comfort of wearing for a prolonged period of time.

As can be seen, a central feature of the sensing device 15 is a six emitter, single receiver sensor array 20a. As depicted, this array includes six emitters, 22a-f, but can include more or less emitters. Likewise, the emitters may be configured for emitting any suitable wavelength of light, such as one or more of those set forth herein above. One or more photoreceivers 23 may also be included for detecting and collecting the wavelengths reflected from the skin after emission by the one or more emitters. In particular embodiments, the wavelengths of the photoemitter array may be configured as a glucose sensor array having photoemitters and photodiodes that are configured for detecting and/or determining glucose values and its effects on the body.

To better determine glucose and/or other biomolecule values, and body responses thereto, a number of other sensor values may be detected and used to perform one or more of the calculations described herein throughout. For instance, the sensor unit 18 may additionally include a temperature sensor 62, as well as a galvanic skin response sensor 69a, 69b. These sensor devices are useful for determining both the condition of the body at the time of measurements, such as skin temperature and/or resistivity, so as to better determine a baseline condition of the wearer but are also useful for better determining the presence of various biomolecules within the body as well as their effects thereon.

As indicated, a PPG sensor device 35 may also be included, such as for detecting biomolecules that are positioned deeper within the body, such as within the interstitial spaces, deeper within the tissues, and/or within the blood and vessels. Data collected from this PPG sensor, therefore, will allow the analytics system to consider a wider variety of variables, such as blood glucose values, oxygenation, blood flow, pulse rate, pulse duration, pulse periodicity, blood volume, expansion and contraction of the vessels, such as during a cardiac cycle. All of this data may be collected and used to define an entire cardiac cycle and/or overall breathing experience, all of which can be used to determine both the presence of biomolecules within the tissues, vessels, and fluids therein and between. Additionally, as indicated above, collected cardiac relevant sensor data can be used to generate a pulse-wave-velocity analysis by which cardiac function and/or vessel health may be assessed. Other cardiac relevant measurements and data can also be generated.

As can be seen with respect to FIG. 5C, the PPG sensor 35 may include an electromagnetic radiation emitter array 36 and an electromagnetic radiation receiver 37, which in this instance are arranged linearly, e.g., in a lateral line, in relation with one another. But, in other instances, the configuration of emitters and receiver, may be arranged differently such as where the emitters 36 at least partially surround the receiver 37, such as in a circle, semi-circle, square, triangle, and the like. Consistent with the iterations above, the emitter/receiver array 35 may include any reasonable number of emitters 36 and receivers 37, deploying any suitable wavelengths for emission and collection.

However, in particular embodiments, the PPG sensor 35 may include three photoemitters 36 and at least one, but up to three, or more, photoreceivers 37. Particularly, as depicted, the photoemitter array includes three electromagnetic radiation emitters, 36a, 36b, and 36c, which can be configured to emit one or more of a visible, near-IR, or IR light wave. Nevertheless, in particular embodiments, the electromagnetic radiation emitters 36 are configured for emitting light within the green, red, near- and/or infrared spectrum, and likewise, one or more photodiodes 37 may be included to detect and receive reflected light within the ranges of the green, red, near- and/or infrared spectrums.

Accordingly, as can be seen with reference to FIGS. 5C and 5D, the PPG sensor 35 may include one or more, e.g., a plurality, of low intensity green, red, near- and infrared light. In certain instances, the electromagnetic radiation emitters may be high intensity green and/or red, e.g., near-infrared or infrared, LED light emitters. Specifically, in various embodiments, the PPG sensor 35 may include one or more photo-emitters, 36a, 36b, and 36c, e.g., LEDs, that are configured for emitting light of a wavelength so as to fall within the green spectrum, as demarcated by the vertical hashmarks in FIG. 5C, and further includes one or more photo-emitters that are configured for emitting light of a wavelength so as to fall within the red to infrared spectrum, as demarcated by the horizontal hashmarks. In such instances, the photoemitters may be cycled so as to emit a combination of various lights within the green spectrum, various lights within the red spectrum, as well as various lights within the near and/or infrared spectrum, such as collectively, e.g., all at once, or sequentially.

Further, as depicted, the PPG sensor 35 includes one or more photoreceivers 37, such as a photodiode, for receiving the emitted light waves. In various instances, the photoreceiver 37 may be adapted for collecting and detecting light in the green, red to infrared light being reflected back from the tissues and vessels. Through the continual irradiation of the underlying tissues with green and red-infrared lights, e.g., from respective photo-emitters, the light may traverse through the skin and the change in fluid volume, e.g., in the tissue and/or vessels, may be determined, such as by measuring the difference in the amount of light either transmitted or reflected and/or refracted back to the photodiode 37.

For instance, since light is more strongly absorbed by blood than the surrounding skin tissues, the changes in blood flow can be detected by the photodiode(s) by the changes in the spectral array and/or intensities of the lights being reflected back from these respective structures. More specifically, with every cardiac cycle, the heart pumps blood to the periphery, which cardiac action causes a characteristic change in the skin and vessels as the blood is pumped through every particular tissue. The pumping of the blood through an area causes a pressure pulse to be transmitted through the vessels, which causes the distention of the arteries, arterioles, capillaries, and surrounding tissues. This pressure pulse is the result of a greater volume being pumped from the heart to the periphery, which pulse distends the vessel walls and surrounding tissues, which in turn, causes a concomitant change in the skin, which can be detected optically.

More particularly, as employed herein, the PPG light signal has several components from which signals several different metrics may be determined, including: volumetric changes in arterial blood, e.g., which is associated with cardiac activity, variations in venous blood volume, which modulates the optical signal, and an AC and a DC component can also be observed. This spectral determined data shows the tissues' optical properties and allows subtle energy changes in the body to be determined. Further, because the skin is richly perfused, the pulsatile component of the cardiac cycle can be determined through reflection, such that the DC component can be determined, e.g., by bulk absorption within the skin, while the AC component may be determined by the variation in blood volume caused by the pulse. From this data the systolic and diastolic, e.g., AC/DC, phases can be determined. As indicated, from this data, both blood oxygenation and cardiac events, e.g., heart rate, can all be determined. Further, as indicated, from this cardiac data a pulse-wave-velocity analysis can be performed by which cardiac function and/or vessel health may be assessed. Other cardiac relevant measurements and data can also be generated.

As can be seen with respect to FIG. 5A, one or more of the photo-emitters and/or photo-receivers 36, 37 may be surrounded by a light blocker so as to prevent electromagnetic radiation from an emitter 36 impinging into the photo-receiver 37 and thereby interfering with the reading of light reflected back from the tissue being monitored. For example, in various embodiments, the light blocking element(s) may be configured as an opaque, e.g., black or white, light dam that can surround the photo-emitter(s) 36 and/or photodiode 37, and functions to absorb and/or prevent light being emitted from the photo-emitter from invading into the photodiode 37 such that only light reflected back from the skin is allowed to hit the photodiode. The light dam may be of any suitable configuration and of any suitable material, such as metal, rubber, foam, although a metal, e.g., aluminum, material may perform better than foam, e.g., because it is less transparent to IR light impingement in that it absorbs and does not refract or allow it to pass through.

Further, as can be seen with respect to FIG. 5B, the sensing and/or monitoring device 18 may additionally include a number of other electronic components 60, such as for generating additional data that may be relevant to more accurately determine biomolecule values and/or their effects on the body 100 and the body tissues, as seen in FIG. 6A. For instance, the sensing and monitoring device 18 may include a temperature sensor 64, such as an infrared temperature sensor, for determining the temperature of the skin and body. Likewise, along with determining skin temperature, it may be useful for further determining skin moisture levels, and so a moisture sensor, such as a galvanic skin response sensor 37 may also be included. Furthermore, one or more of the sensing devices, and other components 60, may be associated with one or more PCBs 42, all of which may be contained within the housing 17. In such an instance, one or more of the devices may be covered by a glass window 21 in the bottom casing member 17b of the housing 17, and in certain instances, the window, as all windows referenced herein, may be plastic or glass and may be coated with an anti-reflective coating, so as to increase the signal to noise ratio.

Furthermore, as can be seen with respect to FIGS. 5D and 5E, provided herein is a non-invasive, continuous sensing and/or monitoring biomolecule detection device that is configured to be positioned on the skin of a body of a user of the device, e.g., a wearer, such as an arm, leg, or back of the user, in a manner such that light, e.g., green, red, near, and/or infra-red light, which as depicted in FIGS. 5D and 5E, may be emitted and directed into and through the skin. In particular implementations, as can be seen with respect to FIG. 6A, the sensing and/or monitoring device may be associated with a framework member 14, e.g., dome, and/or attachment structure 12, such as to form a patch 11 that can be positioned on the body 100, such as on the back of the arm between the shoulder and the elbow.

Specifically, as described herein, in various embodiments, the sensing and monitoring device 15 may be configured for continuous biomolecule monitoring, such as up to 1, 2, 5, 10, 14, 21, or 28 days or more, and for these purposes, the device may be attached to the body, e.g., via a patch or wristband structure, at an active site for a prolonged period of time for observation, detecting, and sensing. As can be seen with respect to FIGS. 5D and 5E, once securely attached to the body, so as to virtually be immobilized thereon, a first step after attachment of the biometric sensing and/or monitoring device 10 may be to run a calibration protocol so as to calibrate the device to the particular user at the particular position the device is applied to the body. Such a calibration and/or signature may be performed by running a routine, which routine may be implemented by one or more processors 45 of the device, which processor(s) may control the energizing and activation of the photoemitters 22, 36, e.g., of the photo-sensor 20 and/or PPG 35 arrays, as well as configuring the photodiodes 23, 37 to receive reflected light waves back. In particular implementations, an on-board or off-board processor 45 may control the sequence, pulsing frequency, duration, and intensity of the photoemitters, in accordance with the generated calibration protocol, such as by directing one or more micro-controllers configured to control each of the elements of the sensor.

Such calibrations may be performed so as to map the internal constituents underlying the tissue upon which the sensing and monitoring device is placed. This mapping may be of cellular structures or fluids within or around those structures or may simply be a signal of a pattern of reflectance with respect thereto. In certain embodiments, the mapping may be of biomolecules, such as glucose, contained therein. Specifically, in particular embodiments, the mapping may be of the reflectance patterns emitted and received by the sensor arrays. Once calibrated, the device may then be configured for sensing biomolecules, such as glucose, and determining their characteristics, such as by taking a number of readings, e.g., measurements, periodically, such as every 5, 10, 15, 20, 30 minutes and/or more, such as every hour 2 hours 4 hours, 8 hours, or 12 hours or more, such as every day.

Once calibrated, the device 15 may then implement in an emittance protocol by which to illuminate the skin and the components therein, in a number of different patterns, so as to detect and determine the presence and values of various biomolecules within the skin, which can be mapped over time, along with the various physiological factors that characterize the body at the time the measurements are taken. As described herein below, all of this data may be fed into a data structure generated by the analytics system whereby a holistic mapping of all factors can be generated, and correspondences between the presence and characteristics of biomolecules within the tissue, the spectral arrays associated therewith, and the individual's physiological response thereto, can be made. And because these measurements are taken continuously, the various connections between these correspondences can be weighted. Consequently, once produced, the data structure, or other analytic framework, can then be used to determine or otherwise predict a number of different characteristic values of the biomolecules measured and/or the state of a body or its tissues in response to thereto.

Specifically, as depicted in FIG. 5E, once the sensing device 15 has been calibrated with respect to its present location on the body. One or more measurements of the underlying tissues may be conducted. In this regard, one or both of the PPG and LED array 20a, 35 may be energized, and the reflected light waves collected pursuant thereto may be analyzed. For instance, as depicted in FIG. 5E, right hand side, a first, photo-array 20a (and/or 20b if included) may be activated, whereby the light waves emitted penetrate to a first depth, before being absorbed or reflected. And subsequent thereto, a first set of data is collected. Then, a second, PPG array 35 may be activated, whereby the light waves emitted penetrate to a second depth before being absorbed and/or reflected, such as where the second depth is greater than the first depth, as depicted in FIG. 5E. In this manner, the biometric sensing and monitoring apparatus 10 of FIG. 5A may allow for a greater amount of data to be collected, such as because, two different spectral arrays 20a and 35 are employed so as to generate and collect different data. It is noted that, with respect to FIG. 5D, when activating the PPG emitters, such as for the illumination of the tissues with one or more of green and/or red to near infrared light, the activation of the emitters 36a, b, c, can be sequentially, or collectively all at the same time, such as for determining blood flow characteristics.

However, in other embodiments, the biometric sensing and monitoring apparatus 10 of FIG. 2B may be useful where a wider spectral array, e.g., larger range of light waves, is preferred to be detected and analyzed. Also, because more sensor devices are included in the embodiment of FIG. 5A, then in the embodiment of FIG. 2B, it will likely have a larger form factor, and thus, where a smaller design is preferred, the embodiment of FIG. 2B may be employed, but where a greater quantity of data is preferred, the embodiment of FIG. 5A may be preferred. Nevertheless, it is noted that the additional sensor units, and other componentry of FIG. 5A, can also be added to the embodiment set forth in FIG. 2B, but in such an instance, the form factor will likely be larger, so as to accommodate for the additional components.

Accordingly, as depicted in FIG. 5E, once the body tissues and underlying structures therein have been illuminated, and the data has been collected, such as from the sensing device of FIG. 2B (left hand side) or from the sensing device of FIG. 5A (right hand side), or both together, the collected data may then be pre-processed and/or processed prior to being transmitted, such as for display or for further processing. For instance, in such embodiments, the sensing device 15 may further include an output device, such as a communications module 43, such as including a communications transmitter 44a and a receiver 44b. Particularly, in certain embodiments, the communications module 43 may include a Bluetooth Low Energy® (BLE) device 44, which may be coupled to the processor module 45 for outputting at least one of the sensed, calculated, and/or determined biomolecule, e.g., glucose, characteristics, e.g., levels, as well as the other collected data, e.g., raw or processed spectral data.

For example, the raw electromagnetic radiation, digital read, and/or determined results data may be transmitted to one or more of a remote server 74 and/or client computing device 73, such as a mobile phone 78, whereby the determined biomolecule levels can be reviewed by the wearer of the device, and one or more actions, e.g., lifestyle decisions, may be suggested by the system in respect thereof. Specifically, as can be seen with reference to FIG. 5E, in various embodiments, the system may include a client computing device 78 that is configured for running a client application 80, such as a downloadable mobile app that is configured for being run on the client computing device 73, through which mobile app 80 the wearer of the sensing and/or monitoring device can receive and view the determined biomolecule, e.g., glucose, results data and view trends relevant thereto, as can be seen with respect to FIG. 5E. As indicated, the sensing and/or monitoring device 15 may be configured for being coupled to a body portion of the user for a prolonged period of time, such as for 14 or more days of use, after which the sensor apparatus 10 may be removed from the body portion 100, recharged, cleaned, reapplied, and recalibrated to begin another period of sensing and monitoring.

As can be seen with reference to FIGS. 4B and 5E, the tissue in the area of the active site, where the light is impinging into the skin, includes an epithelial layer of the skin 102, the interstitial fluid 104, and the blood vessels 106, e.g., capillaries, carrying the blood and intervascular fluid. Consequently, the photo-emitters, e.g., LEDs, shine light into the skin 100, and the light penetrates through the various different layers, whereby some of the light is absorbed by each of the various different layers, and some of it is reflected back to be picked up and read by the photodiode of the monitoring device. In one implementation, it is the light that impinges into the interstitial fluid and is reflected back that is used to determine glucose levels. In this regard, the amount and/or level of reflectivity of the light back from interstitial and other fluids is dependent, in part, by the number of biomolecules, such as glucose, present therein, e.g., in the interstitial fluid and/or blood within the vessels.

Therefore, pursuant to calibration, a first pass may be performed so as to obtain a first, base level reading. Then a second, third, fourth, or more passes can be performed, and the results thereof can be compared, such as where each pass may be performed at a different depth of impingement and/or under different conditions, such as where each light being shown into the skin is emitted at a wavelength so as to penetrate into different layers of the tissue before being reflected back. In such embodiments, glucose may be present in some layers, such as in the interstitial fluid, but not present in others, such as in the epithelial cells themselves. Likewise, the photoemitter(s) of the PPG can be engaged, such as to penetrate more deeply into the tissue such as to reach into the blood vessels, wherein glucose within the blood may be detected. Particularly, the PPG emitter may be configured to emit light of a wavelength and/or intensity that goes deeper through the layers and into the blood, where it is then reflected back.

In the embodiments set forth herein, the monitoring and/or sensing devices may be configured to collect a number of sets of reflected light data from the interstitial fluids, vessels, and blood, and/or surrounding tissues, via the photo-array and PPG sensor array. All of these data points are useful because there may be biomolecules, e.g., glucose, both in the interstitial fluid as well as the blood, and light emitted from each sensor penetrates to different levels and therefore performs reads on glucose levels at the several, e.g., two, different layers. Further, as indicated above, other data may be collected and transmitted to the analytics system, such as via a wireless network connection, where such other data may include the skin temperature at the surface of the sensor as well as the galvanic skin response, which data may be incorporated as an input into the biomolecule, e.g., glucose, conversion algorithm, such as an ANN algorithm. (As stated elsewhere herein, certain aspects of the disclosed technology are described with respect to an ANN system and/or one or more ANN algorithms; however, the disclosed technology is not so limited and can include, implement, use, and/or apply any desired or useful machine learning technologies and/or systems or any combination thereof.) This allows the algorithms herein to account for conditions when the user is exercising or during sensor signal acquisition as these scenarios may impact measured glucose values due to the potential effect temperature and/or skin resistivity may have on glucose spectral absorption or glucose-mediated skin response.

Accordingly, in view of the above, a mix of sensor elements is useful because it allows the analytics module to build a data structure, such as a knowledge graph, decision tree, nearest neighbor graph, an artificial neural network, and the like, whereby the various sensed and other data collected may be input and used to accurately determine various different biomolecule values, such as glucose levels and/or concentrations. Particularly, the more relevant data is entered into a data structure the better the resultant calculations will perform. For instance, in one embodiment, the data structure may be an ANN whereby the greater the amount and/or variety of data entered into the structure, the better and more accurate the calculations will be.

In this regard, having a sensor unit with a single array of emitters and receivers is useful, but in some embodiments, having a plurality of such sensor arrays may be better, and likewise, substituting, or otherwise adding, one or more of the sensor arrays with a PPG sensor adds additional utility and efficiency. Further still, additionally including other sensing devices, such as temperature, galvanic, motion, and other sensors, may further increase the accuracy of the predictive models being generated and/or the calculations being derived thereby. These results may be superior to having only a single photo sensing array or PPG sensor all on its own.

Consequently, marrying the photo-sensor array, e.g., for glucose measurements, together with PPG readings derived from the PPG sensor, as well as the Galvanic Skin Response and temperature data, gives a plethora of data by which the above referenced data structures, for performing one or more of the measurements and calculations discussed herein throughout. As a basic rule, the more data considered, the more accuracy there will be when employing a data structure to perform measurements, make calculations, and then better determine conditions of the body with respect to the detections and measurements made herein. In this regard, in one embodiment, the data structure may be configured as an artificial neural network (ANN) that can be employed to determine overall glucose values and conditions of the body in response thereto. Thus, accuracy of the ANN can be increased by employing a large amount of data by which to perform calculations and make determinations.

Specifically, the ANN may function better in a data-rich environment that can be used to define the various different nodes in a graph-like or other structure. Hence, a feature of the system is the implementation of a data structure, such as by an AI module 72 of an analytics system 71. In some embodiments, the analytics module 71 may be instantiated on-board the sensing and monitoring device 15 itself, such as by a processing module 45 thereof, but, in other embodiments, the analytics module 71 may be remote from the device. In such instances, the electronics components 60 of the sensing and/or monitoring device 15 may include a wired and/or wireless communications module 43, such as a communications module that implements a Bluetooth® or BLE protocol for wireless data transmission. And, as indicated, in such instances, the data, calculations, and/or results thereof may be transmitted to a remote mobile computing device 73, such as a smart phone 78 running a client application 80 for analyzing the data and/or displaying the results thereof.

As can be seen with respect to FIGS. 6A and 6B, an important feature of the system 1 is an attachment apparatus 10 that is configured for coupling the above referenced biometric sensing and monitoring device 15 in close proximity to the skin 100 so that the calibrations and measurements disclosed herein can be taken in a consistent, uniform manner that does not change with the various movements of the body. Consequently, presented herein is a light emitting and sensing device 15 for use in continuous biomolecule, e.g., glucose, monitoring, which device 15 may be configured for being maintained in close proximity to the skin 100. In such instances, the sensing and monitoring apparatus 10 may be configured as a patch 11 or may be configured as a watch-like 90 device.

However, in the watch-like 90 instance, the determining of biomolecule, e.g., glucose, levels is computationally difficult because the watch 90 relative to the skin 100 is constantly moving as the wearer moves, and such movements may affect the light absorption and/or reflectance measurements. The analytics system 71 may make up for these difficulties computationally. Nevertheless, in other embodiments, these difficulties can be corrected for physically, such as by affixing the sensing and monitoring device 15 in proximity to the skin in a manner so that the device 15 is substantially prevented from moving, such as in a patch-like apparatus 11. Accordingly, to correct for this problem, the continuous biomolecule, e.g., glucose, monitoring device 15 may be composed such that it can be fitted within a patch 11 that includes an adhesive layer that is designed to hold the light emitting and/or sensing device 15 close to the skin 100 in a manner such that movement of the one relative to other is minimized. Such a patch-like 11 mechanisms are useful because the performing of such measurements is highly sensitive to the very specific area to which the device, e.g., within the patch, has been positioned.

Accordingly, in view of the above, provided herein is a non-invasive, continuous biomolecule monitoring device and apparatus 10 that collectively together may be configured as a wearable device 11, 90 that employs a transcutaneous biomolecule sensor unit 18 to sense and measure the spectral signals of one or more biomolecules, e.g., glucose, present within the skin. Particularly, the biomolecule sensing and monitoring device 15 is configured for generating data, such as one or more of raw electromagnetic reflected radiation data and/or digital read data that represents a spectral array of reflected light produced by the light directed into the skin from the photoemitters being received back to respective photoreceivers. These data may then be processed on- and/or off-board, such as by a computing system 70, whereby the data pertaining to the collected spectral signals may be analyzed so as to determine estimated levels and/or values of observed biomolecules and one or more conditions provoked thereby.

As discussed above, there are two main ways by which a patch-like structure 11 may be employed to hold the sensing and monitoring device 15, in close proximity to the skin 100 in an immobilized manner that prevents substantial movement. Particularly, by preventing substantial movement is meant less than 5 mm, less than 3 mm, less than 2 mm, less than 1 mm of movement. For example, the patch-like devices 11 set forth herein can prevent movement of greater than 1 mm, greater than 0.5 mm, greater than 0.1 mm. As indicated, the first patch-like structure 11 presented herein includes three general components of the referenced biological sensing apparatus 10, these include the biological sensor and/or monitoring device 15, a framework or encasement member 14, and an attachment structure 12.

In this regard, as can be seen with respect to FIGS. 3A and 3B, the biological sensor and monitoring device 15 may be received within the framework member 14, which framework member may be configured as an encasement or dome 14 into which the sensing device 15 may be snuggly and securely retained. The attachment structure 12 may be configured as circular or square-like member to which the dome 14 may be coupled, as shown in FIG. 2A. In particular embodiments, the attachment structure 12 may be at least partially composed of double-sided tape, which may be coupled to both the framework member 14 and the body 100 of the individual. As shown in FIG. 6A, the attachment structure may be configured so as to form an adhesive patch 11 that can be applied to the user's body 100 so as to position the sensing and monitoring device 15 into close proximity with the surface of the skin of an area of the body to which the apparatus 10 is to be applied. This configuration is useful in embodiments such as depicted in FIG. 2B, where the sensing and/or monitoring device 15 may have a disc-like shape and a low profile.

Hence, in various embodiments, the framework member 14 may be configured in a manner to include a low-profile receptacle, e.g., dome, into which the sensor 15 may be inserted, and may further include a flat, ledge or surface forming an attachment interface 16 to which an attachment structure 12 may be coupled, such as through a suitably configured attachment element 13, such as an adhesive, e.g., glue, as set forth in FIG. 2A. In such instances, the attachment interface 16 is more retracted, such as less than about 2 cm, less than about 1 cm, less than about 0.5 cm. In various of these embodiments, the attachment structure 12 may be configured as a patch, sleeve, band, bandage, or the like, to which the dome 14 and/or sensor unit 15 are to be coupled.

In such embodiments, where useful, an adhesive may be added to a skin contacting surface of the attachment structure 12, such as where the adhesive is biocompatible with sufficient consistency so as not to degrade too quickly over time. The materials from which the adhesive may be composed may be any fluid, tacky or sticky material capable of being associated, e.g., layered, sprayed, or otherwise be coupled with the framework 14 and support 12 members, and functions to keep them, and an associated sensor and/or monitoring device, firmly in place against the body part to which the apparatus 10 is to be attached. Where the framework member is configured as a dome 14, e.g., a circular encasement, the attachment interface or ledge member 16 may be configured as a circumferential surface that extends normal to a side-wall or bounding member of the dome so as to form an exterior lip with which the dome 14 and attachment structure 12 may be coupled together, such as by use of an adhesive. In such an instance, the ledge member may form an “L” shape with respect to the side bounding member of the cavity formed by the dome 14.

However, in other embodiments, a framework member or dome 14, along with its L shaped ledge member 16 can together function to maintain the sensing device 15 securely attached to the body 100, without the need for a separate attachment structure 12. In such an instance, the L-shaped ledge member 16 may be extended outwards, laterally away from the cavity of the dome 14 in a manner so as to form an attachment structure itself, but that is made of one-piece with the dome 14. In such an instance, the elongated circumferential surface of the ledge member 16 that surrounds the dome 14 need not simply be an attachment interface to which the attachment structure 12 may be coupled, rather, it may be the surface that gets adhered to the body directly, such as by the addition of an attachment element 13, e.g., a glue, to a sin facing surface of the attachment interface 16.

Accordingly, the attachment interface 16 may not only be extended, but it may also be elongated so as to form the attachment-like structure itself. In such instances, the configuration of the elongated attachment interface 16 may have four extended and elongated, opposed sides. In certain instances, the four sides may be of equal length, so as to form a square, and in other instances two sides may be longer than the other two, so as to from a rectangle. In such instances, the sides of the attachment interface 16 may be both extended and elongated so to be about 2 cm to about 10 cm, such as about 4 cm to about 8 cm, including about 5 cm to about 7 cm in length, e.g., similar to the attachment structure 12.

Hence, in particular instances, the dome 14 and attachment interface 16 may be of a single piece, and an adhesive can be added to a bottom surface of the elongated attachment interface 16 so that the dome can thereby be coupled to the body 100 directly. Hence, in various embodiments, the ledge member 16 may not only be extended, but it may also be elongated many centimetres or inches laterally away from a circumferential bounding wall forming the dome. In such instances, the elongated “L” shaped surface of the framework member 14 may form a base layer to which the adhesive element 13 may be added, such as at a bottom surface thereof so as to securely attach the elongated dome 14 to the body 100. In such embodiments as this, the framework member 14 may be a singular entity into which the sensor device is inserted.

The configuration of the framework member 14 and/or attachment structure 12 is important because, in various embodiments, part of the process of determining the effects of the biomolecule being observed on the body involves determining the changes to the body's tissues, fluids, and spaces therebetween that occur within the skin, vessels, and spaces thereof, when in the presence of the biomolecule. For these purposes, the sensor unit 16 may direct electromagnetic, or other radiation, into the skin, and may then receive reflected and/or refracted waveforms back. Specifically, as depicted in FIGS. 2B and 5A, the sensing device 15 may include a plurality of sensing arrays having a number of light emitters positioned in proximity to one or more photodiodes, such as where together a collection of emitters and receivers form an array. In certain embodiments, a dual array configuration is implemented, each having one or more photodiodes that are in line with or otherwise surrounded by a cluster of three, four, or six emitters, although eight or even ten or twelve light emitters may also be included.

As described in detail above, the emitters emit electromagnetic radiation into the skin, and the photodiodes receive the unabsorbed reflected energy back so as to generate read or spectral data. From this data, the device and/or system may formulate a map of the field of view of the observable skin and tissue spaces, such as based on the reflectance and/or refraction of various wave forms, e.g., light or sound, being directed into the observation area form which various measurements may be made. As these measurements are made repeatedly over time, so as to determine the change in the body over one or more periods, it is useful to hold the sensing unit in place for a prolonged period. Movement of the sensor unit 15 relative to its original placement of the skin, even by a small fraction, can disrupt its calibration, and throw the measurements off. Consequently, as indicated, a feature of the apparatus is a framework member 14 with or without an attachment interface 16, upon which interface an attachment structure 12 may be positioned and/or otherwise coupled, as shown in FIGS. 3A and 3B.

However, in other embodiments, as shown in FIGS. 3C and 3D, a framework member or dome 14 need not be included. Rather, in certain embodiments, all that is needed to secure the sensing device 15 to the body 100 is the attachment structure 12 itself. For example, in particular instances, the attachment structure 12 may be configured as an elongated surface member that is defined by an outer perimeter member. In such an instance, the attachment structure 12 will have both a top and a bottom surface.

Likewise, the attachment structure 12 will have both an inner portion and an outer portion. This outer portion may be defined the perimeter, which may include a single surface, such as where the attachment structure 12 has the shape of a circle, or it may include a plurality of sides, such as where the shape is a triangle, square, rectangle, and the like. Further, the attachment structure 12 may include an inner portion of the elongated surface, but where the inner portion is defined by an opening passing from the top surface to the bottom surface of the elongated surface. In such an instance, therefore, the elongated surface would also have an inner perimeter that defines the opening.

Hence, in various embodiments, as can be seen with reference to FIGS. 3C and 3D, the attachment structure 12 may be configured to include an opening, or hole, such as corresponding to the circumference and shape of the sensor device 15. In this manner, the sensor 15 may be inserted through the opening of the attachment structure 12, and together the apparatus may be coupled to body 100 so as to be held closely to the surface of skin in a manner so that the sensor unit 15 does not move relative to the skin 100. For example, such an attachment can be effectuated by the addition of an attachment element 13, e.g., an adhesive, to the bottom surface of the attachment structure. As the attachment structure 12 is configured for direct placement on the body, the material from which the attachment structure 12 and/or attachment element 13 are made should be non-toxic, biocompatible, and safe for use in contact with the body.

In any of these instances, the attachment structure is configured for maintaining a bottom surface of the sensor unit 15 in close proximity to the skin at the site of observation in a manner so that the sensor device is substantially prevented from moving. To better effectuate this positioning, in certain instances, a stiffened or otherwise inflexible attachment support 8 may also be included. In various embodiments, the support 8 may be a plurality of elongated members that extend longitudinally and/or laterally away from the inner perimeter of the attachment structure and are either integral therewith or can be added on top or beneath the surface thereof. However, in various instances, the attachment support may be formed as an attachment ring 8 that functions to provide structural support and positioning to the interior perimeter portion of the circle through which the sensing device 15 is inserted.

In particular embodiments, the attachment ring 8 may be configured to function as a mounting device, positioned so as to circumscribe the interior portion of the attachment structure 12, through which the sensing device 15 is inserted in a manner so as to be mounted with, or otherwise upon, the mounting ring 8. In such an instance, a top part of the ring may be flat, but the circumferential portion may have a thickness thereto, but with a rounded configuration. Together the flat top surface and rounded circumferential portion form a contoured center such that the hosing of the sensing device 15 may be mounted thereupon or be otherwise engaged with in a manner that locks the sensing device 15 substantially immovably in place. In various embodiments, the locking mechanism may be a tooth in groove coupling. In other embodiments, corresponding magnets may be included, such as with opposite polarity. Likewise, together the attachment ring 8 in combination with the attachment structure 16 enables the disc-shaped housing of the sensing device 15 to adhere to a specific location on a person's arm, abdomen, buttocks, or some other part of the body.

The mounting ring 8 may be coupled to, or otherwise be formed with, the attachment structure 12 in any suitable manner such as being formed, molded, or woven therein, or glued or otherwise attached thereon. For instance, in one embodiment, the mounting ring may be threaded to a top, bottom, or circumferential portion of the attachment structure 12. This will allow the ring 8 and/or sensing device 15 to be removed from the attachment structure 12, such as for recharging and/or replacement. As indicated the entire apparatus 10, or a portion thereof, e.g., the sensing device 15, can be removed from the body at any time. To reattach the device, a new mounting ring 8 may be re-secured to, or otherwise within, an attachment structure 12 along with the sensing device 15, and collectively the assembly 10 can be attached to the body. In some instances, the mounting ring 8 may need to be threaded to the attachment structure 12 and/or device housing 17. Other attachment mechanism, as set forth herein, such as a clip, can also be used. For instance, a portion of the ring can contain clip points that match corresponding clip points in the attachment structure 12 so that the mounting ring can be clipped to the attachment structure 12. Such clips may be positioned on the side of the substrate of the attachment structure 12 rather than the bottom so as to minimize the overall height of the apparatus 10.

The attachment structure 12 may be made of any material forming a substrate to which the sensing and monitoring device 15, and in some instances attachment framework or dome 14, may be coupled, and the entire apparatus 1 can then be attached to the body. In this regard, the dome may be composed of a plastic material, such as a polyethylene terephthalate glycol (PETG). PETG is useful for forming the dome material for it efficiently prevents water exposure, such as can occur while the wearer of the patch-configured monitoring device, is working out, sweating, showering, swimming, or it is raining. Thus, the dome, when included is configured and composed of a material so as to prevent exposure of the encased sensing device from the outside elements.

In particular embodiments, the dome may be removably or permanently attached to the attachment structure 12 or patch 11, e.g., adhesive band, for positioning the sensing device in close proximity to the skin, or other base member, in a manner so as to be stably positioned thereby. However, in other embodiments, the housing 17 of the sensing and monitoring device 15 is waterproof. For instance, one portion of the housing 17, such as a top member 17a may have a tongue or tooth-like element, e.g., circumscribing a portion or all of a perimeter portion of the housing 17a, and the other portion of the housing 17, such as a bottom member 17b, may have a corresponding groove like-element, e.g., circumscribing a portion or all of a perimeter portion of the housing 17b. A compressible element, such as a foam or O-ring, may be positioned within the groove such that as the tongue fits into the groove, or channel, the compressible element is compressed, thereby forming a waterproof sealing therebetween. The housing may farther include one or more latches for securing the sealing.

With regard to the attachment structure 12, such as where a dome 14 is not included, the elongated member forming the attachment structure 12 may be composed of any suitable material, such as metal, an alloy, aluminium, titanium, plastic, acrylic, or other stiff material. However, I other instances, the attachment structure 12 may be composed of a flexible or semi-flexible material, such as made from a malleable plastic, rubber, silicone, plastic or fiberglass containing mesh, a woven blend, any other form of mesh, or may be composed of a foam material. In a particular embodiment, the attachment structure substrate may be composed of a double-sided tape such that one surface of the double-sided tape attaches to the framework member 14 and/or sensor housing 17, and the other surface is then capable of being attached to the tissue, e.g., skin, of the body portion thereby covering the tissue where the electromagnetic observation is to take place.

Regardless of the manner of coupling, the contact of the attachment structure 12 with the sensing device housing 17 to form the apparatus 10, and the attachment of the apparatus 10 with the skin should be such that it locks the sensor unit in place above the action area where the electromagnetic radiation is to be directed into the skin and the measurements are to take place. In this manner, the sensor unit may be retained within a position for sensing the presence of the biomolecule as well as for determining its levels and/or bioactivity, such as in or around the biological tissues of the action area. Accordingly, in a manner such as this, the biomolecule sensor and/or monitor may include a number of electromagnetic, e.g., light emitters, that are configured and positioned within the sensor unit so as direct visible, near infrared (NIR), infrared light, and/or other radiation, such as sound waves, into the skin on the user's tissues, such as on the back of the user's arm.

As depicted in FIG. 6A, the wearable sensing and/or monitoring device 15 may be coupled with a framework dome member 14 and/or an attachment structure 12 that may be in the form of a patch that is employed so as to attach the sensing and monitoring device 15 proximate the user's skin 100. As depicted, the sensing and monitoring device can be formed as a small circular disc having a top 17a and a bottom 17b, where the bottom includes a light transmissive area, such as an opening or window 21, which may be formed of a transparent material, such as acrylic, and which may have one or more portions coated with an anti-reflective layer.

As indicated above, the framework member 14 may be configured as a receptacle, such as an encasement or a dome, which is configured for receiving the circular disc shaped sensing and monitoring device 15 within it. In such an instance, the dome 14, therefore, may include a circular bounding member that serves the same purpose as the mounting ring 8. Accordingly, in either embodiment, either with an encasement dome 14 or without, the apparatus should be configured so as to position the sensing device 15 within the center of the opening of the attachment structure 12, which as shown in FIG. 6A, may be configured as a patch-like attachment element 11.

As indicated, the patch 11 may be square, but in various instances, such as illustrated in FIGS. 3C and 3D, the patch may be configured as a donut, having a central opening through which the disc-shaped sensing device is inserted. In such an instance, the boundary of the opening, or a bottom side of the disc housing, may have an engagement member, e.g., a tooth or lip, so as to prevent the sensing and monitoring disc from being passed all the way through the opening. In various embodiments, once inserted through the opening in the attachment structure 12, a light transmissive layer may be covered over the opening and/or transmissive window area of the sensing device housing 17b. In particular instances, the transmissive layer may be a transparent sheet, which may be composed of a plastic or acrylic material. However, in various embodiments, such an additional transmissive covering layer need not be included.

In any of these embodiments, the patch 11 is adapted for positioning the retained, and/or encased, sensing and monitoring disc 15 in proximity to the surface of the skin and ensuring that the disc device does not move relative to the movements of the wearer. This stable positioning is useful for allowing the sensor device 15 retained within the disc housing 17 to perform its calibrations and to take its measurements, such as by holding the transmissive surface 21 close to the skin 100, whereby the photoemitters within the housing 17 may then direct electromagnetic radiation into the skin, and the photodiodes may receive and analyze reflected and/or refracted electromagnetic radiation back from the skin, such as in the performance of a calibration, mapping, sensing, and/or monitoring operations.

As set forth herein, the housing 17 of the sensing and/or monitoring device 15 may be composed of two halves forming a top surface 17a and a bottom surface 17b that can be joined, e.g., via a tongue and groove, snap, or other fitting, together to form a disc-like shape having a cavity therebetween wherein the electronics for performing the herein disclosed measurements may be retained. In various embodiments, a compressible gasket can be fitted between the two halves of the housing, so as to make the coupling waterproof. Accordingly, the top surface part of the housing may be flat with rounded sides but having a rounded or contoured center to make space for the referenced device electronics, including a PCB (printed circuit board), rechargeable lithium-ion battery, as well as the electronic sensor components. Likewise, the bottom surface part of the housing may be a correspondingly flat-rounded disc with a corresponding contoured center. The two-disc portions may have corresponding attachment mechanisms, like a tongue and groove, opposed corresponding ledge elements, e.g., “L” shaped teeth, and the like.

In the implementation depicted in FIG. 2B, the circumference of the disc is about 1 cm, about 2 cm, about 3 cm up to about 5 cm in diameter, and may be approximately 3 mm-7 mm up to about 10 mm to about 20 mm or more in height, such as where the top and bottom surfaces the housing are relatively planar or flat. However, in various embodiments, such as where the top and bottom surfaces are curved, the perimeters may be high, so as to give the disc a thin profile, such as less than 3 mm, such as less than 2 mm, such as less than 1 mm. Likewise, the length of one or more of the sides of the housing may be about 10 mm to about 20 mm, up to about 30 mm in length. Hence, the length and height of the sides may be relatively equal, so as to give the sensor device a square or rectangular shape. In other instances, where the device has a circular shape, the circumferential bounding member height may be equal the diameter, but in other instances, the perimeter height may be less than the diameter, such as substantially less than, so as to give the device the shape of a discus.

In any of these instances, the opening of the attachment structure 12, and/or the dome 14 if included, may be configured out of a flexible material, so as to conform to the contours of the disc being inserted therethrough, but may be slightly larger thereto so that the disc can be fitted snugly therein, but in a manner so as to adhere the disc to a specific location on a user's body, such as their arm, leg, back, abdomen, buttocks, or some other part of the body. This is useful because, as indicated above, a center portion of the bottom part of the disc may be a thin transparent plastic sheet, e.g., window 21, so as to allow the electromagnetic radiation to pass from the photoemitters, e.g., LEDs, mounted on a PCB encased within the housing out from the transparent plastic sheet 21 forming the bottom of the disc. The emitted electromagnetic radiation will then pass into the skin, and likewise a certain amount of electromagnetic radiation will be reflected and/or refracted back out of the skin and through the transparent plastic sheet, e.g., acrylic window 21, and into the housing. Once reflected and/or refracted back into the housing 17, the corresponding PCB 42 mounted photodiodes may then receive and read the reflected and/or refracted radiation.

Accordingly, in particular embodiments, the attachment structure 12, as set forth in FIG. 3C, may be configured so as to have an opening, such as to receive the disc therethrough. However, in various embodiments, the opening may be covered after insertion of the disc, such that a surface of the attachment structure 12 completely covers the bottom of the disc, such as where the covering is transmissive to light. In such an instance, the transmissive covering should be a circular portion that aligns with the transmissive portion or opening on the bottom surface of the disc housing 17. Such a covering can be employed to waterproof that apparatus or for sanitary reasons.

However, in various instances, the attachment structure 12 may not have a transparent plastic sheet covering the opening, but rather, may simply have an opening. In such as instance, the attachment structure may be configured so as to not completely cover the bottom of the disc. In one particular embodiments, the transmissive covering may be configured as a small circular opening portion that corresponds to the opening, e.g., about 1 cm, in the bottom portion of the disc housing 17b, so as to be aligned with the transmissive portion or on the bottom surface of the disc housing 17b. This is useful because light from the emitter passes from the disc device, through the patch, and into the skin and back without interference.

As can be seen with respect to FIG. 5B, in various embodiments, the sensor unit 15, may be configured for being pressed securely, but firmly against the skin 100. This is useful, not only for the sensor device emitting and receiving electromagnetic radiation into and from the skin, but also for collecting other data characterizing the skin, for which skin contact is beneficial or necessary. For instance, the sensing and monitoring device 15, and the housing thereof, may be configured to include a temperature sensor 64. In one embodiment, the temperature sensor may be in contact with the skin, so as to take skin temperature measurements. However, in other embodiments, the temperature sensor may be an infrared temperature sensor 64 that is configured for directing an infrared light into the skin, receive reflectance back, and from the spectral array determine skin temperature. In such an instance, the sensor may have an IR emitter and diode offset from the surface of the housing, but covered with a transmissive window, which window may have an anti-reflective coating thereon.

Likewise, the housing may be configured to include one or more skin interfacing surfaces, configured as corresponding electrodes of a galvanic skin response sensor unit 69. For instance, the galvanic skin response sensor 69 may include a first skin interfacing surface 69a, which may be implemented as a first electrode positioned on one side of the bottom surface 17b of the sensing and/or monitoring device 15, while a corresponding second skin interfacing surface 69b, which may be implemented as a second electrode positioned on the other side of the bottom surface 17b. Together the two electrodes can pass a current therebetween so as to determine the body's skin response, from which a galvanic skin response measurement may be taken, and the results thereof can be fed into the data structure, so as to give a measurement of the skins resistivity, which in turn can be used to better determine the presence of a biomolecule of interest as well as the body's response thereto.

Further, it is noted that the electrodes 69a and 69b can also be used by themselves or in addition with a pair of other electrodes 66, which may be configured as EKG electrode interfaces, 66a and 66b. In other instances, the electrodes 66a and 66b may be employed by themselves, such as for taking an EKG reading. A further set of corresponding electrodes 66c and 66d may also be included in the housing and can be used for charging the battery 49. In particular embodiments these electrodes are configured for interfacing with, e.g., contacting, the surface of the skin, whereby the various measurements disclosed herein may be made. Further, the sensor device 15 may include a communications module 43, having a transmitter 44a and receiver 44b, by which communications, such as instructions may be sent and received, and data may be transferred. In such embodiments, a communications interface may be built into the housing 17.

With respect to pressing the sensor unit 15 securely, but firmly, against the skin 100, such as for the taking of skin temperature, measuring a galvanic skin response, and/or taking an EKG reading, in such an instance, the attachment structure 12 may be a compressible, foam support member configured for being compressed when pressed against the skin by an applying force. As depicted in FIG. 3D, the attachment structure 12 may be configured so as to have a compressible donut shape. The donut shaped attachment structure 12 may have a central opening through which the sensor device housing 17 may be inserted.

In such instances, the attachment structure 12 may include a mounting support 108 that may be positioned at the interface between an interior perimeter portion of the attachment structure 12 and the sensor device housing 17, so that the sensor device 15 can be securely mounted within the donut and be pressed firmly against the skin. In this manner, the mounting support 108 member may be configured to prevent translational movement, e.g., left to right and forwards and backwards, and yet the flexible foam attachment structure 12 may remain flexible enough to accommodate body part movement. It is especially useful that the interaction between the sensing device housing 17, the attachment structure 12, and the mounting support 108, are configured to prevent rotational movement of the sensing device. This may be due to the rigid material, shape, and configuration of the mounting support 108, such as where they have corresponding corner features that prevent rotational movement of one with respect to the other.

However, in certain instances, the form factor of the sensing and/or monitoring device 15 may not be formed as a disc member, such as to be inserted within an attachment patch 11, as described above. Rather, as set forth in FIG. 6B, the sensing and/or monitoring device 17 may be configured as a watch 90. Particularly, in some implementations, the wearable device can be in the form of a watch-configured device or may be retrofit to existing wearable technologies. In such an instance, a watch-shaped sensor device 15 can be added to the back side of a watch, by an encasement structure 94 that functions to couple the sensor unit 15 to the watch. For instance, the encasement structure 94 is configured to couple the sensor unit 15 to the back of a smart watch and/or its writs-band 96, whereby the sensor 15 can communicate with the smart watch, e.g., wirelessly, such as via an RFID or BLE communications protocol. Particularly, in various embodiments, the electronics of the apparatus 10 may include a wireless communications module 43, such as a Bluetooth®, BLE, Wi-Fi, or other wireless transmitter.

In such instances, once collected by the sensor 15 and/or watch, the sensed data may be analyzed data and/or may be transmitted. For example, an analytics system 71 of the disclosure may be configured as an “on board” computational unit 45, which may further be in communication with a decentralized analytics module 71, such as a cloud based artificial intelligence system 72. The onboard sensed data and/or results may be transmitted wirelessly, such as to a remote server 74 or client computing device 73, e.g., a smart mobile phone 78 of the user, whereby the user may pull up and view the sensed and/or analyzed data from the wirelessly coupled sensing and/or monitoring watch-configured device 15 and/or to an associated mobile smart phone 78. In particular instances, the on-board computing system 45 may be in communication with a remote server system 74 through which the various analyses described herein may be performed. Likewise, the onboard computing system 45 may be in communication with a remote client computing device 73, such as a mobile computing device 78, for transmitting the read and/or analyzed data as well as the results of the analytics system, based on the readings attained by the biological sensor and/or monitor 15.

In particular instances, the device 90 can be configured as a watch and include a wristband peripheral 96 so as to be coupled the wrist of a user, as shown in FIG. 6B. In various instances, the wristband 90 may be an elastic or silicone band that stretches to allow for application to the wrist but then compresses so as to hold the sensor 15 behind the watch firmly in place, while worn. In such instances, the watch and sensor device combination may be waterproof so as to be worn for prolonged periods of time. In such instances, the band 90 or other attachment structure 12 may be configured to position the wearable electronic apparatus 15 on a body part of the user, or specifically, to position the sensor part 15 of the device in proximity with the skin and/or a blood vessel therein of the user.

For instance, in a specific embodiment, the sensing and/or monitoring device 15 may be configured as a watch 90, such as a smart watch, and in such instances, the band 96 can further include one or more connectors, such as for associating the smartwatch with the band 96 and/or the band with the wrist. In some implementations, the band can include a connection to receive a smartwatch, such as a pin connection. In various other instances, the attachment structure 12 for the watch may be an expandable and compressible sleeve, a watchband, a headband, and the like. In other instances, the sensor device 15 may be configured as a necklace, a bracelet, an anklet, a ring, a pendent, or the like, where an adhesive may or may not be needed to form an attachment. In any of these instances, the sensing and/or monitoring device 15 may include an output device, such as a display, which may be in communication with at least one of the one or more onboard processing elements, for reflecting any of the readings, ratings, and/or outputs to the individual wearer. However, in certain embodiments, a display may not be included, such as where it is omitted to preserve battery life and/or duration during which the apparatus is applied to the body.

Accordingly, regardless of the form factor of the herein disclosed non-invasive, continuous biomolecule sensing and monitoring device 15, e.g., regardless of being in a patch-like 11 or watch-like 90 assembly 10, in one aspect, provided herein, is a method for monitoring and assessing an individual's biomolecule levels, such as blood glucose levels. For instance, in particular implementations, the method may include one or more steps of providing a wearable electronic sensing and/or monitoring device 15 as described herein, such as where the wearable electronic device 15 includes one or more sensing devices and/or sensor units 18, e.g., including a photoemitter and photosensor, at least one processor 45, and/or a wired or wireless communications module 44 for data transmission.

The method may include positioning the wearable electronics apparatus 10 on the skin 100 of an individual, such that the sensor unit 15 is in a position so as to direct generated energy into an area of the body, where the area is sensitive to biomolecule, e.g., glucose, within the tissues and fluids in the sensitive area, such as depicted in FIG. 5E, right hand side. Next, the method may include illuminating the skin 100 with energy from the photoemitter, and receiving an amount of energy back, e.g., at a photoreceiver, so as to take simultaneous measurements of one or more of heart rate, cardiac activity, and collecting reflected light from the photoemitter. Then, once the data, e.g., reflected energy data, is collected, the at least one processor can be employed to convert the heart rate, pulse-wave-velocity (PWV) measurements, and reflected and/or refracted light data, e.g., spectral analysis data, into a prediction about one or more characteristics about the biomolecule being observed. For instance, one or more estimates about the level, concentration, PWV effect, and/or effects of the biomolecule, e.g., glucose, on the body may be made.

Additionally, an output reflecting any of the readings, ratings, and/or characteristics may be displayed, e.g., through the output/display module, or may be transmitted to a suitably configured external display device of the individual. In some implementations, the taking of the simultaneous measurements step may be accomplished autonomously and/or automatically on a periodic basis, such as using a timer. In certain instances, the output of the data readings and analysis can be subject to a tagging and/or auto-tagging program where the apparatus 10, or associated analytics system 71, can determine the individual's behavior during an activity (such as eating, sleeping, working out, or during an episodic stress event) at a time point and attach an electronic/digital tag to that event. In some instances, the output of the data readings or measurements and further analysis can provide a health trajectory for the individual to predict a future state of health and/or what the effects of a remedial intervention will be.

Consequently, in view of the above, provided herein is a non-invasive, continuous biomolecule monitoring (NICBM) method for measuring one or more biomolecules, such as glucose, non-invasively through the skin via an electromagnetic radiation detection device, such as employing an optical, e.g., photonic, and/or Radio Frequency (RF), e.g., microwave, sensor unit(s), such as by photonic and/or RF (microwave) spectroscopy, such as microwave, radio waves, visible, near-infrared, and infrared spectroscopy. These methods are non-invasive in that they do not use finger pricks or a chemical laden sensor unit that includes a filament that gets inserted within the skin. Rather, the sensor devices employed in the present methods disclosed herein use electromagnetic radiation that is directed into the skin and then measures transcutaneous light signals that are reflected back. From these detected light signals, a level, concentration, and/or change, e.g., trend, in an interstitial biomolecule, e.g., glucose, can be determined and/or predicted.

For instance, from the reflectance, refraction, absorption, scattering, and/or polarization of microwave, RF, visible and/or near-infrared (NIR) and/or infrared (IR) light directed into the skin, an estimated and/or predicted biomolecule, e.g., glucose, value can be detected and/or determined. For example, using one or more electromagnetic radiation generating arrays in the performance and/or determination of glucose is useful because it is non-invasive and, therefore, does not have a subcutaneous measuring component, e.g., insertable filament. Further, once one or more readings or measurements have been obtained, accurate estimated glucose values may be determined and calculated, e.g., based on electromagnetic radiation sensor signal inputs. As indicated above, such a determination may be performed by a pre-trained and/or locked Artificial intelligence module, such as employing a machine learning and/or inference engine. For example, in particular implementations, the machine learning component may be embodied within an artificial neuro-network (ANN), as described herein below.

Use of an AI mediated data structure, such as an ANN set forth herein, is useful for overcoming the aforementioned positioning problems, such as through the calibration processes set forth herein below. For example, another problem with typical amperometric sensor devices is that based on the internal positioning required of the needles and/or filaments of the sensor unit, its options for placement on the body is limited to larger body structures, such as the abdomen, buttock, or the like. However, this is a benefit of the sensing and monitoring devices presented herein, because based on the use of an electromagnetic radiation generation array and its non-invasive nature, the herein presented devices may be comfortably positioned on a number of different areas of the body, such as on the arm, wrist, finger, ear, leg, or the like. Hence, the present devices are flexible in terms of their placement on the body.

One problem, however, is caused by the movement of the sensing and monitoring device and/or apparatus, after a calibration process has been performed. This problem, however, can be overcome by the analytics system presented herein. For instance, the analytic system herein is adaptable so as to account for such problems by being adaptable to changes in positioning by being able to take into consideration of the different structures of the body when generating a topographical mapping of the tissues, internal structures, spaces, and fluids therein that make up the field of view of the sensor's illumination array when moved from one place to another on the body. Specifically, in various embodiments, to account for this difference in sensor application site, the sensor software, e.g., being run by the on- or offboard computing systems, is adaptable such that it can be trained in a manner that does not depend on body placement, but, nevertheless, can accommodate for it by the mapping process, which mapping is capable of accounting for various different changes in placement.

Hence, once placed and positioned on the body, the device in its placement may be calibrated, the calibration may be mapped, categorized, and/or characterized as to body position, and once calibrated, the system should not need to be recalibrated again, unless the positioning or placement is changed, whereby a previous mapping can be used to identify the new positioning. Hence, such calibrations are useful because they allow the software and optical units to account for any variation from the new placement location to the other, such as by small movements and/or larger placements to a different part of the body. In particular embodiments, the calibration may include determining a correspondence of sensed values with determined blood glucose values, e.g., from a blood glucose monitor.

Further, because the analytics system does not need to include physical elements, e.g., filaments and/or needles, which need to be inserted within a tissue of the body for the purpose of determining characteristics and/or levels of biomolecules, the configuration of the internal electronics are also adaptable so as to make room for a larger battery, and because no analytes are involved that need to be changed, the present devices can be continuously used over prolonged periods of time, without the need for repeated calibration, such as up to 7 days, up to 21 days, up to one or more months, even up to one or more years. In this regard, the battery may be configured for being recharged, such as in a wireless (or wired) manner when the device is or is not being worn. In various embodiments, a non-rechargeable battery, such as a lithium manganese dioxide battery, may be used, which is not reusable or rechargeable. However, in certain embodiments, a non-rechargeable battery should not be used. Instead, a rechargeable lithium-ion battery may be included.

The biomolecule sensing and monitoring device 15 senses, collects, and tracks electromagnetic radiation, such as photonic and RF (including microwave) spectral, e.g., reflectance, absorbance, etc., and other data over a prolonged period of time. This data may then be preprocessed and be transmitted, e.g., wirelessly, to a remote computing system 70, such as a cloud-based server system, running or otherwise being associated with an Artificial Intelligence (AI) Module 72, such as instantiating a deep learning Artificial Neural Network (ANN) that has been trained to map the collected readings or measurements to a specific biomolecule, e.g., glucose, measurement. Particularly, in certain embodiments, the data collected, collated, and/or amalgamated through the device 15 can be subject to an analytics module, such as including “machine learning” systems and methods to provide for predictive analysis for the individual, configured in a format so as to assist the individual in achieving personal health and wellness objectives, not necessarily alone, but in collaboration with their healthcare professionals, such as through the system application 80. In various embodiments, the data collected by the sensing and monitoring device can be employed by the analytics system to apply physical health and/or psychometric data analysis to assist the individual in achieving their personal health and wellness objectives.

Accordingly, in view of the above, as can be seen with respect to FIG. 7, a process for determining the presence, level, and/or concentration of one or more biomolecules, such as a metabolite, e.g., glucose, in the interstitial fluids or blood, such as using the non-invasive, continuous biomolecule sensing and/or monitoring device, disclosed herein above. Generally speaking, as set forth in FIG. 7, at step 110, the process may largely include collecting spectral and biometric data of the user, such as using the detecting, sensing and/or monitoring device set forth herein. The sensor device includes at least one energy emitter, e.g., a light emitting diode (LED) or microwave emitter, and at least one energy receiver, but may typically have 3, 4, 6, 10, or more, such as 20, 30, or even 50 or more electromagnetic radiation emitters, as well as having 1, 2, 3, 4, 6, 10, or more, such as 20, 30, or even 50 or more electromagnetic radiation receivers. In such instances, the various, e.g., 10 energy emitters, may direct light energy into the tissues of the body, whereby a portion of the emitted light impinging into the skin will be absorbed, e.g., at specific depths, and some will be reflected back so as to be collected by the various, e.g., 2, photodiodes.

Then at step 110b, the collected reflected spectral data may be pre-processed such as by at least being converted from raw analog data into digital read data via an analog to digital converter, ADC. Specifically, the ADC processes the LED and/or RF spectral data, current data, and photodiode and/or energy detector intensity data, which, once pre-processed, the data may be stored in an onboard memory, such as a flash memory. The pre-processed data can then be transferred to an on- or off-board computing system whereby the data may be evaluated and/or be subjected to a reinforced, DVRL, protocol, and be subjected to neural network filtering. Then at step 110c the data, e.g., filtered data, can then be integrated within a data structure and be processed, such as by an artificial neural network, whereby the individual's biomolecule values, e.g., glucose levels, may be calculated.

The collected data, therefore, may include a measurement of a level of a biomolecule, which may be indicative of a health condition, such as for determining a glucose value. Then at step 110d, the results of the analysis, such as including glucose concentration levels, as well as a prediction about the health of the individual may be output, such as to a mobile computing device, e.g., a smart phone, of the individual, for display thereby, such as where the smart phone is running a client application of the system. The outputted day may further include various trend and/or pattern data determined by the system over several hours, e.g., 1, 3, 6, 12, 24 hours, over days, e.g., 2, 3, 4, or weeks, or even months. All of this data may also be uploaded into the cloud, e.g., for storage and or further processing, and/or may be transmitted via the client application to a computing system of a healthcare professional so that the individual may receive help and guidance in meeting their health goals and wellness objectives.

More particularly, as can be seen with respect to FIG. 8, in various embodiments, the NICBMS may include a mobile device, such as communicationally coupled with the sensing and monitoring device, such as where the mobile device is capable of running a downloadable application of the disclosure. For instance, a downloadable mobile application may be included whereby the application is configured for working with one or more processors of the mobile device so as to generate a graphical user interface, which user interface may display an interactive dashboard display screen. In various embodiments, the mobile application may be configured as a Software as a System application whereby a system user may view biomolecule, e.g., glucose, and/or other health data, can see values over time, can view graphs and trends, and through which they may receive health recommendations.

Besides showing the biomolecule and/or health data, as well as analytic results relevant there to, the software application may also be used to set up and/or remotely configure the sensing and monitoring device, e.g., for first-time users, and to calibrate the sensor, e.g., for every application and reapplication, such as to start/stop the automatic measurements, and/or shut off the sensor, as need may be. For instance, when a user first receives the sensing and monitoring device, they will set up a user account, input their personal physiological information, and register their sensor. In particular embodiments, the registration date for the sensor may be important because at 1-year post-registration, the onboard software may automatically shut off the sensor to ensure the device is only used for a designated sensor life.

Specifically, once downloaded, the mobile application may be used to not only set up and configure the sensing device, but it may also be used to calibrate the system to each specific user and/or for each specific position on the user. For example, once the sensing device is fully charged, and the application downloaded to the user's smart phone or watch, the sensor device may be coupled, e.g., via BLE, RFID, etc., to an associated mobile computing device of the user, and the mobile application for running the software of the system may run the user through a set up and calibration protocol. First, a user account with a user profile can be set up and registered, individual characteristics about the user, their background their family background, health and psychological history can all be entered into the system, such as in response to a system generated interview. This is important for determining characteristics about the user so as to generate a user profile.

Once the account has been set up and a specific sensing device to be used has been coupled to the mobile application, then the device may be applied to the body. First, the user will position the senser unit for insertion into the attachment structure or dome to form the apparatus. Then, any backing material may be removed from a skin-interfacing surface of the attachment apparatus and/or dome. The apparatus with the protective glass of the sensor facing outward, e.g., downward, may then be positioned on to the skin so that sensor units of the device will be in contact with skin once applied. The user can then press firmly against the attachment structure to ensure the adhesive is securely placed. After the sensor and apparatus are placed on the body, e.g., the back of the arm, the user can then use the mobile application to communicate with the device, and vice-versa, so as to initiate and run a calibration protocol, which may include taking, or otherwise entering, a blood glucose measurement, such as with another pin-prick style device. Upon calibration, the user may select to start measurements and the sensor will autonomously transmit data from the sensor to the software platform. The user can view their estimated glucose value, 1-, 3-, 6-, 12- and 24-hour daily glucose trend graphs, time-in range (TIR), and glucose trends over the last 7 and 30 days in the software platform. After the life cycle of the device, e.g., one, two, three, five years of use, the sensor may automatically shut off, and the user may then apply a new sensor device to the body and recycle the old device.

Following device setup, the user may follow the application's calibration instructions to input their blood glucose values, such as may be measured by the system themselves, or with an auxiliary blood glucose monitoring device, so as to set a baseline reading prior to starting the sensor measurements. Further, during the sensor's life cycle, the mobile software application may also notify the user with alerts and alarms if and when the device needs to be recharged and reapplied with a new adhesive bandage, e.g., at the end of a 14, or 21, or 28-day wear period during an application cycle. Alerts and alarms may also be used if the software application detects signal loss, e.g., photodiode signal loss, BLE communication loss, sensor failure, transmitter failure, and/or if excessive temperature is detected by the sensor. When these alerts and alarms are triggered, the software may guide the user to resolve/troubleshoot the issue with prompts.

Hence, once such biometric and spectral, e.g., RF and optical, data has been collected by the sensing and/or monitoring device, the collected data may be transmitted to an associated computing system, whereby a biomolecule, e.g., a glucose, level, and its effects on the body of the user may be calculated. In various embodiments, the calculations may be performed using one or more of on an on-board or offboard computing system, such as implementing, or otherwise being associated with, a machine learning and/or inference engine, e.g., based on the collected data. And, finally, the process may include outputting at least one of the calculated results, such as by transmitting the results to one or more server systems and mobile computing device running a biomolecule sensing and/or monitoring application configured for displaying such results. All of these steps may be performed non-invasively in a manner that does not physically harm the individual.

In view of the above, a key component of the device is what it does not contain, and that is the device may be configured for using spectroscopy, such as light or radio frequency spectroscopy, to measure biomolecule levels without any portion of the device, or an associated apparatus, penetrating and/or otherwise impinging into the skin. Specifically, the device may be needle-free in that it employs spectroscopic light and/or radio-frequency (RF) and/or microwave techniques to detect the presence of biomolecules, e.g., glucose, in dermal tissue layers, such as where the biomolecule is a photo active, e.g., infrared active, and/or radio-active (including microwave) component. In this regard, at Step I, the device may be placed on a surface of the skin and is configured for directing visible, near infrared (NIR), infrared light, RF, and/or microwave emissions into the skin, and further is configured for receiving and detecting the light, RF, and/or microwave signals reflected back.

Particularly, in various embodiments, the transcutaneous sensor and monitor may include a number, such as up to 10 or more photo-emitters, e.g., Light Emitting Diodes, that direct visible, NIR, and/or infrared light at the skin in a pattern of different light frequencies, intensities, and durations employing the LED array. Further, in various embodiments, the electromagnetic radiation generator may be a microwave sensor unit that includes a power generator, a microwave structure configuration, and a power detector, such as where the microwave sensor unit is configured for transmitting a current through the microwave structure, which results in the production of a fringe field that is directed so as to impinge within the skin and is propagated from the generation side of the microwave structure to the power detections side of the microwave structure.

Hence, in various instances, the referenced emitters may be one or more radio frequency or microwave emitters, and the receiver may, therefore, be an RF or microwave receiver. So being, in such an instance, the emitter and/or receiver may be configured as an antenna array. Accordingly, the device may include a number, such as one or two or more, light receivers, such as photodiodes, which are configured for receiving the light reflected back from the skin, and as indicated, in various embodiments, the receiver may be an antenna array, such as functioning as an RF or microwave receiver. More particularly, the spectral signal detected by the sensor's photodiodes or RF/Microwave receivers is affected by the complex relationship of biomolecules, e.g., glucose, with light and/or RF/Microwave frequency, intensity, and exposure time, which may represent a “biomolecule mediated skin response,” such as a glucose-mediated skin response.

For instance, the referenced spectral analysis may be performed in accordance with a number of different principles. First, in one iteration, it has been determined herein that the presence of glucose, and other biomolecules, such as metabolites, within the skin and/or interstitial fluid, changes its reaction to electromagnetic radiation, such as light and/or radio and/or micro-waves, such as by changing color or transduction through the skin, thereby evidencing a spectral shift, e.g., on the near-infrared, RF, and/or microwave spectrum. For instance, in various instances, a corresponding interaction can be determined using a RF and/or microwave transmission. In either instance, light and/or sound and/or micro-waves may be absorbed and/or reflected differently in the skin and surrounding fluids based on the biomolecules present therein. Consequently, when energy is transmitted into the skin the skin may react in certain characteristic ways to that energy, such as in an observable manner.

Particularly, as discussed herein below, it has been determined that by bombarding the skin and tissues with electromagnetic radiation, e.g., light, RF, or Microwaves of different wavelengths and frequencies, while in the presence of an analyte of interest, it is possible to determine the concentration of an analyte of interest, such as within the interstitial fluids, based on how that analyte affects the ability of the skin to interact with that light. It has been determined herein that various analytes affect the manner by which the skin and tissues respond to electromagnetic energy, e.g., light, when electromagnetic radiation of different wavelengths, frequencies, durations, intensities, etc. is impinged within the skin so as to cause a number of permittivity effects within the tissue, which effects can be corresponded to the analyte concentration in a concentration dependent manner.

Specifically, by varying the impingement of electromagnetic radiation, such as light, RF, and/or Microwaves, within the tissues, for instance, with regard to the wavelengths, frequencies, amplitudes, durations, and the like of the emitted electromagnetic radiation, a number of permittivity effects can be produced within the tissues whereby the extent to which these permittivity effects are evidenced can be measured and correlated to the analyte concentration.

As described herein below, permittivity is a property of all substances that alters the electromagnetic radiation, e.g., light, passing through that medium, in this instance the skin and tissues. These permittivity effects, therefore, happen naturally from the electromagnetic radiation, e.g., light, being directed into the skin and tissue and interacting with the constituents therein in a manner that can be detected, determined, and to some degree predicted. In essence, in the case of a photonics array, by shining light into the skin and tissues, the skin or tissue is actually changed in a minor but detectable way, such as by changing the various permittivity effects set forth herein. Specifically, the reason the skin and tissue changes is because as the light passes through the skin, some of the light is absorbed, some of the light is scattered, some is refracted, some is polarized, and of course some of this light gets reflected back by the skin itself. And when the light gets absorbed, refracted, reflected, and the like, the physical properties of the skin and tissue are actually altered at the point that the light is shone therein such as in a characterized manner with respect to the way the light is affected by these changes to the permittivity properties. And these permittivity properties are unique to every single frequency and every single substrate and with respect to every different analyte.

Accordingly, presented herein, are multi-sensing detection devices having a number of electromagnetic radiation (optical, RF, and/or Microwave) emitters that are configured for directing electromagnetic radiation into the tissues in a number of different patterns of emittance, including varying wavelengths, frequencies, amplitudes, energy levels, intensities, e.g., luminosities, durations, and the overall emission schema so as to produce and explore these aforementioned permittivity effects, because the system is trying to generate a model to determine how much the light is being affected by the presence of the analyte within the tissue in the current context, so as to use that context and those resultant effects to map the permittivity effects to the concentration of the analyte, so as to determine the concentration of the analyte.

By varying various of the properties of the electromagnetic radiation being emitted and directed into the tissue, the energy levels of the energy can be manipulated. Further, by manipulating the energy levels being delivered to the tissue, these permittivity properties may be altered, but in a manner that is mediated by the concentration of analytes, e.g., glucose, being present within the skin and tissues. For example, by increasing the amplitude of the waveform more energy can be delivered into the tissue, without necessarily changing the frequency of the electromagnetic wave. Thus, by changing the pattern of energy response in a manner that is characteristic of the analyte concentration, these properties affecting the response of the tissue and skin can be determined and be correlated to that analyte concentration. Therefore, measuring the different permittivity responses within the tissue provides concentration dependent information about the analyte and how it is affecting the tissue because those permittivity properties are altered differently depending on the level of that analyte within the tissue.

More particularly, these permittivity effects can be used to produce one or more fingerprints that can in turn be used to generate a signature as to how the tissue is responding to light in the presence of the analyte, and thus, the fingerprint(s) and/or signature can be used to predict the level of the analyte within the tissue. In essence, the emittance of the electromagnetic radiation into the skin and tissues, in accordance with a determined pattern of emittance, provokes the aforementioned permittivity effects, each of which can produce a unique pattern of response that can be characterized much like a fingerprint.

Collectively these fingerprints, such as in the case of light, can evidence patterns of absorbance, refraction, scattering, polarization, and reflectance responses, one or more of which can be used as fingerprints so as to generate a unique signature that can then be equated with the concentration of an analyte of interest. Therefore, these permittivity properties form one or more patterns that can be measured whereby the pattern is unique to the state of the skin in the presence of an analyte, such as glucose, at the times the measurements are taken. Thus, the pattern of these permittivity effects from fingerprints that one or more of, e.g., collectively, from a signature from which the concentration of the analyte, e.g., glucose, can be derived. Hence, the devices, systems, and their methods of use disclosed herein may be configured for detecting and quantifying such characteristic “permittivity” changes in the skin and tissues.

Further, in various embodiments, detection of biomolecules within the skin and spaces therebetween may be guided in part by Beer-Lambert's law, which is based on light absorption being directly proportional to the concentration of light absorbing elements being present within the skin, their concentration, and the optical path length traversed by the light signal. Consequently, Beer-Lambert's law may roughly be attempted to be mechanized so as to account for the presence and/or differences in levels of biomolecules in the skin based on light wave absorption, reflectance, and the time from emittance to reception, while accounting for changes in the optical path, such as by using one or more of light and/or laser-based spectroscopy. However, the mechanization of Beer-Lambert's law is not a straightforward process, as the law in and of itself is unable to fully capture the nuances of photo-based biomolecule, e.g., glucose, detection without the devices, mechanics, and calculations performed by the methods disclosed herein.

More specifically, in order to account for a number of different skin types, pigmentation, absorption characteristics, biomolecule features, light wave affectations, and other such variables, use of an artificial intelligence has been developed to account for the variance in such changing conditions. In particular embodiments, these methods are useful for determining a level of glucose, such as in the interstitial space, which in turn is useful for monitoring and/or modulating hyper glycemia, diabetes, and other health conditions. Such measurements and determinations have been attempted, but have heretofore been unsuccessful because, as discussed above, it is difficult to maintain a consistent topology of the dermal layers within which the measurements are taken. The present technology overcomes such difficulties by using an array of photo-, RF-, and/or microwave emitters, stably locking the sensing device immovably in a singular position on the body, e.g., preventing lateral and rotational movement, and/or correcting for variable inconstancy, such as minimal movement, via a suitably configured data structure, such as an Artificial Neural Network, as herein described.

Specifically, in various embodiments, an AI module employing machine learning and inference generation may be used so as to develop one or more models to take the various different datapoints and variables, e.g., measurements, as well as changes thereto into account by building a data structure, such as an artificial neural network, by which to make a determination of a biomolecule level within the skin, vessels, and interstitial spaces, in a manner that one or more unhealthy, e.g., disease, conditions can be sensed, monitored, and/or tracked over time. Data, e.g., measurement data, to be entered into such data structures may be collected by using a variety of different energy emitters and receivers so as to produce a Photonic-, RF-, and/or Microwave-spectral array, along with other biological data, that can be analyzed in accordance with a number of different principles set forth herein. With this in mind, the present sensing and monitoring devices have been developed to work in conjunction with a suitably configured analytics platform so as both sense and determine a biomolecule, e.g., glucose, mediated skin response, as well as to analyze the same, such as by using a deep learning-trained, locked data structure, such as implemented as an ANN.

For example, in performing these procedures as set forth at FIG. 7, at Step 1 at 110a, energy, such as light and/or radio or micro-wave energy, may be emitted into the skin, the biomolecules present therein and around then react to that energy, as described above, whereby, some of the light or radio and/or microwaves are absorbed, some of the energy may be refracted, scattered, polarized, and some of the energy is reflected back, such as to the appropriately configured receiver, e.g., photodiodes or RF or micro-wave receivers. Consequently, at Step 2 at 110b, the reflected energy, e.g., light or RF or microwave, signals are collected and preprocessed, and then transmitted, e.g., wirelessly, to one or more of remote computing systems and/or associated client computing devices, such as a mobile computing device, e.g., for display thereby. Specifically, in various embodiments, the sensing and/or monitoring device may be configured to automatically emit and capture reflected energy signals, as well as the permittivity effects associated therewith, every 1, 2, 3, 4, or 5 or more minutes.

Specifically, at Step 2, from the detected spectral signal, the analog-to-digital converter (ADC) coupled to the PCBA may then preprocesses the spectral data. For example, in an exemplary embodiment, the emitter may be a photo-emitter, such as an LED, and the receiver may be a photodiode. In such an instance, the data collected by the photodiode may include one or more of the LED capacitance, the current, voltage, amplitude, and/or the photodiode intensity, which may be saved to a local, or remote data storage 76, such as in a first-in-first-out (FIFO) flash memory. In certain instances, the same may be similarly true for detecting RF or microwave frequencies and wavelengths.

In any of these instances, the onboard memory may be configured to store such data for a prolonged period of time, such as at least 7, 14, 21, 28 or more days, including 1 or 2 or even 3 or more months, such as encapsulating multiple, 1, 2, 3, 4, or more wear periods, all of which can be stored on the local flash memory. Also, during the aforementioned preprocessing steps, measurements with poor quality, broad wavelength interference, including measurements derived from bad transmission, incomplete signal, electrical interference, excessive pressure on the sensor, an obscuring substance on the skin, or other factors, may be filtered, such as using an onboard AI, system, such as filtering sub-system, such as a data valuation with reinforcement learning (DVRL) neural net. For example, in certain instances, this preprocessing DVRL neural net filtering may be trained.

Following signal preprocessing, at Step 4 at 110d, the PCBA's communications module, e.g., miniature radio transceiver, communicates this information, e.g., via Bluetooth®, BLE, Wi-Fi, or other communications protocol, to an associated computing device, such as to a software platform downloaded to and being run on a smart device. In such instances, as can be seen with respect to FIG. 8, the mobile application software platform can convert the reflected energy's spectral signal using the AI module, e.g., ANN algorithm, to derive an estimated biomolecule value, such as glucose values.

More particularly, in specific instances, such as where the biomolecule of interest is glucose, the sensing and monitoring device may be specifically attuned to estimate glucose values such as in a concentration ranging between 40-400 mg/dL, e.g., based on the glucose-mediated skin response, described herein, to the directed visible, NIR light, RF, and/or microwave emissions. As indicated above, the PCBA sensor and/or monitor may have any reasonable number of energy emitters, such as 1, 3, 4, 6, 8, 10, or more, so as to direct energy, such as visible, NIR, and/or IR light or RF and/or microwave energy into the skin. As indicated, in a particular embodiment, the emitter group may include or otherwise be associated with a an optical emitter group and/or RF or microwave emitter, e.g., an antenna-based emitter group, such as including a microwave structure.

In certain embodiments, each emitter may transmit energy of different wavelengths, such as where the energy wavelength is of a length and frequency to produce a reaction in the biomolecule, and/or surrounding tissues, e.g., permittivity effects, which may change based on the received energy characteristics, e.g., wavelengths and frequencies, being directed into and/or reflected back from the skin. In various instances, as the electromagnetic energy is received within the skin and/or tissues, it provokes a series of reactions, e.g., primitivity effects, within the skin and tissues. These changes can be used to determine biomolecule value, such as where the resultant energy shift and/or change in intensity may be determinable based on the concentration of one or more analytes being present within the skin. In other words, the variable impingement of electromagnetic energy into the skin and tissue provokes a response within that tissue with regard to how that tissue and/or the interstitial milieu interacts with that electromagnetic radiation in the presence of one or more analytes therein. In such instances, various properties, such as absorption and/or refraction and/or reflection, of that radiation within the tissue is changed in a concentration dependent manner based on the amount of a given analyte, such as glucose, being present therein. Hence, the number of emitters, e.g., LEDs, RF, or microwave, the wavelengths they emit, and their pattern of admission may vary dependent on the molecule, e.g., analyte, being observed, and the energy required to produce the observable energy, in some instances spectral, shift(s).

In such embodiments, the analytics system may be configured to observe wavelength emittance, duration, amplitude, and intensity, such as luminosity, as well as the affects, e.g., permittivity effects, thereof on the body, and can then weight and/or bias the wavelength, duration, amplitude, intensity, etc. based on the observable effect, and/or can change the amplitude, intensity, duration, and/or the wavelength itself, such as to provoke the evidenced permittivity effects and/or desired energy, e.g., spectral, shifts so as to better make and evaluate the concentrations of the biomolecule and its effect on the body tissues. From this data, and in accordance with the procedures disclosed herein, the most effective array of emitters for producing the desired response for taking measurements may be determined, the effective emitters can be selected, and their emission and wave characteristics can be set so as to make the requisite measurements, such as to generate a number of energy arrays from which an accurate analyte value may be determined. For instance, where glucose is the biomolecule of interest, at Step 1, when the sensor performs a measurement of a subject's glucose level, the electromagnetic radiation emitters, e.g., LEDs, RF, and/or Microwave structures, are activated, such as in a determinable sequence, e.g., from lowest to highest wavelength or frequencies, or vice versa, or may be activated in a non-sequential manner, e.g., randomly, in a pattern of varying electromagnetic radiation wavelengths, frequencies, intensities, and amplitudes, as well as electromagnetic radiation activation times, sequences, and durations.

For example, in implementing this step for detecting and monitoring a user's biomolecule, e.g., glucose, levels, at 110a, the method may include one or more of the following steps. First, an emitter, e.g., a photoemitter, RF, or microwave emitter, or the like, may be energized, such as by supplying power to an associated capacitor, and a first emission, e.g., of light, at a first wavelength or frequency may be emitted and directed into the skin of the wearer of the device, e.g., user. Additionally, the same process can be repeated so that the first emitter is charged and activated again or a second emitter is charged so as to emit a second emission, e.g., of light at a second wavelength of frequency, likewise, a third emitter may emit a light at a third wavelength, and the same for the emittance of a fourth light at a fourth wavelength, the emittance of a fifth light at a fifth wavelength, likewise a sixth light at a sixth wavelength, and the same for as many emitters are selected for emitting wavelengths, such as seventh, eighth, nineth, tenth, or more.

In particular embodiments, where the emissions of electromagnetic radiation are light emissions, the photo, e.g., light, emitters may be configured for emitting, and the photo receivers, e.g., photodiodes, may be configured for receiving light from a broad-spectrum, such as in the range from about 500 nm to about 1000 nm, and/or from about 1000 nm to about 1700 nm to about 2000 nm to about 2500 nm to about 3000 nm or more. For instance, in various embodiments, two optical arrays of photoemitters may be employed, such as where the first optical array is configured as a broad-spectrum array that includes a number of photoemitters that are adapted for emitting light in the range from about 500 nm to about 1000, and further the second array may be adapted as a broad-spectrum array that includes a number of photoemitters that are configured for emitting light in the range from about 500 nm to about 1000 nm to about 1700 nm to about 2500 nm. In various instances, an optical array of three emitters, e.g., configured for emitting light with a wavelength within the range of infra-red, red, and green light may be include in substitution for or in addition to one or more of the other arrays, such as where the three-emitter array may be a photoplethysmography (PPG) sensor unit.

In some embodiments, the first optical array may include any number of emitters (e.g. three emitters, four emitters, six emitters, etc.), which emitters can be positioned proximate to, e.g., surround one or more energy receivers, such as a photodiode. Each emitter can emit light of a different wavelength, and each wavelength can be in a range from about 500 nm to about 550 nm, a range from about 550 nm to about 600 nm, a range from about 600 nm to about 650 nm, a range from about 650 nm to about 700 nm, a range from about 700 nm to about 750 nm, a range from about 750 nm to about 800 nm, a range from about 800 nm to about 850 nm, a range from about 850 nm to about 900 nm, a range from about 900 nm to about 950 nm, and/or a range from about 950 nm to about 1000 nm. A given range can correspond to a single emitter, such that a maximum of one emitter can have a wavelength in any one of the aforementioned wavelength ranges. Alternatively, two or more emitters can be configured to emit light of a wavelength within a single of the aforementioned ranges (e.g., a first emitter emits light at approximately 610 nm and a second emitter emits light at approximately 640 nm). As a more specific example, the various emitters of the first array can collectively emit light of a first wavelength, which may be about 550 to about 649 nm; light of a second wavelength, which may be about 650 nm to about 849 nm; light of a third wavelength, which may be about 850 nm to about 939 nm; and light of a fourth wavelength, which may be about 940 nm to about 1040 nm.

The second array may include any number of emitters (e.g., six emitters), which can surround one or more receivers. Each emitter can emit light of a different wavelength, and each wavelength can be in a range from about 1000 nm to about 1050 nm, a range from about 1050 nm to about 1100 nm, a range from about 1100 nm to about 1150 nm, a range from about 1150 nm to about 1200 nm, a range from about 1200 nm to about 1250 nm, a range from about 1250 nm to about 1300 nm, a range from about 1300 nm to about 1350 nm, a range from about 1350 nm to about 1400 nm, a range from about 1400 nm to about 1450 nm, a range from about 1450 nm to about 1500 nm, a range from about 1500 nm to about 1550 nm, a range from about 1550 nm to about 1600 nm, a range from about 1600 nm to about 1650 nm, a range from about 1650 nm to about 1700 nm, a range from about 1700 nm to about 1750 nm, and/or a range from about 1750 nm to about 1800 nm or more.

Alternatively or in addition, one or more emitters can emit light having a wavelength in a range between approximately 1800 nm and approximately 3000 nm. For example, a given emitter can be configured to emit light having a wavelength in a range from about 1800 nm to about 1850 nm, a range from about 1850 nm to about 1900 nm, a range from about 1900 nm to about 1950 nm, a range from about 1950 nm to about 2000 nm, a range from about 2000 nm to about 2050 nm, range from about 2050 nm to about 2100 nm, a range from about 2100 nm to about 2150 nm, a range from about 2150 nm to about 2200 nm, a range from about 2200 nm to about 2250 nm, a range from about 2250 nm to about 2300 nm, a range from about 2300 nm to about 2350 nm, a range from about 2350 nm to about 2400 nm, a range from about 2400 nm to about 2450 nm, a range from about 2450 nm to about 2500 nm, a range from about 2500 nm to about 2550 nm, a range from about 2550 nm to about 2600 nm, a range from about 2600 nm to about 2650 nm, a range from about 2650 nm to about 2700 nm, a range from about 2700 nm to about 2750 nm, a range from about 2750 nm to about 2800 nm, a range from about 2800 nm to about 2850 nm, a range from about 2850 nm to about 2900 nm, a range from about 2900 nm to about 2950 nm, or a range from about 2950 nm to about 3000 nm.

A given range of electromagnetic radiation can correspond to a single emitter, such that a maximum of one emitter can have a wavelength in any one of the aforementioned wavelength ranges, or dependent on the type of emitter, the wavelength can be in the microwave and/or RF wavelength ranges. Alternatively, two or more emitters can be configured to emit electromagnetic radiation, e.g., light, of a wavelength within a single of the aforementioned ranges (e.g., a first emitter emits light at approximately 1410 nm and a second emitter emits light at approximately 1435 nm). As a more specific example, the various emitters of the second array can collectively emit light of a first wavelength, which may be from about 1200 nm to about 1299 nm; light of a second wavelength, which may be from about 1300 nm to about 1399 nm; light of a third wavelength, which may be from about 1400 nm to about 1499 nm, including about 1450 nm; light of a fourth wavelength, which may be from about 1500 nm to about 1549 nm; light of a fifth wavelength, which may be about 1550 nm to about 1649 nm; and light of a sixth wavelength, which may be from about 1650 nm to about 1750 nm.

One, two, or more arrays can be included in the multi-sensing detection device (e.g., biometric sensing and/or monitoring device 15) and can each include any particular number of emitters. For example, two arrays can include a total of 9 emitters (e.g., 3 emitters in a first array and 6 emitters in a second array), or may include a total of 10 emitters (e.g., 4 emitters in a first array and 6 emitters in a second array), or any sub-combinations thereof.

Regardless of the specific array, it should be noted that the frequency exposure for emitters, e.g., LEDs, is typically not a specific value. Instead, LEDs typically emit light with a central value and some bandwidth that includes the central value and presents as a normal distribution around the mean. For example, many LEDs marketed, advertised, or otherwise indicated as being configured to emit light at a particular frequency are, in fact, configured to emit light within a range that is the particular frequency value ±33 nm; however, this range can vary according to the specific emitter, LED (e.g., model), and/or manufacturer. Accordingly, the various light emission frequencies discussed here should be understood to contemplate any frequency variations that can typically occur according to current acceptable manufacturing variations and/or tolerances. As non-limiting examples, any one of the light sources can be an LED, and/or the wavelength of the emitted light may vary by ±about 10, about ±10 nm to about ±25 nm, about ±25 nm, about ±50 nm, about ±100 nm, and/or the like. In certain embodiments, a filter may be placed over one or more photoemitter and/or over photo-receiver, e.g., photodiode, such that each photoemitter and/or each photodiode can emit and/or receive and detect a very narrow range of wavelengths. In some implementations, each filter may be a physical device consisting of a small piece of plastic, glass, or other semi-transparent material that has band gap filtering properties. The band gap filtering properties can be from the filter material itself, or from additional materials added to a base material. The filter material may include an anti-reflective coating and may be engineered to only allow passage of light within a narrow bandwidth.

The various electromagnetic radiation, e.g., light, emissions can be emitted according to a specific light emission pattern. The emission pattern can include a particular order of electromagnetic radiation emissions such that emissions of particular wavelengths are emitted at a particular energy band, e.g., a particular luminosity and/or amplitude (whether constant or variable/dynamic during a given emission), in a particular order and according to a particular timing and duration. The timing element(s) of the electromagnetic radiation, e.g., light, emission pattern can refer to emission duration(s) (e.g., the amount of time a given emission is emitted), intermission(s) (e.g., the time between the end of one emission and the start of a next sequential emission), emission start/stop times (e.g., as determined with reference to an initial start time), and/or a total emission duration (e.g., the time from the start of the first emission to the end of the last emission).

Each light emission can be emitted for a particular duration. Alternatively or in addition, each emitter can be configured to emit light for a particular light emission or intermission duration (e.g., a predetermined duration) independently or collectively, such as in a predetermined sequence of emissions. The light emission duration and/or intermission can be the same for some or all of the emitters. For example, the light emission duration or intermission can be the same for all emitters of a given array and/or for all emitters regardless of array. Alternatively or in addition, the light emission or intermission duration for emitters of a given array can be different from the light emission duration for emitters of the other array. Alternatively or in addition, the light emission or intermission duration can be the same for some emitters and different for other emitters. Alternatively, the light emission or intermission duration can be different for every emitter of a given array and/or for all emitters regardless of array. As a non-limiting example, a given emission or intermission duration can be in a range from about 100 ms to about 5000 ms. As more specific non-limiting examples, a given emission duration can be in a range from about 250 ms to about 500 ms, a range from about 500 ms to about 750 ms, a range from about 750 ms to about 1000 ms, a range from about 1000 ms to about 1250 ms, a range from about 1250 ms to about 1500 ms, a range from about 1500 ms to about 1750 ms, a range from about 1750 ms to about 2000 ms, a range from about 2000 ms to about 2250 ms, a range from about 2250 ms to about 2500 ms, a range from about 2500 ms to about 2750 ms, a range from about 2750 ms to about 3000 ms, a range from about 3000 ms to about 3250 ms, a range from about 3250 ms to about 3500 ms, a range from about 3500 ms to about 3750 ms, a range from about 3750 ms to about 4000 ms, a range from about 4000 ms to about 4250 ms, a range from about 4250 ms to about 4500 ms, a range from about 4500 ms to about 4750 ms, or a range from about 4750 ms to about 5000 ms.

The light emissions of particular wavelengths can be arranged in any particular order. For example, the various LEDs can be configured to emit light sequentially such that only a single emitter emits light at a given time, regardless of the array in which a given emitter is arranged. Alternatively, the various LEDs of a given array can be configured to emit light sequentially such that only a single emitter emits light at a given time, such that a given emitter of a first array and a given emitter of a second array can (but not necessarily must) emit light at times that do not overlap, but, in other instances, the light from the first and/or second arrays may be emitted at overlapping times. In various instances, the various emitters may be configured to emit light in a “popcorn” style, which may not be random, but may be in a series of different patterns wherein light from emitters from the various different arrays are emitted in a pattern that does not particularly depend from which array the emitter is associated.

The light emission pattern can include an intermission between a given pair of sequential emissions. A given emission can be simultaneously included in two pairs of sequential emissions. To illustrate, an example sequence can include a first emission ending, a first intermission occurring, a second emission starting, the second emission ending, a second intermission occurring, and a third emission starting; in this illustrative example, the first and second emissions can form a first pair of sequential emissions, and the second and third emissions can form a second pair of sequential emissions. The intermission between each pair of sequential emissions can have the same duration, such that the timing between each sequential emission is constant. Alternatively, one, some, or all of the intermissions between pairs of sequential emissions can have a different duration. As a non-limiting example, a given intermission can be between 1 ms to about 2000 ms. As more specific non-limiting examples, a given intermission can be in a range from about 1 ms to about 250 ms, a range from about 250 ms to about 500 ms, a range from about 500 ms to about 750 ms, a range from about 750 ms to about 1000 ms, a range from about 1000 ms to about 1250 ms, a range from about 1250 ms to about 1500 ms, a range from about 1500 ms to about 1750 ms, or a range from about 1750 ms to about 2000 ms.

Alternatively or in addition, there can be no intermission between a given pair of sequential electromagnetic radiation emissions. For example, the end of a first emission can occur simultaneously (or substantially simultaneously, taking into consideration communication and/or hardware delays, for example) with the start of a second emission. In various embodiments, some or all emissions can overlap with one or more different emissions, and as such, the electromagnetic radiation emission pattern can include a start time and an end time for each emission, with each start time and end time being determined with reference to some initial start time (e.g., a first emission can start at T=0 ms and end at T=500 ms and a second emission can start at T=150 ms and end at T=900 ms). Furthermore, the electromagnetic radiation emission pattern can include both one or more intermissions (e.g., between one or more pairs of sequential emissions) and one or more overlapping emissions. To illustrate, an example sequence can include a first emission starting, the first emission ending, a first intermission occurring, a second emission starting, a third emission starting, the second emission ending, a second intermission occurring, a fourth emission starting, the third emission ending, and the fourth emission ending, and so forth.

As an example, the electromagnetic radiation, e.g., light, emission pattern can include a predetermined number of emissions. Each emission can be emitted by a corresponding emitter, such that a given emitter provides one emission per electromagnetic radiation emission pattern. Alternatively, one or more emitters can be configured to emit electromagnetic radiation, e.g., light, at a given wavelength two or more times within one emission pattern. If a light emission pattern includes multiple different emissions from a single given emitter, two or more emissions from the single given emitter can have the same duration, and/or two or more emissions from the single given emitter can have different durations.

Each light emission can be emitted at a particular luminosity and/or amplitude. The luminosity or amplitude of a given emission can be a constant energy output for the duration of that particular emission. One, some, or all emissions can have the same luminosity and/or amplitude of emitted light wave. One, some, or all emissions can have different luminosities or amplitudes. The luminosity and/or amplitude of a given emission and/or wavelength can be variable and/or dynamic. For example, the luminosity or amplitudes can be adjusted for a given emission by adjusting the current supplied to the corresponding emitter. Stated differently, different or variable amounts of energy can be outputted during a given emission according to a particular corresponding luminosity and/or amplitude function. For example, a given emission can start at an initial luminosity (e.g., 0 mW, 2 mW) and can incrementally increase to a maximum output value (e.g., 10 mW, 20 mW, 30 mW, 40 mW, 50 mW). The initial luminosity can be zero or can be a non-zero value (e.g., 5 mW, 10 mW).

It should be noted that the amount of current is an indicator of the amount of electromagnetic radiation presented, and if the emitter (e.g., LED and/or corresponding circuit) were modified, the current values could be different. Accordingly, the exemplary current values provided herein are provided as non-limiting examples of expected values, e.g., for a typical LED currently available. In various embodiments, the luminosity output and/or amplitude of the emitted wavelength can be increased or decreased to compensate for the particular hardware configuration (e.g., increasing the luminosity or the amplitude of the emitted wavelength to compensate for the inclusions of a filter, such as a polarizing filter). The luminosity and/or amplitude of emitted wavelength can incrementally increase, incrementally decrease, or increase and/or decrease according to any useful function (e.g., linear, quadratic, piecewise, and/or any other type of function). A “sweep” of a given light emitter or set of light emitters (described more fully herein) can include a predetermined number of measurements (e.g., 20 measurements); some or all of the measurements can correspond to different light emission luminosities and/or amplitudes of emitted wavelengths (e.g., each measurement can correspond to an incrementally higher or lower luminosity of emitted light than that of one or more previous measurements, as dictated by the pertinent light emission pattern).

The light emission pattern can include a particular luminosity or energy output for each emission and/or the pattern may include the emission of wavelengths with determined amplitudes. The luminosity and/or amplitude can be constant throughout a given emission. Alternatively or in addition, the luminosity and/or amplitude can be variable for a given emission. One, some, or all emissions can have the same luminosity and/or amplitude function, and/or one, some, or all emissions can have different luminosity and/or amplitude functions. The maximum luminosity and/or amplitude value can be determined based on the hardware used for each emitter or can be otherwise predetermined. As a non-limiting example, emitters can be configured to emit energy in short emission bursts (e.g., approximately 500 ms to approximately 5000 ms) and can be configured to emit a predetermined number of bursts per sweep or reading (e.g., 20 bursts per sweep). A given sweep by a given emitter can include iteratively incremented bursts (e.g., bursts that incrementally increase in emission time) with each burst following a predetermined luminosity and/or amplitude pattern (e.g., start at an initial luminosity and/or amplitude and increasing to a maximum luminosity and/or amplitude value during the corresponding burst). Alternatively or in addition, each individual burst can be emitted at an approximately constant luminosity and/or amplitude and each iterative burst can change in luminosity and/or amplitude.

After the emitters emit energy, one or more energy receivers (e.g., photodiodes or power detectors) can collect reflected or transduced energy (e.g., light reflected from or by the wearer's body or biomolecules therein, such as after having experienced the referenced permittivity effects), and the reflected or transduced energy can provide a voltage response that is digitized by the device. The presence of particular biomolecules of interest (e.g., blood glucose) in the skin can alter the energy response (e.g., IR response) of the reflected light or transduced energy. Particularly, by varying the patterns of electromagnetic radiation being emitted and directed into the skin and tissue, with regard to varying the wavelengths, frequencies, luminosity, amplitudes, durations, and the like of the emitted electromagnetic radiation, as set forth above, a number of permittivity effects can be produced within the tissues. And as discussed herein below, the extent to which these permittivity effects are produced by the interaction of the electromagnetic radiation with the tissue, e.g., due to the presence of one or more analytes therein, can be measured and correlated to the analyte concentration.

Specifically, all substances have a number of permittivity properties that alter the electromagnetic radiation, e.g., light, passing through them. These permittivity properties, therefore, happen naturally from the light (or RF or microwaves) being directed into the tissue and interacting with the biomolecules, e.g., analytes, therein in a manner that can be detected, determined, and predicted. Accordingly, by emitting electromagnetic radiation into the tissues, it actually changes the tissue in a minor but detectable way, such as by changing the various permittivity properties set forth herein.

More particularly, the reason the tissue changes is because as electromagnetic radiation passes through the skin thereof, some of the radiation is absorbed, and where the electromagnetic radiation is light, some of the light is scattered, some is refracted, some is polarized, and of course, some of this light gets reflected back by the tissue itself. And when the electromagnetic radiation, e.g., light, gets absorbed, transduced, refracted, reflected, scattered, polarized, and the like, the physical properties of the tissues are actually altered at the point that the electromagnetic radiation impinges within it, such as in a characterized manner with respect to the way the electromagnetic radiation is affected by these changes to the permittivity properties of the skin and tissue. Hence, once the electromagnetic radiation, e.g., light, has entered the skin and tissue, a portion of the electromagnetic radiation, e.g., light, will be absorbed, some will be refracted, some of it will be scattered, some of the light will be polarized, and some of the electromagnetic radiation will be reflected back.

In these regards, upon receipt of the transduced and/or reflected energy back by the multi-detection sensing device, the returned signal will be converted from analog to digital signal and then the digital signal may be transmitted to the analytics platform of the system, such as where the digital signal data will be cleaned up, pre-processed, and then analyzed, as described in detail herein below. For instance, in preparation for analyzing the data, it may be pre-processed, as described herein, and then be fed into a data structure, such as a decision tree or neural network by which the signal data can be organized, such as into a collection of reads, and then be processed. For these purposes, the systems and/or methods of the disclosed technology (e.g., decision trees, neural net systems, and/or other ML systems) can be configured to identify patterns in the altered energy response and estimate the presence and/or concentration of one or more biomolecules of interest (e.g., blood glucose) based at least in part on the altered energy response patterns, as described more fully herein. Of note, the disclosed systems and methods need not directly measure concentrations of the biomolecule(s) of interest; rather, the altered energy response patterns may be used to estimate the presence and/or concentration of the biomolecule(s) of interest, such as based on the permittivity properties disclosed herein.

While the various operating parameters of the measurement device have been largely discussed heretofore as being preprogrammed, the disclosed technology is not so limited. On the contrary, the disclosed technology includes systems and methods for dynamically adjusting the operating parameters of the device (e.g., the number, duration, cycling, pattern, and/or other characteristics of energy emissions emitted by the device). As a non-limiting example, the disclosed technology includes applying one or more reinforcement learning techniques to dynamically determine (e.g., optimize) the operating parameters of the device that produce better results (e.g., more accurate).

Stated differently, the machine learning algorithms and reinforcement learning technique(s) discussed herein can learn and/or identify different operating parameters of the hardware to improve the biomolecule estimates of the disclosed technology (e.g., as determined by a neural net system and/or other ML system). If it is determined that better results could be achieved by applying operating parameters different from the current operating parameters of the device, the disclosed systems and methods can include automatically adjusting the operating parameters of the device to match the newly identified (e.g., optimized) operating parameters. Accordingly, such techniques can provide real-time (or near-real-time) parameter modifications to energy output, e.g., illumination, levels, quiet (intermission) times, and/or the number of measurements obtained in a sweep, as non-limiting examples.

Hence, in view of the above, after emission, the process may further include receiving, at a first, second, third, etc. energy receiver (e.g., photoreceiver and/or power detector), at least a portion of the first electromagnetic radiation, e.g., light or other energy, reflected from or transduced through the skin of the user, and at least a portion of the second electromagnetic radiation, e.g., light, reflected from a skin of the user, and at least a portion of the third electromagnetic radiation, e.g., light, reflected from a skin of the user, and at least a portion of the fourth electromagnetic radiation, e.g., light, reflected from a skin of the user, and at least a portion of the fifth electromagnetic radiation, e.g., light, reflected from a skin of the user, and at least a portion of the sixth, seventh, eighth, nineth, tenth, and so on, electromagnetic radiation, e.g., light, reflected from the skin of the user. For example, once the electromagnetic radiation, e.g., light, has entered the skin, a portion of the electromagnetic radiation, e.g., light, will be absorbed, and where the electromagnetic radiation is light, some will be refracted, some of it will be scattered, some of the light will be polarized, and some of the electromagnetic radiation will be reflected back and thereby received by 1, 2, or more energy, e.g., photodiode sensor, receivers on the PCBA, which photoreceptors should be attuned to cover the range of potential LED wavelengths, and the reflected light may be measured after each LED activation.

Multiple measurements (e.g., individual data points indicating measurements of reflected light) can be obtained during a given emission. For example, 5, 10, 15, 20, 30, 40, 50, or any other number of measurements can be obtained during a given emission. The same number of measurements can be obtained during each emission of a given emission pattern, or a different number of measurements can be obtained for one, some, or all emissions. For the sake of simplicity, the performance of an entire electromagnetic radiation, light, emission pattern for a given emitter can be referred to as a “full sweep” by that emitter, and a full sweep of all emitters can be referred to as a “reading.” During a full sweep, several measurements can be obtained (e.g., one or more measurements or data points each corresponding to measurements of light reflected from one or more particular emissions).

However, not all measurements will be “good”; that is to say, some measurements may include errant data due to some hardware issue or some other cause, such as drift. In any event, one or more pre-processing, filtering, and/or cleaning steps, as discussed herein below, can be performed to remove or correct for “bad” or errant data prior to analyzing the data to determine the presence and/or concentration of the biomolecule of interest. As described herein below, there are a number of preprocessing methodologies by which such data may be cleaned and/or corrected for prior to being transmitted from the detection device itself and/or after being received within the analytics system.

The detection device (e.g., biometric sensing and/or monitoring device 15) can be configured to perform one or more reads up to a full sweep on a regular basis or schedule. For example, the device can be configured to perform a number of reads or a full sweep once every predetermined period of time, such as once every 30 seconds, once every minute, once every 2-3 minutes, once every 5 minutes, once every 10 minutes, or any other period of time. The frequency of sweeps can be determined to balance a sufficiently constant data flow with a sufficiently long battery life of the device. If a given sweep or reading is determined to include erroneous data (e.g., due to hardware error and/or external factors such as vibration or external sensor interference), a second sweep or set of one or more readings (e.g., re-sampling) can be performed, which may be performed with an appropriate filter being applied.

The various readings, e.g., measurements, can be used to determine changes in electromagnetic radiation, e.g., light, absorption (e.g., IR absorption) of the user's body during each emission and/or each full sweep. As explained herein, for each detection device (e.g., biometric sensing and/or monitoring device 15), there can be some base parameters (e.g., a base absorption and/or reflectance pattern) that are complex and embedded within the analytics system. Effectively, these base parameters can be used to determine the response of the skin (and/or other bodily aspects, such as the blood, organ(s), and/or interstitial fluid) to different electromagnetic radiation, light, emissions in view of the biological responses of a particular user at a particular skin location of the device and/or the present blood glucose concentration for the particular user.

Hence, as described herein, the light absorption and/or reflectance values corresponding to the various emissions can form a user-specific (and/or device-location-specific) pattern. The disclosed systems and methods can be configured to determine a correspondence, correlation, and/or association between the pattern of a user's light absorption and/or reflection levels and the tissue and/or analyte levels (e.g., such as the user's tissue or blood glucose levels). Where necessary, the system can correct for errors in the readings, such as by the performance of one or more pre-processing, filtering, and/or data cleaning methodologies. Additionally, where necessary, the disclosed systems and methods can also correct for drift issues, such as sensor drift (e.g., changes in the measured light absorption response over time due to hardware changes such as component degradation, temperature, electronic characteristics, and/or current power levels, as non-limiting examples) and/or skin drift (e.g., changes in the measured light absorption response over time due to environmental and/or biological factors such as temperature, humidity, and/or biological factors affecting the skin structure and composition, as non-limiting examples). After preprocessing and/or correction, if necessary, the system can then process the data, such as within the data structure, and map the inputs to single output from which the concentration of an analyte in question can be determined.

For instance, the disclosed systems and methods can include evaluating trends in a particular user's light absorption and reflectance response over time (e.g., performing a statistical analysis thereon) and can evaluate the light absorption and/or reflectance response for a particular user (e.g., current responses and/or historical responses) in view of corresponding data (e.g., current data and/or historical data relating to component age, expected working life for various components, current component health, historical component health, current software version, current/historical temperature and/or humidity data, current/historical biological factor data, and the like, and/or any combination thereof). The various correspondences, correlations, mappings, and/or associations between and among the patterns of the user's electromagnetic radiation, e.g., light, absorption and/or reflection levels and the analyte, e.g., blood glucose, levels can provide a readout, such as a prediction or estimation of what a present or future glucose concentration is or will be. However, in various instances, to better effectuate such predictions, estimations, and/or other analytic results, it may be useful to first determine a preliminary calibration and/or “signature” for the particular user and/or the particular skin location of the device.

For example, the analytics system (e.g., AI module or any other system or module configured to determine the presence and/or concentration of a biomolecule of interest) can be configured to accurately estimate the presence and/or concentration of a biomolecule of interest based on as few as one reading, which can refer to data indicative of a full sweep of all energy, e.g., light, emitters (e.g., a full progression of the light emission pattern for each corresponding light emitter) and any additional sensor data (e.g., body temperature data, ambient temperature data, galvanic skin response data, photoplethysmography (PPG) sensor data, electrocardiogram data). As explained elsewhere herein, a “reading” can include a “full sweep” for each energy, e.g., light, emitter, with each “full sweep” of a given light emitter including a plurality of “measurements” or induvial light absorption data points (e.g., data points measuring the skin's light absorption response to a corresponding light emission pattern for the corresponding light emitter).

As indicated above, upon receipt, at Step 2 at 110b of FIG. 7, the received sensed data can be preprocessed, such as by an on-board analytics module. Specifically, once the reflectance has been emitted and received by the device, the next step in the process may include determining a first reading corresponding to the amount of the first light being absorbed and/or reflected by the tissues and interstitial fluids, blood, and/or biomolecules therein within the skin layers of the active site under observation. Likewise, a second reading corresponding to the amount of the second light being absorbed and/or reflected by the skin and its components, and a third reading corresponding to the amount of the third light being absorbed and/or reflected back, and the same for a fourth, fifth, sixth, etc. readings being made corresponding to the amount of the respective light being absorbed and/or reflected back by the skin and its components.

Further, in various instances, as needed, the detection device may include programming, e.g., firmware, for determining if and when one or more of the sensor units may be affected by internal, e.g., mechanistic, or outside influences, such as caused by jostling. Thus, for various mechanistic and/or mechanical reasons, errors in the readings may be produced. In such instances then the device and/or analytics system may repeat various measurements having been taken or may attempt to perform error correction to correct for them. Such corrections may include taking more readings within a shorter period of time and/or including a flag for readings that are questionable, and as described in greater detail below, the analytics system may attempt to correct for them.

Finally, once the data has been collected, one or more characteristics of one or more molecules within the skin of the active area may be calculated. For example, in one embodiment, the system may be configured for employing one or more of the methodologies set forth herein for calculating a wearer's analyte, e.g., glucose, levels. In various embodiments, such calculations may be performed by an associated AI module of the system, such as implementing a data structure, e.g., an artificial neural net. Consequently, after the sensed data is preprocessed by the sensor and/or the analytics system itself, at Step 3, at 110c, the preprocessed data may be further transmitted and received, or otherwise be accessed by the analytics module of the server system, such as where the pre-processed signal inputs may be run through the analytics system, such as a data structure like an ANN, whereby the estimated glucose values may be calculated.

Additionally, in various embodiments, a number of other different characteristics may also be measured, and accounted for in the calculations, such as the skin temperature or hydration level, e.g., at the surface of the sensor, pulse rate, e.g., derived from photodiode signals, and user physiological information (e.g., age, gender, sex) may also be incorporated as inputs into the analyte, e.g., glucose, conversion process as the processing and consideration of such additional factors may impact the glucose spectral signal. Furthermore, in a further Step 4 at 110d of FIG. 7, an output of the analysis may be an analyte, e.g., glucose, measurement, which may be displayed to the wearer of the device, which may include the temperature and pulse rate signal data collected. In such instances, the user can view the current estimated glucose value, the glucose rate of change, daily and historical glucose trend graphs, and time within the range, such as within the mobile application.

Accordingly, in one aspect, as set forth at 110c, provided herein is an analytics module, which may include, or otherwise be associated with, an Artificial Intelligence (AI) module, which AI module may instantiate a data structure such as for implementing an Artificial Neural Network (ANN), Convolutional Neural Network (CNN), or other data structure, along with one or more machine learning algorithms, as set herein below. For instance, herein presented is an AI module that may include one or more machine learning engines as well as one or more inferences engines, such as for accurately determining biomolecule values, levels, concentrations, as well as the health conditions associated with the same. More specifically, as described here in detail below, a machine learning sub-module may be provided, such as to build a data structure, e.g., ANN and/or CNN, from which one or more inference sub-modules may further be provided so as to employ the data structure to generate one or more inferences and/or predictions, such as to the presence, value, e.g., concentration, and/or effects of a biomolecule on the body tissues, as well as with regards to any associated health conditions that may result with respect thereto. For example, in a particular implementation, the machine learning and/or inference generating sub-modules may implement or otherwise instantiate one or more of a decision tree, a support vector machine, one or more of a polynomial regression, Rectified Linear Unit (ReLu), and/or a sigmoid activation function, an artificial neural network, an adaptive logic network, a Bayesian network, a convolutional neural network, and/or the like, such as for determining and analyzing trends, e.g., with respect to one or more characteristics of a biomolecule and its effects on the body, and providing intelligent insights with respect thereto.

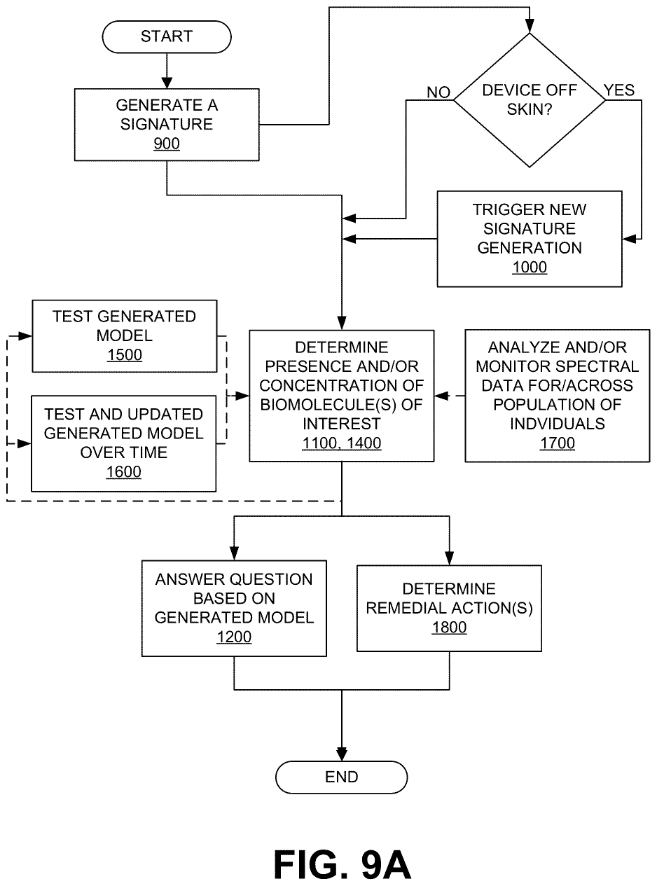

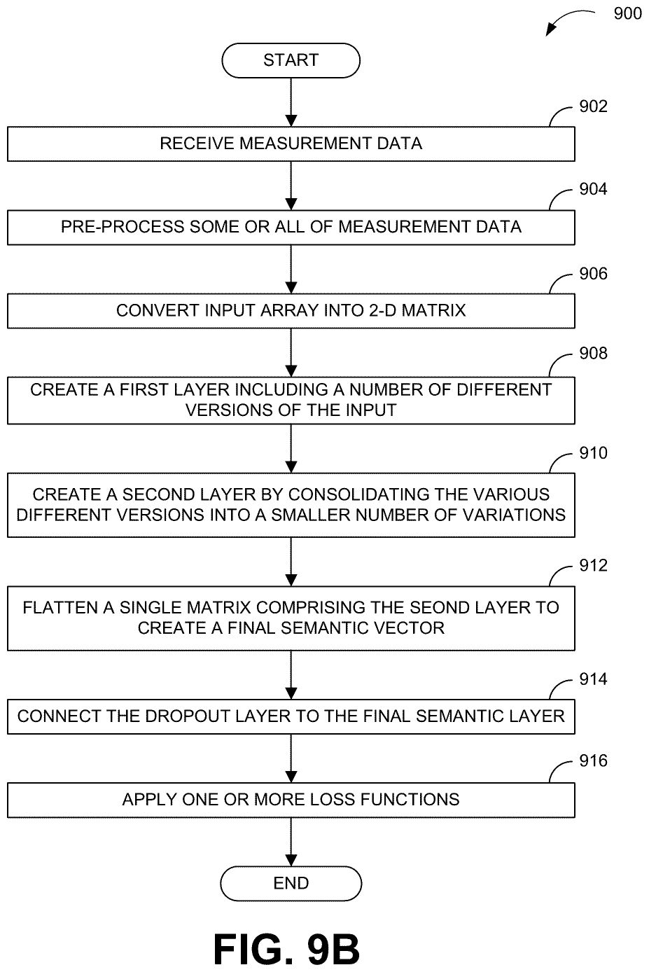

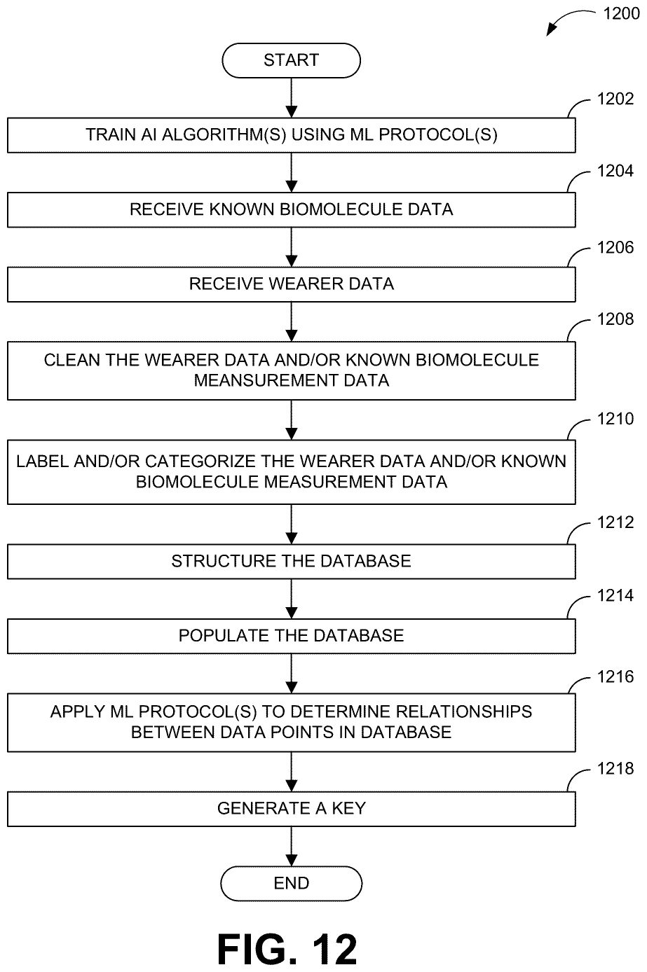

Particularly, in one implementation, in computing the captured measurement data and corresponding the results thereof to an analyte concentration, the analytics system may implement aa decision tree. A decision tree is a flowchart-like model that uses a tree structure with nodes and branches to make decisions, classifications, and/or predictions, e.g., about a permittivity effect or concentration, by asking a series of simple, hierarchical questions about the data, which may then be used to effectively map “if-then” rules to classify or estimate the measurements and other values. The decision tree algorithm learns via supervised learning whereby the model is trained on measurement data where the outcome is known, and the measurement values are calculated in a manner so as to derive the known outcome, as described herein with reference to FIG. 12. And where the outcome is not derived and error function is employed, determined loss is redistributed, and the function is run again, as described herein with reference to FIG. 9B.

In these regards, the decision tree typically includes a root node, an internal or decision node, branches, and leaf or terminal nodes. The root node is the starting point and represents the first question as well as the entire dataset. The decision node represents instances where a calculation or test is performed on the data or an attribute thereof, e.g., a feature. The branches are the lines connecting the nodes and represent the various possible outcomes, e.g., answers to the tests. And the leaf nodes represent the final endpoint(s), e.g., analyte concentration value, which provides the prediction or final decision (outcome). In this instance, the decision tree is useful in the supervised machine learning described herein for corresponding input measurement data to known analyte concentration values. Likewise, the decision tree is further useful in an unsupervised process, also as described herein, for corresponding input measurement data to unknown analyte concentration values, such as in comparison to a previously generated calibration. However, in various instances, the system should be configured to keep the structure as simple as possible to avoid the over fitting that occurs when the data tree becomes too complex. This can be accomplished by implementing a suitably configured pruning function.

Likewise, there are a number of regression models that can be used such as a linear regression, a polynomial regression, a random forest regression, and the like. These models analyze historical measurement and data to understand how the input features e.g., permittivity effect measurements, correlate with the output, e.g., analyte concentration. More particularly, these algorithms create a mathematical equation, such as for linear regression, which best describes the relationship between the data in a manner that minimizes errors. Once trained, they use the equation to predict a new, continuous output value, such as when contemplating new, unknown, input data. With respect to linear regression, this model uses a straight line to predict an outcome. For a polynomial regression, this model fits curved lines, e.g., polynomials, so as to discern more complex, non-linear patterns by which to make predictions. A Random Forest Regression, on the other hand, may be used to combine multiple decision trees so as to provide for more robust predictions, this is particularly useful for analyzing the complex datasets produced herein.

Another function that can be employed for these and other such purposes is a support vector machine (SVM). A SVM is a supervised machine learning algorithm that can be used for regression as well as classification. In this regard, implementation of the SVM as a regression model is useful for predicting continuous, e.g., linear or curved, numerical values, which in this instance, are measurements, e.g., analyte effect values. It essentially draws the “best fit” line, plane, or curve through data so as to predict a corresponding outcome, e.g., concentration. A SMV works by finding an optimal decision boundary, e.g., a line or plane(s), to separate different classes of outcomes in the high dimensional feature space being generated herein. This process may be performed in a manner that maximizes the distance (margin) between the boundary and the nearest (neighbor) data points, e.g., support vectors, which facilitates the efficient classification of each measurement datapoint. This allows for the most robust predictions to be made.

In various embodiments, the SVM defines a linear equation that performs as the core of a linear model whereby associated nodes or neurons learn the weights and biases to position and orient the bounding line(s) and/or plane so as to optimally distinguish differences between the various measurement values. In essence, the primary function of the SVM is to divide up the input space, e.g., within the data structure, so that measurements on one side belong to one class and measurements on the other side belong to another. In such instances, the data structure may be a representation of a 1D or 2D space, such as a line or plane. For example, the line may be defined by a set of data points forming a 1D object, whereas the plane may be defined by a set of data points spanned by two linearly independent vectors, e.g., forming a two-dimensional vector space. In this regard, the Cartesian plane can be described as the set of all measurements that can be reached by taking linear combinations of two non-parallel vectors that originate from the same single point. Adding a third such vector can then create a 3D Cartesian space.

How this works, therefore, is that the SVM may be configured to assess the measurements and, where appropriate, organize them in such a way as to start from a given point and therefrom produce a linear extension of datapoints that form a line, such as along a first axis. Then as the data permits, a second and/or a third set of lines, e.g., vectors, may also be constructed. Ultimately, the SVM may be configured to find the optimal hyperplane with the largest distance, or margin, between the closest measurements to ensure good generalization of the data. As described herein below, neural networks (like perceptrons) use these hyperplanes to divide linearly separable data. Likewise, deeper networks, e.g., convolutional networks, can then be engaged to combine multiple hyperplanes, e.g., through non-linear activation functions, such as a polynomial regression, to better analyze the complex, non-linear data points collected herein.

A polynomial regression is a non-linear activation function that may be employed by the machine learning algorithm herein so as to configure a model that functions to extend non-linear relationships in the measurement data by adding polynomial terms (e.g., exponential functions) to features which would otherwise be a linear regression model. In this regard, the polynomial may be an algebraic expression that is separated by an addition or subtraction function. This allows the data to be fit within a curved pattern instead of forcing the pattern into a straight line. In essence, the polynomial regression function introduces curves and complex patterns that allow the neural network (described below) to learn non-linear relationships and/or patterns within the measurement data, rather than being restricted to the simple straight lines. The polynomial function works by transforming features into higher powers and then applies a standard linear regression technique to the new higher feature.

This sub-process effectively captures complex curves to better predict outcomes, such as where higher degrees allow for more complex curves and lower degrees allow for more straight lines. However, this requires a careful selection of the polynomial degree to avoid overfitting, e.g., too high of a degree, or underfitting, e.g., too low of a degree. This is important because the degree determines the complexity of the curve being used to model the non-linear relationships within the data. Particularly, the problem with overfitting is that the model becomes overly flexible, allowing the fitting of training data perfectly but including noise, which makes it challenging for the model to generalize to new data. On the other hand, in underfitting, the model might be too simple to capture the true relationships, e.g., patterns, and thus, may perform poorly on both training and test data. Hence, choosing the right degree involves a balancing between model simplicity and complexity in such a manner as to capture underlying patterns without increasing noise.

In these regards, non-linear activation functions are useful herein because the collected measurements are fluid and dynamic. This fluidity is due to the fact that the captured measurements represent the flux of analytes that is occurring within the interstitial milieu and blood flow, whereby the very nature of this flux is typically non-linear. Thus, such non-linear activation functions can be employed to create curved decision boundaries that enables the various models generated herein to more accurately classify these complex inputs, thereby turning a deep learning network, e.g., with multiple layers, into a universal approximator or estimator that can learn and map any function regardless of the complexity to the desired output, e.g., analyte concentration. They basically function by applying a transform that is more than a simple scaling or shifting, but rather, they add complexity to the network's output. In essence, non-linear activation functions introduce conditional logic, e.g., IF-THEN, into the network, which allow different neurons or nodes to activate or deactivate based on specific, determinable input patterns.

Another such non-linear activation function is a Rectified Linear Unit (ReLu). A ReLu operates in a neural network that considers and outputs an input directly if it's positive, but outputs zero for any negative input, which may be defined as f(x)=max(0, x). By doing this the ReLu introduces non-linearity that, as discussed above, helps deeper networks learn complex patterns in a manner that is computationally efficient. Particularly, the way it works, for example, is that if the input x is greater than 0, the ReLU will return an x, but if the input x is less than or equal to zero (negative), then the ReLU returns 0. This allows the neural network to efficiently learn complex, non-linear relationships in data because it's fast to compute involving just a comparison, unlike functions with exponentials (like sigmoid). Therefore, the ReLU speeds up the training of the model, and likewise, its linear nature for positive inputs prevents gradients from shrinking too much, which mitigates potential gradient vanishment and helps train deep network(s) effectively. Basically, in an Artificial Neural Network the ReLU may be applied to the output of a neuron (or layer) and functions to decide its final activation, adding a bend (non-linearity) that facilitates deep learning.

As mentioned, the Sigmoid function can also be used to produce a smooth “S” (sigmoid) shaped curve by mapping any real-value inputs to an output that is between 0 and 1, or in certain configurations, sometimes −1 and 1, making the function good for determining probabilities. This “S” shaped curve starts low, rises steeply in the middle, and flattens out at the top, where the midpoint is 0.5, e.g., where an input of 0 yields an output of 0.5. The sigmoid function, therefore, is useful in machine learning, such as for logical regression and neural networks, such as for converting outputs into probabilities. In essence, the sigmoid function maps a linear combination of inputs to a probability (e.g., 80% chance of a measurement being a member of a given permittivity class). Specifically, the sigmoid function is particularly useful making binary classifications to help decide if a measurement belongs to one of two classes, e.g., based on the probabilities, such as a high or low classification. More particularly, in making decisions, the Sigmoid function applies a threshold, such as 0.5, whereby it determines the final class, such that if the output is >0.5, it's one class, otherwise it is the other. And, as an activation function, it adds non-linearity to neural networks, thereby allowing the network to learn complex patterns.

As discussed herein, in analyzing the collected measurement data so as to derive an analyte concentration, the analytical system may generate a data structure by which the measurement data may be input and an analyte concentration may be output. In this regard, one such data structure that may be used for these purposes is a Neural Network. A neural network, implemented by a computer (therefore: an Artificial Neural Network (ANN)), is a type of brain-inspired data structure and machine learning model that operates in a manner similar to the human brain so as to process data, in this instance permittivity measurement data or just measurement data.

Essentially, the ANN includes a number of interconnected nodes that function like neurons so as to process information and thereby learn patterns form the measurement data. Here it may be employed by the AI module to recognize patterns and make decisions, such as by processing the measurements through a series of interconnected layers of artificial neurons. More specifically, the structured layers of the ANN include an input layer, one or more “hidden” or computational layers, and an output layer. Hence, each layer includes an artificial neuron, or node, that receives the data, processes it, and then transmits the results to the next layer for further processing.

However, the connections between the neuronal layers and/or nodes have weights associated with them, which weights determine the influence of one node's output on the next output. Biases can also be added to shift the activation threshold of each neuron. In essence, ANNs learn from large datasets, adjusting these weights and biases to improve their accuracy in making predictions, which in this case can be predictions about correlating the collected measurements to ultimate analyte concentrations. More specifically, during training, the network analyzes labeled examples, in the case of known measurements, so as to learn what features are important, such as for determining analyte concentration levels. It then adjusts its weights and biases to minimize the difference between its predictions and the correct answers. Hence, by learning to transform the raw measurement data into meaningful patterns, the artificial neural network can identify the complex, non-linear relationships that are necessary for correlating the processed measurements to concentration levels. This configuration is important because these patterns are very complex and will be missed by traditional algorithms.

One form of ANN that can be implemented is an adaptive logic network (ALN). An ALN is a type of neural network with weightless logic units that uses a tree-like structure, which is built with Boolean neurons (e.g., ON/OFF or I/O) and logic gates (AND/OR/NOT). For instance, the logic units or perceptrons form the leaves, whose output feed into branches of logic gates. In this regard, like the other networks described herein, the AL network is configured for receiving scalar and vector inputs as leaves, e.g., representing the sensor readings, and each leaf node processes the data and outputs a simple threshold unit such as a 0 or 1 (or ON/OFF). These outputs then can be combined through AND/OR gates up the tree. The network, therefore, learns to configure these gates and perceptrons to map inputs to a determined output, effectively approximating complex functions with simpler logical pieces, such as without necessitating the use of weights and/or biases.

Hence, the adaptive logic network learns by adjusting its internal logic and extracting piecewise linear functions, which approximate a predicted complex outcome. It processes data using Boolean logic so as to take complex systems and represent them as simple states (ON/OFF). Particularly, it takes real-valued, e.g., permittivity measurement, inputs and produces a binary (logic 0 or 1) output, representing whether the input falls on or below the graph of a function. In this manner, the AL network learns to approximate complex, real-valued functions by breaking them down into piecewise linear functions. This creates a tree of simple binary classifiers (e.g., perceptrons) and logic that is capable of handling complex relationships and control tasks in real-time. This type of network is especially useful for its ability to manage nonlinear systems and provide interpretable, fast decisions. In essence, by offering a blend of neural network learning power and logical interpretability the AL network can be configured to model and determine outputs by learning logical rules derived from the measurement data.

A further ANN that can be implemented is a Bayesian neural network (BNN). In some manners the BNN is much like an ANN, but it uses weights. Particularly, the BNN is a neural network that may be used to weight the measurements whereby the weights (and/or biases) are treated more like probability distributions rather than single (absolute) values. What this means is that unlike standard NNs, which use a fixed-point estimate of weightings, here the weighting represents a probability, e.g., Gaussian, distribution. Specifically, where the standard NN, described above, is configured for implementing a single function, the BNN implements a probability distribution function. This allows the analytics system to consider a variety of plausible models (reducing overfitting) so as to better quantify uncertainty in predictions such as by providing a range of possible outcomes, which makes the calculations more accurate.

Hence, instead of making a single “best” set of weight calls, the BNN can incorporate prior knowledge and learn a distribution range of weights. Particularly, prior knowledge and/or beliefs about weights can be defined and continually updated to form posterior weight distributions, which is useful for generating results even when considering limited or few data. What this means is that the predictions being made may be averaged over a number of possible sample networks. Bayes' theorem may then be applied so as to update the generated beliefs as the measurement data is collected by the system and input into the matrix. This leads to better generalization and risk assessment even when the datasets are small.

Another ANN that can be implemented is a Convolutional Neural Network. A CNN is a type of AI model that is configured to automatically learn and extract features from the measurement data through a process of convolution, e.g., where a filter, in this instance, a kernel, slides across the inputs and then generates a feature map. Particularly, the CNN uses a specific architecture that includes one or more convolutional, ReLU, and/or pooling layers that may be used for performing automatic feature extraction. In this regard, as the ReLU is a non-linear activation function that may be applied after the convolution operation so as to set any negative values to zero, while keeping all positive values as they are. This adds non-linearity to the computations thereby speeding up the training and the learning of complex patterns. Likewise, this architecture is useful because it allows for weight sharing (which allows the same set of learned filter weights to be applied across the entire set of inputs) during encoding and/or decoding (described below), which increases efficiency and effectiveness. Classification employing one or more, e.g., all, of the connected layers can then take place, such as after feature selection.

In view of the forgoing, the AI technology disclosed herein may be configured for determining the characteristics of one or more analytes of interest, e.g., glucose molecules, and their effects on the body over time, such as based on the quantity of the analyte being present, e.g., glucose levels. In such an instance, in determining a biomolecule level, a data structure, such as an artificial neural network (ANN) and/or one or more Machine Learning (ML) algorithms, described above, may be generated and/or trained, so as to populate a data structure with measurement data that is used to build and train the neural net on the specific biomolecule of interest. In various embodiments, depending on the amount of data and the analyses to be performed, a number of different neuro-nets, or layers, may be produced, each structured and/or focused on a specific biomolecule(s), such as glucose, and/or with regard to one or more specific individuals. And likewise, a number of machine learning algorithms may be employed to better achieve these purposes. As such, the training and/or analysis may be performed on continuous, real-time, data collection and analysis, such as for one or a number of readings and/or sweeps, for one or a number of subjects over a prolonged period of time.

Accordingly, in one aspect, provided herein is an analytics system 71, which may include an AI module 72, wherein the AI module may be configured so as to implement one or more algorithms for receiving sensed data, and using that data to make one or more measurements from which a determination of a biomolecule or of a biological condition may be made. In using the device, the AI, e.g., a machine learning sub-module, may first be trained, and once training is complete, an inference engine may be employed so as to make one or more determinations of one or more characteristics of a biomolecule of interest being present within the body tissues, as well as to one or more conditions that may be experienced because of the presence of the detected biomolecule.

With respect to the artificial intelligence module, in one aspect, one or more local and/or cloud accessible artificial intelligence modules are provided and are configured for being communicably and operably coupled to one or more of the other sub-systems of the biomolecule detecting, sensing, and/or processing pipeline disclosed herein. For instance, the one or more AI modules disclosed herein may work closely with a suitably configured analytics management system so as to efficiently direct and/or control the various methods and processes of the analytics system disclosed herein. Accordingly, provided herein is an analytics system that may include one or more, e.g., a plurality, of AI modules, which are configured for acting as an interface between one or more observable characteristics of an individual, one or more artifacts within their tissues, interstitial fluid, and/or blood, and one or more measurements being performed so as to determine the presence and/or effects of those artifacts on the body.

The analyses to be performed by the AI modules of the system may be directed to detecting the presence and/or characteristics of one or more biomolecules within the body, the effects their presence has on the body, and further, for determining one or more remedial actions that can be taken in light of the presence of the biomolecule and its effects. For instance, in various instances, the system may be configured for performing a number of measurements, e.g., within the skin and tissues of an individual, as disclosed herein, so as to derive electromagnetic radiation, e.g., spectral, data therefrom, along with other biological condition data, from which data, the analytics system can use the raw and/or pre-processed data to determine the presence of a biomolecule within the tissues of the individual as well as a condition of that individual due to the degree of presence or absence of that biomolecule, e.g., with respect to its amount and/or concentration. For performing this analysis, the data structure may be generated and populated with biomolecule, e.g., measurement, data, user condition data, and/or characteristic data, as well as, with spectral data from the individual, all of which may form nodes in a graph or neural network structure.

With respect to the photonic and/or RF and/or microwave spectral data being generated herein, the electromagnetic radiation data may be of two kinds: electromagnetic radiation data that has been correlated with known levels of a biomolecule, e.g., known electromagnetic radiation data, such as control data, and electromagnetic radiation data that has not been so characterized, and thus, is unknown. In this regard, where glucose is the biomolecule of interest, a blood glucose meter/analyte sensor that is known to give accurate results, e.g., blood glucose measurements, e.g., invasively, can be used to generate a known presence and concentration of glucose value, which values can be employed herein as control or test measurement data. Specifically, this known glucose value data may then be correlated with the condition and electromagnetic radiation read data so as to produce the known electromagnetic radiation read data, such as where it is known, regardless of the read data measurements, whether glucose is present and at what concentration, because that value has been determined by use of the invasive glucose monitoring system. Consequently, this data may then be used to populate a data structure, whereby the unknown electromagnetic radiation data may be tested and/or determined as compared to the known electromagnetic radiation data.

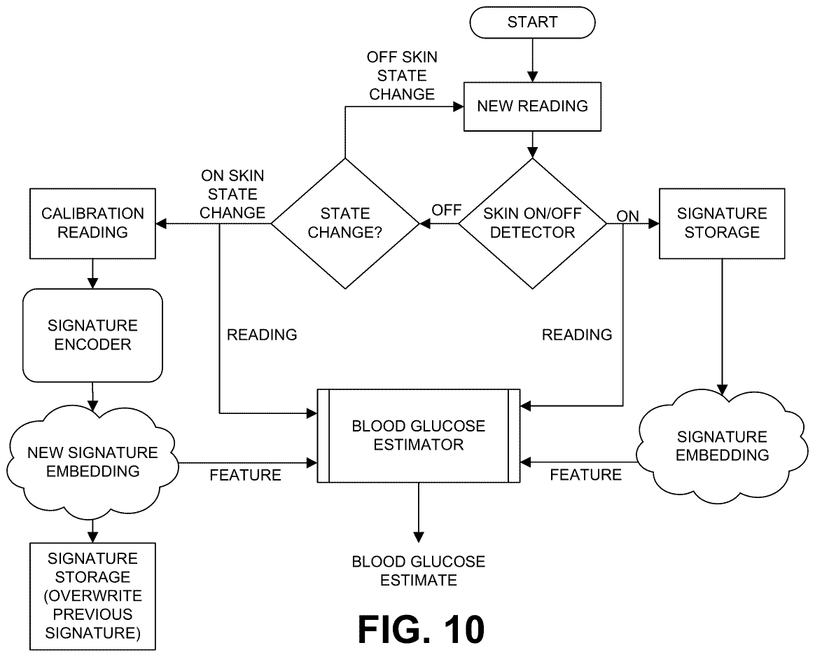

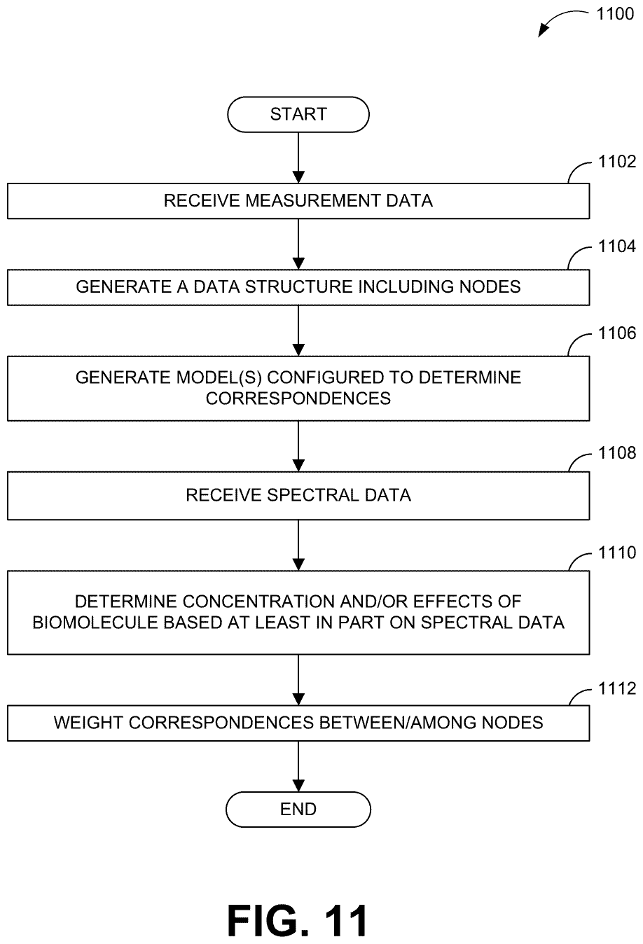

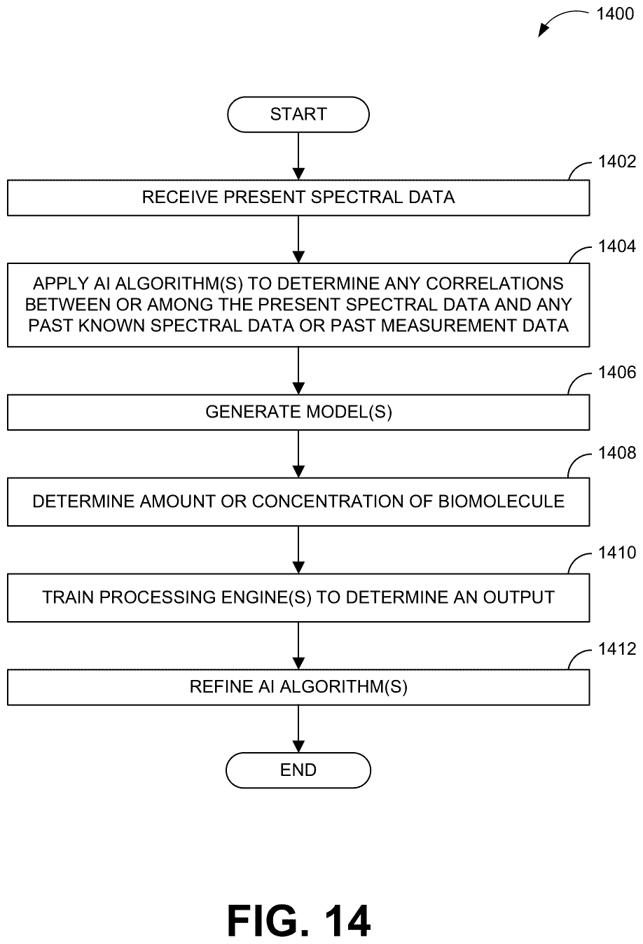

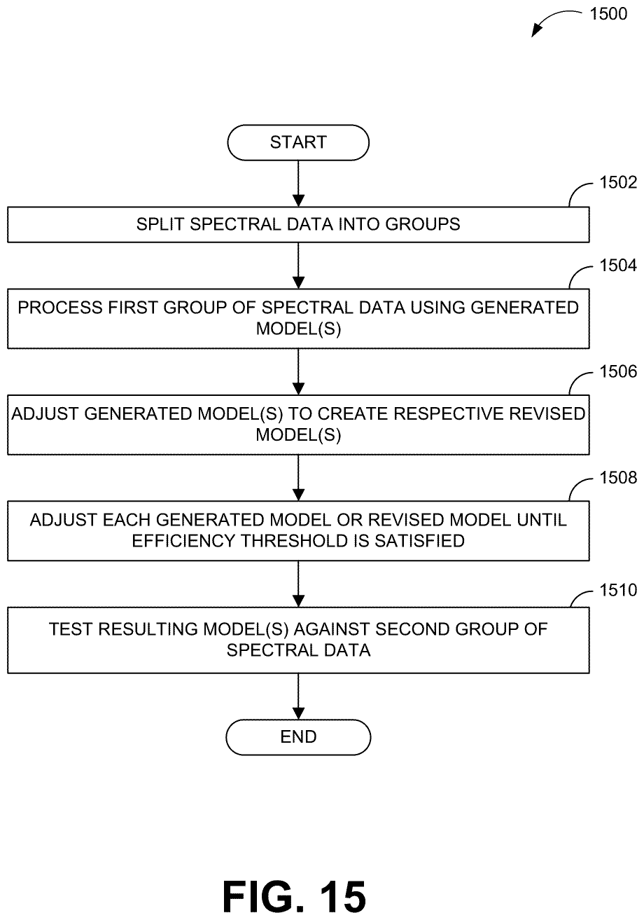

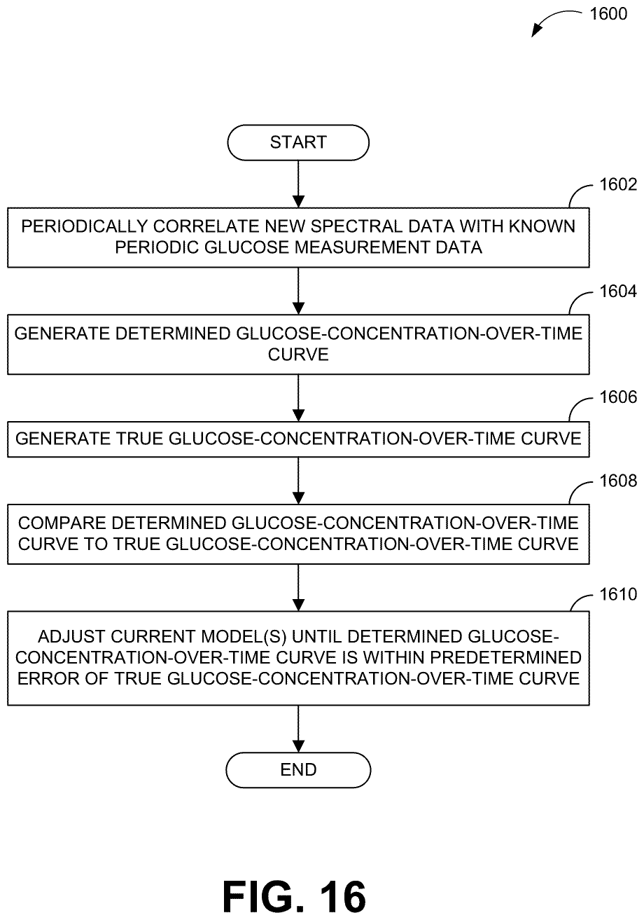

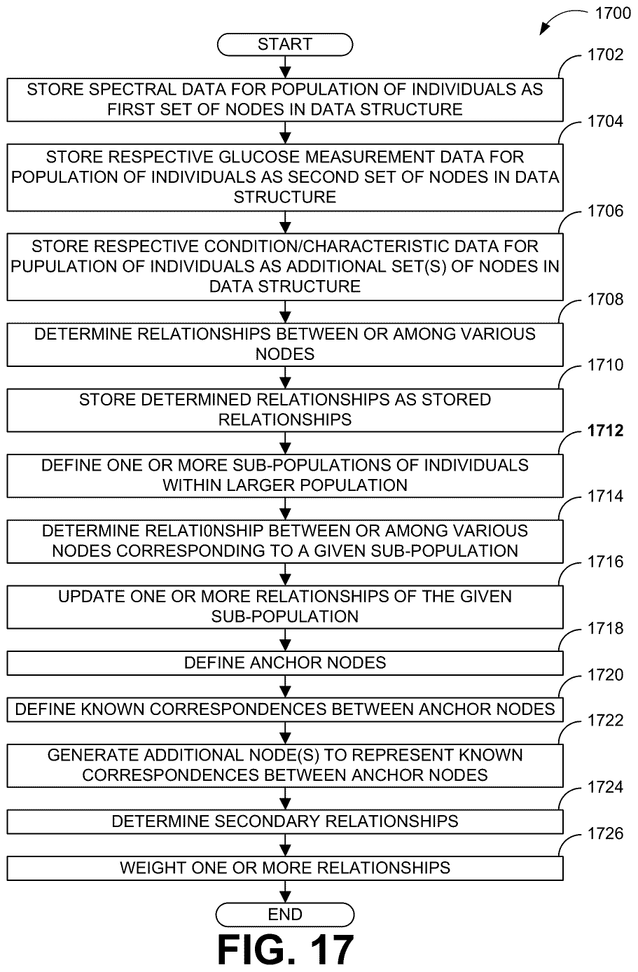

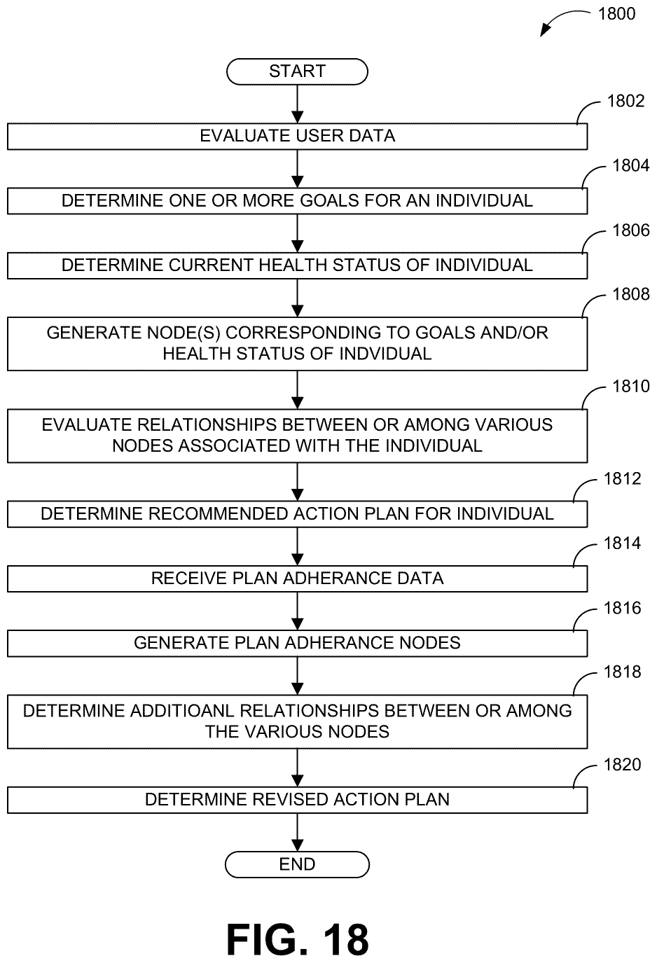

The disclosed technology includes systems and methods for analyzing unknown, non-invasively obtained electromagnetic radiation data for a given user and determining the level of an analyte of interest, e.g., glucose (or a level of another biomolecule, analyte, or the like) based on various determinations that are founded at least in part on the spectral and/or RF and/or Microwave data. The various systems and methods of the disclosed technology can include multiple processes and/or sub-processes. For example, and as illustrated in FIG. 9A, the disclosed technology includes multiple processes described herein in more detail, including, but not limited to:
generating a signature (e.g., process 900, illustrated in FIG. 9B);
triggering new signature generation (e.g., as illustrated in FIG. 10);
determining the presence and/or concentration of one or more biomolecules within a wearer's body (e.g., process 1100, illustrated in FIG. 11);
answering a question based on information and/or correspondences stored in one or more data structure(s) (e.g., process 1200, illustrated in FIG. 12);
estimating the current presence and/or concentration of a biomolecule of interest for a given wearer without contemplating present known measurement data (e.g., process 1400, illustrated in FIG. 14);
testing a generated model (e.g., process 1500, illustrated in FIG. 15);
testing and updating a testing model over time (e.g., process 1600, illustrated in FIG. 16);
analyzing and/or monitoring spectral data for or across a population of individuals (e.g., process 1700, illustrated in FIG. 17); and/or
determining one or more remedial actions (e.g., process 1800, illustrated in FIG. 18).

It is to be understood the specific order or operations illustrated by FIG. 9A is merely one example, and the disclosed technology can include systems and methods that include one, some, or all of the disclosed process; includes at least some (but not necessarily all) of the disclosed processes in the example order illustrated by FIG. 9A; or includes at least some (but not necessarily all) of the disclosed processes in an order different from the specific example illustrated by FIG. 9A (e.g., testing and updating a generated model (e.g., process 1600) after determining one or more remedial actions for a wearer or user (e.g., process 1800)). These and other aspects of the disclosed technology (e.g., with respect to analyzing non-invasively obtained spectral data for a given user and/or generating models to estimate the presence and/or amount or concentration of a biomolecule of interest based on various determinations that are based at least in part on the spectral data) are described in more detail below (and/or elsewhere herein), with reference to examples in the figures.

Consequently, in view of the foregoing, provided herein are devices, systems, and their methods of use for generating and taking measurement data, feeding those measurements into an analytics system, such as including an AI module, and then using AI and ML to correspond (map) those measurement, e.g., read data, to analyte(s) concentration data. For instance, as described in greater detail here below, FIG. 9B presents a general overall process for calibrating a multi-sensing detection device of the system onto the skin, and then using an AI generated data structure to map measurements collected from the detection device to a concentration of an analyte, e.g., glucose, based on an analysis of those measurements. As depicted, the calibration process involves taking a number of different measurements: both with the multi-detection device of the system (e.g., here a test device) as well as a control device, e.g., blood glucose monitor, that is known to give accurate measurements, such as using a finger-prick measurement system. And as set forth above, a number of different AI based algorithms can be used in this process so as to generate a calibration, e.g., a signature, of the skin at the particular moment in time the calibration is taken with that particular concentration of analytes being present within the extracellular milieu during the moment when the calibration is being performed. This calibration can then later be used to normalize later measurements an/or to predict concentration outcome.

In using AI to perform such a calibration, a data structure, such as one of the artificial neural network described above, may be constructed, by which the measurement data collected by the dual sensor detection device may be mapped to a concentration outcome. Here, in the case of a calibration, the concentration may be of a collection of analytes within a given region of the tissue to which the multi-sensor detection device is positioned. In performing the referenced mapping, the data structure, as discussed above, may be composed of one or more layers, such as an input layer, an output layer, and one or several hidden, e.g., computational, layers.

In particular embodiments, the first input layer can be comprised of different sub-portions or sublayers, where different versions of the measurements to be input may be entered into the data structure, e.g., ANN, such as where one or more of the inputs can be considered as records. As indicated, in one particular implementation, the ANN may be a Convolutional Neural Network (CNN). As referenced above, a CNN is a particular type of ANN, whereby instead of having a single input layer that directly goes to the final output, the data structure may employ a multiplicity of active intermediary layers, which layers may be processed by an estimator that may include a kernel function. In various embodiments, these active intermediate layers can be configured as subdomains of about two by two, three by three, four by four, five by five, etc., wherein the particular kernels can be convolved across the various sub-domains until the entire input is processed.

What this means is that the kernel function, within a subdomain, may be multiplied at one point by the entire input matrix, thereby generating an aggregated signal for that domain. Subsequent to this, the kernel function can be moved one stride over, and then be multiplied by the entire matrix again, so as to generate another output at the next point, such as for the next, e.g. adjoining, subdomain. And these steps can be repeated again and again for the entire sub-domain, such as row by row, column by column, for the entire input matrix, and for every single kernel. The reason that this is useful herein is because by performing these processes a pattern in the signal, much like image recognition, e.g., for fingerprint detection, can be recognized, e.g., for one or more subdomains, and then the repetition of that pattern subsequently can be analyzed so as to better understand and characterize the output, which in this instance may be a “signature”. Thus, this pattern recognition process can be used to increase the intelligence of the overall machine learning techniques employed herein.

Accordingly, once the first layer has been constructed, the second layer can further be built, such as by consolidating, e.g., pooling, various of the original input measurements. In essence, the detection device generates a lot of different sensed and measurement data, whereby various of the algorithms and functions set forth herein can be used to analyze all of this different data, perform a number of different manipulations thereto, and thereby identify and recognize one or more patterns therein. And once such a pattern(s) has been recognized and determined, then the system can use that pattern(s) in making the various determinations set forth herein, or in some embodiments, one such pattern can be used to derive other patterns, e.g., given other data sets, to better understand the outputs generated by the system.

Particularly, in various embodiments, the raw read data can be entered into a data structure, such as a two-by-two, etc. matrix consisting of columns and rows, such as where the emittance generation values may be placed into rows traversing horizontally over the reflectance reception values, which may be positioned vertically in columns, whereby through insertion into the data structure, in this instance, a table, the characteristics of emittance can be compared against the characteristics of reflectance in a series of reads and sweeps. In a manner such as this, a number of permittivity factors, as described herein, can be analyzed and patterns with respect thereto can be determined in like manner.

Specifically, in certain particular embodiments, the multi-sensor detection device set forth herein may include a number of optical sensor arrays having a plurality of emitters, where each emitter may be controlled so as to emit a light wave at a predetermined frequency and wavelength, having a preset amplitude, intensity, luminosity, emission duration, and in a programmed cycle of emissions, where each cycle of emittances from the set of emitters constitutes a sweep. In various instances, it is each emittance from each single emitter that can be used to fill in the emittance data structure, e.g., table. However, in other instances, it is the entire set of emittances that constitute a complete sweep that are integrated and entered into the data structure, e.g., table, as a single value.

In either of these instances, the data structure sets forth the measurement values obtained from the signals returned at the photoreceivers of the photonic detection device. These values represent the state of the underlying tissue with respect to how it responds to light in the presence of the analyte of interest. As indicated herein above, the skin responds differently to the presence of analytes therein in a concentration dependent manner. These values, therefore, can be corresponded to the concentration of glucose.

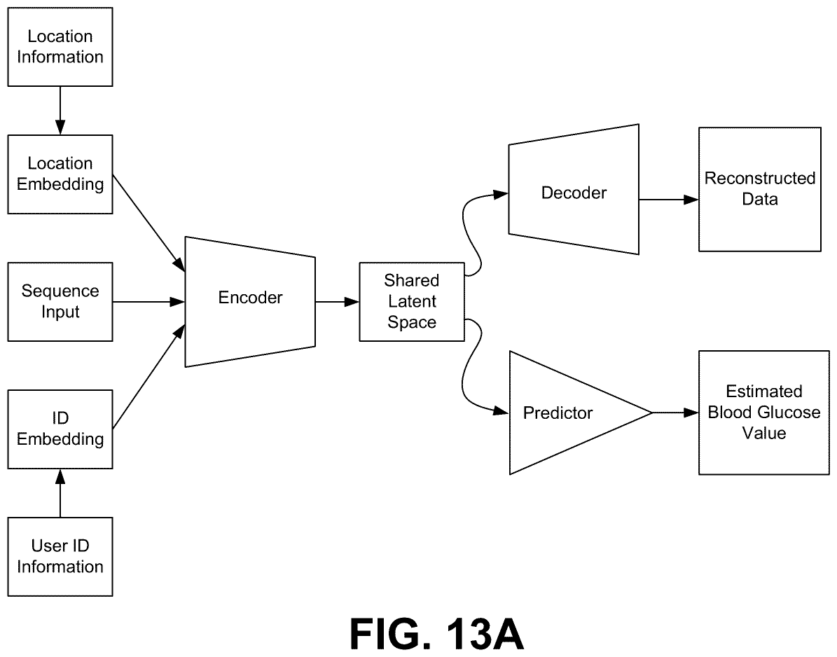

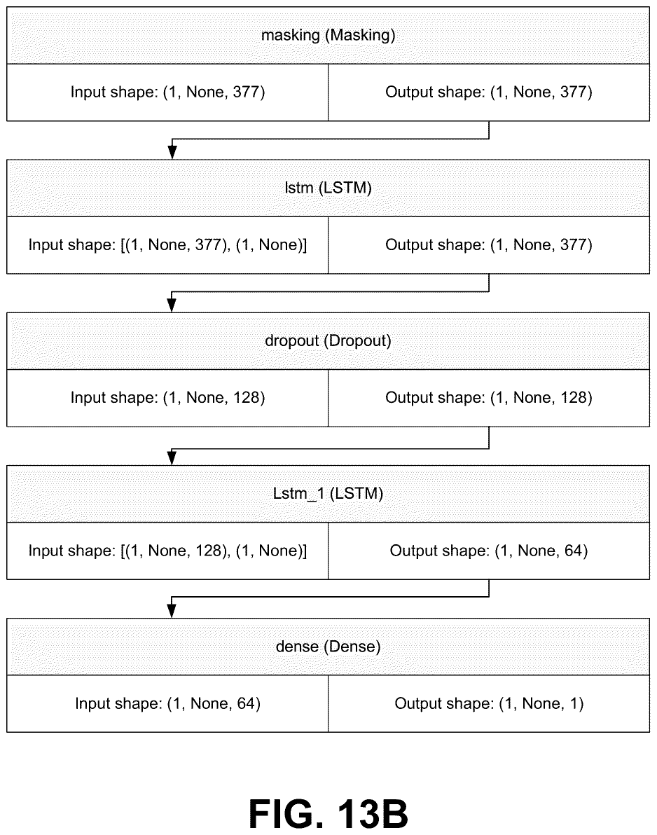

More particularly, a number of the received photonic measurement values can be integrated in a manner whereby the concentration of the analyte of interest, e.g., glucose, can be derived thereby, such as by mapping a selection of these values to the final concentration output value. As indicated herein, in various embodiments, the multi-sensing detection device can be operated in a supervised (See FIG. 12) and/or an unsupervised (See FIG. 13) manner. For instance, when initially calibrating the device, such as to generate an initial signature, as set forth with respect to FIG. 9B, the device may be employed in a test mode, such under a supervised protocol.

So being, in a supervised assessment, measurements from the multi-sensing detection device, in this instance the “test” device, may be taken at the same time as measurements are being taken from a control device, e.g., a finger-prick blood glucose monitor, which is known to give accurate measurements. In such instances, therefore, the true concentration of the analyte of interest, e.g., glucose, will be known due to the corresponding measurements being taken at the same time by the control device. In this manner then, the measurement values captured by the test device can then be corresponded to the known concentration values. This is useful, for example, for training one or more of the models and/or functions disclosed herein. Once the models and/or functions, e.g., activation functions, have been trained, then they can be employed for use in analyzing the measurements captured by the multi-sensing detection device. However, now the sensing device is no longer a test device, and by applying the models to the collected measurement data, the analytics system may then make a call as to what the concertation of the analyte is, but this time in an unsupervised fashion, that is without recourse to the known concentration values. Although, in various instances, a comparison may still be made, such as with the calibration generated in accordance with the process set forth at FIG. 9B.

However, the skin does not respond equally to the same degree by the light emitted by each of the photoemitters, and thus, not all measurements contribute equally to determining the ultimate analyte concentration determination. Rather, only a number of the values significantly contribute to the concentration determination, and therefore, the analytics system must determine which measurements from which photoemitters are the ones to which the tissue is actually responding. The analyte system, therefore, by applying different weights and biases to the collected measurements, must find out the most efficient way to proportion the contribution of each input measurement to the final outcome, e.g., concentration level. To make this determination, the system generates a function, such as set forth above, by which to calculate the measurement values in a manner so as to derive the unknown (known if in a supervised testing mode) concentration value. But in order to do this, the system must initially attribute a weight and/or bias to each measurement predicting the contribution that measurement makes to the final calculation. Collectively these weighted and/or biased measurements may be called parameters, which parameters may then be used to generate a parameterized function that can then be employed by the system to map the various measurement inputs collected herein to the analyte concentration value.

As discussed above, when the test detection device collects measurements at the same time the control device also collects the known concentration values, e.g., in a supervised manner, the calculations performed by the analytical system to correspond the measurement values to the concentration value(s) are termed learnings. These calculations are typically performed by the Machine Learning module of the system and the whole process is referred to as supervised learning. However, once the model is generated, then the measurements may be made in a manner whereby the ultimate concentration of the analyte is not known, but must be inferred, such as by the inference engine, e.g., calculator, applying the model to the measurements and then mapping the results to an unknown glucose concentration that is to be inferred. This form of learning and/or calculation is referred to herein as unsupervised.

Accordingly, during an initial set of runs, e.g., during the model generation process of a supervised learning procedure, the generated measurement inputs from the test device may be mapped to the known concentration values derived from the control device, e.g., in a supervised learning process. And, once the model has been generated, a similar process can then be employed to map the measurement values to an unknown output that needs to be inferred from applying the model, once generated, to those measurement values, so as to derive an answer, but this time in an unsupervised manner. Hence, once trained, the model can then be used to map the collection of measurement values to the ultimate concentration value even when that value is unknown but needs to be inferred. As described herein below, in a first instance, this process can be performed so as to derive a signature by which the ultimate concentration can be determined, e.g., predicted.

Hence, as stated, initially during the training process, this learning is referred to as unsupervised learning. The model, therefore, must be trained to analyze the various weighted light measurements, e.g., parameters, and determine a function by which the concentration of one or more analytes of interest can be calculated. In this regard, therefore, the data structure and the model form a parameterized function that can then be used to infer the analyte concentration from the collected measurements, such as in an unsupervised process.

As discussed herein above, in particular embodiments, the data structure may be a multi-dimensional structure such as a neural net that acts as a function that interacts with the model to recognize a pattern by which the weighted measurement inputs, e.g., parameters, may be mapped to the output, in this instance, a concentration value. The identifying of such a pattern is useful for translating inputs into estimates. More specifically, as explained in greater detail herein below, such patterns may represent the series of weights and/or biases that are attributed to each of the measurements where each weight represents an estimated percentage to which each measurement contributes to the final concentration calculation. Hence, the pattern is the attribution of all of the weights and/or biases of the measurements within the network that correctly maps the inputs to the concentration output.

However, as indicated, not all measurements contribute or to the same extent as every other measurement, and so the measurements that do contribute and their extent of contribution has to be determined. Nevertheless, as can be imagined, given the sheer quantity of measurements over such a short period of time, the input space can become very, very complicated making the identification of a pattern and the derivation of a specific set of estimates therefrom extremely challenging, especially when there are problems with the data.

For example, when the sensor device is placed on a body, an initial flood of information is generated, and subsequent measurements may form batches, which cause the developing pattern to shift and/or jump around. These shifts can happen where the data clumps around a particular domain, e.g., when the measurements become domain specific. Such domain specific features can modify the pattern, causing it to shift around resulting in increased noise. The machine learning mechanism, therefore, needs to be trained to distinguish the true signal from the background and noise, which in turn, should make pattern identification easier. For these purposes, as described herein below, a number of mechanisms have been developed and employed herein to distinguish and separate the noise from the accurate signal coming in.

One methodology that is used to overcome these challenges is the manner by which one or more functions of the machine learning module is trained to recognize true measurements, which function(s) when trained can be fine-tuned to adjust the measurements and parameters derived therefrom so as to better assess the most pertinent measurements by which to navigate through the parameter space to map the parameters to the outcome. Particularly, a number of machine learning tools, e.g., including a number of differentiable equations, may be implemented and used to clean the signals, reads, and data so as to better calculate the parameters and thereby move from the input to the final output calculating the parameter data with decreased noise. For instance, all of these differentiable equations may be formed into a network or pipeline of calculations of the various weights and/or biases of each of the reads and/or swaths that move forward from the inputs all the way to the final output, e.g., in a forward pass, and then, in various instances, a backwards pass, moving from the final output back to the original inputs may also be calculated. In these regards, the calculating of the various weights moving from the inputs to the final output from which a determination of the concentration may be performed by a calculator function, such as an estimator and/or encoder.

However, as the calculator moves, e.g., descends, from the input to the output, in the case of training the model to the known output, there is likely to be a certain amount of loss, unless the model is completely accurate. This loss occurs when too much or too little weight, e.g., too much bias, is given to a measurement than is consummate with the contribution that measurement actually makes to the final concentration sum. Where the attribution is greater than the true contribution a loss is incurred, and when the attribution is smaller than the true contribution a positive loss is incurred. Accordingly, this loss, in part, represents an error in the weightings of the measurements, which is embodied in a difference, e.g., a loss, between the signal that is accurately represented by its attributed weight, which is desired, e.g., the weight of the measurement is accurate, and the signal whereby the attributed weighting, e.g., by estimation, is not accurate, e.g., the weight does not accurately reflect the contribution of the measurement to the final analyte concentration calculation.

When faced with such loss, the inference engine of the system, e.g., calculator, will change the weighting and/or biases of the measurements within the data structure by re-distributing the loss among a number, e.g., all, of the parameters being calculated, such as in an optimized manner. In such instances, the calculator may be considered as an optimization estimator, which may be an algorithm that performs a gradient descent that tweaks the weights and/or biases of the various measurements of the feature space, e.g., initially randomly, until a pattern is discovered, so as to better determine a pathway through the data structure that results in a final answer that best corresponds with the expected, e.g., inferred, outcome. This new path results from better tweaking the weights and/or biases of the various parameters by assigning the loss value to a more accurate distribution of features within the space. Hence, it is through this loss function that a more representative, more accurate, or better, set of parameters are derived, which parameters with revised values better leads to the true concentration output.

In various embodiments, however, these determinations do not necessarily end here, however. For example, in some instances, once an output is derived or estimated, then the system may analyze semantic space once more, but this time in a backward manner, such as during a backward propagation process. This backward propagation may be performed in instances where the true outcome is known, and the back propagation is performed so as to test how the estimator is functioning. Or, in like manner, such a backward propagation can be performed such as where the outcome is known, but the estimator did not arrive at the correct conclusion. In such a manner the back propagation can be performed so as to determine what must have gone wrong to lead to the original error in weight contribution that led to the wrong answer being called and how that error occurred. This information may then be used to determine what the correct pattern should be that leads to the changes, e.g., in the weightings, that results in deriving an accurate value. Once this process has been performed, then another, forwards, propagation can be rerun.

In these regards, the measurements that are captured by the emission of each electromagnetic pulse from each emitter that engages with the tissue and is transmitted, e.g., reflected back, to the photoreceivers (in the case of the photonic sensor arrangement) or power detector (in the case of the RF or microwave emitter) may be entered into a data structure and be processed, e.g., calculated, so as to estimate an analyte concentration output. Hence, in interpreting the meaning of this data, e.g., in relation to determining the concentration of a specific analyte being present within the tissue, a matrix, table, graph, and/or other data structure, may be formed so as to better visualize what is taking place within the tissue, but as represented numerically, such as algebraically and/or geometrically. An inference engine may then run an estimator function through this numerical data in a manner so as to reveal a pattern therein, and this pattern may then be used to estimate an ultimate analyte concentration value.

In various embodiments, therefore, to graph this data, each measurement may be represented as a scalar and a plurality of measurements may be represented as a vector. In various instances, all of the measurements may be represented simply by one, two, or three vectors, although there could be more. In such instances, as there may be 3, 4, 6, or more emitters for each array, e.g., in an optical configuration) of the detection device, each read may include 3, 4, 6, 7, 9, 10, 13 or more measurements, where each measurement may be represented as a scalar. And where a microwave array is included, up to about 150 measurements may be taken, which measurements may be represented by 150 different scalars. Hence, there could be about 150-163 or more measurements being represented as scalars, depending on how many and the type of sensor arrays included within the detection device and how many emitters are included within each array.

Collectively, it is expected that because the tissues respond to the impingement therein by electromagnetic radiation differently based on the concentration of analytes, e.g., glucose, within the tissue. Then collectively the measurements collected herein, as represented by one or more, e.g., 3, vectors, when analyzed, can be corresponded to the analyte concentration. Specifically, in specific embodiments, these three vectors can be used to determine the presence and concentration of analytes, such as glucose. Consequently, the analytics system may be configured for deriving a function, e.g., estimator function, by which these various readings, e.g., measurements, may be formed into a model that can then be equated with the concentration of glucose, for example.

More particularly, alternatively or in addition to, as discussed herein above, in particular embodiments, this estimation process may be configured as a regression model. In particular embodiments, to better understand the correspondence between the readings of the measurements and the analyte concentration, a plurality of sets of readings, which may be represented by one or more vectors, e.g., in one or more cycles or sweeps, may be collected and analyzed as a regression. Performing a plurality of sweeps is useful because there are very subtle variations in the structure of tissue that can have radically different effects on the glucose estimate that can be captured and seen more clearly through a number of reads, especially when those reads may be graphed and compared to one another. In various instances, these variations and the effects of the analytes, e.g., glucose, upon them can become embedded in the inputs of the data structure, separating this data other therefore, will help determine the concentration of the analyte, e.g. glucose. So, during the calculation process, at the very end of the network, the output of the network can be represented a plurality of neurons, e.g., output neurons, which can be compared to determine which better maps the inputs to the analyte, e.g., glucose, value.

In interpreting the meaning of this data, e.g., in relation to determining the concentration of a specific analyte being present within the tissue, a matrix, table, a graph, and/or other data structure may be formed so as to better visualize what is taking place within the tissue, but as represented numerically, such as algebraically and/or geometrically, e.g. as embodied by Cartesian and/or Euclidean Geometry. In this regard, the validity of a model, as explained in greater detail below, may be assessed based on how the inputs and/or outputs may be mapped to a graph. For example, a plurality of the measurements may be transformed so as to be represented not only in a two-dimensional table, as described above, but also as having coordinates so as to be represented in the semantic space as scalars and/or vectors (when various measurements are combined) that can be graphed.

Specifically, each measurement itself may be represented as a scalar, and further, each output from the data structure may also be represented as a scalar, which scalar will be representative of the final analyte estimate. The vectors, on the other hand, may be represented as a set of scalars, whereby if the calculator, e.g., estimator function) is doing a good job at determining the true contribution each measurement makes to the final measurement outcome, and is reflective of the true glucose reading, then the various different output values from a series of reads in a sweep will start to cluster together on the graph. In these regards, a scalar represents a measurement, which is essentially a number, and a vector may be represented by a plurality of numbers.

The parametric space within the neural network may be likened to our galaxy, whereby geometric structures, e.g., clusters of scalars and/or vectors, may be formed within the space. Particularly, like our galaxy is composed of stars that form constellations, the scalars (and vectors) of the graphed parametric space may from clusters, whereby the measurements and their respective weightings become all grouped, e.g., clustered, together, which would be expected if those measurements contribute to an actual analyte, e.g., glucose, concentration. And as the parametric space is calculated, the results may be reduced to a vector constituting three outputs, which can then be mapped, e.g., graphed in Euclidean space.

This clustering happens through the first pass simply because of the dynamics of the natural world we live in. Thus, one of the purposes of the machine learning module is to produce the particular clustering, as it reveals itself in the processing of the measurement data, because what is being sought through the data is a function that appropriately attributes the measurements and weights to the same or different clusters, with as minimal cluster loss as possible. Hence, when the calculator, which in this instance, may be an automated estimator, traverses through the data structure, from the initial inputs to the concentration output, calculating the measurements, a graph of one or more clusters may be formed. Then the various different clusters can be analyzed so as to determine the individuation of each cluster as well as to ensure that each result is attributed to the correct cluster group.

Such analyses can be employed to determine the loss parameter, or centroid loss, such as to determine if the placement of the result within a given cluster was what was expected given the determined estimate, or if the placement was off from the estimate and/or also off from where it was predicted to be. This can be measured as centroid loss, whereby an iterative loss algorithm, e.g., encoder/decoder, can be applied to the clustering and loss may be determined, and where the loss is above a certain set point, a re-clustering algorithm may be employed to perform a re-clustering of the results so as to end up with a set of different attributable losses. Consequently, this process results in the generation and training of a function that positions these analyzed parameters, e.g., weights and/or biases, in semantic space in a comprehensible manner. And further, the endpoint of the operation of the function may be the identification of one, two, three, or more of the most important clusters or neurons, or subdomains thereof, which have the most information and contribute the most to appropriately attributing the correct weighting to the measurements so as to clearly resolve the estimate and thereby determine the correct concentration lovel.

Likewise, as the graphing and clustering are performed, e.g., by a suitably configured encoder/decoder operation, the weights and measurements attributed to a given cluster can be color coded, such as based on a high concentration and/or medium and/or a low concentration of the analyte of interest, such as where one color can mean a high analyte, e.g., blood glucose, value, but another color can represent a low blood glucose value. Hence, one or more, e.g., every single point, can be run through the whole data structure, and be graphed to a particular point in the graph, reflective of the parametric space. Consequently, where a large selection of the measurements are mapped, then clusters may be formed, such as where those clusters can be color coded, e.g., based on the concentration of the analyte being measured.

If the network is smart and working well, all of a first, e.g., red, color of points will be clustered together so as to from an all “red-based” cluster, e.g., like stars in a galaxy, and further, all of a second, e.g., green, color of points will be clustered together so as to from an all “green-based” cluster, such as where the red cluster represents instances when the analyte, e.g., glucose, levels are high, and the green color represents instances when the glucose levels are low. A medium “yellow” cluster can also be formed. Other colors, of course, can be used in similar fashion. This is all a function of the mapping and calculating taking place through the data structure, e.g., neural network. And what the machine learning module is configured to do is find and determine a way to make that mapping work so as to clearly form distinct clusters out of the measurements. And when the mapping is going well, and the clusters are clearly separated, because of this clear separation, the machine learning module can more easily convert the mapped readings into accurate concentration, e.g., glucose, values. In this manner, the operations discussed herein can start with a single point, e.g., measurement, that was never seen before, this point can be combined with a series of other such measurements, e.g., in a cluster, they can all be run through the matrix, they can be mapped and/or graphed and then they can be used to determine the concentration.

In other words, as the glucose concentration of the subject changes over time, such as due to eating or working out, so as to increase or decrease, respectively, then the scalars, when graphed, will form a plurality of clusters, such as a cluster representing high glucose values, e.g., which occurs as a spike immediately after eating, or low glucose values, which occurs as a decrease in glucose when working out, and/or as a third cluster that may be representative as an equilibrium, e.g., baseline or medium, state. Essentially, as the readings are being collected, they are run through the data structure, and the auto encoder (decoder in back propagation) can then calculate the measurements, where necessary re-attribute the scalars, and one or more vectors thereof that can then be re-graphed.

More Particularly, when determining the concentration of an analyte, such as using a neural network or other data structure, an auto-estimator and/or auto-encoder may analyze a series of final output scalars to generate a semantic vector that represents the entire semantic space, which can then be equated with the overall concentration of the analyte of interest. For instance, in a first pass, the auto estimator may be employed to attribute scalars into clusters, and then in a subsequent pass, the auto-encoder can be used to reattribute those scalars as necessary. In these manners, as indicated above, the various measurements may be narrowed down to three variables, by which they may be represented as a semantic vector and can thereby be graphed within a Cartesian coordinate system. The shape of any one of these semantic vectors can be analyzed, such as to determine one or more trends with respect thereto, e.g., the shape of the semantic vector can evidence how the semantic space is emerging relative to the different analyte, e.g., glucose, values being measured.

For example, if the scalar clusters and/or semantic vectors that are being generated from the inputs are such that there a clusters (or vectors) forming, and there is clear segregation and separation between the readings at different times, then the auto-estimator and/or encoder, is performing well. But if there are overlaps between these semantic vectors within the space, then there may be a problem with the estimator/encoder that needs to be corrected, such as by being further trained. This differentiation, segregation, and organization is useful to see because it illustrates how well the data being analyzed reflects the true biological conditions of the subject.

In various instances, to help better perform clustering, as well as to aid in generating separation between the clusters, a K-Nearest Neighbor (KNN) algorithm may be trained and applied to the data. KNN is a supervised learning algorithm that can be applied for both classification and regression. Being trained on labelled data, the KNN function can then take a first, new instance of an unclassified data point, e.g., a scalar or a vector, define it, and then assign that data point to the “K” nearest neighbor, e.g., the most similar data points or clusters (neighbors) to the newly defined scalar or vector, where “K” is a number of scalars or vectors that can be graphed closest to the defined scalar or vector. For this purpose, the algorithm takes a majority vote among the “K” neighbors, e.g. clusters, and the new data point is assigned to the cluster, e.g., class, that is most represented among its neighbors. In this regard, the algorithm is a regression that takes the average of the target values of the “K” neighbors. However, this process can be computationally expensive for very large datasets because it needs to compare a new point to every point in the set, e.g., training set during training. This new data point may then be assigned to the most frequent class among those “nearest” neighbors.

To perform these tasks the KNN uses a distance metric, e.g., a Euclidean distance metric, to determine which data points are “nearest,” where the value for “K” and the distance metric may be pre-defined. In this instance “K” is the number of neighbors to be considered. Hence, for new, unclassified measurements, this ML algorithm can be configured to find the “K” closest points to it in the training dataset based on the chosen distance metric. The value of “K” here is important because a low “K” value can lead to high variance causing overfitting to noise, while a high “K” value can lead to high bias resulting in oversimplifying the model. It is to be noted that in these instances, the vectors being generated and graphed do not necessarily need to be represented in three-dimensional space because the mathematics are not confined to Euclidean geometric space. Hence, there can be several different dimensionalities represented, e.g., 5, 10, 32, 50, even 100 or more. For ease of visualization, however, it is useful for the generating and mapping of the graph to take place in three-dimensional space.

Consequently, once training has been accomplished, the KNN algorithm can be run in an unsupervised manner so as to perform clustering. KNN is useful, therefore, for classifying and categorizing the various collected datapoints, such as with respect to relatively high, medium, low, and trending values. Further, along with the other ML algorithms disclosed herein, KNN can be employed to run through the data structure, e.g., in a forward direction, look at the parametric space, decide how to label the datapoints, and then separate the various measurements into the appropriate clusters. In this manner, the unlabeled datapoints of the data structure may be assigned to a cluster and thereby be labelled. Where a number of clusters are clearly formed and distinguishably separated, this evidences that the multi-sensing detection device and the analytic system are working appropriately.

In certain instances, however, cluster loss may be experienced, such as where new datapoints are poorly fitting into their local neighborhood (or the overall cluster structure generally). This can be determined by measuring the distance between the datapoints in question to the centroid of the relevant cluster. In such instances, such distortion can be minimized and/or class separation can be enhanced, by applying a K-means algorithm along with the KNN algorithm. In this regard, the K-means algorithm can partition and/or group, or regroup, hard to assign, unlabeled datapoints into more appropriate “K” distinct clusters. It performs this function by iteratively assigning data points to the nearest cluster or centroid and it may then recalculate the centroids until convergence is achieved. Finally, a discussed herein below, once the clusters have been formed, and the regression has been run and fed into the output layer, a result can be determined and output. Where the analyte in question is glucose, and the result is a concentration, then the output can be a number, such as a number that is between 0 and 400, which is reflective of the glucose concentration. However, in various embodiments, the data structure can be configured to classify the outcome, such as in one of a number of classes, for instance, in a high, medium, or low class of concentration values.

As indicated, through this regression process, the system allocates a set of weights and/or biases to the collected measurements that appropriately reflects their contribution toward the final determination of the analyte, glucose, value, which accurately reflects how the tissue responded to the electromagnetic radiation with regard to a particular set of emission criteria. Specifically, through this regression process (and/or back propagation process described below), the automated estimator (and/or encoder) is defining a function that can map a selection of the most pertinent inputs to the final clusters and/or final output, so as to produce a good estimate of the concentration of the analyte of interest. Once this function has been determined, and the neural network has been trained, the estimator of the trained neural network, can then calculate the values and map them to one or more clusters and/or to a final output, dependent on the system configuration. As indicated, this estimator and the training may include back propagation, but when the estimator is actually running correctly, back propagation need not be performed, as it is the forward pass that will map to the final glucose concentration call. Hence, after training, the methodology for redistributing loss throughout all of the parameters in a manner that appropriately apportions reads to the correct outcome, once determined, can then be employed again and again, such as for use as a calibration. Consequently, once the pattern has been determined, a model can be generated, whereby use of the pattern on the same inputs will lead to the same outcome results.

However, as originally generated, the pattern may be input specific. The pattern therefore will need to be modified to be universalized. Specifically, in order for the pattern to be universalized, a model, based on the pattern needs to be generated, whereby the pattern, embodied within the model, can then be universally applied to new datasets. In essence, through the training processes set forth herein, a general pattern recognition technique or model may be generated, whereby when new datasets are uploaded, they may be fed into the data structure, the new pattern within the data structure may then be recognized by the model, and the forward mapping via the estimator, in view of the model, can be performed, and a new analyte concentration can be predicted, such as now in an unsupervised process with minimal to zero loss. These calculations, however, are very complicated, as the parameters in the network can include hundreds, thousands, even millions of measurements, which all contribute little or minimal adjustments to the final concentration value but still have to be calculated and assessed to determine the correct allocation of their weighting so as to derive the accurate concentration value.

In performing this process, therefore, a first operation is to initially form an input layer into which the initial measurements, e.g., signals or reads, may be entered. With respect to the optics-based measurement devices set forth herein, each optical emitter of each array may perform a measurement at a determined time, for a determined duration, and at a determined intensity. Thus, with respect to the sensor arrays employed by the detection devices herein, the arrays may include any number of photoemitters, such as 3 photoemitters, e.g., in a PPG sensor array, or four, or six emitter optical arrays, etc. Likewise, for the radio frequency and/or microwave sensor unit(s), the signals within a sweep may be up to about 150 readings, e.g., all at different frequencies.

Hence, the number of signals being generated can range from 3 to 4 to 6 up to 150 or more, which signals represent reads being taken at the energy receivers, e.g., photodiodes or power detector, and which will be fed into the data structure so as to perform the calculations set forth herein and thereby derive the final analyte concentration value. Consequently, in various implementations, such as where the detection device includes an optical device, 3, 4, 6, 9, 10, 12, or more photoemitters may be used to generate the data, e.g., measurements, which may then be inputted into the data structure, such as a neural network, from which a final glucose concentration can be determined. In such an instance, any given neuron within the neural net may receive and/or encode several different inputs from the sensor arrays.

For example, in one embodiment, in an initial implementation of the neural network, a first neuron may receive all of the 3, 4, 6, 9, 10, 12 or more inputs. The estimator will then attribute an initial weighting, e.g., randomly, to the measurement values input into this first neuron. Hence, in an initial step, the weighting of each of the inputs may randomly be attributed a weight as to what its predicted contribution to the overall outcome will be. This weighting converts the input into a parameter, and these parameters can then be calculated so as to determine the pattern by which the response of the tissue to electromagnetic radiation may be determined based on the presence and concentration of an analyte of interest being present within the skin and tissues, which then will allow the system to determine the concentration of the analyte by determining this concentration dependent tissue response.

In particular instances, one or more of the weightings may be multiplied by one or more factors, and then, the estimator may calculate the sum of one or more, e.g., all, of the weighted (and factor multiplied) measurements, which may then further be modified by the application of an activation factor thereto. Hence, each neuron may receive multiple inputs, a weight may be applied to each possible input, the inputs may be summed, and then the activation function may be applied thereto. However, each activation function may be different depending on how the neuron is configured to behave.

For example, in one implementation, the activation function may be a Rectified Linear Unit (ReLU) function. With respect to the neural networks set forth herein, a ReLU function, as described above, is an activation function, such as where f(x)=max(0,x), whereby as the estimator traverses through the data structure performing its calculations, if the weighting of the parameter is positive, then the sum is directly output, but if the sum is negative, then the output is set at zero (0). As discussed above, the ReLU is useful for introducing non-linearity into the analyses, which allows the neural network to better learn the complex patterns resulting from the interactions of the tissue with the electromagnetic, e.g., light, waves being measured such as within the system herein.

These relationships and the patterns they form are often so complex that they simply cannot be appreciated by a mere linear model. Hence, different configurations of activation functions may be employed so as to achieve different conformations of the data being analyzed, thus allowing the data to be analyzed in different manners for a variety of different purposes, such as depending on whether linear or nonlinear behavior is to be assessed. Therefore, in these instances, by applying the max(0,x) ReLU function, this converts the non-linear relationships within the data structure into a non-linear relationship between the input and the output of the neuron thereby allowing the network to model the complex, non-linear response data generated by the tissue's reaction to electromagnetic radiation. In this regard, by applying this ReLU activation function to the data structure, the initial random weighting of the parameters can be determined more precisely so as to determine the actual percentage by which the measurement reflects the contribution of the tissue's response to electromagnetic radiation at that particular wavelength within those particular emission criteria, which may be expressed by its true determined weighting.

Particularly, as the estimator starts to reveal the pattern, then using the identified pattern, a more specific and accurate attribution of the proper weighting and/or biases of each input can be made by the system. For instance, every time the estimator performs a forwards propagated pass calculating the sums so as to reach the final output, where the calculated outcome does not match the known or predicted outcome, then there will be a positive or negative loss, as described above, which loss must then be redistributed, and the calculation assessing the newly generated parameters will be performed again. In various embodiments, part of this redistribution process may involve a backwards propagation. During this back propagation, the estimator works its way through the data structure back to the beginning re-attributing the loss, but this time in a more informed manner, which thereby causes the weighing (and biasing) of each parameter to be adjusted, but this time in a non-random, but calculated manner. However, in various instances, in substitution for or in addition to the ReLU activation function, a number of other activation functions may also be employed such as a sigmoid, or GLu, or some other activation function may be used where they all have different kinds of effects.

Accordingly, in view of the above, the arrays of the multi-sensing detection device may be configured for directing electromagnetic radiation into the tissues, and for collecting the responses therefrom, which responses become the inputs into the data structure to be calculated thereby, such as by the artificial neural network described above. Particularly, depending on the configuration of the sensor arrays, e.g., including three, four, six, or more or less emitters, each emitter may be activated for a determined time, in a determined sequence, for a determined duration, and at a determined intensity, whereby these emissions cause a series of responses within the tissue, depending on the concentration of the interstitial milieu therein, which responses affect the reflectance, e.g., refraction, absorbance, scattering, and polarization, of the impinging electromagnetic radiation, which reflectance may then be directed back to the detection device so as to be detected thereby.

Specifically, each emitter at each array produces a skin response which affects the reflectance of the return signal, which is then measured by the photodiodes of the system, are converted into digital read, e.g., measurement, data, which are then transferred to the analytics platform of the system to be entered into one or more data structures, e.g., of the neural network, whereby their ultimate values may be determined in relation to and contribution with all of the other emitter results. Consequently, each read becomes an input into the data structure that gets weighted, calculated, and backpropagated, for each collection of reads taken for each circuit of each sweep of each cycle of the emittances of the arrays.

Such cycles can include 1, 5, 10, 20, 25, 50, 100 or more cycles, of all emitters, e.g., 3, 4, 6, or more emitters from each array, which produces a very large amount of input data being generated. So, for each array, 3, 4, 6, or more or less reads are generated for each run of a cycle, thereby generating a corresponding number of reads, e.g., frequency inputs, per run. However, where the sensor array includes a microwave sensor unit, each emittance cycle from a single microwave emitter may include about 150 frequencies or inputs per cycle. In various embodiments, a variety of other data, such as demographic and other pertinent user data, as well as sensor related, e.g., location and position, data may be collected and added to the measurement data so as to be considered by the machine learning module. Other of such data may include the user's health data, their blood pressure, exertion levels, stress levels, arterial stiffness, temperature, moisture (galvanic skin response) data, movement and acceleration data, orientation, and the like.

As described above, in various embodiments, the data structure architecture, e.g., artificial neural network, may include several different layers. For instance, a various number of the reads may initially be input into a first, input, layer. A second, hidden layer, may also be included, wherein a number of processing operations may take place, e.g., the hidden layer may be a processing layer. In various instances, the second (hidden) layer may include a set of processing layers, whereby tens to hundreds to thousands of parameters may be analyzed and assessed. However, the greater number of parameters to be assessed in this hidden layer(s), the longer and more complicated the calculations will be, but, on the other hand, if the computational layer is too small, a correct pattern may not emerge and an accurate assessment may not be made, and thus, a tradeoff has to be considered. And finally, after “N” number of hidden layers, where a majority of the processing occurs, there is a final output level.

Accordingly, there are many different components to the neural network architecture that also must be constructed and tested a long with the measurement data, so as to best map the inputs to the outputs. In other words, it is not just the methodology for calculating the inputs that needs to be optimized, but the architecture itself, such as with regards to how many processing layers the hidden layer will include, how many parameters will be processed by each processing layer and which processes they will include, and what the size and configuration will be of each processing layer. For example, a first processing layer may include all of the initial set of parameters to be calculated, however, after a first pass, a certain number of the parameters, not arising to a cutoff point of probability, may be excised from the sample set, and a new hidden layer will be generated with a new set of parameters, whereby this second layer has a smaller area then the initial processing layer.

Such processing steps, through the various neuronal layers, may be repeated a number of times until the ultimate pattern is revealed, and the final outcome parity is achieved. In this regard, the rate by which these calculations are performed, and thus, the movement from one layer to the next, may also be determinable, or in other words, the learning rate may vary, and in this regard the number of epochs to be considered, e.g., in a training regime, may also vary. In this instance, one complete pass of an entire training dataset through the machine learning model may constitute a single epoch, and dependent on the system configuration, the training of the machine learning module may include one or more epochs.

Consequent to the various arrangements of the data structure is the determination of how much of the collected data will be allocated to test versus validation data, which forms the dimensions of the first, input layer, which will then inform the configurations of all subsequent computational layers. Hence, in many instances, the very architecture of the data structure may be dynamic and may be made to change as the consideration of the measurements change, e.g., in other words, the active space may be variable based on the calculations being performed and in which contexts. For example, when the layering of the data structure is performed, there can be a small hidden layer or a medium, larger, or very large hidden layer that may be produced, and then the next layer could be smaller or larger, and then the next layer could be smaller and larger, and so on. Typically, a first, large hidden layer is generated, and then as the various calculations are performed, the subsequent hidden layers are brought down to smaller and smaller and smaller layers, making the network smaller. Thus, even though the layers become smaller, the computational space, en mass may be expanded out to a very big parameter space.

But as the estimator moves through the layers, e.g., searching for the pattern of parameters that will lead to the correct final value, the semantic space is condensed down all the way until a single output value is derived, e.g., a final regression estimate is called. In essence, the estimator is calculating and searching the inputs within a given context to find the right mix of parameters the calculation of which will create a function that maps the input to the final output. This, in essence, is what is being sought for, e.g., a function that can generalize and map from the known, input space, to the unknown output space. However, there is a temporal component that also needs to be considered, because before the measurement is taken, the tissue is in one state, but by taking the actual measurement, the state of the tissue is changed by the very measuring process.

What this means is that the measuring environment, throughout the measuring process, is in a continual state of flux. But, not only that, one measurement device will inevitably be slightly different from all other measuring devices, such as through artifacts being incorporated therein through the manufacturing process. Likewise, the tissue of one person being measured will also be different from the tissue of another person being measured. All of these factors cause differences in the measuring system that all need to be accounted for when trying to generalize a measurement process that is applicable to all devices performing measurements on all people, regardless of the state they are in. Hence, what is desired is that the measurement devices measure the same factors regardless of the particular devices making the measurements, regardless of the person, regardless of the location, regardless of the position of the device on the body, regardless of the present condition of the body, essentially regardless of any other factors. To ensure the measurements being taken are as accurate as can be, without interference caused by the aforementioned factors, it is useful to generate as much contexts surrounding the measurements as possible so as to better account for and discount various of the potential interfering factors.

Therefore, a variety of contextual data may be collected at the same time the measurements are being taken and can be fed into the data structure as further inputs so as to allow the estimator to take account of the contextual circumstances in which the measurements are taken along with calculating the measurements themselves. Calculating the contextual data along with the measurement data is useful because, in various instances, the measurements can be ambiguous such that a closely aligned, e.g., relatively the same, set of inputs, taken at different times, can be matched to the same set of glucose values. Consideration of the various contextual data can be useful in distinguishing between these different but aligned parameter data. Hence, what is desired is to understand what the context is, because it is useful to know what happened to the last signal, and what happened to the last signal before that, and further what happened to the last signal before that, and specifically, it is useful to take account of all of that information in order to be able to make an interpretation of the past and current contexts so as to better distinguish the difference and meaning between the collected parameters.

In addition to having a multiplicity of layers, the calculation space of the data architecture may further include a plurality of dimensions. More particularly, in various embodiments, the data architecture may be a precise type of artificial neural network such as the convolutional neural network (CNN) discussed above, which may be configured in one, two, three, or more dimensions. In such instances, the analytic space may be multi-dimensional. Hence, there are different ways to organize how the inputs travel through the neural network, what data is to be included in the calculations, and how the data is calculated, changed, and manipulated as the inputs travel through the neural network.

However, as indicated above, initially, the building of the data structure and the calculations taking place therein is in a supervised fashion, such as for training the model, where the final outcome value, e.g., analyte or glucose concentration, is known. These are the contexts and conditions whereby the pattern can be recognized, and in which the model may be formed. Through this process of supervised learning, the machine learning module may be trained.

In accordance with this supervised process, the various test and known result input data may be correlated but separated into different groups, such as by one or more of the functions described herein above, where each group may be categorized and labelled, e.g., differently. In various instances, part of the collected data may be labelled test data, the other data may be labelled control data, and still other data may be labelled validation data. In such instances, the test data may be mapped, in iterative fashion, to the control data, e.g., to the known output values, so as to recognize the pattern and generate and train the model. Then the validation data may be mapped, via the model, to the output data, so as to validate the model. What this means, in particular embodiments, is that as the model is trained, the encoder is attuned so that as the estimator traverses through the data structure, it accurately maps the inputs to the known output value.

In this regard, the estimator may be a function that has been trained to accurately align a generated series of measurements to a set of corresponding known analyte, e.g., glucose, measurements, and this process of training the estimator may be referred to herein as a calibration. Once the estimator has been calibrated, it may be used to map new, unknown measurement values, along with any relevant contextual determinators, to unknown glucose values, which glucose values can be inferred from the application of the estimator to the new and unknown measurement input data. Because the ultimate analyte values are not known, e.g., this estimation process is performed in an unsupervised manner, a backward prorogation will not be as informative here as it is when those actual values are known.

Such backwards propagation, as described above, in a supervised learning paradigm, is useful because when the estimator is being trained, the first, e.g., training, pass, allocates a preliminary weighting to all of the initial measurement values, e.g., converting them into parameters. The system can then test this initial allocation of weighting and/or biasing of the parameters because the actual analyte, e.g., glucose, concentration value is known.

However, as indicated above, when the estimator calculates each of the values within the matrix to derive a final analyte, e.g., glucose, estimate, and when the estimated concentration is not the same as the known concentration, then a loss value occurs. This loss may then be redistributed among all, or a selection of, the weights of the original parameters, and a second, or more, pass may be performed until the weighting is such that the estimator accurately maps to the known concentration value. In essence, when in a training mode, when the original pass results in a loss, then a back propagation can be performed, and a loss function can be employed to reallocate the loss throughout various of the parameters of the data structure. Subsequently, a new run can be performed, whereby the result should be either a lower loss or an accurate call. Thus, this process can be performed again for the entire epoch until the actual concentration is derived by the estimator. However, where the final concentration value is not known and a back propagation cannot be performed, because a first calibration has already been performed, then the ultimate determinations of the unlabeled parameters to be calculated can be evaluated with respect to comparison with the calibration data.

As set forth herein above, the deep learning data structure being described may be an artificial neural network (ANN). For example, in various embodiments, the artificial neural network may be implemented as a Convolutional Neural Network (CNN), whereby the CNN can be configured to map all of the inputs into what's called a latent semantic space, e.g., so as to form a latent semantic vector. More specifically, as discussed above, a CNN is a type of deep learning architecture that is designed for analyzing data, e.g., data that can be represented graphically, such as the analyte based reflected electromagnetic radiation data set forth herein.

In this regard, the CNN may be instantiated within a number of specialized neuronal layers by which the analytics system may automatically and adaptively learn the features represented by the collected data such by clustering them into spatial hierarchies. For example, in an initial implementation, the CNN may start in an early, convolutional, layer by defining simple features like patterns that form a center or boundary, which in later, pooling, layers may be combined into more complex groupings so as to better detect more complex patterns in deeper layers. In these regards, the middle, e.g., convolutional, layers form the core of the CNN.

In various embodiments, these convolutional layers may be composed of one or more kernels, or filters, which may be employed to generate and/or analyze the data structure so as to detect patterns in various of the features inherent to the measurement data. Particularly, these convolutional layers employ filters that may be configured to construct the weight matrix when generating the parameters, and then an encoder may be used across the entire input so as to reduce the number of parameters, while at the same time as allowing the network to detect the same features within a pattern regardless of where it appears in the pattern The result of this process is the generation of a feature map. The pooling layers may then down-sample the feature maps, thereby further reducing the spatial size and computational complexity, and creating a compact “latent space” representation, e.g., a context vector, which contains the most essential information. In this manner, the number of convolutional layers may be pooled so as to reduce the spatial dimensions, e.g., X, Y, and Z dimensionality, and a feature map(s) including all of the most important features may be generated. This pooling, therefore, helps to decrease computational complexity making the network more robust to small variations in the positioning of the features forming the pattern. The output of this process is a feature map that highlights the most important features of the identified pattern(s).

However, even though the various layers may be fully connected with one another, each neuron in the convolutional layer(s) may actually only be connected to a small, local region of the previous layer. The job of the encoder, therefore, is to systematically reduce the spatial dimensions of the input data while extracting and abstracting the important features. This better focuses the network on determining local patterns. After the convolutional and/or pooling layers have extracted and condensed the feature data, the final layers may take the high-level features forming the pattern and use them to derive the final output, e.g., to take the patterned feature and measurement data and make a final prediction (or classification) as to what the concentration is.

Once this process has been performed, the data compressed, one or more patterns identified, and an output generated, it may be back tested, so as to see if when reversing the pattern, moving backwards from the output, the original measurement data may be derived. Therefore, once a forward pass has been made, then a reverse pass using the same or another neural network may be formed to run the matrix in reverse, via a decoder, from the output back to the input. In such instances, the decoder takes the compressed latent representation from the encoder and progressively expands it to reconstruct the target output, which often has the same dimensions as the original input. This process, therefore, is the reverse of the encoder.

In these instances, the process of employing a calculator, e.g., an encoder, so as to convert the measurement inputs into a concentration output may be conceptualized as an encoding process, because the goal is to move from the highly complex parameter space to identifying the code that encapsulates the meaning of the collected signal data. The results of this process is a data structure embodying a parameter space that then needs to be decoded. Therefore, once the pattern has been encoded, it may then be decoded by the movement of the calculator, e.g., decoder, now in the reverse direction, through the data structure, moving from the generated output, through the data structure, to the initial inputs through the various layers of the CNN. Hence, once the first, forward pass has been performed, then a back pass, reverse decoding process, can be performed so as to derive the original signal. This is useful because, if the original signal can be derived, e.g., with minimal loss, that means that the encoder is doing a good job encapsulating the true signal into the parameters being calculated, e.g., encoded and decoded.

In various embodiments, the forwards pass and the backwards may be performed on the same or different neural net, and/or on the same or different layers. For example, in one embodiment, the backwards pass may be structured as a regressor model that operates on the code, e.g., in reverse, and these two networks can be trained as two completely separate training processes. As indicated, the reason for this backwards pass is because it gives insight into whether or not the mapping is working or not, and together these matrices define and shape the semantic space.

Particularly, as indicated, if the mapping is working well the measurements may be graphed and visualized, and hopefully clustered together, dimensionally, such as in accordance with their Cartesian coordinates. What this means is that if the subject is experiencing low blood glucose, then the inputs will all map generally to the same Cartesian quadrant, while when their blood glucose values are high, then the inputs should map generally to the opposite Cartesian quadrant. Thus, the clustered separation between the low and high measurements show that the models are working, which means that the neural net is figuring out how to decide between one set of (low) values versus the other set of (high) values (any number of medium values may also be included).

However, if there is no clear separation between the measurements, and they are all mixed up, then that's bad, because it means that the neural net is not being able to find the pattern and clearly delineate the measurements in accordance therewith. In such an instance, further training may be required, and as discussed above, there are various different techniques that may be employed to more precisely separate the data. For instance, one or more other regressor models may be trained and be employed to learn the relationship between the features of one or more inputs and its contribution to the output and based thereon may make one or more predictions with respect thereto. In various embodiments, this and other such models and/or methods may be employed so as to analyze the independent measurement variables or values (e.g., regressors), identify relationships (e.g., patterns) therebetween, and then make predictions with regard to defining discrete classifications and/or categories of the variables dependent on the independent variables (e.g., concentration prediction).

In certain implementations, such classifications and/or categories may be determined by suitably configured classification models, which are particularly useful when analyzing measurement data collected by a microwave-based sensor, and in various instances, behave differently from regression models in that they allow the consideration of outputs from a plurality of neurons, e.g., where each neuron represents a different class and/or categorization. In particular embodiments, one or more of the classification networks may be directed to identifying and characterizing one or more trends in the collected, e.g., measurement, data, such as for identifying and monitoring such trends as glucose concertation going up, glucose concertation going down, or glucose concertation staying the same. This is useful information because identifying this trend indicates when blood glucose averages may be moving up or down or staying the same. However, as stated, in other embodiments, a regression model may be employed and configured to make larger, more general predictions, such as based on the input data, e.g., features, where the output value is a numerical outcome.

Additionally, as discussed above, in various instances, the data structure where the machine learning takes place may be, or may also include, a decision tree, e.g., a Light Gradient Boosting Machine (GBM), or Twin-Networks. Other functions that may be employed in these regards may be a M Least Partial Squares, PCA, and other such algorithms, so as to better effectuate the determinations set forth herein. For instance, a Light GBM provides a gradient boosting framework that is based on a decision tree algorithm that may be employed for determining labelling, classification, regression, ranking, and the like. In this regard, the Light GBM may be employed to discretize the measured values into histograms, whereby a leaf-wise strategy is used to split the leaves of the trees with the maximum loss reduction. This leads to a deeper, asymmetrical tree that achieves lower prediction errors in a faster manner.

Further, a twin-network, such as a Siamese neural network, may be employed such as where the data structure is replicated, whereby the input and other values therein may be compared so as to assess their similarity and difference, and to test different hypotheses and models. In various instances, the deep learning architecture may be composed of a plurality of data structures that share the same (Siamese) or different (non-Siamese) parameters and weights, and instead of classifying inputs into predefined categories, the Siamese network can be configured to learn a similarity function by comparing pairs of inputs, so as to determine how alike the various inputs are. In this regard, the two identical sub-structures will be processed independently by the similarity function to produce a plurality of embedded feature vectors, whereby a distance metric can then be used to calculate the similarity between the two different vectors. So being, during training, the loss function can be employed in such a manner as to minimize the distance between similar pairs and maximize the distance for dissimilar pairs, and opposite for the non-Siamese data sub-structures.

Another methodology that may be employed to train the machine learning module is a Support Vector Machine (SVM), which implements a supervised learning process for performing both classification and regression tasks by finding an optimal hyperplane that separates the data into different classes. The SVM does this by defining a hyperplane, a primary vector, and then maximizing that distance between the hyperplane and the closest datapoints, e.g., support vectors, on either side of the hyperplane. For example, the hyperplane may define a decision boundary, e.g., a line in two-dimensional space, that separates the data points into different, e.g., two, classes (which in multi-dimensional space may form a plane). In such instances, the support vectors may be represented by the data points that map the closest to the hyperplane, e.g., line, and they define the position and orientation of the hyperplane, thus, moving the support vectors moves the hyperplane. In such instance, a margin may be created, such as where the margin defines the distance between the hyperplane and the support vectors, and consequently, the SVM may be configured to find the hyperplane with the largest possible margin so as to maximize its predictive accuracy.

Here the use of an SVM is useful because the hyperplane can be defined as the line that represents the optimal glucose values, whereby a high glucose value can be represented by a support vector that maps above the line, and a low glucose value can be represented by a support vector that maps below the line. In this manner, the reads that are collected and evaluated may further be classified as pertaining to high, normal, or low glucose values, and thus, the concentration of glucose can not only be determined, but it can be qualified, and its trending high or low can also be determined. Likewise, the SVM may further be used to form a regression whereby the identification and continuation of one or more trends, e.g., in glucose values, can be predicted regardless of categorization. Further, the SVM can be used to detect and identify values that our outliers, and thus, are anomalies in the data set and therefore can be excluded from calculation within the data structure. Additionally, where a regression model is implemented, a structural equation modeling (SEM) process may be implemented whereby various causal relationships between the collected data may be estimated by performing one or both of factor and regression analyses. In this regard SEM provides a statistical framework that can be used by the machine learning module to analyze the relationships between observed, or known, variables and unobserved, or latent, variables.

Along with the SVM a kernel function may be instantiated whereby the kernel may be used as a ML classifier and may further be configured as a similarity function that takes two inputs and outputs their similarity. Thus, such a kernel function may be configured to add to the SVM by allowing the system to manipulate non-linear data by transforming it into a linearly separable expressions thereof. In other words, such a kernel function may be configured as a similarity function that calculates the relationship between two points of measurements so as to implicitly map the parameters into a higher-dimensional space where they may become linearly separable. In this manner, non-linear relationships may then be resolved using linear models like the above referenced Support Vector Machines (SVMs).

Particularly, the kernel function may be adapted to take two data points and return a scalar value that represents their similarity regardless of the higher dimensional space within which they are represented. Hence, by mapping the similar parameters to a higher dimensional space, a linear decision boundary can be created whereby the data can be further resolved, separated, and be represented linearly. In this regard, such a kernel function is useful because it can transform the complex parameter data into a simpler, more manageable format, without the computational cost of explicitly computing the higher-dimensional space. For example, in one instance, to better resolve the dimensionality of the various parameters and associated data, the system can look at the kernels themselves and see, what kind of kernels are forming, determine to what extent the kernels are distinct from each other, and can thereby better visualize their shape so as to better define their boundaries, e.g., graphically, to better assess their differentiation from each other.

In other embodiments, the data structure may further be implemented by a Bayesian network, whereby the machine learning module implements a directed acyclic graph (DAG) to represent the probabilistic relationships between the measurement values collected herein. In this regard, the data structure is configured as a graph where each variable, e.g., each collected measurement, constitutes a node on the graph, whereby the relationships between the nodes are defined by a set of edges, which represent conditional dependencies between these nodes. Specifically, in the graph each node may represent a random measurement variable, and arrows, which represent the direct probabilistic dependencies between the nodes, may be used to point from a parent node to a child node, where a cycle need not be iterated.

What this means is that one node can be seen to be in relationship to another node when the presence of the first node probabilistically determines the outcome of the subordinate node. Or in other words, the graph is a conditional probability table (CPT) where each node has a CPT that defines the probability of a child variable taking on a certain value dependent on the value of the parent variable. In this regard, the graph is useful for determining the dependencies between the various different measurement variables, thereby helping to visualize how the various variables influence or are influenced by each other. In this manner, the manner by which the various different variables affect, e.g., are conditioned upon or otherwise influence, the other variables may be graphed.

Hence, this network may be used to model the uncertainty between the variables and thereby make a prediction about an outcome wherein the precise relationship between the variables is unknown. This allows the network to reason out, e.g., infer, relationships between incomplete or uncertain data points, and allows the system to run through a variety of what if scenarios whereby the probability of different unknown outcomes can be predicted based on the known outcomes or evidence. Therefore, this graph is useful for explaining how an outcome was achieved, e.g., once the outcome did in fact occur.

In various embodiments, because the collected data sets of each layer of each epoch can be so large, it may be useful to reduce the number of factors, e.g., measurements, to be calculated. In such instances, a Principal Component Analysis (PCA) may be performed to reduce the dimensionality of the dataset(s), such as by transforming the large number of measurements into smaller sets of new measurements labelled as principal components. In this regard, PCA is an unsupervised process that can be implemented so as to identify key measurements that evidence the greatest amount of independence, e.g., non-correlation, which can be formed of linear combinations of the original variables.

This analysis will result in increased data compression at the same time as greater noise reduction with better data visualization. For example, by reducing the high dimensionality of the measurements, such as to two or three dimensions, as described herein, the measurement data can more easily be plotted and, therefore, visualized. In this manner, the collected measurements, once combined and linearly reduced, can be graphed, such as two or three-dimensionally, whereby the measurements may then be more easily visualized, such as where the various different parameters, e.g., measurements, may be seen to form clusters within the graph, thereby making it easier to see and understand what is occurring within the body based on what is occurring with respect to the graphed measurements,

In this manner, the complex measurement data may be simplified, while at the same time as maintaining the most important information and retaining the greatest variance. Specifically, once identified, the principal components may then be ordered by the amount of variance captured by the measurements, whereby the first component captures the most variance, while the second components capture the second most variance, the third captures the third, and so on. On the other hand, those measurements that embody the least amount of variance, e.g., they evidence the greatest amount of dependency, can be discarded leaving only those measurements that are the most significant for explaining the data, which will result in a reduction of the data set. In these manners, by focusing on the measurements and other components with the greatest variance, and discarding the measurements that have the greatest interdependence, noise can better be reduced.

Consequently, by creating new, less correlated features, e.g., via superior feature extraction, the data can better be interpreted and correlated to the final outcome, while at the same time as increasing the speed of processing and reducing computational costs. In this regard, the PCA can act as an estimator, but primarily functions to combine the measurement inputs in a manner to re-express them by a set of principle components that reduces the number of variables so as to embody them in a component that reflects the maximal amount of independence. Basically, what this means is that the various measurements that are the most similar can be combined and represented by a number of principal components that result in a minimal collection of components that expresses the greatest amount of differentiation between them.

Ideally, through one or more series of analyses, all of the different components can be reduced down to two or three principal components, such as to form a Cartesian matrix, whereby the various different parameters can be mapped to a minimal number of clusters within the graph or matrix, where the distance between the clusters is maximized, so as to better visualize the distinct properties between the measurements within each of the separate clusters. This occurs when the measurements can be analyzed and reduced down to a set of coordinates that can then be grouped along with a set of other similar measurements, which may be mapped to the same or similar spots, so as to produce a cluster within a specific area within a graph or matrix. This would be expected when the underlying analyte being assessed falls within a given level, e.g., concentration, within the tissue, and thus, it would be expected that the various measurements would naturally cluster together. And in situations where the analyte values are in flux, such as immediately after eating, then the measurements would be expected to scatter, until homeostasis is once again achieved, and in such instances, the trends related thereto, as homeostasis is re-achieved, may be identified, measured by the system, and followed by the data collection device.

Hence, the PCA process can be implemented as a mechanism for reducing the extent of the analysis domain making it easier to derive understanding from the conceptualized parameter space. In this manner, all of the different parameters may be reduced, clustered, and mapped to a conceptual space, whereby the various different measurements can be mapped and clustered together and based on this clustering meaning may be attributed to the clustered readings, such as where the readings equate with a distribution of high, normal, or low levels of the analyte within the body, and an identification of what the trend with respect thereto is, e.g., how is it trending. Not only does this make understanding of the measurements easier, it makes the processing of new measurements throughout the space much faster. Grouping or clustering the different parameters in this manner makes it easier and faster to go through the same space and arrive at the same result using less processes.

In view of the above, all or a selection of these processes can be employed to reduce the computational space within the data structure, regardless of what form of data structure is being employed. For instance, regardless if the data structure is a a decision tree, neural net, or other such data structure these processes can be used to normalize and batch the data as well as to tweak the data structure to limit the number of trees, limit the lengths and branching factors, as well as to cluster the inputs in a more meaningful way. In this manner, interpreting the data and identifying trends becomes easier, and along with this, making predictions about what should be occurring also becomes more precise.

Accordingly, in view of the above, and with reference to FIG. 9B, an exemplary process 900 for generating a signature is discussed. As discussed in more detail herein, a user's “signature” refers to a mapping of quantifiable parameters (e.g., determined relationships or correlations between and/or among various quantifiable, measurable parameters), which can be used to estimate or determine (e.g., indirectly measure) the presence/non-presence and/or concentration of a biomolecule of interest within the body of a particular wearer or user. A given signature is user-specific and can even be skin-location specific for a given user. Stated differently, the “signature” can assist in calibrating a device (e.g., biometric sensing and/or monitoring device 15) at the start of wear period or after movement of the device (e.g., to a different skin location). As certain estimating techniques can be sensitive to the starting state (e.g., the wearer's biological characteristics, the device being used, the skin location of the device), the signature can be or include a learned constellation of scalars and/or learned vector of values that can be determined by taking a series of one or more calibration readings at the time the device is situated on the wearer (e.g., initially situated, after the device has moved skin locations for any reason). The calibration reading from the device can be taken along with obtaining known measurement data (e.g., measurements from or by an invasive measurement system, such as a CGM or a finger prick glucose reading). The calibration reading and the known measurement data can then be processed via analytic, e.g., calibration, operations, such as those described herein, and the calibration operations can provide the signature, which can be or include a calibration constellation or vector. The calibration vector can be applied to subsequent readings from the device (e.g., while the device is located at the same skin location of the same user) to inform the various systems and/or methods described herein regarding user-specific context (e.g., user-specific information) to increase the accuracy of subsequent determinations (e.g., determinations as to the presence and/or concentration of one or more biomolecules of interest).

The disclosed technology can include calibrating a given device at a given skin location on a given user (e.g., generating a signature) using a single calibration reading. As explained elsewhere herein, a “reading” can include a “full sweep” for each light emitter, with each “full sweep” of a given light emitter including a plurality of “measurements” or induvial light absorption data points (e.g., data points measuring the skin's light absorption and reflection, e.g., permittivity, responses to a corresponding light emission pattern for the corresponding light emitter). Alternatively or in addition, the disclosed technology can include using multiple calibration readings to generate the signature. Stated differently, the disclosed technology can include using a sequence of calibration readings (e.g., taken over a predetermined time and/or at predetermined intervals) to establish reading patterns, which may increase the accuracy of subsequent determinations regarding the presence and/or concentration of the biomolecule(s) of interest.

Every person can have different, even slightly different, skin (and, accordingly, different skin characteristics), and every skin location on a given person can have different, even slightly different, absorption, reflection, and/or permittivity characteristics. These differences can be due to skin structure, vascularization, difference in analytes and metabolites for a given person or skin location, as non-limiting examples. Further, while various measures are taken during manufacturing and quality control to ensure each device conforms to standard operation metrics and performance, a given sensing and monitoring device can have slightly different light illumination and/or detection characteristics (e.g., due to component variation).

Therefore, to help obtain an accurate reading at some particular skin location on a given individual, a user-specific “signature” or calibration can be useful to determine one or more glucose measurements in view of the topological characteristics and/or internal characteristics of the skin location (e.g., correlate or correspond unknown spectral data obtained at the particular skin location with the known measured blood glucose values) and the performance of the specific detection device (e.g., biometric sensing and/or monitoring device 15). Thus, the process 900 can include generating a set of parameters that can be used to inform all other measurements for a specific wear period during which the skin location of the patch (e.g., patch 11) does not change, and the process 900 can include generating a mapping of those parameters, as discussed above, which can be referred to herein as a user's (or wearer's) “signature” or as the “signature parameters.”

The process 900 can include determining signature parameters (and mappings of those parameters) using a neural net architecture, such as employing an auto-estimator and/or autoencoder. The auto-estimator/encoder can be a neural net system trained to compress the high dimensional space of the sensor array (e.g., sensor array 20) into a comparatively small set of parameters that represents the essential semantic information of the detection device's (e.g., biometric sensing and/or monitoring device 15) sensor array (e.g., sensor array 20) output.

The process 900 can include receiving 902 spectral data (e.g., raw and/or preprocessed spectral data measured in, from, on, or near structure(s), tissue(s), and/or fluid(s) of the wearer, such as one or more absorbed and/or reflected waveforms measured in response to one or more outputted waveform being emitted and directed toward the corresponding structure(s), tissue(s), and/or fluid(s)), measured biomolecule data (also referenced herein as “known measurement data” or “measurement data”) (e.g., known or control or true biomolecule measurements, such as measurements from or by an invasive continuous glucose monitor (CGM) or a finger prick glucose reading), and/or spectral data that has been correlated with known levels of a biomolecule (“known spectral data”) (e.g., spectral data that has been correlated with “known measurement data” or “measurement data”).

The process 900 can include pre-processing 904 some or all of the spectral data (e.g., one, some, or all of the aforementioned types of data). For example, the pre-processing technique(s) can include applying the mean and standard deviation of the dataset, applying min-max scaling, applying cyclic transformation for time features, and/or applying mini-max scaling for true biomolecule data only.

Alternatively or in addition, certain data can be “augmented” (e.g., due to scarcity of that type of data and/or scarcity of that type of data within a certain range). For example, blood glucose data (and/or corresponding spectral data) can be scarce for a higher range of blood glucose values, and as such, the pre-processing technique(s) can include interpolation techniques to provide additional, estimated data. The interpolation technique(s) can include one or more of a spline, an Akima spline, a cubic spline, a linear interpolation, a pad interpolation, a forward fill, or a backward fill, as non-limiting examples.

Alternatively or in addition, the pre-processing 904 can include applying one or more filters to remove erroneous data. For example, a set of heuristics can be used to filter out erroneous spectral data. Alternatively or in addition, the one or more filters can include a stacked filter that includes a heuristic filter (e.g., as a non-limiting example, a checks min step, a max values step, and an intercept step), an unsupervised approach (HDBSCAN), and a fluctuation filter.

Alternatively or in addition, the pre-processing 904 can include a Data Valuation using Reinforcement Learning (DVRL) technique. DVRL can help address problems with quantifying data importance for specific tasks and can use reinforcement learning to determine the worth of each data point for a specific task. A data value estimator can analyze each data point and assign a value based on how much it helps a learning model improve. By receiving a reward when the model performs well, the estimator learns to highly value relevant data, which results in the DVRL prioritizing important data, improving learning efficiency, and even discovering unexpected results (e.g., valuable yet easily overlooked data or relationships).

Alternatively or in addition, the pre-processing 904 can include upsampling, which is a data pre-processing technique used in ML to address class imbalance and improve the performance of models, particularly in classification tasks. Class imbalance can occur when one class of data significantly outnumbers the other(s), which can lead to biased model predictions. Upsampling helps balance the dataset by increasing the representation of minority classes. This technique can be particularly helpful in training a model to correctly encounter scenarios in which minority classes are of high interest, such as predicting sudden, high rises in blood glucose levels, which can be otherwise difficult due to a scarcity of such data.

Alternatively or in addition, the pre-processing 904 can include sorting data (e.g., spectral data) by timestamps associated with each corresponding data point (e.g., timestamp metadata). The sorted data can be split into a plurality of data sets, such as a training data set, a validation data set, and/or a test data set. A more detailed discussion of this pre-processing 904 technique or process is provided immediately below:
1. Receive data (e.g., spectral data, known spectral data, known measurement data, and/or unknown measurement data, as non-limiting examples).
2. Sort the various data (e.g., by PatchID and/or data timestamp), and split the data into different “runs” or “segments,” which refer to a group of consecutive readings from a single wear session (readings from a device located at the same skin location on the same user without having been disturbed or moved) that occur between predefined bookend time gaps (e.g., within 2 hours, within 3 hours, within 4 hours, etc.) of the most recent reading. A time gap is the time between subsequent readings, and a bookend time gap refers to a time gap that is larger than a predetermined threshold (e.g., 2 hours, 3 hours, 4 hours, etc.). Thus, a bookend time gap can trigger the end of the pending run and the start of a new run. For example, a run can end when a time gap larger than the predetermined threshold occurs between subsequent readings. As a further example, if the predetermined threshold is 2 hours, and 2.5 hours occur between subsequent readings, the previously pending run is ended (with the reading occurring 2.5 hours ago being the final reading in the previous run), and a new run is started (with the current reading being the first reading in the new run)
3. Perform a function to determine variations in the dataset features (e.g., voltage data and/or current data corresponding to measured light absorption responses) to thereby identify any significant changes in light absorption response measurements over time.
4. Perform a rolling average operation with a window of 10 readings (e.g., to reduce or eliminate noise in the data).
5. Split the data into training, validation, and test sets (e.g., using “train test split” method from scikit-learn). A predetermined portion (e.g., 50%) of each run can be reserved for fitting a transformer (e.g., a spline transformer), such that this reserved subset can represent a comprehensive sample for spline fitting without over-relying on any single segment.
6. Transform selected features (e.g., using spline_transformer(n_knots=10, degree=3), which can apply cubic spline interpolation). This can add non-linear transformation to the input features and/or can capture trends and cyclical patterns within each run or segment.
7. Normalize the data (e.g., on a per run basis, where the data for every run is scaled with its own individual StandardScaler). The scaler can be fitted on train data to avoid data leakage and improve generalization. A separate scaler is created for each run's data.
8. Create two dictionaries: processed_dataset (which can store normalized data sets) and scalers (which can store scalers per run).

The model can be trained per run with a batch size of 1, which can thereby train the model in a stateful manner and/or can ensure that every run is of the same size (e.g., by padding smaller runs by values of −100, with the model being configured to ignore input values of −100).

The structure of the autoencoder includes an encoder component and a decoder component. The encoder component can be configured to evaluate the input data in various ways such that the information contained in the input is compressed into a small vector. For example, the process can include considering 906 the input array as a two-dimensional matrix. For example, each row can be a list of a certain number of values (e.g., twenty values). The final row can include the demographic data and the blood glucose value taken at that location at the time of the signature reading (e.g., during the calibration process). The rest of the row can be padded with zeros (e.g., can consist of zero values).

The reading(s) used for the signature generation (e.g., calibration) can be taken (e.g., using a device providing non-invasive, spectral readings, such as the various systems and devices described herein, e.g., test device producing test measurements) contemporaneously (e.g., at the same time or substantially the same time, such as within overlapping time frees, within the same time period, within a period of 1 hour, within a period of 30 minutes, within a period of 15 minutes, within a period of 10 minutes, within a period of 5 minutes) with a blood glucose reading from a glucose monitoring system, such as an invasive continuous glucose monitor (CGM) or a finger prick glucose reading (e.g., control measurements), as non-limiting examples.

The signature-generation or calibration process can produce one or more parameters (e.g., 10 parameters) that are input as features into the neural net (e.g., steps 906-914 discussed below). These parameters can be stored in memory and reused on all subsequent readings after the initial calibration or signature generation (e.g., until the device is removed or shifted from the specific skin location at which the device is located during the calibration process). The features can include various data, such as data relating to the wearer (or user or individual) and/or data relating to the data collection process (e.g., spectral data). For example, as non-limiting examples, the features can include demographic information (e.g., age, height, weight, BMI, gender, ethnicity), mean response values from one or more (e.g., each) emitter (e.g., LED), mean rate of change of responses from one or more (e.g., each) emitter, and/or coefficients of a polynomial fit (e.g., second degree, third degree) on the response values.

The neural net can be pre-trained to consider the calibration values at every point where a full measurement is made. Stated differently, the signature-generating process ensures the signature is usable for all possible values during normal use. A combination of the full measurement and the calibration parameters can be used as inputs to drive the neural net-based blood glucose estimate.

The process can include using one or more ANNs, such as a sequential set of convolutional neural network (CNN) layers (e.g., the autoencoder can be configured to use a sequential set of CNN layers). Layer 1 can be or include an array of a predetermined number of filters used to create a predetermined number of different versions of the input. Stated differently, the process 900 can include creating 908 a predetermined number of different versions of the input using an array of a predetermined number of filters. For example, Layer 1 can include 40 3×3 filters used to create 40 different variations of the input. Alternatively or in addition, Layer 2 can be configured to consolidate the various different versions from Layer 1 to a smaller number of variations. Stated differently, the process 900 can include consolidating 910 the different versions of Layer 1 into a comparatively smaller number of variations.

In neural networks, the transformation of features across layers is determined generally by the previous layer's outputs. Each layer can expand or reduce the number of features by adjusting the number of neurons, for example. This transformation is guided by the parameters (e.g., weights and biases) of the network, which can be optimized through gradient descent and backpropagation, as non-limiting examples. During training, an optimizer can adjust these parameters to minimize the loss function (e.g., as described in more detail herein), thereby finding or estimating the best configuration for accurate predictions.

As such, Layer 2 can be configured to consolidate the 40 variations of Layer 1 into 20 variations. Layer 3 can be or include a single matrix that is flattened, and a dropout layer can be applied and connected densely to the final semantic vector. Stated differently, the process 900 can include flattening 912 a single matrix comprising the data of Layer 2 to create a final semantic vector and connecting 914 the dropout layer to the final semantic vector. Flattening can refer to the process of converting a multi-dimensional array of neurons into a single vector.

This step can be important for transitioning from convolutional or pooling layers to fully connected layers in a neural network. By flattening, the data's dimensionality can be reduced, making it suitable for classification or output layers. Flattening can thusly facilitate the final stages of computation for accurate categorization or prediction. Moreover, dropout layers can randomly deactivate a subset of neurons during each training iteration, which can introduce noise into the system, thereby helping to prevent overfitting by reducing the risk of the model converging to local minima or maxima. By encouraging the system to rely on various paths, dropout layers can improve the generalization of the model. These layers can be used throughout the system, not just in the final semantic vector, to enhance the robustness and performance of the training process.

Alternatively or in addition, the process can include one or more CNNs and/or one or more different machine learning methodologies (e.g., LightGBM, Twin Networks, PCA-KNN, Partial Least Squares, LSTM, and/or Transformers). Alternatively or in addition, the process can include a CNN configuration that differs from the sequential set of layers described in the preceding paragraph.

To decode the final semantic vector, a network can be configured to perform an exactly reverse operation of the process 900, to therefore decode the semantic vector into the original input.

The process 900 can include applying 916 one or more loss functions, which for an autoencoder, generally refers to the reconstruction loss that compares the input values to the final output values. Sequential models can require a sequence of readings for training, and an additional metric for reducing overall validation loss during the training (e.g., feeding a small, unseen dataset fed while training to test on only). Alternatively or in addition, the reconstruction loss can be reduced or minimized (e.g., for a variational autoencoder (VAE)) to train.

In accordance with the disclosed technology, the loss function can include adding a multiplier of (2−person(input,output)), which can help ensure the input and output have match patterns. The loss function can include a multiplier of alpha*abs(BG value), where alpha is a high number (e.g., 100) to ensure that the blood glucose value is trained. The loss function can include Huber's equation, which uses both a MSE (mean squared error) and a MAE (mean absolute error) to compare all the inputs to the outputs. Alternatively or in addition, any other loss function can be used, such as cross entropy, mean squared error, mean absolute error, Kullback-Leibler divergence loss, quantile loss, log-cosh loss, regression, and/o hinge loss, as non-limiting example.

Other configurations and loss functions are contemplated. For example, for the LSTM Composite Autoencoder (which can include two decoder parts, namely a reconstructor and a predictor), it can be useful to apply a reconstruction loss function and a mean squared loss/error function for training. Alternatively or in addition, a custom-built calibration loss functions and a directional loss function can be applied to help make use of any available calibration data and the direction of the predicted blood glucose values, respectively. Other examples can include LightGBM, MLP, CNN, Transformer, PCA-KNN, Partial Least Squares, and Transformers, as non-limiting examples.

As additional, non-limiting examples, the loss function(s) can include a mean directional loss function or one or more custom loss functions. For example, a custom loss function can include one or both of Equations 1 and 2:

$$Loss = ( 2 - Pearson ) * α + α * MDA + α * Huber \qquad ( 1 )$$

$$Loss = ( 2 - Pearson ) * MDA * Huber * α \qquad ( 2 )$$

Once a signature has been generated for a particular user or wearer (and a particular skin location of a specific device on the particular wearer), the disclosed technology can include processes for detecting the presence and/or characteristics of one or more biomolecules within the wearer's body, the effects the presence/concentration of one or more biomolecules has on the wearer's body, and/or determining one or more remedial or prophylactic actions that can or should be taken in light of the presence/concentration of the biomolecule(s) and the corresponding effects.

On the other hand, and referring now to FIG. 10, when a device is moved from its position or skin location (e.g., by a user removing and later wearing the device again, by unintended shifting of the device to a different skin location even if only by a few centimeters), the spectral data can behave differently. To combat this, a new mapping can be necessary to the inform the model that there is a change in data behavior. This can be viewed as a subset of “Data Drift,” which can include creating a signature with a calibrating blood glucose, such that, as soon as the device is put on by the wearer, a calibration event is automatically triggered. The calibration event can include three stages: (1) the user initialized calibration of the device, (2) the signature model creates a mapping/signature, and (3) confirmation that the predictions at the calibration event and the next reading match the measured blood glucose values (e.g., the true blood glucose value).

To create the signature embedding and/or new signature embedding, the signature encoder can be configured to map a set of readings to a semantic vector, as described more fully herein. The signature embedding can be, include, or act as encoded environmental parameters (e.g., device behavior data, skin data, location data), which is encoded in such a way that the system can use the encoded data to understand the current environment and map incoming spectral data (and/or other data) to provide general knowledge about the current environment (e.g., the presence and/or concentration of a biomolecule of interest). The signature embedding is important at least because the skin properties at one skin location can be slightly different from the skin properties at another skin location, which can cause the skin to behave differently at those two locations. The signature embedding can extract and encode those differences for a given set of circumstances (e.g., skin location) such that mappings of the measured data (e.g., spectral data) can be normalized and used to estimate the presence and/or concentration of a biomolecule of interest at that skin location, for example.

FIG. 10's “feature” references can refer to the outputs of each signature embedding, which can be used to make estimates based on the received data or “readings” from the sensor(s) (e.g., sensor array 20).

Alternatively or in addition, the initialization of the calibration event can be automated, such as by detecting if there is an anomaly in the spectral data before transmitting the spectral data for training and testing. The term “state change” can refer to a spectral reading that was obtained when the device produced good readings after bad ones, which can refer to the occurrence of a calibration event, thereby giving unique mappings until the device was removed or the device/system detected a bad reading. The spectral data can be input into a Conditional Variational Autoencoder (CVAE) and HDBSCAN model to determine whether a calibration event is necessary and/or to provide a functional skin on/off detector. An example illustration of the signature process, including the skin on/off detector is shown in FIG. 10.

Referring to FIG. 11, the disclosed technology includes a process 1100 for determining the presence and/or concentration of one or more biomolecules within a wearer's body (e.g., based on spectral data obtained from a wearer of a device, such as a biometric sensing and/or monitoring device 15). The process 1100 can be configured to estimate the presence or concentration of a biomolecule of interest based on correspondences or correlations between or among (1) present unknown spectral data of a particular user and (2) present known or past known spectral data (e.g., “known spectral data,” which can refer to spectral data to be or previously correlated or corresponded to known measurement data) and/or past known measurement data (also referenced herein as “known biomolecule data”). Stated differently, the process 1400 can be configured to estimate the current presence and/or concentration of a biomolecule of interest for a given wearer without contemplating present known measurement data (e.g., known measurement data obtained contemporaneously with the present spectral data). In such an instance, this process 1100 can be performed after the model has been trained, and thus, by applying the model to the data structure, an estimation, e.g., prediction, about the concentration of the biomolecule of interest may be made, e.g., without needing to be compared to presently or past known concentration data. The process 1100 can include receiving 1102 spectral data. The spectral data can be or include spectral data measured in, from, on, or near structure(s), tissue(s), and/or fluid(s) of the wearer (e.g., one or more reflected waveforms measured in response to one or more outputted waveforms being emitted and directed toward the corresponding structure(s), tissue(s), and/or fluid(s)). Alternatively or in addition, the spectral data can be or include other data from which the spectral data can be derived and/or processed. As disclosed herein, the raw and/or pre-processed spectral data can be used to determine the presence of a biomolecule within the body of the wearer and/or a condition or health status of the wearer based at least in part on the presence, absence, and/or concentration of the biomolecule of interest (e.g., with respect to the amount and/or concentration of the biomolecule of interest).

The received spectral data can be or include spectral data that has been correlated with known levels of a biomolecule (e.g., “known spectral data”). For example, the term “known spectral data” can refer to spectral data that was obtained during, is derived from, or is otherwise associated with, the signature creation process 900. Alternatively or in addition, the spectral data can include data that was not obtained during, is not derived from, and/or is not otherwise associated with, the signature creation process 900, and thus, is unknown.

As disclosed herein, the signature generation process 900 can include correlating known glucose value data with received spectral data to produce the known spectral data or “signature,” which signature can be used as, or in conjunction with, a concentration model. Consequently, this data may then be used to populate a data structure, whereby unknown spectral data can be tested and/or determined.

The process 1100 can include generating 1104 a data structure that can include biomolecule data (e.g., known or true biomolecule measurements, such as measurements from or by an invasive continuous glucose monitor (CGM) or a finger prick glucose reading), user condition data (e.g., social data, medical records, exercise records, social media or other network connections, family history data, present environment data, psychological condition data, mental health data), and/or characteristic data (e.g., measured biological data regarding the wearer that is not spectral data or measured biomolecule data, such as heart rate, blood pressure, oxygen saturation, or the like when the biomolecule of interest is blood glucose). The data structure can include some or all of the received spectral data (e.g., from the measurements taken by the device with regard to the wearer). Generating 1104 the data structure can include creating nodes within the data structure for one, some, or all of the biomolecule data, the user condition data, the characteristic data, and/or the received spectral data. For example, the various data can form nodes within the data structure, such as a table, a tree, a graph, a neural net, and the like.

As a non-limiting illustrative example, generating 1104 the data structure can include comparing various data with one with another and determining correspondences or correlations between or among the various data (e.g., correspondences can be determined between two data types or values, collectively between various data types or values, and/or with respect to each individual factor). For example, the data structure can include multiple different databases, which at least some databases having a relational architecture and/or being configured to include one or more constructions (e.g., one or more table structures). Stated differently, the process 100 does not necessarily compare similarity between scalars and/or vectors (as a non-limiting examples); rather the process 100 can include determining a mapping of vectors that are similar to a common label, category, or classification (e.g., the variable that is being trained, such as the presence and/or concentration of a biomolecule of interest). In particular, many scalars and/or vectors (e.g., data inputs) that are similar may map to different labels, categories, or classifications. That is because the behavior of the skin can be context dependent, meaning the interpretation of a scalar or vector should take into account the state of the skin and the location of the detection device to be able to map the value correctly under that particular set of circumstances.

As a more specific example, the process 1100 can include generating and/or populating a data structure that includes a first table with at least some of the wearer's observed spectral data, at least some of one or more evaluations of, or determinations based on, the observed spectral data (e.g., evaluations or determinations made over time), and at least some of one or more biomolecules. The process 1100 can include generating and/or populating a second data structure, e.g., table, with at least some of the wearer's measured biomolecule data (e.g., biomolecule data that was collected or otherwise determined at the same time as the spectral data was collected, such as during the calibration process 900). The process 1100 can include generating and/or populating a third data structure, e.g., table, with at least some known biological characteristic data (e.g., data regarding the wearer that is not spectral data or measured biomolecule data) that was collected at the same time as the wearer's spectral data or measured biomolecule data. The process 1100 can include generating or populating one or more additional data structures, e.g., tables, with at least some of the spectral data, at least some of the measured biomolecule data, and/or at least some of the biological characteristic data for one or more individuals different from the wearer (e.g., individuals with at least one of the same or similar conditions or characteristics in response to the presence of the same or similar biomolecule with the same or similar values). The process 1100 can include comparing the various data structures, e.g., tables, and thereby deriving one or more relevant correlations therebetween, such as with regard to one or more of the spectral data, the measured biomolecule data, and/or the biological characteristic data. The process can include generating and/or using a key to correlate the tables.

The key can be subsequently used to provide information about the wearer, the determined presence or amount of biomolecule, or the like (e.g., in response to receiving a question, prompt, or command, e.g., via a system user interface). The key can be or include any common identifier, such as a name, a number, a nickname, a handle, a phone number, and the like, by which one or more of the data structures or tables may be accessed, correlated, and/or a calculation performed by use thereof. Hence, use of data structures, such as tables may be challenging, which may be made even more so by the time it takes for the system to search for, pull up the tables, retrieve the key, and search the tables so as to look up answers to questions, and as such other data structures other than tables may be useful and are contemplated.

Particularly, when implementing a table-based architecture, at Step 1104, generating a table data structure can include generating a relational database, such as a Structured Query Language (SQL) database (e.g., which may be implemented via a relational workflow and/or database management system, such as the WMS). In some instances, the SQL database can be a relational and/or table-based database, such as where one or more tables (e.g., look up tables (LUT)) form a structure configured to store data and facilitate searching, relationship determinations, and answering of queries using the data stored therein.

Accordingly, in particular embodiments, with respect to Steps 1104, 1105, 1106, 1108, and 1110, a table-based database may be generated, searched, and used to determine relationships from which answers to one or more queries may be determined.

For example, a table (e.g., a calibration table) can correlate (e.g., via the process 1100) the wearer's spectral data to one or more biomolecules suspected of being present within the tissues, one or more conditions suspected of being related to the presence of the biomolecule, and/or measured biomolecule data. The correlation can be useful for predicting the presence and/or concentration of the biomolecule of interest based on the spectral data and comparing the results of that prediction to the measured biomolecule data. Likewise, the table can be used to correlate the progress of the wearer, across time, towards improvement of one or more of their biomolecule values and/or conditions related thereto. However, as mentioned, because of the time typically needed to implement a table-based data structure, a further data architecture, can be used to structure a database of the system, such as a data tree structure, wherein various data elements can be stored in a compressed, but correlated fashion, and/or in a hash table. Accordingly, in various embodiments, the data structure can be or include a root-tree-branch database to determine the results for one or more of the queries, as discussed herein.

Alternatively or in addition, a knowledge graph and/or neural network-based architecture (e.g., comprising an artificial neural network architecture) can structure the database, so as to enhance the performance of computational analyses executed using the database.

Hence, generating 1104 the data structure can include applying one or more algorithms, discussed herein, whereby the algorithm can be configured to structure the infrastructure of a relational database to thereby enable more efficient and accurate searching (e.g., generate a searchable data structure for storing the collected and/or analyzed data in a relational architecture). For example, the disclosed technology can include performing one or more graph-based or neural network analyses and/or for performing table- or tree-based analyses. As discussed herein, the data structure, or data therefrom, can be transmitted to or via a suitably configured API (or data can be received into the data structure therefrom) to help facilitate simple and efficient data searching and/or data updates.

Alternatively or in addition, generating 1104 the data structure can include constructing an artificial neural network (ANN) to determine or identify correlations, correspondences, and/or associations between and/or among varying data points and/or data types, and the ANN can be constructed such that any particular data point can form a node.

The ANN (or other graph) can store processed or raw spectral data and known biomolecule data (e.g., data from a finger prick test strip or analyte-measured glucose data, also referenced herein as “known measurement data”). The spectral data (e.g., processed or cleaned, raw) can form a first set of nodes, and the known biomolecule data can form a second set of nodes.

The ANN can include a third set of nodes between the first set of nodes and the second set of nodes, and the third set of nodes, can be indicative of (or relate to, or otherwise be associated with) one or more conditions, one or more properties, one or more characteristics, one or more personality traits, one or more health factors, etc. of the wearer of the sensing and monitoring device. The disclosed technology can include inferring and/or determining relationships (e.g., correlations and/or correspondences) between the two perimeter node sets (e.g., the first set of nodes and the second set of nodes) to thereby determine how the first node is related to second node. Accordingly, the ANN can determine and/or establish relationships between these first and second sets of nodes. For example, when building the ANN (or other data structure, such as knowledge or nearest neighbor or graph, as non-limiting examples), the various user data (e.g., the wearer's observed spectral data, known/measured biomolecule data, biological characteristic data) can be received and inputted into the ANN. Some or all of the user data can be indicative of measurements collected at roughly the same time (e.g., spectral data collected at approximately the same time that the measured biomolecule data was collected). As described herein, the disclosed technology includes encrypting and/or receiving various user characteristic data (e.g., social data, medical records, exercise records, social media or other network connections, family history data, present environment data, psychological condition data, mental health data). A user profile (unique to the user) can be generated based on one, some, or all of the user data and/or can form one or more additional nodes each based at least in part on one, some, or all of the user data. And as will be discussed more fully herein, the disclosed technology can include determining and/or identifying relationships between and/or among the various nodes.

Consequently, a first correspondence between the presence and concentration of the biomolecule, the condition being experienced by the wearer, and the measured spectral data can be determined. Stated differently, the process 1100 can include automatically generating 1106 one or more models configured to determine correspondences between the nodes. For example, the process 1100 can include creating one or more models according to one or more model architectures; implementing one or more hyperparameter tuning methods (e.g., grid search, random search, Bayesian optimization), and/or using one or more AutoML tools, to automatically find the best hyperparameters for a given model; training the given model such as by calculating the loss and updating model weights using an optimization algorithm (e.g., Stochastic Gradient Descent, Adam); evaluating the given model's performance on a validation dataset after each epoch to monitor for overfitting and/or using metrics such as accuracy, precision, recall, and/or F1-score to asses model performance; and updating and/or retraining the model as necessary based on model performance. Regardless, and as explained herein, these resulting correspondences can be used to evaluate the subsequent spectral data (e.g., unknown spectral data) and determine the presence, concentration, and/or the effects of the biomolecule of interest.

The process 1100 can include receiving 1108 unknown spectral data and estimating or determining 1110 the presence, concentration, and/or effects of the biomolecule of interest by applying the unknown spectral data to the one or more models (e.g., the model(s) generated at step 1106). The process 1100 can include determining 1110 the presence, concentration, and/or effects of the biomolecule of interest by evaluating current unknown spectral data using the one or more models. Alternatively, the process 1100 can include determining 1110 the presence, concentration, and/or effects of the biomolecule of interest by evaluating the current unknown spectral data using the one or more models and one or more past readings (e.g., one or more instances of unknown data evaluated by the one or more models), as past readings can affect current readings under certain circumstances.

For example, the process 1100 can include evaluating the current unknown spectral data in conjunction with the previous 3 readings, the previous 5 readings, the previous 10 readings, the previous 20 readings, the previous 50 readings, the previous 100 readings, the previous 500 readings, the previous 1000 readings, or the like. Alternatively or in addition, the process 1100 can include evaluating the current unknown spectral data in conjunction with some or all readings obtained in a particular (e.g., predetermined) preceding timeframe (e.g., all readings within the preceding hour, all readings within the preceding 2 hours, all readings within the preceding 6 hours, all readings within the preceding 24 hours, all readings within the preceding 3 days). As will be appreciated by those having skill in the art, by applying the one or more models to subsequently received, unknown spectral data, the process 1100 enables a wearer to continuously and non-invasively monitor his or her presence, concentration, and/or effects of a given biomolecule of interest (e.g., blood glucose).

The process 1100 can include weighting 1112 correspondences between the nodes. These process 1100 can include strengthening pre-existing correlations between two or more nodes in the data structure. For example, the process 1100 can include strengthening or increasing in weight a given pre-existing correlation, providing a comparatively lower weight (e.g., with respect to the aforementioned, given pre-existing correlation) to one or more new non-correlations, and initially weighting (e.g., providing a weight that is lower than the given pre-existing correlation) any new correlations. Stated differently, the process can include more heavily weighting correlations that include data known to be accurate and/or more lightly weighting correlations that are new or otherwise unknown, such that the resulting algorithms rely more so on existing, known data and/or existing, known correlations between different data.

Alternatively or in addition, the disclosed technology can include determining how much each feature is contributing towards the predictions, and to do so, various methods can be used, such as a permutation importance technique (e.g., holding out one feature at a time to see differences in prediction), a Gradient-weighted Class Activation Mapping (GradCAM) technique, a SHAP (Shaply Values), a gain-based importance technique, and/or a split-based importance technique, as non-limiting examples.

One or more steps of the process 1100 can be performed or managed by a workflow manager system (WMS). For example, the WMS can be configured to generate 1104 the data structure, develop and/or generate 1108 the model(s), and/or determine 1112 the presence, concentration, and/or the effects of the biomolecule of interest. The WMS can be configured to receive and analyze the individual characteristic data, known biomolecule data (also referenced herein as “known measurement data”), received spectral data, and/or direct blood glucose data (e.g., one or more readings from a glucose monitoring system, such as an invasive continuous glucose monitor (CGM) or a finger prick glucose reading, as non-limiting examples), the wearer's signature, and/or additional wearer data, such as data indicative of biological characteristics and/or biomolecules that are different from the biomolecule of interest (e.g., sweat data, heart rate data, heart rate variability data, blood pressure data, oxygen saturation data, respiration rate data, sleep level data, and/or activity level data). The WMS can be configured to enter some or all such data into the data structure and observe or otherwise determine a correlation or correspondence between one or more nodes of the data structure.

The WMS can be configured to demarcate correspondences between two or more nodes as a relationship between the two or more nodes. By applying the various methods and processes described herein (e.g., process 900 and/or process 1100), it is not uncommon to identify relationships between nodes that would otherwise not be expected. Stated differently, the disclosed technology regularly identifies correspondences and relationships between different data values and/or types of data that would not be apparent or expected to one having ordinary skill in the art. Accordingly, the disclosed technology commonly provides unexpected results, particularly when compared to existing technologies. For example, spectral data obtained according to the systems, devices, and methods described herein (e.g., obtaining spectral data using the light emission patterns, light emission rates and/or timings, and/or light emission wavelengths described herein), while not necessarily directly measuring or directly indicating the presence or concentration of a given biomolecule, provides the basis for estimating or otherwise determining the presence or concentration of the given biomolecule, based on various correlations or correspondences between and among the various spectral data and/or other data as described herein.

For example, the disclosed technology can include correlating a wearer's personal characteristic data to one or more conditions the wearer may be experiencing, and the disclosed technology can include correlating data indicative of such a personal characteristic-condition correlation to spectral data (e.g., measurements taken in, on, or from the wearer) and/or known measurement data characterizing that biomolecule (e.g., with regard to its presence and concentration), to thereby determine a relationship between the presence and concentration of the biomolecule with a known condition of the individual. Accordingly, the disclosed technology includes inferring that the biomolecule of interest is known to be present at a problematic concentration and can thus be related to the onset and/or progression of the condition.

The WMS can be configured to perform a plurality of workflows related to a plurality of spectral measurements performed with respect to the same wearer or with respect to multiple different wearers of multiple different, corresponding devices. Accordingly, the disclosed technology includes analyzing spectral data for a single person (e.g., a single skin location for a single person, a plurality of particular skin locations for a single person, any skin location for a single person) or multiple people (e.g., a common and/or approximated skin location for multiple people, a plurality of particular approximated skin locations for multiple people, any skin location for multiple people) and/or related to a single biomolecule or a plurality of biomolecules, and with respect to a single condition or a plurality of conditions. The data can be or include a single measurement or a plurality of measurements (e.g., multiple measurements performed over a prolong period of time for the single wearer or for a plurality of wearers).

As can be seen with respect to FIG. 12, the disclosed technology includes a process 1200 for querying the data structure(s) and returning a result or answer based on information and/or correspondences stored in the data structure(s) (e.g., using one or more artificial intelligence (AI) algorithms). Implementing AI technologies in this manner and in this context can be useful at least because it can provide a more comprehensive analysis on generated and/or collected data and/or results based at least in part on such an analysis.

For example, the process 1200 can include training 1202 the AI algorithms using one or more machine learning (ML) protocols. That is to say, the process 1200 can include implementing one or more ML protocols on stored data attained (e.g., biomolecule data, the user condition data, the characteristic data, and/or the received spectral data) and/or any known correlations or correspondences known with respect to any such data, to thereby teach or train the AI algorithms to identify one or more correlations (e.g., between the various spectral data and known measurement data that is generated or otherwise collected or received, between spectral data of the same wearer or another individual at the same or different times, and/or with regard to the same or different biomolecules). The AI algorithms can include one or more models, such as a Vector Quantized Variational Autoencoder (VQVAE).

Because the VQVAE is a complex model, however, it can require a large number of trainable parameters, which can be difficult when data is scarce, such as when initially collecting data. As such, other models can be used, such as Light Gradient Boosting Model (LightGBM), which can require fewer features as compared to many other neural net-based models). LightGBM is a tree-based model that can train using a gradient boosting algorithm and can provide various features, such as mean diode output for each LED (e.g., emitter), mean rate of change of diode output for each LED, square/cube of mean diode output for each LED, square/cube of mean rate of change of diode output for each LED, change in diode prediction by a best line of linear/polynomial fit (e.g., a degree of 2, 3, 4, or 5), and/or time offset. Alternatively or in addition, other models can be used, such as a LSTM Composite Autoencoder (e.g., a sequence-based model) or a combined version of a sequence-based model (e.g., LSTM Composite Autoencoder) and LightGBM as a final layer, which can enable the use of gradient boosting for making predictions.

A high-level diagram of an example LSTM Composite Autoencoder is illustrated in FIG. 13A. As illustrated, the example LSTM Composite Autoencoder can be configured to receive user identification information, which can be used to find an associated “ID embedding” or user-specific signature, as described herein. Alternatively or in addition, the example LSTM Composite Autoencoder can be configured to receive location information (e.g., a skin location at which a patch 11 and/or biometric sensing and/or monitoring device 15 is located), which can be used to find an associated “location embedding” (e.g., the signature is associated with a particular skin location). The LSTM Composite Autoencoder can be configured to combine two different concepts to provide a machine learning method that can estimate the presence and/or concentration of a biomolecule of interest while also being able to use sequences of information to increase the probability of making a match with regard to the estimated presence and/or concentration of the biomolecule of interest. To that end, LSTMs generally refer to a type of recurrent neural network in which the output of the neural network is fed back into the neural networks as an input, and Autoencoders generally refer to a structure configured to train the behavior of the encoding process to map a vector space with specific properties.

Here, and as illustrated in FIG. 13A, the LSTM Composite Autoencoder can be configured to receive inputs including spectral data obtained from the particular user (e.g., “sequence input”) associated with the user-specific signature (e.g., “ID embedding”) and/or at the skin location indicated by the location information (e.g., “location embedding”). Some or all of such data can be received by the autoencoder and/or, which can provide a map of the vector space. Stated differently, the shared latent space can refer to the possible values that a semantic vector might take, and the decoder, which can be or include the trained embedding, can be the latent space. Once the autoencoder is trained, the decoder can create a semantic vector with trained properties (e.g., “reconstructed data”). The mapping (or “reconstructed data”) can be used by the LTSM (e.g., “predictor”) to find relationships (e.g., correlations or correspondences) between different domains and enable transformations between the different domains; that is to say, the LTSM can apply the mappings from the Autoencoder to incoming data to thereby estimate a presence and/or concentration of a biomolecule of interest, such as blood glucose.

Stated differently, the Composite Autoencoder can include an encoder, a decoder configured to reconstruct inputs, and/or a predictor configured to predict a single value for the given input. As a non-limiting example, the predicted single value is the estimated blood glucose level or concentration. The model can include several LSTM layers and a Multihead Attention Layer that can help in learning a pattern when data (e.g., spectral data) is received by the model.

FIG. 13B illustrates the structure of an exemplary model with the input and output shapes for various layers. As shown, the LSTM model can be or include a stacked LSTM model, which can be designed to evaluate sequential data and/or can have an architecture tailored to predict a single continuous output based on time-series inputs. For example, the output can be blood glucose concentrations (or concentrations of any other biomolecule of interest). Known measurements of the value and concentration of the biomolecule of interest (e.g., via a test device known to detect the actual presence and return the actual concentration value for the biomolecule of interest, such as from a prick and stick or analyte sensing measuring device) can be used as the target variables to help the LSTM model learn patterns of light absorption responses associated with the biomolecule concentration changes. A masking layer can be applied to manage missing data points within sequences. For example, any input values set to −100 can be ignored by the model, preventing those values from influencing the model's learning and allowing the model to handle variable-length sequences within batches. The LSTM layer can include 128 units with a hyperbolic tangent (tanh) activation function, and the units can be set to return sequences. These layers can be configured as stateful, meaning the model can retain its internal state between batches, making the model suitable for capturing long-term dependencies across sequences in a consistent manner. A dropout layer (e.g., with a rate of 0.2) can be added (e.g., to reduce the risk of overfitting). The final layer can be or include a dense layer with a single output unit, which can provide a continuous output value. The LSTM model can use an ordinary MSE loss function, as a non-limiting example.

Accordingly, the LSTM model can thus predict blood glucose levels (or the concentration of any other biomolecule of interest). Accordingly, the stacked LSTM model can be configured to incorporate features include, but not limited to, mean response values corresponding the light emitted from each light emitter, mean rate of change of response values corresponding the light emitted from each light emitter, and/or spline difference features. The stacked LSTM model can capture long-term dependencies, such as by maintaining the hidden state across runs, meaning it can carry over learned information from one run to the next, which can be particularly useful in time-series data where patterns can span long sequences. The statefulness of the stacked LSTMs can capture these dependences more effectively than a standard LSTM (which typically resets the state after each batch or run). Alternatively or in addition, the stacked LSTM does not “forget” the sequence context when moving from one batch or run to the next, and this continuity can be valuable when the entire sequence cannot fit into a single batch, meaning the model is better equipped to understand where it left off in the sequence, which is particularly beneficial for sequential predictions, such as blood glucose concentrations or other biomolecule concentrations where information from previous steps can influence future steps.

Returning to FIG. 12, the process 1200 can include configuring the AI algorithms to receive a plurality of inputs (e.g., spectral read data from a device, known measurement data collected at the same time the reads have been performed such as from a blood glucose monitor) and building and/or structuring a database for a specific wearer based on the plurality of inputs. The process 1200 can, alternatively or in addition, include configuring the AI algorithms to collect, receive, and/or input health and/or characteristic data relating to the wearer into the data structure.

The process 1200 can include receiving 1204 one or more known measurements of the value and concentration of the biomolecule of interest (e.g., via a test device known to detect the actual presence and return the actual concentration value for the biomolecule of interest, such as from a prick and stick or analyte sensing measuring device). The process 1200 can include receiving 1206 wearer data (e.g., characteristic data of the wearer, one or more of conditions of the wearer, and/or characteristics of the biomolecule of interest, such as from the biometric sensing and/or monitoring device 15).

The process 1200 can include cleaning 1208 the one or more known measurements of the value and concentration of the biomolecule of interest and/or the wearer data. As will be appreciated, the process 1200 may not perform 100% correctly all the time. To address this, the process 100 can include various heuristics and/or machine learning methods that are trained to detect bad records, and these bad records can be removed from the training data sets and/or the final results. For example, cleaning 1208 can involve applying human-derived heuristics to the data, such as by applying one or more statistical methods to one or more pluralities of measurements, one or more full sweeps, and/or one or more readings to determine patterns that represent “bad behavior,” which can refer to inconsistent measurements, full sweeps, or readings that represent some error or hardware failure. Once the bad behavior has been identified, one or more cleaning algorithms or programs can be created to automatically identify and/or eliminate subsequent instances of the bad behavior. The cleaning algorithms can thus be used on all future data, including, but not limited to, training data, validation data, test data, and/or field data.

The process 1200 can include labelling and/or categorizing 1210 the one or more known measurements of the value and concentration of the biomolecule of interest and/or wearer data (e.g., with respect to one or more classifications, such as in relation to one or more states or conditions in relation to the presence of one or more biomolecules). “Labelling” can refer generally to the estimate concentration of the biomolecule of interest. The input spectral data values can be aligned with the label, which is derived from another process. Categorizing information can be added, such as activity data for the user corresponding to the label and/or food consumption of the user at a time that may impact the label.

The process 1200 can include structuring 1212 the database, such as by implementing a skimmer (e.g., for providing a relational structure to the database), and populating 1214 the database with data (e.g., one or more known measurements of the value and concentration of the biomolecule of interest and/or wearer data) in accordance with determined or inferred relationships.

The process 1200 can include applying 1216 a ML protocol to determine relationships between data points entered into the database. Such relationships can be determined based on known facts, and as such, the AI learning can be considered “supervised learning” (e.g., where the data, such as spectral and/or biological response data, is entered into the database and categorized in accordance with one or more categories and/or labels). The process 1200 can include generating 1218 a key based on the known factors by which the correspondence between the two data sets can be determined. In this regard, known factors, e.g., functioning as a key, can be used to label, categorize, and store the newly defined known read data, and the key can subsequently be used to inform a given search query, so as to make the return of results faster than certain other methods.

Alternatively or in addition, as discussed in detail herein above, the learning can be inferred, and as such, the AI learning can be considered “unsupervised learning.” For example, the data to be stored and structured, or the result of processing thereof may not be known, relationships between the data may not have been determined, and/or the query to be answered may not yet be received or otherwise derived. In such instances, the data to be stored is “unsupervised,” and any patterns in the data to be stored and their relationships, such as commonalities or correspondences between data points, remain to be determined. In a first instance, it is the discovery of one or more of these patterns to which the devices, systems, and their methods of use are focused. And, in a second instance, once such patterns are determined, the disclosed technology can then use such patterns (e.g., commonalities or correspondences) in forming the architecture of the data structure, which data structure may then be processed to apply the one or more patterns to the received measurement data so as to correlate and/or correspond that data to a present analyte concentration of the individual wearing the multi-sensing detection device. Consequently, a first step in pattern discovery, may be the manner by which those measurements are produced in the first place.

For instance, as discussed herein above, in various embodiments, in determining the presence and/or concentration of an analyte within the skin and tissue, the analyte itself need not be directly detected. Rather, its presence and concentration can be inferred from its effects within the tissue. Therefore, in part, the dual sensing detection device may be configured for generating the conditions that produce these effects, e.g., permittivity effects, within the tissue, such as by generating and directing electromagnetic radiation that impinges within the skin and tissues, which then respond thereto by producing the permittivity effects herein disclosed.

Particularly, as discussed above, presented herein are multi-sensing detection devices having a number of electromagnetic radiation emitters that are configured for directing light (and/or RF or Microwaves) through the skin and into the tissues in a number of different patterns of illumination, including varying wavelengths, frequencies, amplitudes, energy levels, intensities, luminosities, durations, and the overall emission schema so as to produce and explore these aforementioned permittivity effects. The production of these permittivity effects is useful because the system is trying to generate a model to determine how much the light is being affected by the presence of the analyte within the tissue in the current context, so as to use that context and those resultant effects to map these permittivity properties to the concentration of the analyte, so as to determine the concentration of that analyte.

By varying various of the properties of the waveforms being emitted and directed into the tissue, the energy levels of the light can be manipulated. Further, by manipulating the energy levels being delivered to the tissue, these permittivity properties may be altered, but in a manner that is mediated by the concentration of analytes, e.g., glucose, being present within the skin and tissues. For example, by increasing the amplitude, e.g., luminosity, of the waveform more energy can be delivered into the tissue, without necessarily changing the frequency of the light wave. Thus, by changing the pattern of energy response in a manner that is characteristic of the analyte concentration, these properties affecting the response of the tissue and skin can be determined and be correlated to that analyte concentration. Therefore, measuring the different permittivity responses within the tissue provides concentration dependent information about the analyte and how it is affecting the tissue because those permittivity properties are altered differently depending on the level of that analyte within the tissue.

More particularly, as explained with reference to FIG. 9B above, these permittivity effects can be used to produce one or more fingerprints that can in turn be used to generate a signature as to how the tissue is responding to light in the presence of the analyte, and thus, the fingerprint(s) and/or signature can be used not only as a calibration, but also to estimate, e.g., predict, the level of the analyte within the tissue. In essence, the emittance of the electromagnetic radiation into the skin and tissues, in accordance with a determined pattern of emittance, provokes the aforementioned corresponding permittivity effects, each of which can produce a unique pattern of response that can be characterized much like a fingerprint.

Collectively these fingerprints, e.g., patterns of absorbance, refraction, scattering, polarization, and reflectance responses can be used to generate a unique signature that can then be equated with the concentration of an analyte of interest. Hence, these permittivity properties form one or more patterns that can be measured whereby the pattern is unique to the state of the skin and tissue in the presence of an analyte, such as glucose, at the times the measurements are taken. Thus, the pattern of these permittivity effects form fingerprints that one or more of, e.g., collectively, form a signature from which the concentration of the analyte, e.g., glucose, can be derived. However, as set forth with reference to FIGS. 9B and 12, in various instances, the data being produced, e.g., raw measurement data, for a number of reasons, may include errors within it. Therefore, it is useful to clean the data prior to its transmission to that analytics system and/or prior to evaluation.

Specifically, a problem in AI, e.g., machine learning, is when a good amount of data is produced, but the data has problems with it. There can be many problems that occur with the data. One is that there can be a lot of noise. Noise can come from a variety of different places, such as from the device itself, e.g., where the device actually produces noise caused by the jostling around of the device, for instance, as at set up. Such jostling, e.g., via device placement and other such mechanical-based problems, can produce noise, which noise can then produce records that are invalid and will have to be cleaned and/or corrected or expunged. The calibration operations of FIG. 9B is one attempt, in part, to correct for such noise.

Particularly, errors can occur from a number of different sources. For example, upon first coupling the detection device to the skin of the wearer, there are a number of mechanical errors that can initially occur due to physical adjustments that happen because of movements of the device, moisture of the skin, erratic body movements, and highly variable readings that transpire until the device is calibrated to the skin. The calibration process of FIG. 9B, in part, is an attempt to correct for such initial errors caused by these instances. Nevertheless, because of this, upon placement on the skin and the commencement of measurements, the first set of measurements to be collected can be discarded, so as to discard the first tens, hundreds, or even thousands of records, such as to discard the first thousand records, which are overly prone to a multiplicity of errors. Thus, a warmup process is useful for minimizing errors that may result from initial device placement.

In various instances, errors may further be derived due to drift over time. For instance, as indicated, when the device is first placed on the skin, the skin has a variety of properties that can change over time. In such instances, the referenced warmup period above, may be necessary such as where the initial measurements may start higher but drift downward until a point of stability is reached as the device warms up to the skin, the difference in temperatures equilibrates, and any moisture at the interface evaporates. This is one reason for deleting the first “n” number of records, e.g., n=1000 records, from the initial onset of readings, due to the drift that occurs from the device equilibrating to the skin.

Drift can also be caused by changes in the skin properties themselves over time, and in such instances, as indicated, the initial records can simply be discarded as the device warms up to the skin. But if drift happens later in the process, e.g., session drift, then the machine learning of the analytics system can be configured to also correct for this type of drift. Such session drift can simply result from the external and/or internal environments of the wearer, such as where the wearer's psychology or physiology affects the body, which then affects the measurements, such as where the wearer is cold and shivering, hot and sweating, stressed out, angry, and the like. Thus, the user's sympathetic and a parasympathetic nervous response can affect drift and thereby affect the measurement data. Hence, due to the possibility of the skin changing relative to the psychology and physiology of the wearer, the machine learning module may be configured to be able to recognize this sort of session drift and account for the localized changes that occur. For instance, as noted, during such periods, the skin can become dehydrated or super hydrated, and this may cause the measurements to drift, but in such instances, the inclusion of contextual data derived form a galvanic skin sensor can be used to determine these skin conditions and in response thereto correct for them. Temperature data can also help in this regard.

Further, drift can happen when the detection device is removed from and then later repositioned on the skin. During the period where the device is removed, the skin is changing, the vessels change, the blood pressure and flow changes, and the interstitial milieu changes. Thus, when the device is removed and then replaced, the skin composition can be very different. And no matter where the device is then repositioned, e.g., even if it is put on the exact same spot, it will still be different, there will be subtle differences. This is problematic because the current machine learning derived measurements will not exactly match the past measurements, and thus, the measurements will have drifted and will need to be corrected for because the subsequent values will not necessarily match the original values.

Therefore, the calibration processes of FIG. 9B, in part, may be implemented in order to correct for these types of issues with drift. Specifically, one such manner to correct for these types of drift is to perform a preliminary calibration set of measurements so as to generate an initial collection of permittivity fingerprints or an initial calibration signature that can then be used to determine instances where suspected interference, drift, and/or loss are occurring. In such instances, once the device is applied to the body, and an initial calibration reading is taken, then if nothing else substantially changes, it would be expected that subsequent readings to be within a range of standard deviations from the calibration reading.

However, where subsequent readings lie beyond an expected range of variance, e.g., there is an offset in the measurements, then drift may have occurred. In such an instance, the current readings can be compared to the calibration readings, and if it is determined that drift has indeed occurred, then a series of further reads may be taken to determine the reason and correct for the offset, so as to bring the measurements back within the calibration range, if possible. In one such effort to correct for this drift, when taking the subsequent readings that appear to be offset, the results of those readings can be subtracted from the calibration readings so as to determine the offset, and then the offset can either be corrected for by changing the emittance configuration, by changing the parameterization, and/or by subtracting the offset values from subsequent read values. Hence, with respect to calibration, the offset can first be determined, and then a subsequent mapping operation, e.g., machine learning protocol, can be employed to re-map the values in this subsequent space to the values in the original space where it is expected those subsequent values should have been. Another form of drift can occur due to the mechanics of the detection device itself, such as where the emitters do not always function in the same manner, such as where intensity and/or luminosity of the emitter, e.g., LED, may degrade over time, usage, and due to heat buildup, and thus, the device itself may be calibrated and re-calibrated over time.

In view of the above, drift can occur in response to several factors, such as calibration drift, sensor drift, environment drift, skin drift, and the like, which drift causes errors in measurements that will then need to be discarded or corrected for. The problem with discarding the data is that not all the records of a given run may have errors, and thus, by discarding such data, the system may be expunging records, which are costly to generate, which include useful information. Thus, in certain instances, it is better to correct for such problems, if possible, rather than merely excising the records. Accordingly, to correct for these issues, one such methodology is to apply a re-calibration and/or classification algorithm to the measurement data to correct for domain specific drift issues so as to better adapt the detection device to the ever-evolving character of the skin and tissue composition. Additionally, the machine learning module may also be configured to implement a number of domain adoption mechanisms so as to separate the semantics from the varying skin properties of the measurement domain, so as to thereby allow the measurements to more closely track and follow the analyte, e.g., glucose, concentrations over time.

Regardless of its source, when noise occurs it causes the signal data to vary widely, such as based on fluctuations of the relative strength of the signal. However, while smaller variations in data can more easily be corrected for, such as with regard to the calibration processes of FIG. 9B, when building and implementing a data structure, such as an artificial neural network, such data structures do not like large variations in the data. Consequently, as set forth with respect to FIG. 12, it is useful to correct and/or smooth out the generated data to minimize the variations of that data, such as across all the inputs, prior to (or in conjunction with) the building of the data structure.

Therefore, in accordance with Step 1208 of FIG. 12, to account for such noise, a series of pre-processing operations can be implemented to account and/or correct for the various variables that result from noise. For instance, normalization and/or other processing can be initiated so as to reshape the collected data, e.g., into a more desired pattern and/or shape. Particularly, a number, or all, of the fields can be normalized, such as in a number of different ways. One form of a standard statistical normalization function that can be employed includes multiplying the collected measurement values by the mean and then dividing by their standard deviation. This normalization function can be applied to the data to smooth out the variance, identify and correct (or delete) outliers, and, therefore, bring all the data to within a more standard range that can then more easily be processed.

Further, at Steps 1206 and 1208, in addition to the measurement data, a variety of other associated data may be included so as to add more context to the measurement data, including demographic information about the device user, their height, weight, activity levels, and other such demographics, which data can also be normalized. Once normalized, the analytics system may correct for errors. Hence, correcting for such errors is useful because it preserves data that may be important, rather than simply deleting records that may not be fully corrupted but merely include a portion of data that includes an error.

Further, in accordance with Steps 1402 and 1404 of FIG. 14, below, a further set of processes can be employed to correct for or remove problematic data, e.g., errors, in the data prior to further processing. For instance, the result of an error occurring can be device related, as described above, but is often that a given measurement, or other data point, is out of bounds as compared to other data being measured, and thus, outlier measurements occur. In such an instance, it may be difficult to determine if such an outlier is a true read or if it is the result of a read error. One or more preprocessing steps, therefore, can be performed so as to better bring the collected measurement data within a standard range, which will then make it easier to make a determination if an error occurred, and if so, to correct for it.

Consequently, upon Step 1402, it may be useful to initially determine if the data being collected and to be fit within one or more of the data structures discussed herein contains one or more errors. In one such manner, a first set of numbers of different records, such as 2, 5, 6, 8, 10, 20, 50, 100, 500, 1000 or more previous records may be identified, and their average can be calculated. Then a second set of numbers of records, such as, 5, 6, 8, 10, 20, 50, 100, 500, 1000 or more subsequent records may be identified, and their average can also be calculated. These averages can be compared, and if they are relatively within the same range, the measurements are likely being accurately measured.

However, if the measurements are not relatively within the same range, then this may be an indication of an error in the measurements having occurred, which in turn is indicative that one or more measurements within the record is likely to be inaccurate, and a correction may need to be made. When an error in one or more measurements is predicted, then the system can run a diagnostic to determine if there is a mechanical, sensor, firmware, software error, or an error in the implementation of an AI protocol. If an error is determined, then it may be corrected for, such as through one or more mechanical adjustments, reboots, or cleaning processes, so as to clean up the data, but in some instances, the erroneous data may simply be deleted.

One such method for cleaning up a potential error in interpreting the measurement data is to perform interpolation operation. For example, an interpolation technique may be employed so as to correct for measurement errors. For instance, in an exemplary run, one measurement may be captured, then a second, and a third, and in many instances, the measurements will fall within the same general range as one another, which may be indicative of the measurement process functioning appropriately. But, in some instances, one of the intermediary measurements may be off and out of range by quite a bit. In such an instance, an error may have occurred during the instance when the outlying measurement was taken, and the read or record may need to be discarded or corrected. However, as the record may still include useful information, it may be more beneficial to correct for the error rather than to discard the measurement and/or the entire record. Hence, to correct for the error in the measurement, an interpolation procedure may be implemented, such as where the erroneous measurement is discarded, and a new interpolated value is substituted in its pace.

In this regard, the interpolated value may be a value that is equivalent to the mean, e.g., halfway, between the measurements before and after the error read, or it may be an average between the two bookend measurements, or some other statistical variation thereof. This is a simple interpolation technique, but in other instances, interpolation can be much more complicated including several more involved steps that are interpolated together, such as where one or a multiplicity of errors occur together. In such an instance, the measurements can be graphed, a pattern or shape can be recognized therein, and then the interpolated value to be substituted for the erroneous measurement(s) can be the next value that continues or completes the shape and/or pattern. The process here is to insert values that replace, e.g., interpolate, other values that are recognized to be incorrect in some way.

Interpolation, therefore, is a technique whereby the system makes its best prediction as to what the range of the correct data should be. In one such instance, where given data appears to be erroneous, such as its values are significantly higher or lower than the other data is to simply use an intermediate between two surrounding data points that appear to be correctly on point. However, in certain instances, it may not be clear as to which values need to be corrected for, and thus, the system may involve performing a series of analyses to determine which factors need to be corrected.

In various embodiments, a particular form of interpolation may be employed, such as linear interpolation, where the pattern being formed appears to be a line, and thus, when determining what the missing value should be, the equation for a line may be employed so as to determine what the interpolated value should be. Hence, when you have two measurements that are offset from one another that would otherwise from a Euclidean straight line except for an outlying intermediate measurement, then the value to be substituted between these two points, e.g., the interpolated value, may be the point along the line that's halfway between the two points that form the line. Since this is in two-dimensional space, basically, the two sets of coordinates can be added up then be divided by two, so as to render the interpolated point. Hence, if an intermediary measurement is a far outlier, then linear interpolation can be used to average the two bounding points and thereby derive the interpolated substitution value.

Pad interpolation can also be used, such as where instead of linearly interpolating the intermediate value to be substituted for the measurement error, the value preceding the erroneous measurement can just be repeated and substituted for the error, and where there are many errors in a row, that preceding value can simply be repeated for each error. In various instances, both linear and pad interpolation can be employed together, such as where the first interpolation value is the mean between two bookend values, but then the mean value is repeated a number of times for each error in a sequence of errors. Another manner by which errors can be corrected for is spine spline interpolation or fitting. This form of interpolation occurs where the bounding points together all form a shape, like a curve, but there is an intermediate outlier in a measurement that does not fit into the shape. In such an instance, a spline may be employed.

In spline fitting, a set of secondary points may be generated and may be applied to the known and accurate measurements, whereby the control points mathematically determine the shape of the curve, e.g., they define the spline. But, where the erroneous data points occur, new values that fit within the spline can then be substituted for the erroneous data so as to complete the shape or pattern. In this instance, the spline is basically additional higher order parameters that provide nonlinear shape to the interpolation. So, in that regard, the spline would be the control point that goes along with the other points to give more shape, character, or description to how the measurements are all related together.

A more complex spline, Akama spline, may be used whereby a cubic interpolation is generated so as to create smooth curves that minimizes what would otherwise be unwanted oscillations, which sometimes occurs in relatively long splines. It's a particular spline technique whereby various segments of the pattern form a polynomial, and the cubic spline uses the polynomial to fit the measurements to the mathematical shape. In essence, the control points of the cubic spline make the pattern of a given number of segments. smoother by slightly modifying the measurements in view of the control points.

Nevertheless, once corrected, through a pre-processing methodology, then the AI platform may begin to perform its routine calculation of the neural network or matrix, such as in a series of runs, as discussed herein. Pre-processing can take place before the data leaves the device, or upon receipt and production of the data structure. For example, there are several different other sensor units, e.g., the galvanic skin response sensor mentioned above, that are included in the multi-sensing detection device, which sensors are continually collecting data, e.g., auxiliary data. This auxiliary data is subject to disruptions due to movements, temperature, moisture, and the like, which factors can be measured by one sensor unit but may be disruptive to a different sensor. Thus, in various embodiments, the detection device may include programming, e.g., firmware, for determining when one or more of the sensor units are being affected by outside influences, e.g., jostling, or by mechanical issues, such as with the emitters themselves, such as becoming too hot. Thus, problems with the detection device itself may produce errors in readings, which the system can detect, determine, and flag. Likewise, there may be issues with the reads themselves, whereby the system may then identify and perform error correction to correct for them, as set forth herein. In such instances, it may simply be useful to take more readings within a shorter period of time and/or including a flag for readings that are questionable.

Additionally, in various embodiments, prior to or after calculating the data structure, e.g., table, matrix, decision tree, and/or neural network, the collected measurement values can be normalized. In other words, one or both of the data represented by scalars and vectors can be normalized. Such normalization can be effectuated along a number of different dimensions. For example, a typical neural network includes several inputs, e.g., measurements, wherein a single measurement may be represented as a scalar, but a number of measurements when combined together can form a scalar. In various embodiments, each of the scalars can be normalized, and thereby, the vector itself can be normalized. In this regard, a normalization function can be applied to the measurements to transform all of the inputs into a standard normal form.

Specifically, a normalization function can be applied to the variations of the captured measurements so as to scale the data to a common range, e.g., 0 to 1, so as to prevent measurements with large values from dominating the training process, especially where those values may be outliers, and thus, subject to errors. By normalizing these values, the data point with the highest value can be expressed as a normalized value of 1, the lowest value can be expressed as a normalized value of 0, and all the intermediate values between 0 to 1 may be expressed as a decimal point. Such normalization, or feature scaling, is useful to ensure that each individual measurement will contribute more equally to the model training.

There are several different methods by which to perform a normalization to rescale the measurement values such as explained above with respect to taking the Min and Max values of the range to a [0,1] interval. In this regard, to find the range of the data set the measurement values can be arranged from smallest to largest and then the minimum value can be subtracted from the maximum value so as to derive the range. Then to normalize each of the measurements, the minimal value can be subtracted, iteratively, from each data point, and then the difference can be divided by the range so as to derive the normalized value. This operation may then be repeated for every measurement value within the matrix so by the end all of the values have been normalized.

In these instances, a wide range of statistical analyses can be performed on the collected measurement data and be used in these normalization and other operations, such as where the analysis can include determining the mean, median, mode, and standard differentiation. For instance, it is expected that as the measurements are collected, they will fall within a distribution range that can be graphed, or made to graph, as a curve, such as bell curve of a standard normal distribution. Such a curve will allow the system to calculate the mean and median of the measurements, and from them calculating the standard deviation.

From these calculations a number of probabilities founded on the data and based on the standard deviation may be determined, and thus, the curve may be referred to as a probability curve. The middle part of the probability curve is the mean, and where the curve forms a normal, e.g., bell curve, distribution the mean should be 0 and the standard deviation may be 1. But, then there are other points that will fall away to the edges, which represent one or more standard deviations away from the mean, such as one, two, or three standard deviations away from the mean.

In this regard, the standard deviation is an expression of how spread out the set of data is from the mean, where a low standard deviation indicates the measurements (or other datapoints) are more consistent and clustered more closely around the mean, while a higher standard deviation indicates the measurements are more inconsistent and spread out over a wider range. Hence, the standard deviation provides a quantitative manner by which the degree to which the data varies can be assessed, and further helps determine which values are true measurements, and which values, e.g., outliers, could be erroneous. The standard deviation can be calculated by taking the square root of the variance, such as by first identifying the mean of the data set, subtracting that mean from each datapoint and squaring the result. Finding the average of these squared differences will return the variance, and the square root of this variance is the standard deviation.

Accordingly, in generating a representation of the measurements in a graphical form, the mean may be defined as a 0 or 1, but the mean of the measurements may be much higher as initially graphed, such as about 10, 20, or 30 or the like, so the values may be normalized and compressed so as to fit within a more standardized curve, such as by subtracting the mean from each value and then dividing by the standard differentiation. This converts all of the input values into the same probability space. In such instances, when determining probabilities the more standard deviations from the mean a value falls, the less likely the factor is to be observed, and thus, farther away from the mean, the deviation determines the shape of the curve.

Another, physical, method for reducing noise to better ensure the accuracy of reads is to provide one or more filters on the energy emitters and/or energy receivers, e.g., photodiodes, so as to modify the signal to create a number of special effects that will result in an enhanced set of readings due to the presence of an intermediary filter. There are a number of filters that may be employed to clarify one or both of the output and/or input, such as polarization, stacked, and/or heuristic filters and the like.

As set forth above, one set of permittivity effects that can be used to quantify and/or qualify concentration data, is polarization of the emitted light caused by its interaction with the tissue in the presence of analytes, such as glucose. Therefore, in various embodiments, a polarization filter can be included, such as on one or more emitters and/or receivers, so as to filter out all the light waves other than those emitted at a particular angle, and thus, enhancing the ability to better detect the polarization effect. Such polarization within the tissues occurs because analytes, such as glucose, have chirality that causes the emitted light as it impinges within the tissue, and/or contacts the glucose molecules, to become polarized in some manner, and the more glucose that is present the greater the polarization effect there will be, as the frequency of the emitted light changes due to its interaction with the analyte within the tissues. More specifically, the angle of light as it is emitted can be measured and compared to the measured angle of the light as it is reflected back from the skin and tissue.

There are other filters that can also be added, such as in a stacked configuration. For instance, one such filter that can be added is a filter that concentrates the emitted light so that rather than the light being scattered, it is more focused. The difference between the focused and scattered light can also be measured so as to better determine the scattering effect. In various embodiments, it may be useful to concentrate the light so as to direct it into a particular location within the tissues. Likewise, although the photodiodes do not produce frequencies of light, they do receive light of a variety of frequencies, and thus they produce a signal response that includes a wide range of frequencies. However, in various instances, it may be useful for a filter to be applied to the emitter to emit and/or the photoreceiver to receive light of a particularly tight band of frequencies, such as + or −100, or + or −50, or + or −25, or + or −10 or 5 or less nanometers of the light wave to be emitted. This is helpful for decreasing the broader dispersion of wavelengths from interfering with light being emitted (or received) in a more concentrated waveform due to the addition of a concentrating filter.

Filtered light in a more narrow frequency (or wavelength) band is therefore easier to read due to less interference. For example, a typical emitter may transmit a dispersed wavelength that actually forms a bell curve of wavelengths that spans a range of 200 nanometers (+ or −100 nanometers), whereby the emitted wavelength has a maximal point but then includes a wide range of wavelengths being present but at lesser energy. This focusing of the light to a much more narrow band of wavelengths is useful because narrowing the range of emitted light more clearly reflects what wavelength is directly having the greatest effect for provoking these permittivity properties, while minimizing the potential for a wider range of auxiliary wavelengths causing interference in the measurements. So, by filtering out a majority, e.g., all, of the other frequencies increases the accuracy of that permittivity measurements. In other instances, a heuristic filter may be employed so as to apply more simple, experienced based rules to the data, which rules have been generated, e.g., by the system, and are grounded upon patterns having been observed in the past and can now be applied to future instances in the same or similar circumstances so as to make distinguishing between various datapoints easier and more reliable. Such heuristics can be used for determining means, minimums, maximums, averages in the data and/or steps in between, as discussed herein.

To these ends and referring to FIG. 14, the disclosed technology includes a process 1400 for determining the presence and/or concentration of one or more biomolecules within a wearer's body (e.g., based on spectral or RF or Microwave data obtained from a wearer of a device, such as a biometric sensing and/or monitoring device 15). Thus, the process 1100 can be configured to perform the same or similar functionality as process 900 (e.g., one or more steps of process 900 can be included in process 1100 or vice versa). The process 1100 can be configured to estimate the presence or concentration of a biomolecule of interest, e.g., analyte, based on correspondences or correlations between or among (1) present electromagnetic radiation, e.g., spectral data, of a particular user and (2) past electromagnetic radiation, e.g., spectral, data (e.g., “known electromagnetic radiation or spectral data,” which can refer to electromagnetic radiation data previously correlated or corresponded to known measurement data) and/or past known measurement data (also referenced herein as “known biomolecule data”). Stated differently, the process 1400 can be configured to estimate the current presence and/or concentration of a biomolecule of interest for a given wearer with or without contemplating present known measurement data (e.g., known measurement data obtained contemporaneously with the present electromagnetic radiation, e.g., spectral, data).

The process 1400 can include receiving 1402 present electromagnetic radiation, e.g., spectral, data that was obtained separately from a known measurement, such that the present spectral data is unknown and its relation to other known spectral and/or known measurement data needs to be determined.

As set forth above, the process 1400 can include removing bad or erroneous data (e.g., electromagnetic radiation, such as spectral, data). For example, the process 1400 can include performing a check, such as by evaluating the ranges of the diode outputs, the initial diode response, the maximum diode response, the rate of change of response, and/or the saturation (e.g., along with the rate of change of responses). In this regard, spectral readings or data points with poor quality, including readings compromised from bad transmission, incomplete signal, drift, electrical interference, or other interfering factors (as non-limiting examples), can be filtered and/or cleaned, such as by using a data valuation with reinforcement learning (DVRL) neural net, as described above, or other such methodologies for cleaning the measurement data. The DVRL neural net filtering can be trained through clinical feasibility studies, as a non-limiting example.

More specific examples for removing bad data include heuristics and/or machine learning methods, as described above, for detecting bad data. Heuristics can refer to rules that extract data that can negatively impact the process's 1400 efficacy. For example, one or more of the following heuristics or rules can be implemented:
Remove data if the last value in LED 8 is greater than or equal to 2500.
Remove data if the value in LED 1 at sweep 17 is greater than 3000.
Remove data if the value in LED 4 at sweep 3 is greater than 1000.
Remove data if the value in LED 5 at sweep 3 is greater than 1000.
Remove data if the value in LED 6 at sweep 3 is greater than 1000.
Remove data if the last value in LED 4 is greater than 5000.
Remove data if the last value in LED 6 is greater than 5000.
Remove data if the value in LED 4 at sweep 1 is greater than 1000.
Remove data if the value in LED 5 at sweep 1 is greater than 1000.
Remove data if the value in LED 6 at sweep 1 is greater than 1000.
Remove data if the value in LED 4 at sweep 4 is greater than 1000.
Remove data if the value in LED 5 at sweep 5 is greater than 5000.
Remove data if the value in LED 4 at sweep 5 is greater than 5000.
Remove data if the value in LED 1 at sweep 17 is greater than 3000.
Remove data if the value in LED 8 at sweep 17 is greater than 800.
Remove data if the voltage value for LED 8 is less than 400 at sweep 20.
Remove data if the voltage value for LED 7 is less than 250 for sweep 20.
Remove data if the voltage value for LED 9 is less than 100 for sweep 20.
Remove data if, for any LED, the value of current is less than set max value as programmed.
Remove data if voltage value for LED 2 is higher than 10K mV for any sweep.
Remove data if voltage value for LED 8 is higher than 10K mV for any sweep.

Alternatively or in addition, various methods can detect or help detect bad data, such as the Data Valuation with Reinforcement Learning (DVRL) technique, which is a self-labeling algorithm that can derive its estimates by examining which kinds of records perform well with training and which ones do not.

Accordingly, the process 1400 can include applying 1404 AI algorithms to determine or identify any correlations between the present electromagnetic radiation, e.g., spectral, data and one or more of past known electromagnetic radiation, e.g., spectral, data and/or past measurement data. Thus, the process 1400 can include generating 1406 a model (e.g., via a set of AI algorithms as set forth above) by which present, unknow electromagnetic radiation, e.g., spectral, data can be compared to known electromagnetic radiation, e.g., spectral, data and/or known measurement data to thereby determine or identify a correlation or correspondence therebetween. The process 1400 can include determining 1408 (e.g., via the AI algorithms), based at least in part on the correlation(s) and/or correspondence(s) and the present electromagnetic radiation, e.g., spectral, data, whether the biomolecule is present and any value related thereto (e.g., amount and/or concentration). The process 1400 can include training 1410 one or more processing engines to rapidly (e.g., instantly) determine an output (e.g., the presence and concentration of a biomolecule such as being present within the wearer's tissues) based on the type and characteristics of the inputs received. Accordingly, the process 1400 can include refining 1412 the AI algorithms by modifying the algorithms based on the learning processes based on received inputs (e.g., via back propagation methods, preprocessing methods, error reduction methods, or the like, or a combination thereof), determined outputs, and any future additional correlations or correspondences determined, to thereby be able to draw correlations more rapidly and accurately based on the initial input of data received and the nature of the data structure employed.

Referring now to FIG. 15, the disclosed technology includes a process 1500 for testing a generated model. The process 1500 can include splitting 1502 electromagnetic radiation, e.g., spectral, data (e.g., previously un-analyzed, but known, spectral data) into separate groups (e.g., two groups). The process 1500 can include applying 1504 the generated model to the first group of electromagnetic radiation, e.g., spectral, data to produce an estimated result. The process can include comparing 1506 the estimated result to known measurement data. The process 1500 can include adjusting 1506 the generated model to create a revised model. For example, the process 1500 can include applying back propagation and/or testing methodologies by comparing the estimated result to the known measurement data, determining a correction that would have provided an estimated result within a predetermined acceptable error range, and recursively adjusting the AI algorithms based on the determined correction. The process 1500 can include adjusting 1508 each generated model and/or revised model until a predetermined efficiency threshold is met or exceed and/or until a predetermined accuracy threshold is met or exceeded. For example, the predetermined accuracy threshold can be 90%, 95%, 99%, 99.9%, 99.99%, 99.999%, or the like, as non-limiting examples. The predetermined efficiency threshold can refer to the computation required to achieve an estimate. For example, some methods can be more efficient that others, and thus, the process 1500 can include computing a computational cost of a given generated model and/or revised model, and/or can include revising or further revising (or recreating) a model if the computational cost is above a predetermined threshold, as a non-limiting example.

The process 1500 can include testing 1510 the resulting model against the second group of spectral data. Steps 1506 and 1508 can be repeated, as necessary, until the predetermined efficiency and/or accuracy threshold is met or exceeded. Accordingly, the process 1500 can be configured to train a generated model, such as for measuring and/or determining the presence of a biomolecule based on spectral data where the true measurement outcome is known (e.g., previously determined, such as by a blood glucose monitor).

Referring now to FIG. 16, the disclosed technology includes a process 1600 that, for example, can help ensure continued accuracy for a given wearer. The process can include periodically correlating 1602 new electromagnetic radiation, e.g., spectral, data with known periodic analyte, e.g., glucose, measurement data (e.g., where a condition, such as diabetes, is known to be experienced by the wearer). The process 1600 can include generating 1604 a determined glucose-concentration-over-time curve (e.g., based on spectral determination) and generating 1606 a known or true glucose-concentration-over-time curve (e.g., based on the known periodic glucose measurement data). The process 1600 can include comparing 1608 the determined glucose-concentration-over-time curve to the true glucose-concentration-over-time curve and, adjusting 1610 the current model (e.g., AI algorithms) to satisfy a predetermined error threshold (e.g., until the determined glucose-concentration-over-time curve is within a predetermined error range of the true glucose-concentration-over-time curve).

The disclosed technology can further include systems and methods for simultaneously determining and monitoring biomolecule data for a plurality of individuals based on continuous and non-invasive spectral data for each individual. As described herein, the disclosed technology includes systems and processes for correlating a large number of data points relating to a wearer of a biometric sensing and monitoring device, the wearer's physiology, physical and/or environmental characteristics relating to the wearer, such as derived from one or more of the auxiliary sensors of the detection device, and additional sensor data relating to the wearer (e.g., different types of biometric sensors, such as sensors configured to detect or measure a characteristic that is different from the biomolecule of interest). The disclosed technology includes generating a user profile unique to a plurality of users and facilitating the collection and analysis of electromagnetic radiation, e.g., spectral, data corresponding to each individual user over time. Each user profile can include baseline data, which can include one or more initial blood glucose measurements taken from an auxiliary measuring device (e.g., such as from a prick and stick or analyte sensing measuring device) and/or answers to questions provided during a profile creation process (e.g., provided to the user via an application interface on the sensing and monitoring device and/or on a separate computing device). The answers can relate to one or more of the user's health conditions, feelings (e.g., mental wellness), and/or goals for future health and/or wellness. Alternatively or in addition, the disclosed technology can include performing (e.g., automatically via a backed computing system) one or more searches available content and/or information about the user (e.g., publicly available data, private health data authorized by the user), which can then be used in addition to the other aforementioned data for generating a user profile associated with, and unique to, the user. Such data can be entered into one or more data structures, such as those described herein.

Accordingly, the disclosed technology can learn and identify the identities and behaviors of various users based at least in part on the user profile and user-specific data (e.g., spectral data, biometric data) associated therewith. Further, the disclosed technology can determine a physiology of each user and determine how various biomolecules or metabolites (e.g., glucose) affect the body and/or health of the user. And by observing and contemplating effects of the biomolecule of interest on the user's body and characteristics (e.g., overall health, physical wellness, mental wellness), the disclosed technology can more accurately and efficiently estimate or determine a current value of the biomolecule of interest. In this regard, the disclosed technology can include incorporating known user characteristic data (e.g., BMI), bodily behavior data (e.g., exercise history), and/or the like into the data structures and AI algorithms described herein. Some or all of such information can be received as user-inputted data, and/or some or all of such information can be obtained from a third-party source (e.g., private health records, an exercise-tracking program). Accordingly, the disclosed technology can include determining or identifying correlations or correspondences between any such data disclosed herein (including, but not limited to, known user characteristic data and/or bodily behavior data) and making determinations based at least in part on such correlations or correspondences.

As described elsewhere herein, generating a data structure (e.g., step 1104) can include constructing decision tree or an artificial neural network (ANN) to determine or identify correlations, correspondences, and/or associations between and/or among varying data points and/or data types, and the ANN can be constructed such that any particular data point can form a node. Each node (sometimes referenced as “neuron”) can store relationships, and typically, an increase in the amount of information included in a model increases the number of required nodes or neurons.

The ANN (or other architecture) can store electromagnetic radiation, e.g., spectral, data (e.g., processed and/or cleaned, raw) as a first set of nodes and known biomolecule data (e.g., data from a finger prick test strip or analyte-measured glucose data) as a second set of nodes. The ANN can include a third set of nodes, which can be indicative or (or relate to, or otherwise be associated with) one or more conditions, one or more properties, one or more characteristics, one or more personality traits, one or more health factors, etc. of the wearer of the sensing and monitoring device.

The disclosed technology includes systems and methods for analyzing and/or monitoring spectral data for and/or across a population of individuals, such as the example process 1700 shown in FIG. 17. For example, the process 1700 can include storing 1702 electromagnetic radiation, e.g., spectral, data from a population of individuals as a first set of nodes in the data structure and storing 1704 respective analyte, e.g., glucose, measurement data that corresponds to that spectral data from the population of individuals as a second set of nodes in the data structure. The process 1700 can include storing 1706 condition and/or characteristic data that corresponds to individuals within the population of individuals as respective or more additional nodes (or additional sets of nodes) as one or more additional nodes in the data structure. The process 1700 can include determining 1708 relationships between and/or among the various nodes by analyzing various potential points of connection and/or relationships between various sets of nodes and storing 1710 the relationships as stored relationships.

Alternatively or in addition, the process 1700 can include grouping individuals (and the data corresponding to such individuals) according to the same or similar electromagnetic radiation, e.g., spectral, data, the same or similar measured biomolecule data, and/or the same or similar condition and/or characteristic data to thereby define 1712 one or more sub-populations of individuals within the larger population. The process 1700 can include determining 1714 relationships between and/or among the various nodes corresponding to a given sub-population and updating 1716 the stored relationships accordingly. Updating 1716 one or more the stored relationships (e.g., relationships associated with the individuals of the sub-population) can include adding one or more new relationships for the given sub-population (e.g., relationships determined at the sub-population level), removing one or more relationships determined at the whole population level, and/or replacing one or more relationships determined at the whole population level with one or more relationships determined at the sub-population level. Accordingly, the disclosed technology can determine or identify correlation, correspondence, and/or association between and/or among various individuals' electromagnetic radiation, e.g., spectral, data, measurement data, and/or condition data at a large population level and/or a sub-population level.

The process 1700 can include defining 1718 the anchor nodes, which represent the bounding elements between which all the various commonalities can be defined and explored. The process 1700 can include defining 1720 all the possible known correspondences between the anchor nodes and generating 1722 one or more additional nodes to represent the known correspondences. The known correspondences between the anchor nodes can be defined by analyzing (e.g., iteratively analyzing) causal effects between and/or among the various nodes to build upon the known correspondences and/or to determine additional known and/or observable relationships between the nodes.

The process 1700 can include determining 1724 secondary relationships based on the known correspondences or relationships. The secondary relationships can be built on inferences derived from the first set of nodes (e.g., the nodes corresponding to the spectral data).

To help increase the accuracy and predictability of outcomes, the process 1700 can include weighting 1726 one, some, or all of the various different relationships (e.g., known correspondences or relationships, secondary relationships). The relationships can be weighted on various factors, such as the degree of certainty (whether estimated or known), the number of commonalities, the number of instances sharing the node, the number of common relationships, and the like.

As will be appreciated, and as a non-limiting example, where there is a correspondence between the relationships of a first individual with the relationships of a second individual within the same population or sub-population (e.g., the nodal relationships for the first individual are the same or similar to the nodal relationships for the second individual), the model for the first individual can be used as an initial model for the second individual (e.g., subject to subsequent refinement), which can help reduce time and processing requirements to perform the process 1700 for the second individual.

Accordingly, the disclosed technology (e.g., the AI algorithms) can be configured to receive input raw data (e.g., electromagnetic radiation, e.g., spectral, data and/or measure biomolecule data) and generate one or more nodes based on the raw data (e.g., personalized to the corresponding individual) within the data structure. The disclosed technology (e.g., the AI algorithms) can be configured to receive data from one or more users of the system (e.g., data pertaining to one or more characteristics of the individual, such as health condition data, and the like) and generate one or more additional nodes corresponding to the particular individual, and the disclosed technology (e.g., the AI algorithms) can be configured to define one more relationships between and/or among the various nodes. Once the architecture of the data structure has been established, the data structure can be continuously (or periodically) updated and/or grown, such as by adding more data and/or more pertinent data, such as from one or more individuals and building, replacing, or otherwise updating additional potential nodes and/or relationships.

The bounding or anchor nodes can be any combination of nodes. As a non-limiting example, the bounding or anchor nodes can be user-inputted (and/or user selectable). For example, the system of the disclosed technology can be selectively accessible by the individual associated with a given set of data or a third party, such as a healthcare professional and/or system administrator overseeing administration of the system, as non-limiting examples. In any case, a user of the system who has access to the individual's data can view, edit, and/or download various aspects corresponding to the individual's data, such as uploading pertinent information into the system, determining or selecting the anchor nodes by which to bound an inquiry (e.g., by clicking or dragging and dropping specific nodes via a graphical user interface), and/or creating a question or prompt to be answered by the system (e.g., by the AI algorithms). The disclosed technology can thusly apply the AI algorithms based on the user input (e.g., query, anchor nodes) to generate a relevant and accurate output for the user (e.g., fully responsive to the query based on the selected anchor nodes).

As will be appreciated, the relationship between two or more properties is unlikely to be linear and is instead likely much more complex. For example, the AI algorithms can be configured to model complex processing of relationships in a manner the same as, or similar to, a neural network, such as in a deep learning protocol. Accordingly, while some relationships may be configured in a linear array to form a direct linkage or correlation between properties, it is much more likely that the relationships are layered one on top of the other so as to form a stacked (e.g., neural) network of information (e.g., in the form of an ANN). Therefore, the relationships, correlations, or correspondences described herein can be formed in (and/or include) multiple stages and/or levels, where one level of information is connected to the next level of information and so on, such as in a deep learning protocol. Alternatively or in addition, relationships between the various properties from a given level to another level can be strengthened (e.g., given greater weight) or weakened (e.g., given less weight) by the ML protocol.

Accordingly, as information is processed and allocated across the properties in the various levels of the data structure (on the same level or across different levels), the weights and/or biases for one or more different points can be adjusted (e.g., given greater or lesser weight) to refine the AI algorithms and increase the efficiency and/or accuracy of outputted information. The AI algorithms can be configured to process information in a layered or multi-staged fashion (e.g., as with deep learning). For example, the AI algorithms can be configured to examine various data, such as when performing a learning protocol, stage-by-stage and/or level-by-level, and can weight and/or bias each connection between data (e.g., based on historical evidence and/or characteristics of the relationships). Typically, the more stages and/or levels of learning that are initiated within the system, the better the weighting and/or bias between junctions will be, and the deeper the learning. Further, collecting and/or uploading data in stages can lead to a greater convergence of data within the system. Particularly, various feature extraction paradigms can be employed to better organize, weight, and/or analyze salient features of the data to be uploaded.

Alternatively or in addition, and with reference to FIG. 18, the disclosed technology includes a process 1800 for determining one or more actions (e.g., remedial and/or prophylactic actions) that can be taken by an individual to improve one or more health conditions of the individual. The process 1800 can include evaluating 1802 user data (e.g., the wearer's observed electromagnetic radiation, e.g., spectral, data, known/measured biomolecule data, biological characteristic data) associated with the individual based at least in part on the determined presence and/or concentration of the biomolecule of interest within the body of the particular wearer or individual. The process 1800 can include determining 1804 one or more goals for the individual. The goals can be determined based entirely on non-user-inputted data (e.g., a goal to reach a typical health status) and/or can be based in whole or in part on user-inputted goals (e.g., an ideal weight, a target blood glucose level). The process 1800 can include determining 1806 a current health status of the individual, which can be or include data indicative of one or more conditions that the individual may be experiencing, such as a condition that may be preventing them from reaching their goals. The process 1800 can include receiving a user-inputted action plan.

The process 1800 can include generating 1808 one or more nodes (e.g., a new set of nodes) within the data structure associated with the individual and corresponding to the one or more goals, the current health status (e.g., a current health condition being experienced by the individual), and/or the proposed action plan associated with the individual. The process 1800 can include evaluating 1810 relationships between and/or among the various nodes (e.g., including the nodes corresponding to the one or more goals, the current health status), according to the technology disclosed herein. Based at least in part on these data nodes and/or determined relationships, the process 1800 can include determining 1812 a recommended action plan for achieving the one or more goals. That is to say, the disclosed technology can generate and/or formulate an individualized plan of improvement for the individual at a predicted or otherwise determined methodology (e.g., with consideration of any health conditions that may limit activity or adherence of the individual to a typical or ideal plan of improvement) to thereby guide that individual toward meeting his or her health goals, such as lowering the presence of problematic biomolecules within his or her body and/or to overcome or ameliorate one or more symptoms of a disease or condition, such as diabetes.

The process 1800 can include receiving 1814 plan adherence data (e.g., data indicative of the individual's attempts to follow the determined plan of improvement and/or otherwise improve his or her health and activities), generating 1816 plan adherence nodes corresponding to such data, and determining 1818 additional relationships between and/or among the various nodes (include the plan adherence nodes). The process 1800 can include determining 1820 a revised action plan (e.g., based on compliance or non-compliance over time).

Alternatively or in addition, the process 1800 can include generating an aggregated, collective data structure including various nodes relating to various data discussed herein, including data relating to improvement steps and/or action plans, for a plurality of individuals, and determining or evaluating relationships (e.g., evaluating step 1810) between and/or among the various nodes, collectively and/or for one or more individuals (e.g., over a long period of time). Relationships between two or more characteristics in a given individual, or between different individuals, can be determined. For example, a given individual's earlier data (e.g., spectral data, conditions, and/or health characteristics) can be correlated with his or her later data (e.g., spectral data, conditions, and/or health characteristics and/or with later data associated with different individuals that are correlated to, or share correspondence with, the given individual). More particularly, the process 1800 can determine a relationship between two properties (e.g., a first property relating to an individual's electromagnetic radiation, e.g., spectral, characteristics and/or health conditions, and a second property, relating to one or more characteristics and conditions of a model based on known biomolecule data). The process 1800 can include determining the action plan and/or revised action plan for a given individual (e.g., steps 1808 and/or 1816) based on the aggregated data structure.

As described herein, the disclosed technology includes systems and methods for continuously measuring or estimating biomolecule values, and in particular glucose values, of a user (e.g., a wearer of a sensing and monitoring device). The sensing and monitoring device can be configured to continuously or regularly direct emitted electromagnetic radiation, such as light, to and/or into the skin of the user according to a predetermined schedule. The predetermined schedule can refer to a plurality of electromagnetic radiation, e.g., light, emissions, and each light emission can have a corresponding predetermined wavelength and a corresponding predetermined emission duration. The predetermined wavelength corresponding to one, some, or all of the light emissions can be different from the other predetermined wavelength corresponding to the other light emissions. Alternatively or in addition, the predetermined emission duration corresponding to one, some, or all of the light emissions can be different from the predetermined emission duration corresponding to the other light emissions. The sensing and monitoring device can be configured to detect and/or measure reflected electromagnetic radiation (e.g., raw spectral data) reflected by skin and/or tissues of the wearer of sensing and monitoring device.

The sensing and monitoring device can include a computing device (e.g., one or more processes and memory) configured to preprocess the raw spectral data on-board (e.g., convert the raw spectral data from an analog signal to a digital signal) and/or transmit the digital spectral data (e.g., over a wireless network connection) to a processing platform, such as a mobile computing device or a remote server device or system. To ensure security of the user's data, the digital electromagnetic radiation, e.g., spectral, data (and other data transmissions) can be encrypted before being transmitted. The mobile computing device and/or remote server system can be configured to receive the encrypted electromagnetic radiation, e.g., spectral, data, decrypt the encrypted electromagnetic radiation, e.g., spectral, data, and build, supplement, append, augment, or otherwise adjust a data structure, such as a neural network (e.g., an ANN) based at least in part on the particular user's electromagnetic radiation, e.g., spectral, data. Building the data structure can include incorporating stored data from one or more different users having one or more characteristics, performances, and/or conditions that is the same as one or more like characteristics, performances, and/or conditions of the particular user. The disclosed technology can include generating one or more nodes corresponding to the particular user's data and/or the data corresponding to the one or more different users having the same or similar data as the particular user.

As described herein, the disclosed technology can include generating a personalized (or semi-personalized, in the event the data corresponding to one or more different users is used to build the data structure) electromagnetic radiation or spectral model for the particular user, and the model can determine relationships between and/or among various nodes in the data structure to thereby identify correlations between the user's data and one or more of past predictions for the user (or different, similar users) and/or known glucose values. The disclosed technology can include an AI analytics system configured to apply or input the received electromagnetic radiation, e.g., spectral, data to the generated electromagnetic radiation, e.g., spectral, model, and the AI analytics system can determine and/or estimate the present glucose values for that particular user (e.g., corresponding to the present spectral data).

These data, measurements, analyses, and resulting outputs, can be continuously or regularly performed, encrypted, and transmitted to a user-associated mobile computing device, whereby present and/or historical glucose values, concentrations, and/or trends can be readily accessed by the particular user (or an authorized third party). As will be appreciated, the outputted glucose values or concentrations can be based on a predictive model, and thus, the disclosed technology can include determining and displaying a confidence value (e.g., on a scale from 0 to 1, a percentage from 0 to 100) with the glucose value or concentration to indicate a calculated confidence that the determined glucose value is the true glucose value. Confidence can be trained by the particular ML method being used, and/or a separate network (e.g., a separate ML method) can be trained to estimate confidence in the accuracy of the estimated data. Alternatively or in addition, confidence can be derived statistically.

To improve the predictive model, the disclosed technology includes automatically adjusting the ML rules used to define relationships and/or adjusting weightings of the various relationships defined between and/or among nodes within the data structure. Such adjustments can be based at least in part on the confidence score and/or other pertinent factors, such as error correction methods. For example, the disclosed technology can include a process for adjusting the ML rules for defining relationships between nodes to be stricter or more lenient, e.g., regarding fitting, as to what data points and/or which relationships will be considered as valid when making a given predictive model (e.g., which nodes can be considered as objects, which nodes can be considered as subjects and predicates, which nodes can be correlated as objects). Alternatively or in addition, other pertinent factors can include determined cohesion and/or coherence. Cohesion can refer to the degree to which readings (e.g., electromagnetic datapoints) can be grouped, whereas coherence refers to the similarity of behavior of points in a group (e.g., a group of spectral data). Alternatively or in addition, other pertinent factors can include external or ambient factors, such as heat, ambient light, and/or error count, as non-limiting examples.

Consequently, once the various relationships have been defined, weighted, and/or biased, the disclosed technology may include receiving a predictive query (e.g., in the form of logical, e.g., “IF/THEN”, statement), wherein a system according to the disclosed technology receives a query (e.g., the “IF” portion of the statement), and the system processes a predictive model to generate a resultant outcome, such as based on a probability outlook (e.g., the “THEN” portion of the statement).

Alternatively or in addition, the disclosed technology can identify one or more trends or one or more recommended actions to improve the health outlook of the particular user. For example, the particular user (or a healthcare professional) can enter the user's conditions and/or characteristics, wellness goals, and/or a proposed improvement plan, and the system can use that data to build or augment a data structure (e.g., build a secondary data structure, such as a knowledge graph), whereby the system can provide a proposed outlook or prediction for the user improving his or her health (or otherwise meeting his or goals) by implementing the improvement plan and/or can suggest alternative or supplemental improvement plans or methodologies for improving the user's health and/or achieving the user's goals.

The system can include a suitably configured integrated circuit, such as an FPGA, ASIC, Structured ASIC, and the like, which can help accelerate certain aspects of the system's artificial intelligence module.

### Experimental Results

To exemplify the unexpected efficacy and accuracy of the disclosed technology, testing results of an early prototype are discussed below. The test data discussed below includes spectral data obtained via an early prototype of a spectral device (e.g., a biometric sensing and/or monitoring device 15), known blood glucose data obtained via a Continuous Glucose Monitoring (CGM) system, and an early prototype of a neural net system for estimating blood glucose levels based on spectral data.

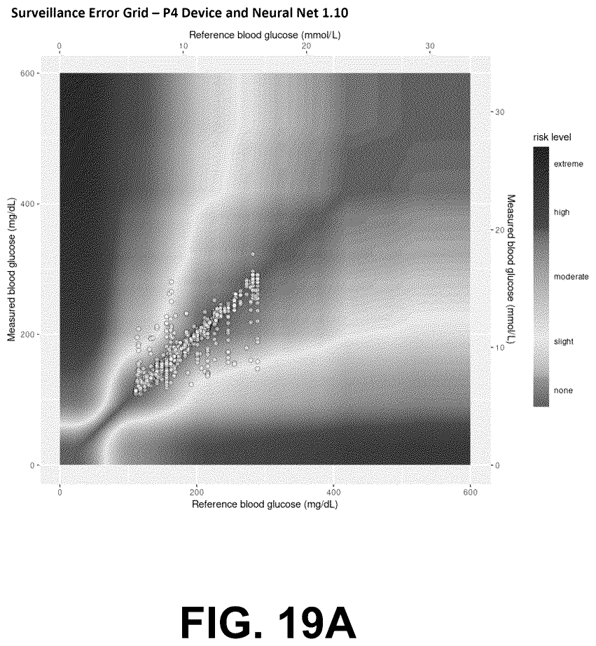

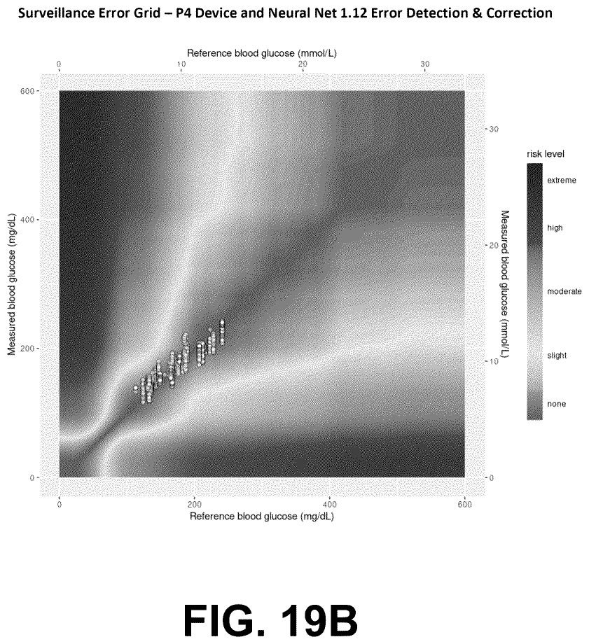

FIGS. 19A and 19B illustrate charts of the Error Surveillance Grid associated with pairings of spectral data and known blood glucose data. This data reflects a Mean Absolute Relative Difference (MARD) of 6.0%. FIG. 19A corresponds to an initial application of the early prototype neural network to the spectral data, and FIG. 19B corresponds to an application of the early prototype neural network to the spectral data after the neural network was refined via error detection and correction steps. Note that FIG. 19A shows “grouping,” which is key indicator of the neural network “finding the signal” (e.g., deriving high quality information from unstructured data). FIG. 19B tightens the correlations and the grouping is consequently tighter.

Examples and/or embodiments of the disclosure and all of the functional operations described in this specification can be implemented in digital electronic circuitry, or in computer software, firmware, or hardware, including the structures disclosed in this specification and their structural equivalents, or in combinations of them. Embodiments of the disclosure can be implemented as one or more computer program products, e.g., one or more modules of computer program instructions encoded on a computer readable medium, e.g., a machine readable storage device, a machine readable storage medium, a memory device, or a machine-readable propagated signal, for execution by, or to control the operation of, data processing apparatus. Further operations may be performed by one or more modules of a suitably trained AI system, without the need for written code.

While certain aspects of the disclosed technology have been discussed herein with respect to a particular device, system, process, or method, the disclosed technology is not so limited. For example, certain aspects of the disclosed technology explicitly described herein as being incorporated in, or performed by, a device can be likewise incorporated in, or performed by, one or more devices in a system and/or can be included as one or more steps of a process or method. Similarly, certain aspects of the disclosed technology explicitly described herein as being incorporated in, or performed by, a system can likewise be incorporated in, or performed by, a device and/or can be included as one or more steps of a process or method. Further, certain aspects of the disclosed technology explicitly described herein as being one or more steps of a process or method can be performed by a device and/or a system.

The term “data processor” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of them. A propagated signal is an artificially generated signal, e.g., a machine-generated electrical, optical, or electromagnetic signal, that is generated to encode information for transmission to suitable receiver apparatus.

A computer program (also referred to as a program, software, an application, a software application, a mobile application, a script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer.

Generally, a processor will receive instructions and data from a read only memory or a random access memory or both. The essential elements of a computer are a processor for executing instructions and one or more memory devices for storing instructions and data. Generally, a computer will also include, or be operatively coupled to, a communication interface to receive data from or transfer data to, or both, one or more mass storage devices for storing data, e.g., magnetic, magneto optical disks, or optical disks. Moreover, a computer can be embedded in another device, e.g., a mobile telephone, a personal digital assistant (PDA), a mobile audio player, a Global Positioning System (GPS) receiver, to name just a few.

Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the disclosure can be implemented on a computer having a display device, e.g., a capacitive sensing touch screen device, including a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the disclosure can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of such back end, middleware, or front end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

Certain features which, for clarity, are described in this specification in the context of separate embodiments, may also be provided in combination in a single embodiment. Conversely, various features which, for brevity, are described in the context of a single embodiment, may also be provided in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Particular embodiments of the invention have been described. Other embodiments are within the scope of the following claims. For example, the steps recited in the claims can be performed in a different order and still achieve desirable results. In addition, embodiments of the invention are not limited to database architectures that are relational; for example, the invention can be implemented to provide indexing and archiving methods and systems for databases built on models other than the relational model, e.g., navigational databases or object-oriented databases, and for databases having records with complex attribute structures, e.g., object-oriented programming objects or markup language documents. The processes described may be implemented by applications specifically performing archiving and retrieval functions or embedded within other applications.

Although a few embodiments have been described in detail above, other modifications are possible. Other embodiments may be within the scope of the following claims.

Various methods and processes are described herein. It is to be understand that the disclosed technology does not necessarily require performance of every step explicitly described herein and may include additional steps not explicitly referenced herein. Stated differently, the scope of this disclosure includes methods and processes that include some, but not all, steps provided herein and/or that include additional steps not explicitly mentioned herein. Furthermore, the scope of this disclosure includes methods and processes that are each a combination of some or all of the multiple methods and processes explicitly laid out herein. As a non-limiting, illustrative example, the scope of this disclosure includes a process that includes one or more steps from process 900 and one or more steps from process 1100.

What is claimed is:

1. A continuous non-invasive sensor system having a multi-sensing detection device, the sensor system for employing spectral related skin response data for determining a concentration of glucose being present in a tissue of a body of a wearer of the multi-sensing detection device, the system comprising:
a multi-sensing detection device being configured for being positioned proximate the wearer's skin, the multi-sensing detection device having a glucose sensor unit for detecting glucose being present within the wearer's tissue, the multi-sensing detection device comprising:
a glucose sensor unit for detecting a spectral related skin response of one or more tissues of the body due to an interaction of light with the body tissue in the presence of glucose, the glucose sensor unit comprising:
a printed circuit board having a first and a second sensor array coupled therewith, each sensor array including a number of photoemitters and one or more photoreceivers, each sensor array being arranged on the printed circuit board within the housing so as to be proximate the user's skin when the multi-sensing detection device is coupled to the user's body, the one or more photoemitters configured to illuminate, in one or more cycles, at a predetermined frequency and duration of light, the tissue of the user's body, and each of the one more photoreceivers configured to receive a return of the light reflected back from the user's tissue and to generate a return signal in response to collecting the reflected light,
the first sensor array comprising a first set of photoemitters configured for being activated individually or collectively and being positioned so as to be proximate at least a first photoreceiver, each individual photoemitter of the first set of photoemitters further being configured for directing light so as to penetrate a first depth within the tissue of the user's body,
the second sensor array comprising a second set of an additional number of photoemitters configured for being activated individually or collectively and being positioned so as to be proximate at least a second photoreceiver, each photoemitter of the second set of photoemitters further being configured for directing light so as to penetrate a second depth within the tissue of the user's body, wherein the at least first and second photoreceivers are configured for collecting the light reflected back form the body tissue so as to generate the return signal, and
the printed circuit board further comprising an analog to digital converter coupled to the at least first and second photoreceivers, the analog to digital converter being configured for converting the return signal to digital signal data, and a communications module for transmitting the digital signal data; and
a server system for receiving the digital signal data from the multi-sensing detection device, the server system comprising a first processing module having a first processor for analyzing the digital signal data so as to produce the spectral related skin response data, and a second processor for analyzing the spectral related skin response data so as to thereby determine the concentration of glucose being present in the tissue of the body of the wearer.

2. The continuous non-invasive sensor system in accordance with claim 1, wherein the first sensor array comprises up to six photoemitters, and the second sensor array comprises up to four photoemitters each of the photoemitters of the first and second sensor arrays being positioned so as to be proximate their respective photoreceivers.

3. The continuous non-invasive sensor system in accordance with claim 2, wherein each of the first and second sensor arrays are further configured for emitting light from their respective photoemitters, wherein the emitted light comprises a wavelength in the range of about 400 nm to about 1650 nm.

4. The continuous non-invasive sensor system in accordance with claim 3, wherein at least the first of the first and second sensor arrays are configured for emitting light from their respective photoemitters, wherein the emitted light comprises a wavelength in the green, red, and infrared light waves.

5. The continuous non-invasive sensor system in accordance with claim 4, wherein the at least first sensor array comprises a PPG sensor assembly.

6. The continuous non-invasive sensor system in accordance with claim 5, wherein the printed circuit board comprises one or more of an accelerometer and an SPO₂ assembly,

7. The continuous non-invasive sensor system in accordance with claim 6, wherein the printed circuit board additionally comprises an ECG module and a plurality of ECG electrodes, at least one of the plurality of ECG electrodes being a ground ECG electrode, the ground ECG electrode being positioned on opposite sides of the first and second sensor array.

8. The continuous non-invasive sensor system in accordance with claim 7, wherein the server system comprises an Artificial Intelligence module, and the Artificial Intelligence module is configured for receiving and analyzing the digital signal data so as to produce the light signature results data.

9. The continuous non-invasive sensor system in accordance with claim 8, wherein the Artificial Intelligence module comprises a Machine Learning module and an Artificial Neural Network (ANN).

10. The continuous non-invasive sensor system in accordance with claim 9, wherein the ANN analyzes the spectral related skin response data so as to produce
light signature results data, and further analyzes the light signature results data so as to determine the concentration of glucose.

11. The continuous non-invasive sensor system in accordance with claim 10, wherein to determine the concentration of glucose the ANN maps the light signature data to the concentration of glucose.

12. The continuous non-invasive sensor system in accordance with claim 11, wherein the mapping comprises comparing an intensity of a number of reflected light wavelengths to a plurality of spectral related skin responses so as determine a pattern of absorption responses, and then analyzing the pattern of absorption responses so as to determine the light signature, whereby a level of concentration of glucose can be determined by the light signature based on a change in the pattern of the skin's absorption response caused by the presence of glucose within the skin.

13. The continuous non-invasive sensor system in accordance with claim 12, wherein the determining of the pattern of absorption response is based on a non-linear relationship between changes of reflectance due to a range of different wavelengths.

14. The continuous non-invasive sensor system in accordance with claim 13, wherein the ANN is further configured for determining a health trajectory of the wearer, from which health trajectory a future health state of the wearer is predicted.

15. A continuous non-invasive sensor system having a multi-sensing detection device, the sensor system for employing skin response data for determining a concentration of glucose being present in a tissue of a body of a wearer of the multi-sensing detection device, the system comprising:
a multi-sensing detection device being configured for being positioned proximate the wearer's skin, the multi-sensing detection device having a glucose sensor unit for detecting glucose being present within the wearer's tissue, the multi-sensing detection device comprising:
a glucose sensor unit being positioned within the housing and being configured for detecting a skin response of one or more tissues of the body due to an interaction of light with the body tissue in the presence of glucose, the glucose sensor unit comprising:
a printed circuit board having a first and a second sensor array coupled therewith, each sensor array including a number of photoemitters and one or more photoreceivers, each sensor array being arranged on the printed circuit board within the housing so as to be proximate the user's skin when the multi-sensing detection device is coupled to the user's body, the one or more photoemitters configured to illuminate the tissue of the user's body, and each of the one more photoreceivers configured to receive a return of the light reflected back from the user's tissue and to generate a return signal in response to collecting the reflected light,
the first sensor array comprising a first set of photoemitters configured for being activated individually or collectively and being positioned so as to be proximate at least a first photoreceiver,
the second sensor array comprising a second set of photoemitters configured for being activated individually or collectively and being positioned so as to be proximate at least a second photoreceiver, wherein the at least the first and second photoreceivers are configured for collecting the light reflected back form the body tissue so as to generate the return signal, and
the printed circuit board further comprising an analog to digital converter coupled to the at least first and second photoreceivers, the analog to digital converter being configured for converting the return signal to digital signal data, and a communications module for transmitting the digital signal data; and
a server for receiving the digital signal data from the multi-sensing detection device, the server system comprising a first processing module having a first processor for analyzing the digital signal data so as to produce the skin response data, and a second processor for analyzing the skin response data so as to thereby determine the concentration of glucose being present in the tissue of the body of the wearer.

16. The continuous non-invasive sensor system in accordance with claim 15, wherein the photoemitters of the first and second sensor arrays are configured for being activated in accordance with a number of predetermined wavelengths, frequencies, intensities, and/or durations, and further wherein the activation of the photoemitters in accordance with the predetermined wavelengths, frequency, intensity, and/or duration results in a pattern of absorption, and the second processor employs the pattern of absorption in determining the light signature from which the concentration of the analyte being present in the tissue of the body of the wearer.

17. The continuous non-invasive sensor system in accordance with claim 16, wherein the server system further comprises an Artificial Intelligence module, and the Artificial Intelligence (AI) module is configured for receiving and analyzing the digital signal data so as to produce the light signature results data.

18. The continuous non-invasive sensor system in accordance with claim 17, wherein the AI module is configured for analyzing the skin response data so as to produce the light signature results data, and is further configured for analyzing the light signature results data so as to determine the concentration of glucose.

19. The continuous non-invasive sensor system in accordance with claim 18, wherein to determine the concentration of glucose the AI Module maps the light signature response data to the concentration of glucose.

20. The continuous non-invasive sensor system in accordance with claim 19, wherein the mapping comprises comparing an intensity of a number of reflected light wavelengths to a plurality of skin responses so as determine a pattern of absorption responses, and then analyzing the pattern of absorption responses so as to determine the light signature, whereby a level of concentration of glucose can be determined by the light signature based on a change in the pattern of the skin's absorption response caused by the presence of glucose within the skin.
